(19) United States
(12) Patent Application Publication
(10) Pub. No.: US 2021/0308089 A1
(43) Pub. Date: Oct. 7, 2021

(54) METHODS OF TREATING AND/OR PREVENTING VIRAL INFECTIONS AND/OR DISEASES CAUSED BY VIRUSES IN A SUBJECT IN NEED THEREOF

(71) Applicant: Amarin Pharmaceuticals Ireland Limited, Dublin (IE)

(72) Inventors: Lixia Jiao, Bridgewater, NJ (US); Richard Louis Dunbar, Voorhees, NJ (US); Rebecca Juliano, Madison, NJ (US)

(21) Appl. No.: 17/224,691

(22) Filed: Apr. 7, 2021

Related U.S. Application Data
(60) Provisional application No. 63/006,621, filed on Apr. 7, 2020.
(60) Provisional application No. 63/018,627, filed on May 1, 2020.
(60) Provisional application No. 63/124,630, filed on Dec. 11, 2020.
(60) Provisional application No. 63/151,964, filed on Feb. 22, 2021.

(51) Int. Cl. A61K 31/232; A61K 31/675; A61K 31/47; A61K 45/06; A61P 31/14

(52) U.S. Cl. CPC A61K 31/232; A61K 31/675; A61P 31/14; A61K 45/06; A61K 31/47

(57) ABSTRACT

In various embodiments, the present disclosure provides methods for treating, preventing, and/or ameliorating symptoms of a viral infection, a disease, and/or a symptom thereof caused by the virus in a subject in need thereof, comprising administering about 4 g to about 20 g of icosapent ethyl to the subject per day. In some embodiments, the virus is SARS-CoV-2. In some embodiments, the disease is COVID-19.

### CROSS-REFERENCE TO RELATED APPLICATIONS

This application claims the benefit of U.S. Provisional Patent Application Nos. 63/006,621 filed on Apr. 7, 2020, 63/018,627 filed on May 1, 2020, 63/124,630 filed on Dec. 11, 2020, and 63/151,964 filed on Feb. 22, 2021. The contents of these provisional applications are incorporated by reference in their entireties.

### BACKGROUND

SARS-CoV-2 is a zoonotic coronavirus that causes COVID-19 respiratory disease in humans. COVID-19 was declared a pandemic by the World Health Organization in early 2020. Humans having the greatest risk for developing COVID-19 in response to a SARS-CoV-2 infection are those over the age of 65 and having comorbidities, such as cardiovascular disease, cancer, and other diseases and/or conditions that render humans more likely to develop an infection and severe symptoms. COVID-19 symptoms include fever, cough, shortness of breath, myalgia/fatigue, pharyngitis, headache, hemoptysis, and gastrointestinal symptoms. While some humans infected with SARS-CoV-2 may not develop COVID-19 or show other symptoms of SARS-CoV-2 infection, those who do develop COVID-19 or show symptoms can rapidly progress to severe disease resulting in death, often due to respiratory issues.

There are no known treatments or preventative agents for SARS-CoV-2 and/or COVID-19. Experimental uses of some existing anti-viral agents and anti-malarial agents designed to treat other diseases have been reported, but the results are mixed. A need exists for treatments and/or preventative agents for SARS-CoV-2 infection and/or COVID-19 and symptoms thereof.

### SUMMARY

The application relates to methods of treating and/or preventing viral infection in a subject by administering to the subject icosapent ethyl. The application also relates to methods of treating and/or preventing a disease or symptoms thereof caused by a virus in a subject by administering to the subject icosapent ethyl.

In some aspects, the present disclosure provides methods of treating and/or preventing SARS-CoV-2 infection in a subject by administering to the subject icosapent ethyl.

In some aspects, the present disclosure provides methods of treating and/or preventing COVID-19 or symptoms thereof in a subject by administering to the subject icosapent ethyl.

In some embodiments, the subject is administered about 4 g to about 20 g of icosapent ethyl per day. In some embodiments, the subject is administered about 6 g to about 10 g of icosapent ethyl per day. In some embodiments, the subject is administered about 4 g of icosapent ethyl per day. In other embodiments, the subject is administered about 6 g of icosapent ethyl per day. In some embodiments, the subject is administered about 8 g of icosapent ethyl per day. In some embodiments, the subject is administered about 10 g of icosapent ethyl per day. In some embodiments, the subject is administered about 20 g of icosapent ethyl per day.

In some embodiments, the subject is administered the icosapent ethyl for a period of time between about 3 days to about 1 year. In some embodiments, the subject is administered the icosapent ethyl for about 3 days. In some embodiments, the subject is administered the icosapent ethyl for about 3 weeks. In some embodiments, the subject is administered the icosapent ethyl for about 1 year.

In some embodiments, the icosapent ethyl is present in a pharmaceutical composition and the icosapent ethyl comprises at least about 96%, by weight of all omega-3 fatty acids in the pharmaceutical composition. In some embodiments, the composition comprises about 4 g of icosapent ethyl.

In some embodiments, the methods comprise monitoring the subject for symptoms of the disease caused by the virus. In some embodiments, the virus is SARS-CoV-2. In some embodiments, the methods comprise monitoring the subject for symptoms of COVID-19. In some embodiments, the subject requires hospitalization.

In some embodiments, administration of icosapent ethyl reduces an incidence of coughing and/or wheezing in the subject. In some embodiments, administration of icosapent ethyl increases bilirubin levels in the subject. In some embodiments, administration of icosapent ethyl reduces inflammation of the mucosal membrane. In some embodiments, administration of icosapent ethyl reduces the risk of systemic inflammatory response syndrome (SIRS) and/or sepsis. In some embodiments, administration of icosapent ethyl reduces leukotrienes levels of one or more leukotrienes selected from the group consisting of LTB4, LTC4, LTD4, and LTE4. In some embodiments, administration of icosapent ethyl reduces neutrophil levels and increases lymphocyte levels.

In some embodiments, the subject is further administered an anti-viral agent, an anti-malarial agent, and/or a biologic agent. In some embodiments, the subject is administered an anti-viral agent, an anti-malarial agent, and/or a biologic agent before administration of icosapent ethyl. In some embodiments, the subject is co-administered icosapent ethyl and an anti-viral agent, an anti-malarial agent, and/or a biologic agent. In some embodiments, the subject is administered an anti-viral agent, an anti-malarial agent, and/or a biologic agent after administration of icosapent ethyl. In some embodiments, the anti-viral agent is remdesivir. In some embodiments, the anti-malaria agent is hydroxychloroquine and/or chloroquine. In some embodiments, the biologic agent includes a peptide and/or a nucleic acid. In some embodiments, the peptide is an antibody. In some embodiments, the biologic agent is a vaccine.

### BRIEF DESCRIPTION OF THE DRAWINGS

FIG. 1 is a schematic of the study design according to an embodiment of the present disclosure.

FIG. 2 is a schematic showing disposition of patients according to an embodiment of the present disclosure.

FIGS. 3A and 3B are representative Kaplan-Meier event curves for the cumulative incidence of the primary composite endpoints. FIGS. 3A and 3B indicate a 25% relative risk reduction (RRR) for the primary composite endpoint over the course of 5 years.

FIG. 4 is a representative forest plot of individual components of primary endpoints analyzed as time to first event of each individual endpoint and indicates that each component, individually, was reduced.

FIGS. 5A and 5B are representative Kaplan-Meier event curves for the cumulative incidence of the key secondary composite endpoints. FIGS. 5A and 5B indicate that there was a 26% RRR for the key secondary composite endpoint over the course of 5 years.

FIGS. 6 and 7 are representative forest plots of primary efficacy outcomes in select prespecified subgroups. FIGS. 6 and 7 indicate that a subject's baseline triglyceride levels (e.g., ≥150 vs. <150 mg/dL or ≥200 vs. <200 mg/dL) did not influence the primary endpoint outcomes.

FIGS. 8 and 9 are representative forest plots of secondary efficacy outcomes in select prespecified subgroups. FIGS. 8 and 9 indicate that a subject's baseline triglyceride levels (e.g., ≥150 vs. <150 mg/dL or ≥200 vs. <200 mg/dL) did not influence the key secondary endpoint outcomes.

FIGS. 10A and 10B are representative Kaplan-Meier curves of primary and key secondary endpoints by achieved triglyceride level at 1 year. FIGS. 10A and 10B indicate that patient's triglyceride levels had no influence on the efficacy of icosapent ethyl as compared with placebo with respect to the primary or key secondary efficacy endpoint outcomes.

FIG. 11 is a representative forest plot of prespecified hierarchical testing of endpoints and indicates that all individual and composite ischemic endpoints were significantly reduced by icosapent ethyl (AMR101).

FIG. 12 is a schematic of the study design according to an embodiment of the present disclosure.

FIG. 13 is a representative bar graph depicting the distribution of first, second, and recurrent ischemic events in patients. FIG. 13 indicates that the first, second, and recurrent ischemic events were reduced in patients randomized to icosapent ethyl (IPE) compared to placebo.

FIG. 14 is a representative overall cumulative event Kaplan-Meier event curve for the primary endpoint indicating that overall cumulative primary endpoints were reduced in patients randomized to icosapent ethyl.

FIG. 15 is a representative cumulative event Kaplan-Meier event curve for the primary endpoint for patients in the secondary prevention cohort, which, similar to FIG. 14, indicates that cumulative primary endpoints were also reduced in patients in the secondary prevention cohort randomized to icosapent ethyl.

FIG. 16 is a representative cumulative event Kaplan-Meier event curve for the primary endpoint for patients in the primary prevention cohort, which, similar to FIGS. 14 and 15, indicates that cumulative primary endpoints were also reduced in patients in the primary prevention cohort randomized to icosapent ethyl.

FIG. 17 is a representative forest plot of the total event for each occurrence of the primary endpoint. FIG. 17 indicates that the times to first, second, third, or fourth occurrences of the primary composite endpoint were consistently reduced in the icosapent ethyl group as compared to placebo.

FIG. 18 includes representative pie charts for the proportion of first and subsequent primary endpoint events, overall and by component.

FIG. 19 is a representative graph depicting the risk difference in 100 patients treated for five years with icosapent ethyl versus placebo of the composite primary endpoint.

FIG. 20 is a representative forest plot of the total event for each occurrence of the primary and key secondary efficacy endpoints. FIG. 20 indicates that the total events for each component of the primary endpoint events were significantly reduced.

FIG. 21 is a representative overall cumulative event Kaplan-Meier curve for the key secondary endpoint indicating that overall cumulative key secondary endpoints were reduced in patients randomized to icosapent ethyl.

FIG. 22 is a representative cumulative event Kaplan-Meier curve for the key secondary endpoint for patients in the secondary prevention cohort, which, similar to FIG. 21, indicates that cumulative key secondary endpoints were also reduced in patients in the secondary prevention cohort randomized to icosapent ethyl.

FIG. 23 is a representative cumulative event Kaplan-Meier curve for the key secondary endpoint for patients in the primary prevention cohort, which, similar to FIGS. 21 and 22, indicates that cumulative primary endpoints were also reduced in patients in the primary prevention cohort randomized to icosapent ethyl.

FIG. 24 is a representative overall cumulative Kaplan-Meier event curve as a function of years since randomization for the primary endpoint indicating that overall cumulative primary endpoints were reduced in patients randomized to icosapent ethyl.

FIG. 25 is a representative overall cumulative event Kaplan-Meier curve as a function of years since randomization for the key secondary endpoint indicating that overall cumulative key secondary endpoints were reduced in patients randomized to icosapent ethyl.

FIG. 26 is a representative Kaplan-Meier curve for recurrent events as a function of years since randomization of the primary endpoint for patients in the secondary prevention cohort indicating that cumulative primary endpoints were reduced in patients in the secondary prevention cohort randomized to icosapent ethyl.

FIG. 27 is a representative Kaplan-Meier curve as a function of years since randomization for recurrent events of the key secondary endpoint for patients in the secondary prevention cohort indicating that cumulative key secondary endpoints were also reduced in patients in the secondary prevention cohort randomized to icosapent ethyl.

FIG. 28 is a representative Kaplan-Meier curve as a function of years since randomization for recurrent events of the primary endpoint for patients in the primary prevention cohort indicating that cumulative primary endpoints were also reduced in patients in the primary prevention cohort randomized to icosapent ethyl.

FIG. 29 is a representative Kaplan-Meier curve as a function of years since randomization for recurrent events of the key secondary endpoint for patients in the primary prevention cohort indicating that cumulative key secondary endpoints were reduced in patients in the primary prevention cohort randomized to icosapent ethyl.

FIG. 30 is a representative graphic depicting EPA as a bioactive lipid that preserves membrane structure and normal distribution of cholesterol.

FIG. 31 is a representative scheme showing how bioactive lipids (e.g., EPA) can inhibit pathogenesis (e.g., microbes).

FIG. 32 is a schematic of the VASCEPA-COVID-19 trial outlining the number of individuals who were assessed for eligibility, randomized, and included in the analyses. Thirteen people declined to participate. Approximately 79% of individuals assessed for eligibility consented to participate and were subsequently randomized. Of the 50 participants randomized to each group, data from 12% (n=6) of the IPE group and 6% (n=3) of the usual care group were not analyzed for the primary endpoint.

FIG. 33 shows baseline COVID-19 symptoms within 72 hours preceding the baseline visit. Although the IPE group was slightly more symptomatic, the entire study population presented with a large number of baseline symptoms. Almost all participants (98% and 94% in the IPE group and usual care group, respectively) had myalgia. Over half of individuals in both groups had the presence of a cough. Within the IPE arm, 50±2% of participants experienced loss of taste, loss of smell, or a fever. These symptoms were present in approximately one-third of the usual care arm. The remaining categories (sore throat and shortness of breath) displayed not more than a 40% prevalence in either group. All between-group comparisons were non-significant. Data shown are for the intention-to-treat population.

FIG. 34 show changes in total and individual domain inFLUenza Patient-Reported Outcome (FLU-PRO©) symptom prevalence and scores. FIG. 34 shows the prevalence of FLU-PRO© symptoms at baseline and follow-up, distributed by total and individual domains. A significant reduction in the total (P=0.005) and body/systemic (P=0.006) domains occurred between groups. A numerically larger reduction in chest/respiratory symptoms occurred in the IPE (69%) compared with the usual care cohort (53%), although not a significant difference. The remaining domains had similar reductions in prevalence between groups. Values shown are based on the number of patients with complete paired data and non-zero treatment compliance. All domains within the usual care and IPE cohorts had significant within-group differences, comparing the number of responders (Yes/No) at baseline versus follow-up, via chi-square analyses: Usual Care (P<0.01 within all domains); IPE (P<0.01 within all domains except the gastrointestinal domain [P=0.049]).

FIG. 35 shows mean change in FLU-PRO© scores from baseline to follow-up, distributed by total and individual domains. A significant score reduction in the IPE group compared with the usual care cohort occurred in the total (P=0.003), body/systemic (P=0.001), and chest/respiratory (P=0.01) domains. A significant score reduction in the usual care group compared with the IPE cohort occurred in the gastrointestinal domain (P=0.026). The remaining domains had non-significant changes in scores between groups. Values shown are based on the number of patients with complete paired data and non-zero treatment compliance. Mean FLU-PRO© scores and associated between-group p-values at baseline, per domain: Total: IPE=0.18, usual care=0.14, P=0.03; Body/Systemic: IPE=0.28, usual care=0.23, P=0.04; Gastrointestinal: IPE=0.01, usual care=0.04, P=0.04; Chest/Respiratory: IPE=0.25, usual care=0.16, P=0.03; Eyes: IPE=0.02, usual care=0.01, P=0.34; Throat; IPE=0.10, usual care=0.08, P=0.59; Nose: IPE=0.10, usual care=0.05, P=0.16.

### DETAILED DESCRIPTION

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to not only include numerical values explicitly specified as limits of a range, but also to include all individual numerical values or sub-ranges encompassed within that range as if each numerical value and sub-range is explicitly specified. For example, a ratio in the range of about 1 to about 200 should be understood to not only include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the ranges described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

The term “derivative,” as used herein when referring to a fatty acid, is meant to encompass any modified form of the fatty acid that was derived, for example, by a chemical reaction from the fatty acid in free acid form (i.e., terminal carboxylic acid functional group). Non-limiting examples of fatty acid derivatives as used herein include alkyl esters such as methyl esters, propyl esters, butyl esters, or ethyl esters; a salt of the fatty acid such as a lithium, sodium, or potassium salt; or a glyceride form of the fatty acid such as a mono-, di-, or triglyceride fatty acid.

In one embodiment, the free fatty acid of eicosapentaenoic acid is administered to the subject and the amount administered is the gram weight sufficient to substantially match the pharmacokinetic profile produced by administration of 4 g of E-EPA per day to a human subject. In another embodiment, a derivative of eicosapentaenoic acid is administered to the subject and the amount administered is the gram weight sufficient to substantially match the pharmacokinetic profile produced by administration of 4 g of E-EPA per day to a human subject. With respect to a dose of 3.7 g per day of eicosapentaenoic acid, a dose-equivalent amount of E-EPA is about 4 g of E-EPA per day.

The phrase “control subject,” as used herein refers to any subject used as a basis for comparison to the test subject. A control subject includes, but is not limited to, any subject who has not been administered the composition, administered a composition other than the test composition (e.g., Lovaza® comprised of 365 mg of E-EPA and 375 mg of E-DHA), or administered a placebo.

The phrase “cardiovascular risk category 1,” as used herein, refers to subjects categorized as having an established cardiovascular disease. Patients from cardiovascular risk category 1 were stratified to the secondary prevention cohort. The designations for patients defined by cardiovascular risk category 1 are collectively referred to as: secondary prevention stratum, secondary prevention cohort, and the primary risk category.

The phrase “cardiovascular risk category 2,” as used herein, refers to a subject categorized as having diabetes (which itself is a risk factor for cardiovascular disease) and at least one additional risk factor for cardiovascular disease but who does not have an established cardiovascular disease. Patients from cardiovascular risk category 2 were stratified to the primary prevention cohort. The designations for patients defined by cardiovascular risk category 2 are collectively referred to as: primary prevention stratum, primary prevention cohort, and secondary risk category.

Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

The phrase “statistical significance,” as used herein, refers to a result from data generated by testing or experimentation is not likely to occur randomly or by chance, but is instead likely to be attributable to a specific cause. Statistical significance is evaluated from a calculated probability (p-value), where the p-value is a function of the means and standard deviations of the data samples and indicates the probability under which a statistical result occurred by chance or by sampling error. A result is considered statistically significant if the p-value is 0.05 or less, corresponding to a confidence level of 95%.

“Comprising” or “comprises” is intended to mean that the compositions and methods include the recited elements but do not exclude others. “Consisting essentially of,” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

List of abbreviations: ANOVA, analysis of variance; ASCVD, atherosclerotic cardiovascular disease; CI, confidence interval; RRR, relative risk reduction; HR, hazard ratio; CV, cardiovascular; DM, diabetes mellitus; HDL-C, high-density lipoprotein cholesterol; HIV/AIDS, human immunodeficiency virus/acquired immune deficiency syndrome; ICD-9, International Classification of Diseases, Ninth Revision; TG, triglyceride; TC, total cholesterol; VLDL-C very low-density lipoprotein cholesterol; Apo B, apolipoprotein B; hs-CRP, high-sensitivity C-reactive protein; hsTnT, high-sensitivity troponin T; RLP-C, remnant like particle cholesterol; LDL-C, low-density lipoprotein cholesterol; MI, myocardial infarction; non-HDL-C, non-high-density lipoprotein cholesterol; PAD; peripheral artery disease; REDUCE-IT, Reduction of Cardiovascular Events with Icosapent Ethyl-Intervention Trial; SD, standard deviation; and HLB; hydrophilic lipophilic balance.

### Compositions

In one embodiment, a composition of the disclosure is administered to a subject in an amount sufficient to provide a daily dose of eicosapentaenoic acid of about 1 mg to about 20,000 mg, about 25 mg to about 10,000 mg, about 50 mg to about 5000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg.

In one embodiment, a composition for use in methods of the disclosure comprises eicosapentaenoic acid, or a pharmaceutically acceptable ester, derivative, conjugate, or salt thereof, or mixtures of any of the foregoing, collectively referred to herein as “EPA.” The term “pharmaceutically acceptable” in the present context means that the substance in question does not produce unacceptable toxicity to the subject or interaction with other components of the composition. In one embodiment, derivatives of EPA include, but are not limited to, methyl or other alkyl esters, re-esterified monoglycerides, re-esterified diglycerides, and re-esterified triglycerides or mixtures thereof. In one embodiment, such derivatives of EPA are administered daily in amounts containing the same number of moles of EPA contained in 4 grams of ethyl icosapentate.

In another embodiment, the EPA comprises an eicosapentaenoic acid ester. In another embodiment, the EPA comprises a C₁-C₅ alkyl ester of eicosapentaenoic acid. In another embodiment, the EPA comprises eicosapentaenoic acid ethyl ester (E-EPA), eicosapentaenoic acid methyl ester, eicosapentaenoic acid propyl ester, or eicosapentaenoic acid butyl ester.

In another embodiment, the EPA is in the form of ethyl-EPA, methyl-EPA, lithium EPA, mono-, di- or triglyceride EPA or any other ester or salt of EPA, or the free acid form of EPA. The EPA may also be in the form of a 2-substituted derivative or other derivative which slows down its rate of oxidation but does not otherwise change its biological action to any substantial degree. Where any particular form of EPA (e.g., eicosapentaenoic acid ethyl ester, icosapent ethyl, or E-EPA) is referred to throughout this application, any pharmaceutically acceptable derivative of EPA can be substituted in its place including icosapent methyl or eicosapentaenoic acid in free acid form. Eicosapentaenoic acid ethyl ester, icosapent ethyl, and E-EPA are referenced interchangeably.

In another embodiment, EPA is present in a composition useful in accordance with methods of the disclosure in an amount of about 50 mg to about 5000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, or about 5000 mg.

In another embodiment, a composition useful in accordance with the disclosure contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight, of docosahexaenoic acid (DHA), if any. In another embodiment, a composition of the disclosure contains substantially no DHA. In still another embodiment, a composition useful in the present disclosure contains no DHA and/or derivative thereof. In one embodiment, derivatives of DHA include, but are not limited to, methyl or other alkyl esters, re-esterified monoglycerides, re-esterified diglycerides, and re-esterified triglycerides or mixtures thereof.

In another embodiment, EPA comprises at least about 70%, at least about 80%, at least about 90%, at least about 95%, at least about 96%, at least about 97%, at least about 98%, at least about 99%, or at least about 100%, by weight, of all fatty acids present in a composition that is useful in methods of the present disclosure.

In some embodiments, the composition comprises at least 96% by weight of eicosapentaenoic acid ethyl ester and less than about 2% by weight of a preservative. In some embodiments, the preservative is a tocopherol such as all-racemic α-tocopherol.

In another embodiment, a composition useful in accordance with methods of the disclosure contains less than about 10%, less than about 9%, less than about 8%, less than about 7%, less than about 6%, less than about 5%, less than about 4%, less than about 3%, less than about 2%, less than about 1%, less than about 0.5% or less than about 0.25%, by weight of the total composition or by weight of the total fatty acid content, of any fatty acid other than EPA. Illustrative examples of a “fatty acid other than EPA” include linolenic acid (LA), arachidonic acid (AA), docosahexaenoic acid (DHA), alpha-linolenic acid (ALA), stearidonic acid (SDA), eicosatrienoic acid (ETA) and/or docosapentaenoic acid (DPA). In another embodiment, a composition useful in accordance with methods of the disclosure contains about 0.1% to about 4%, about 0.5% to about 3%, or about 1% to about 2%, by weight, of total fatty acids other than EPA and/or DHA. In one embodiment, fatty acids other than EPA include derivatives of those fatty acids. Derivatives of the fatty acids include, but are not limited to, methyl or other alkyl esters, re-esterified monoglycerides, re-esterified diglycerides, and re-esterified triglycerides or mixtures thereof of the fatty acids.

In another embodiment, a composition useful in accordance with the disclosure has one or more of the following features: (a) eicosapentaenoic acid ethyl ester represents at least about 96%, at least about 97%, or at least about 98%, by weight, of all fatty acids present in the composition; (b) the composition contains not more than about 4%, not more than about 3%, or not more than about 2%, by weight, of total fatty acids other than eicosapentaenoic acid ethyl ester; (c) the composition contains not more than about 0.6%, not more than about 0.5%, or not more than about 0.4% of any individual fatty acid other than eicosapentaenoic acid ethyl ester; (d) the composition has a refractive index (20° C.) of about 1.0 to about 2.0, about 1.2 to about 1.8, or about 1.4 to about 1.5; (e) the composition has a specific gravity (20° C.) of about 0.8 to about 1.0, about 0.85 to about 0.95, or about 0.9 to about 0.92; (f) the composition contains not more than about 20 ppm, not more than about 15 ppm, or not more than about 10 ppm heavy metals; (g) the composition contains not more than about 5 ppm, not more than about 4 ppm, not more than about 3 ppm, or not more than about 2 ppm arsenic; and/or (h) the composition has a peroxide value of not more than about 5 meq/kg, not more than about 4 meq/kg, not more than about 3 meq/kg, or not more than about 2 meq/kg.

In some embodiments, a composition for use in accordance with the disclosure is a self-emulsifying composition. In some embodiments, the self-emulsifying composition comprises at least one compound selected from the group consisting of an omega-3 fatty acid and derivative thereof (e.g., pharmaceutically acceptable salt and/or ester). In another embodiment, the composition comprises an emulsifier. In some embodiments, the emulsifier has a hydrophilic lipophilic balance (HLB) of at least about 10. Non-limiting examples of emulsifiers include polyoxyethylene hydrogenated castor oil, polyoxyethylene sorbitan fatty acid ester, polyoxyethylene castor oil, polyethylene glycol fatty acid ester, polyoxyethylene polyoxypropylene glycol, sucrose fatty acid ester, and lecithin. In another embodiment, the omega-3 fatty acids or derivatives thereof are present in an amount of about 50% to about 95% by weight of the total weight of the composition or by weight of the total fatty acids of the total composition. In some embodiments, the omega-3 fatty acid is EPA and/or DHA. In some embodiments, the EPA is present in amount at least about 95%, by weight, of all fatty acids present in the self-emulsifying composition. In another embodiment, the composition contains substantially no DHA. In yet another embodiment, the composition contains substantially no ethanol.

In another embodiment, the composition is a self-emulsifying composition comprising about 50% to about 95% by weight of the total weight of the composition with at least one compound selected from the group consisting of omega-3 polyunsaturated fatty acids and derivatives thereof (e.g., pharmaceutically acceptable salt and/or ester). In another embodiment, the composition comprises about 1% to about 20% by weight of the total weight of the composition, a sucrose fatty acid ester as an emulsifier having an HLB of at least about 10. In another embodiment, the composition comprises glycerin. In another embodiment, the composition comprises about 0% to about 5%, by weight of the total composition, ethanol. In another embodiment, the self-emulsifying composition comprises about 50% to about 95%, by weight of the total weight of the composition, at least one compound selected from the group consisting of omega-3 polyunsaturated fatty acids and derivatives thereof; about 1% to about 20%, by weight of the total weight of the composition, a sucrose fatty acid ester as an emulsifier having an HLB of at least about 10; glycerin; and about 0% to about 4%, by weight of the total weight of the composition, ethanol. In another embodiment, the sucrose fatty acid ester is one or more of: sucrose laurate, sucrose myristate, sucrose palmitate, sucrose stearate, or sucrose oleate. In another embodiment, the omega-3 polyunsaturated fatty acid is one or more of EPA, DHA, or derivatives thereof. In yet another embodiment, the omega-3 polyunsaturated fatty acid is ethyl-EPA and/or ethyl-DHA.

In another embodiment, the composition is a self-emulsifying composition comprising about 50% to about 95% by weight of the total weight of the composition, at least one compound selected from the group consisting of omega-3 polyunsaturated fatty acids and derivatives thereof (e.g., pharmaceutically acceptable salt and ester); and about 5% to about 50%, by weight, of the total weight of the composition an emulsifier having an HLB of at least about 10; wherein ethanol content is up to about 4% by weight of the total weight of the composition. In some embodiments, the omega-3 polyunsaturated fatty acid is EPA and/or DHA. In another embodiment, the composition does not contain ethanol. In another embodiment, the emulsifier is at least one member selected from the group consisting of polyoxyethylene hydrogenated castor oil, polyoxyethylene sorbitan fatty acid ester, polyoxyethylene castor oil, polyethylene glycol fatty acid ester, polyoxyethylene polyoxypropylene glycol, sucrose fatty acid ester, and lecithin. In another embodiment, the emulsifier is at least one member selected from the group consisting of polyoxyethylene hydrogenated castor oil, polyoxyethylene sorbitan fatty acid ester, polyoxyethylene castor oil, and sucrose fatty acid ester.

In another embodiment, the hydrogenated castor oil is at least one member selected from the group consisting of polyoxyethylene (20) hydrogenated castor oil, polyoxyethylene (40) hydrogenated castor oil, polyoxyethylene (50) hydrogenated castor oil, polyoxyethylene (60) hydrogenated castor oil, or polyoxyethylene (100) hydrogenated castor oil. In another embodiment, the polyoxyethylene sorbitan fatty acid ester is at least one member selected from the group consisting of polyoxyethylene sorbitan monooleate, polyoxyethylene sorbitan tristearate, polyoxyethylene sorbitan monostearate, polyoxyethylene sorbitan monopalmitate, and polyoxyethylene sorbitan monolaurate. In another embodiment, the sucrose fatty acid ester is at least one member selected from the group consisting of sucrose laurate, sucrose myristate, sucrose palmitate, sucrose stearate, and sucrose oleate.

In some embodiments, the composition contains a lecithin selected from the group consisting of soybean lecithin, enzymatically decomposed soybean lecithin, hydrogenated soybean lecithin, and egg yolk lecithin. In another embodiment, the composition contains a polyhydric alcohol, wherein the polyhydric alcohol is propylene glycol or glycerin. In another embodiment, the composition contains at least one member selected from the group consisting of EPA, DHA, and/or derivatives thereof (e.g., their pharmaceutically acceptable salt and ester), wherein the composition contains ethyl-EPA and/or ethyl-DHA. In another embodiment, the composition comprises an emulsifier having an HLB of at least about 10 and is about 10 to about 100 parts by weight in relation to 100 parts by weight of the at least one compound selected from the group consisting of omega-3 polyunsaturated fatty acids and/or derivatives thereof (e.g., pharmaceutically acceptable salt and/or ester).

In another embodiment, the self-emulsifying composition comprises about 70% to about 90%, by weight, eicosapentaenoic acid ethyl ester as a first medicinal component. In some embodiments, the composition further comprises about 0.5% to about 0.6%, by weight, water. In some embodiments, the composition comprises about 1% to about 29%, by weight, polyoxyethylene sorbitan fatty acid ester as an emulsifier. In another embodiment, the composition comprises about 1 part to about 25 parts, by weight, lecithin in relation to about 100 parts, by weight, eicosapentaenoic acid ethyl ester. In yet another embodiment, the composition comprises pitavastatin, rosuvastatin, or a salt thereof as a second medicinal component. In another embodiment, ethanol and/or polyhydric alcohol constitutes up to about 4% by weight of the total weight of the composition. In another embodiment, the composition comprises about 0.01 part to about 1 part, by weight, of pitavastatin or its salt in relation to about 100 parts, by weight, of the eicosapentaenoic acid ethyl ester, or about 0.03 part to about 5 parts, by weight, rosuvastatin or its salt in relation to about 100 parts, by weight, eicosapentaenoic acid ethyl ester as a second medicinal component. In some embodiments, the composition is encapsulated in a hard capsule and/or a soft capsule, wherein a capsule film of the soft capsule may contain gelatin. In another embodiment, the self-emulsifying composition further comprises polyoxyethylene hydrogenated castor oil and/or polyoxyethylene castor oil. In another embodiment, the emulsifier comprises polyoxyethylene sorbitan fatty acid ester and polyoxyethylene castor oil. In some embodiments, the pitavastatin, rosuvastatin, or a salt thereof is pitavastatin calcium or rosuvastatin calcium. In another embodiment, the lecithin is soybean lecithin. In another embodiment, the polyoxyethylene sorbitan fatty acid ester is polyoxyethylene (20) sorbitan monooleate.

In some embodiments, the self-emulsifying composition comprising E-EPA has improved bioavailability as compared to a standard E-EPA formulation. A standard E-EPA formulation is a formulation that is not self-emulsifying. In some embodiments, a self-emulsifying composition comprising about 1.8 g to about 3.8 g of E-EPA has substantially equivalent bioavailability to about 4 g E-EPA that is not formulated as a self-emulsifying composition. In some embodiments, the self-emulsifying composition comprising E-EPA is assessed for a bioequivalence to about 4 g E-EPA that is not formulated as a self-emulsifying composition using for example, U.S. Food and Drug Administration (FDA) guidelines.

In another embodiment, compositions useful in accordance with methods of the disclosure are orally deliverable. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration. In one embodiment, the composition is present in a capsule, for example, a soft gelatin capsule.

A composition for use in accordance with the disclosure can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered once to a plurality (e.g., 1 to about 10, 1 to 8, 1 to 6, 1 to 4 or 1 to 2) of times per day, or as many times as needed to elicit a therapeutic response.

In one embodiment, compositions of the disclosure, upon storage in a closed container maintained at room temperature, refrigerated temperature (e.g., about 5 to about −10° C.), or frozen for a period of about 1, 2, 3, 4, 5, 6, 7, 8, 9, 10, 11, or 12 months, exhibit at least about 90%, at least about 95%, at least about 97.5%, or at least about 99% of the active ingredient(s) originally present therein.

### Therapeutic Methods

In some embodiments, the disclosure provides methods for treatment and/or prevention of a viral disease. In some embodiments, the disclosure also provides methods for treatment and/or prevention of a disease or symptoms thereof caused by a virus in a subject.

In some embodiments, the disclosure provides methods for treatment and/or prevention of an upper respiratory infection (URTI). URTIs are viral, or, less commonly, bacterial, infections that affect the nose, throat, pharynx, larynx, and bronchi, including, for example, the common cold, sinusitis, pharyngitis, laryngitis, epiglottitis, tracheobronchitis, and bronchitis. Non-limiting examples of viruses that can cause URTI include rhinovirus, adenovirus, respiratory syncytial virus, and influenza virus. Common symptoms of URTIs include coughing, sneezing, nasal discharge, nasal congestion, runny nose, fever, scratchy throat, sore throat, headache, pain, wheezing, and fatigue.

In some embodiments, the disclosure provides methods for treatment, prevention, or amelioration of one or more symptoms and/or diseases associated with a SARS-CoV-2 infection. Non-limiting examples of diseases associated with the SARS-CoV-2 infection include COVID-19. As used herein, the terms “SARS-CoV-2,” “coronavirus,” “corona,” “2019 novel coronavirus,” “2019-nCoV,” and “COVID-19” are used interchangeably throughout the present disclosure.

The term “treatment” in relation a given disease, disorder or viral infection, includes, but is not limited to, inhibiting the disease, disorder or viral infection, for example, arresting the development of the disease, disorder, or viral infection; relieving the disease, disorder, or viral infection, for example, causing regression of the disease, disorder, or viral infection; or relieving a condition caused by or resulting from the disease, disorder, or viral infection, for example, relieving or treating symptoms of the disease, disorder, or viral infection. The term “prevention” in relation to a given disease, disorder, or viral infection means: preventing the onset of disease, disorder, or viral infection development if none had occurred; preventing the disease, disorder, or viral infection from occurring in a subject that may be predisposed to the disorder, disease, or viral infection but has not yet been diagnosed as having the disorder, disease, or viral infection; and/or preventing further disease/disorder/infection development if already present.

In some embodiments, the methods comprise administering to the subject about 4 g to about 20 g of icosapent ethyl per day. For example, about 4 g, about 5 g, about 6 g, about 7 g, about 8 g, about 9 g, about 10 g, about 11 g, about 12 g, about 13 g, about 14 g, about 15 g, about 16 g, about 17 g, about 18 g, about 19 g, or about 20 g of icosapent ethyl per day.

In some embodiments, the methods comprise administering to the subject the icosapent ethyl for a period of time between about 3 days to about 1 year. In some embodiments, the subject is administered the icosapent ethyl for about 3 days, about 4 days, about 5 days, about 6 days, about 1 week, about 1.5 weeks, about 2 weeks, about 2.5 weeks, about 3 weeks, about 1 month, about 2 months, about 3 months, about 4 months, about 5 months, about 6 months, about 7 months, about 8 months, about 9 months, about 10 months, about 11 months, or about 1 year.

In some embodiments, the methods comprise administering to the subject about 6 g to about 10 g of icosapent ethyl per day for a period of time between about 3 days to about 1 year. In some embodiments, the subject is administered about 6 g to about 10 g icosapent ethyl per day for about 3 days, about 4 days, about 5 days, about 6 days, about 1 week, about 1.5 weeks, about 2 weeks, about 2.5 weeks, about 3 weeks, about 1 month, about 2 months, about 3 months, about 4 months, about 5 months, about 6 months, about 7 months, about 8 months, about 9 months, about 10 months, about 11 months, or about 1 year.

In some embodiments, the methods comprise administering to the subject about 6 g of icosapent ethyl per day for a period of time between 3 days to about 1 year. In some embodiments, the methods comprise administering to the subject about 7 g of icosapent ethyl per day for a period of time between 3 days to about 1 year. In some embodiments, the methods comprise administering to the subject about 8 g of icosapent ethyl per day for a period of time between 3 days to about 1 year. In some embodiments, the methods comprise administering to the subject about 9 g of icosapent ethyl per day for a period of time between 3 days to about 1 year. In some embodiments, the methods comprise administering to the subject about 10 g of icosapent ethyl per day for a period of time between 3 days to about 1 year.

In some embodiments, the subject is administered a “loading dose” of icosapent ethyl fora period of 3, 4, 5, 6, 7, 8, 9, 10, 11, 12, 13, 14, 15, 16, 17, 18, 19, 20, or 21 days, followed by a lower “maintenance dose” of icosapent ethyl. In some embodiments, the loading dose is from 4 g to 20 g per day, for example 4 g, 5 g, 6 g, 7 g, 8 g, 9 g, 10 g, 11 g, 12 g, 13 g, 14 g, 15 g, 16 g, 17 g, 18 g, 19 g, or 20 g per day. In some embodiments, the maintenance dose is from about 1 g to about 4 g per day. In some embodiments, the maintenance dose is administered to the subject for a period of weeks, months, or years, for example, 1, 2, 3, 4, 5, 6, 7, or 8 weeks; 3, 4, 5, 6, 7, 8, 9, 10, or 11 months; or 1, 2, 3, 4, or 5 years.

In some embodiments, the subject infected with SARS-CoV-2 has COVID-19 and/or symptoms of COVID-19 and is an elderly subject (e.g., 60 years or greater), an infant, or an immunocompromised subject.

In some embodiments, the icosapent ethyl is administered orally or intravenously to the subject.

In some embodiments, the methods further comprise monitoring the subject for evidence of SARS-CoV-2 infection, COVID-19, and/or symptoms of COVID-19. Non-limiting examples of symptoms of SARS-CoV-2 infection and/or COVID-19 include coughing, wheezing, fever, tiredness, and difficulty in breathing.

In some embodiments, the methods comprise administering to a subject infected with SARS-CoV-2, having COVID-19 and/or symptoms thereof, about 4 g to about 20 g of icosapent ethyl per day, wherein the subject exhibits a reduction in coughing, wheezing, fever, tiredness, and difficulty in breathing.

In some embodiments, the methods comprise administering to the subject infected with SARS-CoV-2, having COVID-19 and/or symptoms thereof, about 4 g to about 20 g of icosapent ethyl, wherein the subject exhibits a reduction in a risk for systemic inflammatory response syndrome (SIRS) and/or sepsis.

In some embodiments, the methods comprise administering to the subject infected with SARS-CoV-2, having COVID-19 and/or symptoms thereof, about 4 g to about 20 g of icosapent ethyl, wherein the subject exhibits a reduction in inflammation. In certain embodiments, administration of the icosapent ethyl reduces markers of inflammation and/or coagulation over a short duration of time (e.g., 14 days or less). In certain embodiments, administration of the icosapent ethyl reduces markers of inflammation and/or coagulation over a longer duration of time (e.g., 30 days or more). Non-limiting examples of markers of inflammation include high-sensitivity C-reactive protein (hs-CRP), erythrocyte sedimentation rate (ESR), and plasma viscosity (PV). Non-limiting examples of markers of coagulation include D-dimer.

In yet another embodiment, the methods comprise administering to the subject infected with SARS-CoV-2, having COVID-19 and/or symptoms thereof, about 4 g to about 20 g of icosapent ethyl, wherein the subject exhibits a reduction in inflammation of the mucosal membrane. In certain embodiments, the methods comprise administering to the subject infected with SARS-CoV-2, having COVID-19 and/or symptoms thereof, about 4 g to about 20 g of icosapent ethyl, wherein the subject exhibits a reduction in leukotriene levels. Non-limiting examples of include LTB4, LTC4, LTD4, and LTE4.

In certain embodiments, the methods comprise administering to the subject infected with SARS-CoV-2, having COVID-19 and/or symptoms thereof, about 4 g to about 20 g of icosapent ethyl, wherein the subject exhibits a reduction in infectious disease events. Non-limiting examples of infectious disease events include furuncle, gingivitis, mucosal inflammation, severe systemic inflammatory response, SIRS, tooth infection, and vulvovaginal mycotic infection.

In certain embodiments, the methods comprise administering to the subject infected with SARS-CoV-2, having COVID-19 and/or symptoms thereof, about 4 g to about 20 g of icosapent ethyl, wherein the subject exhibits a reduction in respiratory conditions. Non-limiting examples of respiratory conditions include atelectasis, bronchiectasis, cough, emphysema, nasopharyngitis, orthopnea, pulmonary edema, and wheezing.

In some embodiments, the methods comprise administering to the subject infected with SARS-CoV-2, having COVID-19 and/or symptoms thereof, about 4 g to about 20 g of icosapent ethyl, wherein icosapent ethyl activates the heme oxygenase pathway thereby reducing and/or inhibiting symptoms associated with COVID-19. Non-limiting examples of symptoms reduced and/or inhibited by the heme oxygenase pathway include sepsis, acute lung injury, hypertension, renal injury, and/or pain.

In some embodiments, the methods comprise administering to the subject infected with SARS-CoV-2, having COVID-19 and/or symptoms thereof, about 4 g to about 20 g of icosapent ethyl, wherein the subject exhibits an increase in the production of inflammatory mediators. Non-limiting examples of inflammatory mediators include tumor necrosis factor alpha (TNF-α), interleukin 1 beta (IL-1β), soluble intercellular adhesion molecule-1 (ICAM-1), vascular cell adhesion molecule 1 (VCAM-1), and interleukin 10 (IL-10).

In some embodiments, the methods comprise administering to the subject infected with SARS-CoV-2, having COVID-19 and/or symptoms thereof, about 4 g to about 20 g of icosapent ethyl, wherein the subject exhibits a decrease in high-sensitivity C-reactive protein, lipoprotein-associated phospholipase A2, oxidized LDL-C levels, and the AA-to-EPA ratio.

In some embodiments, the methods comprise administering to the subject having a URTI and/or symptoms thereof about 4 g to about 20 g of icosapent ethyl, wherein administration of the icosapent ethyl improves patient-reported outcome measures (e.g., those designed to quantify symptom severity in influenza and other URTIs). The symptoms can be associated with various viruses across multiple body systems over the course of the disease within and across subgroups. In some embodiments, administration of the icosapent ethyl improves patient-reported outcome measures in specific domains of total scores, body/systemic scores, and/or chest/respiratory scores.

In some embodiments, the methods further comprise administering the subject an additional agent. In some embodiments, the additional agent is an anti-viral agent, an anti-malarial agent, and/or a biologic agent. In some embodiments, the additional agent is an agent used to treat malaria (e.g., an anti-malarial agent), SARS, MERS, and/or an autoimmune disorder. In some embodiments, the anti-viral agent is remdesivir and/or vitamin C. In some embodiments, the anti-malarial agent is hydroxychloroquine and/or chloroquine. In some embodiments, the biologic agent includes a peptide and/or a nucleic acid. In some embodiments, the peptide is an antibody. In some embodiments, the biologic agent is a vaccine.

In some embodiments, the subject is administered an anti-viral agent, an anti-malarial agent, and/or a biologic agent before administration of icosapent ethyl. In some embodiments, the subject is co-administered icosapent ethyl and an anti-viral agent, an anti-malarial agent, and/or a biologic agent. In some embodiments, the subject is administered an anti-viral agent, an anti-malarial agent, and/or a biologic agent after administration of icosapent ethyl.

In some embodiments, the methods comprise administering to the subject infected with SARS-CoV-2, having COVID-19 and/or symptoms thereof, about 4 g to about 20 g of icosapent ethyl, wherein administration of icosapent ethyl reduces an amount of time the subject is required to spend on a ventilator. In some embodiments, administration of the icosapent ethyl reduces the amount of time the subject is required to spend on a ventilator by at least about 1 day, at least about 2 days, at least about 3 days, at least about 4 days, at least about 5 days, at least about 6 days, at least about a week, or at least about 1 month.

In some embodiments, the methods comprise administering to the subject infected with SARS-CoV-2, having COVID-19 and/or symptoms thereof, about 4 g to about 20 g of icosapent ethyl, wherein administration of icosapent ethyl reduces an amount of time the subject is required to spend in an intensive care unit (ICU). In some embodiments, administration of the icosapent ethyl reduces the amount time the subject is required to spend in the ICU by at least about 1 day, at least about 2 days, at least about 3 days, at least about 4 days, at least about 5 days, at least about 6 days, at least about a week, or at least about 1 month.

In some embodiments, the methods comprise administering to the subject infected with SARS-CoV-2, having COVID-19 and/or symptoms thereof, about 4 g to about 20 g of icosapent ethyl, wherein administration of icosapent ethyl reduces an amount of time the subject is required to spend in a hospital. In some embodiments, administration of the icosapent ethyl reduces the amount of time the subject is required to spend in the hospital by at least about 1 day, at least about 2 days, at least about 3 days, at least about 4 days, at least about 5 days, at least about 6 days, at least about a week, or at least about 1 month.

In some embodiments, the methods comprise administering to the subject infected with SARS-CoV-2, having COVID-19 and/or symptoms thereof, about 4 g to about 20 g of icosapent ethyl, wherein administration of icosapent ethyl decreases the subject's mortality rate.

In some embodiments, the methods comprise administering to the subject infected with SARS-CoV-2, having COVID-19 and/or symptoms thereof, about 4 g to about 20 g of icosapent ethyl, wherein the subject has acute respiratory distress syndrome (ARDS).

In some embodiments, the methods comprise administering to the subject infected with SARS-CoV-2, having COVID-19 and/or symptoms thereof, about 4 g to about 20 g of icosapent ethyl, wherein the subject has sepsis.

In some embodiments, the methods comprise administering to the subject infected with SARS-CoV-2, having COVID-19 and/or symptoms thereof, about 4 g to about 20 g of icosapent ethyl, wherein administration of icosapent ethyl prevents the subject from progressing to SIRS.

In some embodiments, the methods comprise administering to the subject infected with SARS-CoV-2, having COVID-19 and/or symptoms thereof, about 4 g to about 20 g of icosapent ethyl, wherein the subject has SIRS and administration of icosapent ethyl prevents the subject from progressing to sepsis.

In some embodiments, the methods comprise administering to the subject infected with SARS-CoV-2, having COVID-19 and/or symptoms thereof, about 4 g to about 20 g of icosapent ethyl, wherein the subject has sepsis and administration of icosapent ethyl prevents the subject from progressing to septic shock.

In some embodiments, the methods comprise administering to the subject infected with SARS-CoV-2, having COVID-19 and/or symptoms thereof, about 4 g to about 20 g of icosapent ethyl, wherein the subject has septic shock and administration of icosapent ethyl prevents the subject from progressing to acute lung injury and/or ARDS.

In some embodiments, the methods comprise administering to the subject infected with SARS-CoV-2, having COVID-19 and/or symptoms thereof, about 4 g to about 20 g of icosapent ethyl, wherein the subject exhibits an increase in an LTB5, anaphylaxis leukotrienes of the C, D, and E series, thromboxane, and/or prostacyclin levels.

In various embodiments, the present disclosure provides methods of reducing a risk of a cardiovascular event in a subject on statin therapy. In some embodiments, the methods comprise (a) identifying a subject on statin therapy and having a fasting baseline triglyceride level of about 135 mg/dL to about 500 mg/dL, wherein said subject has established cardiovascular disease or has a high risk of developing cardiovascular disease; and (b) administering to the subject a composition comprising about 1 g to about 4 g of eicosapentaenoic acid (free acid) or derivative thereof (ethyl or methyl ester) per day. The terms “composition” and “pharmaceutical composition” as provided herein are referenced interchangeably.

In various embodiments, the present disclosure provides methods of reducing a risk of a cardiovascular event in a subject on statin therapy. In some embodiments, the methods comprise (a) identifying a subject on statin therapy and having a fasting baseline triglyceride level of about 80 mg/dL to about 1500 mg/dL, wherein said subject has established cardiovascular disease or has a high risk of developing cardiovascular disease; and (b) administering to the subject a composition comprising about 1 g to about 4 g of eicosapentaenoic acid (free acid) or derivative thereof (ethyl or methyl ester) per day. In some embodiments, the reduction in a risk of a cardiovascular event is not correlated to a reduction in the subject's triglyceride levels.

In some embodiments, the present disclosure provides methods of reducing a risk of a cardiovascular event in a subject on statin therapy with or without an associated reduction in a baseline triglyceride level of the subject. As such, a reduction of cardiovascular events is not correlated to a reduction in the subject's triglyceride levels. Accordingly, regardless of whether the subject exhibits a reduction in triglyceride levels, the subject experiences a reduction in a risk of a cardiovascular event. In some embodiments, the methods comprise administering to the subject a composition comprising eicosapentaenoic acid or derivative thereof, wherein the subject does not exhibit a statistically significant change in fasting triglyceride levels for a period of time after administration of the composition. In some embodiments, the period of time is about 1 year to about 5 years, about 1 year to about 6 years, about 1 year to about 7 years, about 1 year to about 8 years, or about 1 year to about 9 years. In another embodiment, the subject exhibits a reduction in fasting triglycerides at a period time of greater than about 5 years, greater than about 6 years, greater than about 7 years, greater than about 8 years, greater than about 9 years, or greater than about 10 years.

In some embodiments, the present disclosure provides methods of reducing a risk of total cardiovascular events in a subject on statin therapy. In some embodiments, the methods comprise administering to the subject a composition comprising eicosapentaenoic acid or derivative thereof. Total cardiovascular events include a first, second, third, fourth, fifth, sixth, seventh, eighth, ninth, tenth, or more cardiovascular event. In some embodiments, the subject has not experienced a cardiovascular event but is at a high risk for experiencing a cardiovascular event. In some embodiments, the subject has experienced multiple cardiovascular events (i.e., a second, third, fourth, or more) and a reduction in a risk of any subsequent cardiovascular event. In some embodiments, the total cardiovascular events are reduced by at least about 20%, at least about 25%, at least about 30%, at least about 35%, at least about 40%, at least about 45%, or at least about 50%. In some embodiments, the total cardiovascular events are reduced regardless of the subject's fasting baseline triglyceride level. For example, total cardiovascular events are reduced in a subject having a fasting baseline triglyceride level in a low, medium, or high tertile. Subjects in the low baseline fasting triglyceride tertile have triglyceride levels between about 80 mg/dL to about 190 mg/dL (median triglyceride level of 160 mg/dL), subjects in the medium baseline fasting triglyceride tertile have triglyceride levels between about 191 mg/dL to about 250 mg/dL (median triglyceride level of 215 mg/dL), and lastly, subjects in the high baseline fasting triglyceride tertile have triglyceride levels between about 251 mg/dL to about 1400 mg/dL (median triglyceride level of 304 mg/dL).

In some embodiments, the present disclosure provides methods of reducing a cardiovascular event in a subject on statin therapy, the methods comprising instructing or having instructed a caregiver of the subject to inquire if the subject has or previously has had atrial fibrillation and/or flutter, assessing or having assessed whether the subject has or has previously had symptoms of atrial fibrillation and/or flutter, monitoring or having monitored the subject for symptoms of atrial fibrillation and/or flutter, and/or providing or having provided guidance to a caregiver of the subject to monitor the subject for symptoms of atrial fibrillation and/or flutter. In some embodiments, the methods further comprise administering or having administered to the subject a composition comprising eicosapentaenoic acid or derivative thereof per day.

In some embodiments, the present disclosure provides methods of reducing an incidence of a cardiovascular event in a subject on statin therapy. In some embodiments, the methods comprise administering to the subject a composition comprising eicosapentaenoic acid or derivative thereof per day, wherein the subject experiences atrial fibrillation and/or flutter and a reduction in or no cardiovascular event. For example, administration of the composition shifts the cardiovascular event to a less medically severe outcome of atrial fibrillation and/or flutter. As such, in some embodiments, the subject experiences atrial fibrillation and/or flutter instead of a cardiovascular event. In another embodiment, the subject exhibits an increase in the symptoms of atrial fibrillation and/or flutter and a reduction in a cardiovascular event as compared to baseline or a placebo control. In some embodiments, the increase in the symptoms of atrial fibrillation and/or flutter are statistically significant as compared to baseline or a placebo control. For example, the symptoms of atrial fibrillation and/or flutter increase by at least about 1%, at least about 2%, at least about 3%, at least about 4%, or at least about 5%. In yet another embodiment, an incidence of atrial fibrillation and/or flutter requiring hospitalization is greater in the subject as compared to baseline or a placebo control. In some embodiments, the subject experiences a reduction in heart rate.

In some embodiments, the present disclosure provides methods of reducing a risk of a cardiovascular event in a subject on low, medium, or high statin therapy. In some embodiments, the methods comprise administering to the subject a composition comprising eicosapentaenoic acid or derivative thereof per day and a low, medium, or high intensity statin therapy. In some embodiments, the low intensity statin therapy includes about 5 mg to about 10 mg of simvastatin. In some embodiments, the medium intensity statin therapy includes about 5 mg to about 10 mg of rosuvastatin, about 10 mg to about 20 mg of atorvastatin, about 20 mg to 40 mg of simvastatin, or about 10 mg to about 20 mg of simvastatin plus about 5 mg to about 10 mg of ezetimibe. In some embodiments, the high intensity statin therapy includes about 20 mg to about 40 mg rosuvastatin, about 40 mg to about 80 mg of atorvastatin, about 80 mg of simvastatin, or about 40 mg to about 80 mg of simvastatin plus about 5 mg to about 10 mg of ezetimibe. In some embodiments, the subject administered the high statin therapy exhibits a greater reduction in a cardiovascular event as compared to a subject in either a low or medium statin therapy. In some embodiments, the subject on a medium statin therapy exhibits a greater reduction in a cardiovascular event as compared to a subject on either a high or low statin therapy. In some embodiments, the subject on a low statin therapy exhibits a greater reduction in a cardiovascular event as compared to a subject on a high or medium statin therapy. In some embodiments, the greater reduction is a reduction of at least about 5%, at least about 10%, at least about 20%, at least about 30%, at least about 50%, at least about 60%, at least about 70%, at least about 80%, at least about 90%, at least about 100%, or more.

In some embodiments, the present disclosure provides methods of delaying an onset of: (a) nonfatal myocardial infarction; (b) fatal or nonfatal stroke; (c) cardiovascular death; (d) unstable angina; (e) coronary revascularization; (f) hospitalization for unstable angina; (g) composite of cardiovascular death or nonfatal myocardial infarction; (h) fatal or nonfatal myocardial infarction; (i) non-elective coronary revascularization representing the composite of emergent or urgent classifications; (j) unstable angina determined to be caused by myocardial ischemia by invasive or noninvasive testing and requiring emergent hospitalization; and/or (k) a composite of total mortality, nonfatal myocardial infarction, and/or nonfatal stroke. An onset of a disease and/or cardiovascular event refers to a first appearance of a sign and/or symptom of the cardiovascular event. In some embodiments, delaying an onset of a cardiovascular event prevents the subject from experiencing the cardiovascular event and/or developing any further symptoms of the cardiovascular event. In some embodiments, the methods comprise administering a composition comprising eicosapentaenoic acid or derivative thereof per day.

In yet another embodiment, the present disclosure provides methods of reducing risk of occurrence of one or more components of a 3-point composite endpoint composed of cardiovascular death, nonfatal myocardial infarction, or nonfatal stroke in a subject on statin therapy or reducing risk occurrence of one or more components of a 5-point composite endpoint composed of cardiovascular death, nonfatal stroke, nonfatal myocardial infarction, coronary revascularization, or unstable angina requiring hospitalization in a subject on statin therapy. In some embodiments, each of the individual components of 3-point composite and 5-point composite endpoints is reduced. For example, each of cardiovascular death, nonfatal myocardial infarction, and nonfatal stroke are reduced within the combination. In some embodiments, the methods comprise administering a composition comprising eicosapentaenoic acid or derivative thereof per day. In some embodiments, the 3-point composite endpoint or the 5-point composite endpoint is reduced by at least about 20%, at least about 30%, at least about 40%, or at least about 50%. In some embodiments, each of the individual components of the 3-point composite endpoint or the 5-point composite endpoint is reduced by at least about 20%, at least about 30%, at least about 40%, or at least about 50%.

In another embodiment, the present disclosure provides methods of reducing a cardiovascular event, the methods comprising administering a composition which comprises EPA or derivative thereof that is formulated such that when administered to the subject, the composition provides an amount of EPA or derivative thereof effective to achieve an efficacy equivalent dose to about a 4 g dose of EPA or derivative thereof but at a lower daily dose of EPA or derivative thereof. In some embodiments, the lower daily dose of the EPA or derivative thereof of is not more than about 3.8 g, not more than about 3.6 g, not more than about 3.4 g, not more than about 3.2 g, not more than about 3 g, not more than about 2.8 g, not more than about 2.6 g, or not more than about 2.5 g. In some embodiments, the lower daily dose of the EPA or derivative thereof is reduced by at least about 10%, at least about 20%, at least about 30%, or at least about 40% in the subject as compared to a baseline or placebo control. In one embodiment, administering the composition to the subject results in an improved pharmacokinetic profile in the subject as compared to a control subject, wherein the subject and control subject are in either a fed or fasting state, and wherein the pharmacokinetic profile is defined by maximum serum concentration (Cₘₐₓ) and area under the curve (AUC). In some embodiments, the control subject is on a statin therapy and administered a placebo or other fatty acid composition such as Lovaza® comprised of 365 mg of E-EPA and 375 mg of E-DHA.

In some embodiments, the present disclosure provides methods of reducing a cardiovascular event in a subject on a statin therapy, the methods comprising administering a composition comprising EPA or derivative thereof, wherein the subject does not experience an adverse event. Non-limiting examples of adverse events include back pain, nasopharyngitis, arthralgia, bronchitis, oedema peripheral, dyspnea, osteoarthritis, cataract, fatigue, constipation, musculoskeletal pain, gout, fall, Type 2 diabetes mellitus, gastroesophageal reflux disease, insomnia, acute kidney injury, hepatic disorders, bleeding related disorders (e.g., gastrointestinal or central nervous system bleeding), newly diagnosed diabetes, newly diagnosed neoplasms (e.g., benign or malignant neoplasms), upper respiratory tract infection, chest pain, peripheral edema, pneumonia, influenza, urinary tract infection, cough, dizziness, pain in an extremity, angina pectoris, and anemia.

In yet another embodiment, the present disclosure provides methods of reducing a cardiovascular event in a subject on a statin therapy and less than about 65 years of age or greater than about 65 years of age, the method comprising administering to the subject a composition comprising EPA or derivative thereof. In some embodiments, the degree by which the cardiovascular event is reduced is dependent upon the age of the subject. For example, in some embodiments, the subject less than about 65 years of age exhibits a statistically significant reduction in a cardiovascular event as compared to a subject greater than about 65 years of age. Conversely, in some embodiments, the subject greater than about 65 years of age exhibits a statistically significant reduction in a cardiovascular event as compared to a subject less than about 65 years of age. As such, in some embodiments, the methods for reducing a cardiovascular event are correlated to the age of the subject.

In some embodiments, the present disclosure provides methods of reducing a cardiovascular event in a subject on a statin therapy, the methods comprising administering to the subject a self-emulsifying composition. In some embodiments, the self-emulsifying composition comprises at least one compound selected from the group consisting of an omega-3 fatty acid and derivative thereof (e.g., pharmaceutically acceptable salt and/or ester). In another embodiment, the composition comprises an emulsifier. In some embodiments, the emulsifier has an HLB of at least about 10. Non-limiting examples of emulsifiers include polyoxyethylene hydrogenated castor oil, polyoxyethylene sorbitan fatty acid ester, polyoxyethylene castor oil, polyethylene glycol fatty acid ester, polyoxyethylene polyoxypropylene glycol, sucrose fatty acid ester, and lecithin. In another embodiment, the omega-3 fatty acids or derivative thereof are present in an amount of about 50% to about 95% by weight of the total weight of the composition or by weight of the total fatty acids of the total composition. In some embodiments, the omega-3 fatty acid is EPA and/or DHA. In some embodiments, the EPA is present in amount at least about 95%, by weight, of all fatty acids present in the self-emulsifying composition. In another embodiment, the composition contains substantially no DHA. In yet another embodiment, the composition contains substantially no ethanol.

In some embodiments, the subject has symptoms of atrial fibrillation and/or flutter. Non-limiting examples of symptoms of atrial fibrillation and/or flutter include heart rate greater than about 100 beats per minute (bpm); heart palpitations; shortness of breath; pain, pressure, tightness or discomfort in chest; dizziness; lightheadedness; or fainting. In some embodiments, the subject has a risk factor for atrial fibrillation and/or flutter including (a) heart failure; (b) previous heart attack; (c) heart valve abnormalities; (d) high blood pressure; (e) thyroid dysfunction; (f) chronic lung disease; (g) diabetes; (h) obesity; and (i) congenital heart disease.

In some embodiments, the methods further comprise monitoring a subject for atrial fibrillation and/or flutter or for symptoms of atrial fibrillation and/or flutter. Non-limiting examples for methods to monitor atrial fibrillation and/or flutter include electrocardiograms (ECGs), implantable pacemakers, implantable cardioverter defibrillators, and/or subcutaneous implantable cardiac monitors.

In some embodiments, the subject has atrial fibrillation and/or flutter or has symptoms of atrial fibrillation and/or flutter and has been determined to have a heart rate of about 80 bpm, about 85 bpm, about 90 bpm, about 95 bpm, about 100 bpm, about 105 bpm, about 110 bpm, about 115 bpm, about 120 bpm, about 125 bpm, about 130 bpm, about 135 bmp, about 140 bmp, about 145 bmp, about 150 bpm, about 155 bpm, about 160 bpm, about 165 bpm, about 170 bpm, about 175 bpm, about 180 bpm, about 185 bpm, about 190 bpm, or a heart rate between about 80 bpm to about 100 bpm, about 90 bpm to about 200 bpm, about 100 bpm to about 175 bpm, about 120 bpm to about 180 bpm, or about 85 bpm to about 200 bpm.

In some embodiments, the present disclosure provides methods of reducing blood pressure in a subject. In one embodiment, administration of 4 g per day of a composition comprising EPA or derivative thereof (E-EPA) for a period at least 1, 2, 3, or 4 years reduces systolic blood pressure by at least about 1 mm Hg and reduces diastolic blood pressure by at least about 0.5 mm Hg, compared to baseline or a placebo control subject.

In some embodiments, the subject has a fasting baseline triglyceride level of about 135 mg/dL to about 500 mg/dL, for example, about 135 mg/dL to about 500 mg/dL, about 150 mg/dL to about 500 mg/dL, about 200 mg/dL to about 499 mg/dL, or about 200 mg/dL to <500 mg/dL. In some embodiments, the subject has a fasting baseline triglyceride level of about 50 mg/dL to about 1500 mg/dL, for example, about 50 mg/dL to about 1500 mg/dL, about 80 mg/dL to about 1500 mg/dL, about 50 mg/dL to about 190 mg/dL, about 80 mg/dL to about 190 mg/dL, about 190 mg/dL to about 250 mg/dL, about 250 mg/dL to about 1400 mg/dL. In one embodiment, the subject has a fasting baseline triglyceride level of about 80 mg/dL to about 1400 mg/dL. In some embodiments, the subject or subject group has a baseline triglyceride level (or median baseline triglyceride level in the case of a subject group), fed or fasting, of about 50 mg/dL, about 55 mg/dL, about 60 mg/dL, about 65 mg/dL, about 70 mg/dL, about 75 mg/dL, about 80 mg/dL, about 85 mg/dL, about 90 mg/dL, about 95 mg/dL, about 100 mg/dL, about 105 mg/dL, about 110 mg/dL, about 115 mg/dL, about 120 mg/dL, about 125 mg/dL, about 130 mg/dL, about 135 mg/dL, about 140 mg/dL, about 145 mg/dL, about 150 mg/dL, about 155 mg/dL, about 160 mg/dL, about 165 mg/dL, about 170 mg/dL, about 175 mg/dL, about 180 mg/dL, about 185 mg/dL, about 190 mg/dL, about 195 mg/dL, about 200 mg/dL, about 205 mg/dL, about 210 mg/dL, about 215 mg/dL, about 220 mg/dL, about 225 mg/dL, about 230 mg/dL, about 235 mg/dL, about 240 mg/dL, about 245 mg/dL, about 250 mg/dL, about 255 mg/dL, about 260 mg/dL, about 265 mg/dL, about 270 mg/dL, about 275 mg/dL, about 280 mg/dL, about 285 mg/dL, about 290 mg/dL, about 295 mg/dL, about 300 mg/dL, about 305 mg/dL, about 310 mg/dL, about 315 mg/dL, about 320 mg/dL, about 325 mg/dL, about 330 mg/dL, about 335 mg/dL, about 340 mg/dL, about 345 mg/dL, about 350 mg/dL, about 355 mg/dL, about 360 mg/dL, about 365 mg/dL, about 370 mg/dL, about 375 mg/dL, about 380 mg/dL, about 385 mg/dL, about 390 mg/dL, about 395 mg/dL, about 400 mg/dL, about 405 mg/dL, about 410 mg/dL, about 415 mg/dL, about 420 mg/dL, about 425 mg/dL, about 430 mg/dL, about 435 mg/dL, about 440 mg/dL, about 445 mg/dL, about 450 mg/dL, about 455 mg/dL, about 460 mg/dL, about 465 mg/dL, about 470 mg/dL, about 475 mg/dL, about 480 mg/dL, about 485 mg/dL, about 490 mg/dL, about 495 mg/dL, about 500 mg/dL, about 1000 mg/dL, about 1100 mg/dL, about 1200 mg/dL, about 1300 mg/dL, about 1400 mg/dL, about 1500 mg/dL, about 2000 mg/dL, about 2500 mg/dL, about 3000 mg/dL, about 3500 mg/dL, about 4000 mg/dL, about 4500 mg/dL, about 5000 mg/dL, or greater than about 5000 mg/dL. In some embodiments, the subject or subject group has a baseline triglyceride level (or median baseline triglyceride level in the case of a subject group), fed or fasting, greater than or equal to 80 mg/dL, greater than or equal to about 100 mg/dL, greater than or equal to about 120 mg/dL, greater than or equal to about 150 mg/dL, greater than or equal to about 175 mg/dL, greater than or equal to about 250 mg/dL, or greater than equal to about 500 mg/dL, for example, about 190 mg/dL to about 250 mg/dL, about 80 mg/dL to about 190 mg/dL, about 250 mg/dL to about 1400 mg/dL, about 200 mg/dL to about 500 mg/dL, about 300 mg/dL to about 1800 mg/dL, about 500 mg/dL to about 1500 mg/dL, or about 80 mg/dL to about 1500 mg/dL.

In some embodiments, the subject or subject group is also on stable therapy with a statin (with or without ezetimibe). In some embodiments, the subject or subject group also has established cardiovascular disease or is at high risk for establishing cardiovascular disease. In some embodiments, the subject's statin therapy includes administration of one or more statins. For example, and without limitation, the subject's statin therapy may include one or more of: atorvastatin, fluvastatin, lovastatin, pitavastatin, pravastatin, rosuvastatin, and simvastatin. In some embodiments, the subject is additionally administered one or more of: amlodipine, ezetimibe, niacin, and sitagliptin. In some embodiments, the subject's statin therapy includes administration of a statin and ezetimibe. In some embodiments, the subject's statin therapy includes administration of a statin without ezetimibe.

In some embodiments, the statin therapy is classified as monotherapies, combinations, and or 3-hydroxy-3-methyl-glutaryl-coenzyme A (HMG CoA) reductase inhibitor combinations. In some embodiments, the monotherapies include simvastatin, lovastatin, pravastatin, fluvastatin, atorvastatin, cerivastatin, rosuvastatin, or pitavastatin. In some embodiments, the combinations include lovastatin and nicotinic acid, simvastatin and ezetimibe, pravastatin and fenofibrate, simvastatin and fenofibrate, atorvastatin and ezetimibe, or rosuvastatin and ezetimibe. In some embodiments, the HMG CoA inhibitor combinations include simvastatin and acetylsalicylic acid; pravastatin and acetylsalicylic acid; atorvastatin and amlodipine; simvastatin, acetylsalicylic acid, and ramipril; rosuvastatin and acetylsalicylic acid; atorvastatin, acetylsalicylic acid, and ramipril; rosuvastatin, amlodipine, and lisinopril; atorvastatin and acetylsalicylic acid; rosuvastatin and amlodipine; rosuvastatin and valsartan; atorvastatin, amlodipine, and perindopril; atorvastatin, acetylsalicylic acid, and perindopril; rosuvastatin, perindopril, and indapamide; rosuvastatin, amlodipine, and perindopril; or atorvastatin and perindopril.

In some embodiments, the statin therapy is a low, medium (i.e., moderate), or high intensity statin therapy. In some embodiments, the low intensity statin therapy includes about 5 mg to about 10 mg of simvastatin. In some embodiments, the medium intensity statin therapy includes about 5 mg to about 10 mg of rosuvastatin, about 10 mg to about 20 mg of atorvastatin, about 20 mg to about 40 mg of simvastatin, or about 10 mg to about 20 mg of simvastatin plus about 5 mg to about 10 mg of ezetimibe. In some embodiments, the high intensity statin therapy includes about 20 mg to about 40 mg rosuvastatin, about 40 mg to about 80 mg of atorvastatin, about 80 mg of simvastatin, or about 40 mg to about 80 mg of simvastatin plus about 5 mg to about 10 mg of ezetimibe.

In some embodiments, the subject's statin therapy does not include administration of 200 mg or more per day of niacin and/or fibrates. In some embodiments, the subject is not on concomitant omega-3 fatty acid therapy (e.g., is not being administered or co-administered a prescription and/or an over-the-counter composition comprising an omega-3 fatty acid active agent). In some embodiments, the subject is not administered or does not ingest a dietary supplement comprising an omega-3 fatty acid.

In some embodiments, the subject has established cardiovascular (CV) disease (“CV disease” or “CVD”). The status of a subject as having CV disease can be determined by any suitable method known to those skilled in the art. In some embodiments, a subject is identified as having established CV disease by the presence of any one of: documented coronary artery disease, documented cerebrovascular disease, documented carotid disease, documented peripheral arterial disease, or combinations thereof. In some embodiments, a subject is identified as having CV disease if the subject is at least 45 years old and: (a) has one or more stenosis of greater than 50% in two major epicardial coronary arteries; (b) has had a documented prior MI; (c) has been hospitalized for high-risk NSTE ACS with objective evidence of ischemia (e.g., ST-segment deviation and/or biomarker positivity); (d) has a documented prior ischemic stroke; (e) has symptomatic artery disease with at least 50% carotid arterial stenosis; (f) has asymptomatic carotid artery disease with at least 70% carotid arterial stenosis per angiography or duplex ultrasound; (g) has an ankle-brachial index (“ABI”) of less than 0.9 with symptoms of intermittent claudication; and/or (h) has a history of aorto-iliac or peripheral arterial intervention (catheter-based or surgical).

In some embodiments, the subject or subject group being treated in accordance with methods of the disclosure has a high risk for developing CV disease. For example and without limitation, a subject or subject group has a high risk for developing CV disease if the subject or subject in a subject group is age about 50 or older, has diabetes mellitus (Type 1 or Type 2), and at least one of: (a) is a male age about 55 or older or a female age about 65 or older; (b) is a cigarette smoker or was a cigarette smoker who stopped less than about 3 months prior; (c) has hypertension (e.g., a blood pressure of about 140 mmHg systolic or higher, or greater than about 90 mmHg diastolic); (d) has an HDL-C level of less than or equal to about 40 mg/dL for men or less than or equal to about 50 mg/dL for women; (e) has an hs-CRP level of greater than about 3.0 mg/L; (f) has renal dysfunction (e.g., a creatinine clearance (“CrCL”) of greater than about 30 mL/min and less than about 60 mL/min); (g) has retinopathy (e.g., defined as any of: non-proliferative retinopathy, pre-proliferative retinopathy, proliferative retinopathy, maculopathy, advanced diabetic eye disease, or history of photocoagulation); (h) has microalbuminuria (e.g., a positive micral or other strip test, an album in/creatinine ratio of greater than or equal to about 2.5 mg/mmol, or an albumin excretion rate on timed collection of greater than or equal to about 20 mg/min all on at least two successive occasions); (i) has macroalbuminuria (e.g., Albustix or other dip stick evidence of gross proteinuria, an album in/creatinine ratio of greater than or equal to about 25 mg/mmol, or an albumin excretion rate on timed collection of greater than or equal to about 200 mg/min all on at least two successive occasions); and/or (j) has an ankle-brachial index of less than about 0.9 without symptoms of intermittent claudication.

In some embodiments, the subject's baseline lipid profile is measured or determined prior to administering the composition to the subject. Lipid profile characteristics can be determined by any suitable method known to those skilled in the art including, for example, by testing a fasting or non-fasting blood sample obtained from the subject using standard blood lipid profile assays. In some embodiments, the subject has one or more of: a baseline non-HDL-C value of about 200 mg/dL to about 300 mg/dL; a baseline total cholesterol value of about 250 mg/dL to about 300 mg/dL; a baseline VLDL-C value of about 140 mg/dL to about 200 mg/dL; a baseline HDL-C value of about 10 mg/dL to about 30 mg/dL; a baseline LDL-C value of about 40 mg/dL to about 100 mg/dL; and/or a baseline hs-CRP level of about 2 mg/dL or less.

In some embodiments, the cardiovascular event for which risk is reduced is one or more of: cardiovascular death; nonfatal myocardial infarction; nonfatal stroke; coronary revascularization; unstable angina (e.g., unstable angina determined to be caused by myocardial ischemia by, for example, invasive or non-invasive testing, and requiring hospitalization); cardiac arrest; peripheral cardiovascular disease requiring intervention, angioplasty, bypass surgery, or aneurysm repair; death; sudden cardiac death, sudden death, and onset of new congestive heart failure. In some embodiments, the cardiovascular event is a first, second, third, fourth, or more cardiovascular event experienced by the subject.

In some embodiments, the subject is administered about 1 g to about 4 g of the composition per day for about 4 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. Thereafter, in some embodiments the subject exhibits one or more of:

(a) a reduction in triglyceride levels compared to baseline or control;

(b) a reduction in Apo B levels compared to baseline or control;

(c) an increase in HDL-C levels compared to baseline or control;

(d) no increase or increase in LDL-C levels compared to baseline or control;

(e) a reduction in LDL-C levels compared to baseline;

(f) a reduction in non-HDL-C levels compared to baseline or control;

(g) an increase in non-HDL-C levels compared to baseline or control;

(h) a reduction in VLDL-C levels compared to baseline or control;

(i) a reduction in total cholesterol levels compared to baseline or control;

(j) a reduction in hs-CRP levels compared to baseline or control;

(k) a reduction in hsTnT levels compared to baseline or control;

(l) a reduction in a risk of cardiovascular death, coronary revascularization, unstable angina, myocardial infarction, and/or stroke as compared to baseline or control;

(m) a reduction in a risk of cardiac arrest as compared to baseline or control;

(n) a reduction in a risk of sudden death as compared to baseline or control;

(o) a reduction in a first, second, third, fourth, or more cardiovascular event as compared to baseline or placebo control;

(p) a reduction in total cardiovascular events as compared to baseline or control;

(q) a reduction in a 3-point composite endpoint of cardiovascular death, nonfatal myocardial infarction, or nonfatal stroke as compared to baseline or control;

(r) a reduction in a 5-point composite endpoint of cardiovascular death, nonfatal stroke, nonfatal myocardial infarction, coronary revascularization, or unstable angina as compared to baseline or control;

(s) an increase in atrial fibrillation and/or flutter as compared to baseline or control;

(t) an increase in symptoms of atrial fibrillation and/or flutter as compared to baseline or control;

(u) a reduction of total mortality (i.e., death from any cause) as compared to baseline or control;

(v) a reduction in a composite of total mortality, nonfatal myocardial infarction, and stroke as compared to baseline or placebo control;

(w) a reduction in new congestive heart failure (CHF) or new CHF as the primary cause of hospitalization as compared to baseline or control;

(x) a reduction in transient ischemic attack as compared to baseline or control;

(y) a reduction in a risk of amputation for peripheral vascular disease (PVD) as compared to baseline or control;

(z) a reduction in a risk of carotid revascularization as compared to baseline or control;

(aa) a reduction in cardiac arrhythmias as compared to baseline or control;

(bb) a reduction in hypertension as compared to baseline or control;

(cc) a reduction in Type 1 or Type 2 diabetes as compared to baseline or control;

(dd) a reduction in body weight and/or weight circumference as compared to baseline or control;

(ee) a reduction in coughing as compared to baseline or control;

(ff) a reduction in wheezing as compared to baseline or control;

(gg) a reduction in inflammation of the mucosal membrane as compared to baseline or control;

(hh) a reduction in a risk of SIRS as compared to baseline or control;

(ii) a reduction in sepsis as compared to baseline or control;

(jj) a reduction in leukotrienes levels selected from the group consisting of LTB4 and the LTC4, LTD4, and LTE4 as compared to baseline or control;

(kk) a reduction in neutrophil levels as compared to baseline or control;

(ll) an increase in lymphocyte levels as compared to baseline or control;

(mm) a reduction in infectious disease events as compared to baseline or control;

(nn) a reduction in respiratory conditions as compared to baseline or control;

(oo) a reduction in the production of inflammatory mediators as compared to baseline or control;

(pp) a reduction in high-sensitivity C-reactive protein as compared to baseline or control;

(qq) a reduction in lipoprotein-associated phospholipase A2 as compared to baseline or control;

(rr) a reduction in oxidized LDL-C levels as compared to baseline or control;

(ss) a reduction in the AA-to-EPA ratio as compared to baseline or control;

(tt) an increase in LBT5 levels as compared to baseline or control;

(uu) an increase in anaphylaxis leukotrienes of the C, D, and E series levels as compared to baseline or control;

(vv) an increase in thromboxane levels as compared to baseline or control; and/or

(ww) an increase in prostacyclin levels as compared to baseline or control.

In one embodiment, methods of the present disclosure comprise measuring baseline levels of one or more markers set forth in (a)-(ww) above prior to dosing the subject or subject group. In another embodiment, the methods comprise administering a composition as disclosed herein to the subject after baseline levels of one or more markers set forth in (a)-(ww) are determined, and subsequently taking an additional measurement of said one or more markers.

In another embodiment, upon treatment with a composition of the present disclosure, the subject exhibits one or more of:

(a) a reduction in triglyceride levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% as compared to baseline or control;

(b) a reduction in Apo B levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% as compared to baseline or control;

(c) an increase in HDL-C levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% as compared to baseline or control;

(d) no increase or an increase in LDL-C levels of less than 30%, less than 20%, less than 10%, less than 5% as compared to baseline or control;

(e) a reduction in LDL-C levels of at least about 5%, at least about 10%, at least about 15%, at least about 20%, at least about 25%, at least about 30%, at least about 35%, at least about 40%, at least about 45%, at least about 50%, or at least about 55% as compared to baseline or control;

(f) a reduction in non-HDL-C levels of at least about 1%, at least about 3%, at least about 5%, at least about 10%, at least about 15%, at least about 20%, at least about 25%, at least about 30%, at least about 35%, at least about 40%, at least about 45%, or at least about 50% as compared to baseline or control;

(g) an increase in non-HDL-C levels of less than 30%, less than 20%, less than 10%, less than 5% (actual % change or median % change), or no increase in non-HDL-C levels as compared to baseline or control;

(h) a reduction in VLDL-C levels of at least about 5%, at least about 10%, at least about 15%, at least about 20%, at least about 25%, at least about 30%, at least about 35%, at least about 40%, at least about 45%, at least about 50%, or at least about 100% compared to baseline or control;

(i) a reduction in total cholesterol levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% as compared to baseline or control; and/or

(j) a reduction in hs-CRP levels of at least about 5%, at least about 10%, at least about 15%, at least about 20%, at least about 25%, at least about 30%, at least about 35%, at least about 40%, at least about 45%, at least about 50%, or at least about 100% as compared to baseline or control;

(k) a reduction in hsTnT levels of at least about 5%, at least about 10%, at least about 15%, at least about 20%, at least about 25%, at least about 30%, at least about 35%, at least about 40%, at least about 45%, at least about 50%, or at least about 100% as compared to baseline or control;

(l) a reduction in a risk of cardiovascular death, coronary revascularization, unstable angina, myocardial infarction, and/or stroke of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(m) a reduction in a risk of cardiac arrest of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(n) a reduction in a risk of sudden cardiac death and/or sudden death of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(o) a reduction in a first, second, third, fourth, or more cardiovascular event experienced by the subject of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(p) a reduction in total cardiovascular events of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(q) a reduction in a 3-point composite endpoint of cardiovascular death, nonfatal myocardial infarction, or nonfatal stroke of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(r) a reduction in a 5-point composite endpoint of cardiovascular death, nonfatal stroke, nonfatal myocardial infarction, coronary revascularization, or unstable angina of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(s) an increase in atrial fibrillation and/or flutter of at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, or at least about 10% as compared to baseline or control;

(t) an increase in symptoms of atrial fibrillation and/or flutter of at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, or at least about 10% as compared to baseline or control;

(u) a reduction of total mortality (i.e., death from any cause) of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(v) a reduction in a composite of total mortality, nonfatal myocardial infarction, and stroke of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(w) a reduction in new CHF or new CHF as the primary cause of hospitalization of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(x) a reduction in transient ischemic attack of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(y) a reduction in a risk of amputation for PVD of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(z) a reduction in a risk of carotid revascularization of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(aa) a reduction in cardiac arrhythmias of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(bb) a reduction in hypertension of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(cc) a reduction in Type 1 or Type 2 diabetes of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(dd) a reduction in body weight and/or weight circumference of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control.

(ee) a reduction in coughing of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(ff) a reduction in wheezing of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(gg) a reduction in inflammation of the mucosal membrane of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(hh) a reduction in a risk of SIRS of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(ii) a reduction in sepsis of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(jj) a reduction in leukotrienes levels selected from the group consisting of LTB4 and the LTC4, LTD4, and LTE4 of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(kk) a reduction in neutrophil levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(ll) an increase in lymphocyte levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(mm) a reduction in infectious disease events of at least about 5%, at least about 10%, at least about 15%, at least about 20%, at least about 25%, at least about 30%, at least about 35%, at least about 40%, at least about 45%, at least about 50%, at least about 55%, at least about 75%, at least about 80%, at least about 85%, at least about 90%, at least about 95%, or at least about 100% as compared to baseline or control;

(nn) a reduction in respiratory conditions of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(oo) a reduction in the production of inflammatory mediators of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(pp) a reduction in high-sensitivity C-reactive protein of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(qq) a reduction in lipoprotein-associated phospholipase A2 of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(rr) a reduction in oxidized LDL-C levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(ss) a reduction in the AA-to-EPA ratio of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(tt) an increase in LBT5 levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(uu) an increase in anaphylaxis leukotrienes of the C, D, and E series levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;

(vv) an increase in thromboxane levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control; and/or

(ww) an increase in prostacyclin levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control.

In one embodiment, the subject or subject group being treated has a baseline EPA blood level on a (mol %) basis of less than 2.6, less than 2.5, less than 2.4, less than 2.3, less than 2.2, less than 2.1, less than 2, less than 1.9, less than 1.8, less than 1.7, less than 1.6, less than 1.5, less than 1.4, less than 1.3, less than 1.2, less than 1.1 or less than 1.

In another embodiment, the subject or subject group being treated has a baseline triglyceride level (or median baseline triglyceride level in the case of a subject group), fed or fasting, of about 135 mg/dL to about 500 mg/dL. In some embodiments, the subject or subject group being treated has a baseline triglyceride level (or median baseline triglyceride level in the case of a subject group), fed or fasting, of about 80 mg/dL to about 1500 mg/dL. In some embodiments, the subject or subject group being treated in accordance with methods of the disclosure is on stable therapy with a statin (with or without ezetimibe). As used herein, the phrase “on stable therapy with a statin” means that the subject or subject group has been on the same daily dose of the same statin for at least 28 days and, if applicable, the same daily dose of ezetimibe for at least 28 days. In some embodiments, the subject or subject group on stable statin therapy has an LDL-C level of about 40 mg/dL to about 100 mg/dL.

In some embodiments, safety laboratory tests of subject blood samples include one or more of: hematology with complete blood count (CBC), including RBC, hemoglobin (Hgb), hematocrit (Hct), white cell blood count (WBC), white cell differential, and platelet count; and biochemistry panel including total protein, albumin, alkaline phosphatase, alanine aminotransferase (ALT/SGPT), aspartate am inotransferase (AST/SGOT), total bilirubin, glucose, calcium, electrolytes, (sodium, potassium, chloride), blood urea nitrogen (BUN), serum creatinine, uric acid, creatine kinase, and hemoglobin A1c (HbA1c).

In some embodiments, a fasting lipid panel associated with a subject includes TG, TC, LDL-C, HDL-C, non-HDL-C, and VLDL-C. In some embodiments, LDL-C is calculated using the Friedewald equation or is measured by preparative ultracentrifugation (Beta Quant) if the subject's triglyceride level is greater than 400 mg/dL. In some embodiments, LDL-C is measured by ultracentrifugation (Beta Quant) at randomization and again after about one year after randomization.

In some embodiments, a biomarker assay associated with blood obtained from a subject includes hs-CRP, Apo B, and hsTnT.

In some embodiments, a medical history associated with a subject includes family history, details regarding all illnesses and allergies including, for example, date(s) of onset, current status of condition(s), and smoking and alcohol use.

In some embodiments, demographic information associated with a subject includes day, month, and year of birth, race, and gender.

In some embodiments, vital signs associated with a subject include systolic and diastolic blood pressure, heart rate, respiratory rate, and body temperature (e.g., oral body temperature).

In some embodiments, a physical examination of a subject includes assessments of the subject's general appearance, skin, head, neck, heart, lung, abdomen, extremities, and neuromusculature.

In some embodiments, the subject's height and weight are measured. In some embodiments, the subject's weight is recorded with the subject wearing indoor clothing, with shoes removed, and with the subject's bladder empty.

In some embodiments, a waist measurement associated with the subject is measured. In some embodiments, the waist measurement is determined with a tape measure at the top of the subject's hip bone.

In some embodiments, an electrocardiogram associated with the subject is obtained. In some embodiments, an ECG is obtained every year during the treatment/follow-up portion of the study. In some embodiments, the ECG is a 12-lead ECG. In some embodiments, the ECG is analyzed for detection of silent MI.

In some embodiments, subjects randomly assigned to the treatment group receive 4 g per day of a composition comprising at least 96% by weight of eicosapentaenoic acid ethyl ester. In some embodiments, the composition is encapsulated in a gelatin capsule. In some embodiments, subjects in this treatment group continue to take 4 g per day of the composition for about 1 year, about 2 years, about 3 years, about 4 years, about 4.75 years, about 5 years, about 6 years, about 7 years, about 8 years, about 9 years, about 10 years, or more than about 10 years. In some embodiments, a median treatment duration is planned to be about 4 years.

In some embodiments, the present disclosure provides a method of reducing a risk of cardiovascular events in a subject. In some embodiments, the method comprises administering to the subject a composition comprising at least 96% by weight of eicosapentaenoic acid ethyl ester. In some embodiments, the subject is administered about 1 g to about 4 g of the composition per day.

In some embodiments, the reduced risk of CV events is indicated or determined by comparing an amount of time (e.g., an average amount of time) associated with a subject or subject group from first dosing to a first CV event selected from the group consisting of: CV death, nonfatal MI, nonfatal stroke, coronary revascularization, and hospitalization (e.g., emergent hospitalization) for unstable angina determined to be caused by myocardial ischemia (e.g., by invasive or non-invasive testing), to an amount of time (e.g., an average amount of time) associated with a placebo or untreated subject or group of subjects from first dosing with a placebo to a first CV event selected from the group consisting of: CV death, nonfatal MI, nonfatal stroke, coronary revascularization, and hospitalization (e.g., emergent hospitalization) for unstable angina determined to be caused by myocardial ischemia (e.g., by invasive or non-invasive testing), wherein said placebo does not include eicosapentaenoic acid ethyl ester. In some embodiments, the amount of time associated with the subject or group of subjects is compared to the amount of time associated with the placebo or untreated subject or group of subjects, which is compared using a log-rank test. In some embodiments, the log-rank test includes one or more stratification factors such as CV Risk Category, use of ezetimibe, and/or geographical region.

In some embodiments, the present disclosure provides a method of reducing risk of CV death in a subject on stable statin therapy and having CV disease or at high risk for developing CV disease, comprising administering to the subject a composition as disclosed herein.

In another embodiment, the present disclosure provides a method of reducing risk of recurrent nonfatal myocardial infarction (including silent MI) in a subject on stable statin therapy and having CV disease or at high risk for developing CV disease, comprising administering to the patient one or more compositions as disclosed herein.

In some embodiments, the present disclosure provides a method of reducing risk of nonfatal stroke in a subject on stable statin therapy and having CV disease or at high risk for developing CV disease, comprising administering to the subject a composition as disclosed herein.

In some embodiments, the present disclosure provides a method of reducing risk of coronary revascularization in a subject on stable statin therapy and having CV disease or at high risk for developing CV disease, comprising administering to the subject a composition as disclosed herein.

In some embodiments, the present disclosure provides a method of reducing risk of developing unstable angina caused by myocardial ischemia in a subject on stable statin therapy and having CV disease or at high risk for developing CV disease, comprising administering to the subject a composition as disclosed herein.

In some embodiments, the present disclosure provides a method of reducing risk of cardiac arrest in a subject on stable statin therapy and having CV disease or at high risk for developing CV disease, comprising administering to the subject a composition as disclosed herein.

In some embodiments, the present disclosure provides a method of reducing risk of sudden cardiac death and/or sudden death in a subject on stable statin therapy and having CV disease or at high risk for developing CV disease, comprising administering to the subject a composition as disclosed herein.

In some embodiments, the present disclosure provides a method of reducing risk of a first, second, third, fourth, or more cardiovascular event in a subject on stable statin therapy and having CV disease or at high risk for developing CV disease, comprising administering to the subject a composition as disclosed herein.

In another embodiment, any of the methods disclosed herein are used in treatment or prevention of a subject or subjects that consume a traditional Western diet. In one embodiment, the methods of the disclosure include a step of identifying a subject as a Western diet consumer or prudent diet consumer and then treating the subject if the subject is deemed a Western diet consumer. The term “Western diet” herein refers generally to a typical diet consisting of, by percentage of total calories, about 45% to about 50% carbohydrate, about 35% to about 40% fat, and about 10% to about 15% protein. A Western diet may alternately or additionally be characterized by relatively high intakes of red and processed meats, sweets, refined grains, and desserts, for example, where more than 50%, more than 60%, or more or 70% of total calories come from these sources.

In another embodiment, a composition as described herein is administered to a subject once or twice per day. In another embodiment, 1, 2, 3, or 4 capsules, each containing about 1 g of a composition as described herein, are administered to a subject daily. In another embodiment, 1 or 2 capsules, each containing about 1 g of a composition as described herein, are administered to the subject in the morning, for example, between about 5 am and about 11 am, and 1 or 2 capsules, each containing about 1 g of a composition as described herein, are administered to the subject in the evening, for example between about 5 pm and about 11 pm.

In some embodiments, the risk of a cardiovascular event in a subject is reduced compared to a control population. In some embodiments, a plurality of control subjects to a control population, wherein each control subject is on stable statin therapy, has a fasting baseline triglyceride level of about 135 mg/dL to about 500 mg/dL, and has established cardiovascular disease or a high risk of developing cardiovascular disease, and wherein the control subjects are not administered the composition comprising about 1 g to about 4 g of eicosapentaenoic acid ethyl ester per day.

In some embodiments, the risk of a cardiovascular event in a subject is reduced compared to a control population. In some embodiments, a plurality of control subjects to a control population, wherein each control subject is on stable statin therapy, has a fasting baseline triglyceride level of about 80 mg/dL to about 1500 mg/dL, and has established cardiovascular disease or a high risk of developing cardiovascular disease, and wherein the control subjects are not administered the composition comprising about 1 g to about 4 g of eicosapentaenoic acid ethyl ester per day.

In some embodiments, a first time interval beginning at (a) an initial administration of a composition as disclosed herein to the subject to (b) a first cardiovascular event of the subject is greater than or substantially greater than a first control time interval beginning at (a′) initial administration of a placebo to the control subjects to (b′) a first cardiovascular event in the control subjects. In some embodiments, the first cardiovascular event of the subject is a major cardiovascular event selected from the group consisting of: cardiovascular death, nonfatal myocardial infarction, nonfatal stroke, coronary revascularization, and unstable angina caused by myocardial ischemia. In some embodiments, the first cardiovascular event of the control subjects is a major cardiovascular event selected from the group consisting of: cardiovascular death, nonfatal myocardial infarction, nonfatal stroke, coronary revascularization, and unstable angina caused by myocardial ischemia. In some embodiments, the first cardiovascular event of the subject and the first cardiovascular event of the control subjects is any of: death (from any cause), nonfatal myocardial infarction, or nonfatal stroke. In some embodiments, the first cardiovascular event of the subject and the first cardiovascular event of the control subjects is any of: death from a cardiovascular cause, nonfatal myocardial infarction, coronary revascularization, unstable angina, peripheral cardiovascular disease, or cardiac arrhythmia requiring hospitalization. In some embodiments, the first cardiovascular event of the subject and the first cardiovascular event of the control subjects is any of: death from a cardiovascular cause, nonfatal myocardial infarction, and coronary revascularization, unstable angina. In some embodiments, the first cardiovascular event of the subject and the first cardiovascular event of the control subjects is any of: death from a cardiovascular cause and nonfatal myocardial infarction. In some embodiments, the first cardiovascular event of the subject and the first cardiovascular event of the control subjects is death (from any cause). In some embodiments, the first cardiovascular event of the subject and the first cardiovascular event of the control subjects is any of: fatal myocardial infarction and nonfatal myocardial infarction (optionally including silent MI). In some embodiments, the first cardiovascular event of the subject and the first cardiovascular event of the control subjects is coronary revascularization. In some embodiments, the first cardiovascular event of the subject and the first cardiovascular event of the control subjects is hospitalization (e.g., emergent hospitalization) for unstable angina (optionally unstable angina caused by myocardial ischemia). In some embodiments, the first cardiovascular event of the subject and the first cardiovascular event of the control subjects is any one of: fatal stroke or nonfatal stroke. In some embodiments, the first cardiovascular event of the subject and the first cardiovascular event of the control subjects is any one of: new coronary heart failure, new coronary heart failure leading to hospitalization, transient ischemic attack, amputation for coronary vascular disease, and carotid revascularization. In some embodiments, the first cardiovascular event of the subject and the first cardiovascular event of the control subjects is any one of: elective coronary revascularization and emergent coronary revascularization. In some embodiments, the first cardiovascular event of the subject and the first cardiovascular event of the control subjects is an onset of diabetes. In some embodiments, the first cardiovascular event of the subject and the first cardiovascular event of the control subjects is cardiac arrhythmia requiring hospitalization. In some embodiments, the first cardiovascular event of the subject and the first cardiovascular event of the control subjects is cardiac arrest. In some embodiments, the first cardiovascular event of the subject and the first cardiovascular event of the control subjects is sudden cardiac death and/or sudden death.

In some embodiments, a second time interval beginning at (a) an initial administration of the composition to the subject to (c) a second cardiovascular event of the subject is greater than or substantially greater than a second control time interval beginning at (a′) initial administration of a placebo to the control subjects to (c′) a second cardiovascular event in the control subjects. In some embodiments, the second cardiovascular event of the subject and the second cardiovascular event of the control subjects is a major cardiovascular event selected from the group consisting of: cardiovascular death, nonfatal myocardial infarction, nonfatal stroke, coronary revascularization, and unstable angina caused by myocardial ischemia. In some embodiments, the major cardiovascular event(s) is further selected from the group consisting of: cardiac arrest, sudden cardiac death, and/or sudden death.

In some embodiments, the subject has diabetes mellitus and the control subjects each have diabetes mellitus. In some embodiments, the subject has metabolic syndrome and the control subjects each have metabolic syndrome.

In some embodiments, the subject exhibits one or more of (a) reduced triglyceride levels compared to the control population; (b) reduced Apo B levels compared to the control population; (c) increased HDL-C levels compared to the control population; (d) no increase in LDL-C levels compared to the control population; (e) a reduction in LDL-C levels compared to the control population; (f) a reduction in non-HDL-C levels compared to the control population; (g) a reduction in VLDL levels compared to the control population; (h) a reduction in total cholesterol levels compared to the control population; (i) a reduction in hs-CRP levels compared to the control population; and/or (j) a reduction in hsTnT levels compared to the control population.

In some embodiments, the subject's weight after administration of the composition is less than a baseline weight determined before administration of the composition. In some embodiments, the subject's waist circumference after administration of the composition is less than a baseline waist circumference determined before administration of the composition.

In methods of the present disclosure in which a time interval is determined or assessed, the time interval may be for example an average, a median, or a mean time interval. For example, in embodiments wherein a first control time interval is associated with a plurality of control subjects, the first control time interval is an average, a median, or a mean of a plurality of first control time intervals associated with each control subject. Similarly, in embodiments wherein a second control time interval is associated with a plurality of control subjects, the second control time interval is an average, a median, or a mean of a plurality of second control time intervals associated with each control subject.

In some embodiments, the reduced risk of cardiovascular events is expressed as a difference in incident rates between a study group and a control population. In some embodiments, the subjects in the study group experience a first major cardiovascular event after an initial administration of a composition as disclosed herein at a first incidence rate which is less than a second incidence rate, wherein the second incidence rate is associated with the rate of cardiovascular events in the subjects in the control population. In some embodiments, the first major cardiovascular event is any one of: cardiovascular death, nonfatal myocardial infarction, nonfatal stroke, coronary revascularization, and hospitalization for unstable angina (optionally determined to be caused by myocardial ischemia). In some embodiments, the first and second incidence rates are determined for a time period beginning on the date of the initial administration and ending about 4 months, about 1 year, about 2 years, about 3 years, about 4 years, or about 5 years after the date of initial administration.

In another embodiment, the disclosure provides use of any composition described herein for treating hypertriglyceridemia in a subject in need thereof, comprising: providing a subject having a fasting baseline triglyceride level of about 135 mg/dL to about 500 mg/dL and administering to the subject a composition as described herein. In one embodiment, the composition comprises about 1 g to about 4 g of eicosapentaenoic acid ethyl ester, wherein the composition contains substantially no docosahexaenoic acid.

In yet another embodiment, the disclosure provides use of any composition described herein for treating hypertriglyceridemia in a subject in need thereof, comprising: providing a subject having a fasting baseline triglyceride level of about 80 mg/dL to about 1500 mg/dL and administering to the subject a composition as described herein. In one embodiment, the composition comprises about 1 g to about 4 g of eicosapentaenoic acid ethyl ester, wherein the composition contains substantially no docosahexaenoic acid.

### EXAMPLES

### Example 1: Impact of Icosapent Ethyl on Reducing Cardiovascular Events in High Risk Statin-Treated Patients

Among patients with cardiovascular risk factors who are receiving treatment for secondary or primary prevention, the rates of cardiovascular events remain high. Even in patients receiving appropriate treatment with statins, a substantial residual cardiovascular risk remains. In such patients, an elevated triglyceride level serves as an independent marker for increased ischemic risk, as shown in epidemiological and mendelian randomization studies. In randomized trials, medications that reduce triglycerides, such as extended-release niacin and fibrates, have not reduced the rates of cardiovascular events when administered in addition to appropriate medical therapy, including statins. Further, contemporary trials and recent meta-analyses of omega-3 fatty acid products have not shown benefit in patients receiving statin therapy. Accordingly, the objective of the present study was to determine if and how icosapent ethyl (referenced interchangeably with AMR101 or VASCEPA®) reduced cardiovascular events in patients with elevated triglyceride levels on a statin therapy.

The following study, also referred to as the REDUCE-IT clinical trial, was a large cardiovascular (CV) outcome trial designed to assess CV risk reduction benefit of AMR101 treatment (commercially known as VASCEPA®) versus placebo on the 5-point primary composite endpoint: CV death, nonfatal stroke, nonfatal myocardial infarction (MI), coronary revascularizations, or unstable angina requiring hospitalization.

A multi-center, prospective, randomized, double-blind, placebo-controlled, parallel-group study was performed to evaluate the effect of AMR101 (4 g per day) on cardiovascular health and mortality in hypertriglyceridemic patients with cardiovascular disease or at high risk for cardiovascular disease. The intended expanded indication of the study was treatment with AMR101 as an add-on to statin therapy to reduce the risk of cardiovascular events in patients with clinical cardiovascular disease or with multiple risk factors for cardiovascular disease.

The primary objective of this study was, in patients at LDL-C goal while on statin therapy, with established cardiovascular disease (CVD) or at high risk for CVD, and hypertriglyceridemia (e.g., fasting triglycerides(TG) ≥200 mg/dL and <500 mg/dL), to evaluate the effect of AMR101 4 g daily on time from randomization to first occurrence of any component of the composite of the following major CV events: CV death; nonfatal MI (including silent MI); electrocardiograms (ECGs) were performed annually for the detection of silent MIs); nonfatal stroke; coronary revascularization; and unstable angina determined to be caused by myocardial ischemia by invasive/non-invasive testing and requiring emergent hospitalization.

The key secondary objective of this study was to evaluate the effect of AMR101 4 g daily on the time from randomization to the first occurrence of the composite of following major CV events: CV death, nonfatal MI (including silent MI), and nonfatal stroke.

Other secondary objectives for this study were to evaluate the effect of therapy on time from randomization to the first occurrence of the following individual or composite endpoints: composite of CV death or nonfatal MI (including silent MI); fatal or nonfatal MI (including silent MI); non-elective coronary revascularization represented as the composite of emergent or urgent classifications; CV death; unstable angina determined to be caused by myocardial ischemia by invasive/non-invasive testing and requiring emergent hospitalization; fatal or nonfatal stroke; composite of total mortality, nonfatal MI (including silent MI), or nonfatal stroke; and total mortality.

The key tertiary objectives for this study were to evaluate the effect of AMR101 4 g daily from baseline and percent change form baseline in fasting triglycerides and LDL-C. Other tertiary objectives for this study were to evaluate the effect of therapy on the following in addition to supporting efficacy and safety analyses:
Total CV events analysis defined as the time from randomization to occurrence of the first and all recurrent major CV events defined as CV death, nonfatal MI (including silent MI), nonfatal stroke, coronary revascularization, or unstable angina determined to be caused by myocardial ischemia by invasive/non-invasive testing and requiring emergent hospitalization;
Primary composite endpoint in the subset of patients with diabetes mellitus at baseline;
Primary composite endpoint in the subset of patients with metabolic syndrome at baseline as defined with waist circumference of ≥35 inches (88 cm) for all women and Asian, Hispanic, or Latino men; and ≥40 inches (102 cm) for all other men;
Primary composite endpoint in the subset of patients with impaired glucose metabolism at baseline (Visit 2 fasting blood glucose (FBG) of 100-125 mg/dL);
Key secondary composite endpoint in the subset of patients with impaired glucose metabolism at baseline (Visit 2 FBG 100-125 mg/dL);
Composite of CV death, nonfatal MI (including silent MI), nonfatal stroke, cardiac arrhythmia requiring hospitalization of ≥24 hours, or cardiac arrest;
Composite of CV death, nonfatal MI (including silent MI), non-elective coronary revascularizations (defined as emergent or urgent classifications), or unstable angina determined to be caused by myocardial ischemia by invasive/non-invasive testing and requiring emergent hospitalization;
Composite of CV death, nonfatal MI (including silent MI), non-elective coronary revascularizations (defined as emergent or urgent classifications), unstable angina determined to be caused by myocardial ischemia by invasive/non-invasive testing and requiring emergent hospitalization, nonfatal stroke, or peripheral vascular disease (PVD) requiring intervention, such as angioplasty, bypass surgery, or aneurysm repair;
Composite of CV death, nonfatal MI (including silent MI), non-elective coronary revascularizations (defined as emergent or urgent classifications), unstable angina determined to be caused by myocardial ischemia by invasive/non-invasive testing and requiring emergent hospitalization, PVD requiring intervention, or cardiac arrhythmia requiring hospitalization of ≥24 hours;
New congestive heart failure (CHF);
New CHF as the primary cause of hospitalization;
Transient ischemic attack (TIA);
Amputation for PVD;
Carotid revascularization;
All coronary revascularizations defined as the composite of emergent, urgent, elective, or salvage;
Emergent coronary revascularizations;
Urgent coronary revascularizations;
Elective coronary revascularizations;
Salvage coronary revascularizations;
Cardiac arrhythmias requiring hospitalization of ≥24 hours;
Cardiac arrest;
Ischemic stroke;
Hemorrhagic stroke;
Fatal or nonfatal stroke in the subset of patients with a history of stroke prior to baseline;
New onset diabetes, defined as Type 2 diabetes newly diagnosed during the treatment/follow-up period;
New onset hypertension, defined as blood pressure ≥140 mmHg systolic or ≥90 mmHg diastolic newly diagnosed during the treatment/follow-up period;
Fasting triglycerides (TG), total cholesterol (TC), low dense lipoprotein cholesterol (LDL-C), high dense lipoprotein cholesterol (HDL-C), non-dense lipoprotein cholesterol (non-HDL-C), very low dense lipoprotein cholesterol (VLDL-C), apolipoprotein B (Apo B), high-sensitivity C-reactive protein (hs-CRP and log[hs-CRP]), high-sensitivity troponin T (hsTnT), and remnant like particle cholesterol (RLP-C; estimated from standard lipid panel, RLP-C=TC−HDL-C−LDL-C [Varbo 2014]), (based on ITT estimands):
   Assessment of the relationship between baseline biomarker values and treatment effects within the primary and key secondary endpoints;
   Assessment of the effect of AMR101 on each marker; and
   Assessment of the relationship between post-baseline biomarker values and treatment effects within the primary and key secondary composite endpoints by including post-baseline biomarker values (for example, at 4 months or at 1 year) as a covariate.
Change from baseline and percent change from baseline in fasting TG, TC, LDL-C, HDL-C, non-HDL-C, VLDL-C, Apo B, hs-CRP, hsTnT, and RLP-C;
Change in body weight; and
Change in waist circumference.

### Study Population

The population for this study were men and women years of age with established CVD, or men and women ≥50 years of age with diabetes in combination with one additional risk factor for CVD. In addition, all patients had atherogenic dyslipidemia defined as on treatment for hypercholesterolemia (but at treatment goal for LDL-C, by treatment with a statin) and hypertriglyceridemia. More details regarding the patient population are listed in the inclusion criteria below. The patients needed to provide consent to participate in the study and were willing and able to comply with the protocol and the study procedures.

### Study Periods

This study consisted of the following study periods:

Screening Period: During the screening period, patients were evaluated for inclusion and exclusion criteria.

At the first visit to the Research Unit (Visit 1), study procedures were performed for evaluation of patient's eligibility in the study. At this screening visit, patients signed an informed consent form before any study procedure was performed; the informed consent form covered the treatment/follow-up period. Based on the evaluation from Visit 1, the following situations occurred:
Patients who were eligible for participation based on the study procedures on Visit 1 returned to the Research Unit for Visit 2 (randomization visit) to start the treatment/follow-up period. This case included, for example, patients at Visit 1 who were on a stable dose of a statin, were planning to stay on the same statin and the same dose of the statin, and who did not need to wash out any non-statin lipid-altering medications.
Patients who were not eligible for participation based on the study procedures on Visit 1 and were unlikely to become eligible in the next 28 days (for example: unlikely to stabilize statin dose, unable to wash out non-statin lipid-altering medications, etc.): these patients were screen failed after Visit 1.
Patients that were not eligible for participation in the study based on the study procedures on Visit 1 could become eligible in the next 28 days: To become eligible, patients returned at the discretion of the investigator for a second optional screening visit (Visit 1.1) at which time the procedures needed for re-evaluation of the previously failed inclusion/exclusion criteria were repeated. This case included, for example, patients who were started on a statin at Visit 1, whose statin dose was changed at Visit 1, and/or needed to wash out non-statin lipid-altering medications. The following applied for these patients:
   Patients with a change in the statin or statin dose on Visit 1 needed to be on a stable statin dose for at least 28 days before the lipid qualifying measurements at Visit 1.1. Other concomitant medications (antidiabetic therapy, for example) could have been optimized or stabilized during this period.
   Patients starting a washout at Visit 1 had a washout period of at least 28 days (only 7 days for bile acid sequestrants) before the lipid qualifying measurements at Visit 1.1.
   Patients at Visit 1 who were on a stable dose of a statin, were planning to stay on the same statin at the same dose, and who did not need any medication washout, but were asked to return for Visit 1.1 to repeat one or more of the other study procedures not related to concomitant medications.
Patients who became eligible for participation based on the additional study procedures at Visit 1.1 returned to the Research Unit for Visit 2 (randomization visit) to start the treatment/follow-up period.

At the end of the screening period, patients needed to meet all inclusion and exclusion criteria before they were randomized. Patients who were not eligible for participation after the screening period (based on study procedures at Visit 1 and/or Visit 1.1) could return at a later date for rescreening. These patients needed to restart with all procedures starting with Visit 1. This included patients who need more time to stabilize one or more conditions or therapies (for example: statin, antidiabetic, antihypertensive, thyroid hormone, HIV-protease inhibitor therapy).

Treatment/Follow-Up Period: Within 42 days after the first screening visit (Visit 1) or within 60 days after the first screening visit (Visit 1) for those patients that had a second screening visit (Visit 1.1), eligible patients entered the treatment/follow-up period. During this period, the patients received the study drug during the planned visits at the Research Site and took the study drug while away from the Research Site.

During the visits, study procedures were performed for evaluation of efficacy and safety. A detailed schedule of the procedures is provided below in Table 1.

TABLE 1
Schedule of Procedures

| | Screening | | | | | | | | | | |
|---|---|---|---|---|---|---|---|---|---|---|---|
| | | If a Visit | | | | | | | | | |
| | | 1.1 takes | | | | | | | | | |
| | | place, | | | | | | | | | |
| | | Visit 1 | | | | | | | | | |
| | | may | | | | | | | | | |
| | | occur up | | | | | | | | | |
| | Up to 42 | to 60 | | | | | | | | | |
| | days | days | Follow-Up (FU)^{[13]} | | | | | | | | |
| | before | before | | 120 ± | 360 ± | 720 ± | 1080 ± | 1440 ± | 1800 + | 2160 ± | Last Visit |
| Study Day | Day 0 | Day 0^{[2]} | 0 | 10 | 10 | 10 | 10 | 10 | 30 | 10 | (LV)^{[15]} |
| Months of FU | | | 0 | 4 | 12 | 24 | 36 | 48 | 60 | 72 | Varies |
| Years of FU | | | 0 | 0.33 | 1 | 2 | 3 | 4 | 5 | 6 | Varies |
| Visit # | 1 | 1.1 | 2 | 3 | 4 | 5 | 6 | 7 | 8 | 9^{[14]} | LV |

Study Procedures:

| | | | | | | | | | | | |
|---|---|---|---|---|---|---|---|---|---|---|---|
| Informed | X | | | | | | | | | | |
| consent | | | | | | | | | | | |
| Medical, | X | | | | | | | | | | |
| surgical & | | | | | | | | | | | |
| family history | | | | | | | | | | | |
| Demographics | X | | | | | | | | | | |
| Evaluate | X^{[1]} | X^{[3]} | X | | | | | | | | |
| inclusion/ | | | | | | | | | | | |
| exclusion | | | | | | | | | | | |
| criteria | | | | | | | | | | | |
| Physical | | | X | X | X | X | X | X | X | X | X |
| examination | | | | | | | | | | | |
| Weight, | X | | X | X | X | X | X | X | X | X | X |
| height^{[4]} | | | | | | | | | | | |
| Vital signs^{[5]} | X | X | X | X | X | X | X | X | X | X | X |
| Waist | | | X | | | X | | | | | X |
| circumference | | | | | | | | | | | |
| 12-Lead ECG | X | | X | | X | X | X | X | X | X | X |
| Urine | X | | X | | | | | | | | |
| pregnancy | | | | | | | | | | | |
| test^{[6]} | | | | | | | | | | | |
| Concomitant | X | X | X | X | X | X | X | X | X | X | X |
| meds | | | | | | | | | | | |
| Randomization | | | X | | | | | | | | |
| Dosing at | | | X | X | X | X | X | X | X | X | |
| the research | | | | | | | | | | | |
| site^{[7]} | | | | | | | | | | | |
| Efficacy events | | | | X | X | X | X | X | X | X | X |
| AE evaluations | | | X | X | X | X | X | X | X | X | X |
| Compliance | | | | X | X | X | X | X | X | X | X |
| check^{[8]} | | | | | | | | | | | |
| Chemistry and | X | X³ | X | X | X | X | X | X | X | X | X |
| hematology^{[9]} | | | | | | | | | | | |
| Fasting lipid | X | X³ | X | X | X | X | X | X | X | X | X |
| profile^{[10]} | | | | | | | | | | | |
| Genetic | | | X | | | | | | | | |
| testing^{[11]} | | | | | | | | | | | |
| Biomarkers: | | | X | | | X | | | | | X |
| hs-CRP, Apo B, | | | | | | | | | | | |
| hsTnT | | | | | | | | | | | |
| Fasting blood | | | X | | X | X | X | X | X | X | X |
| sample | | | | | | | | | | | |
| for archiving ^{[12]} | | | | | | | | | | | |

^{[1]}Includes procedures and (fasting) blood samples (for example, hs-CRP, calculated creatinine clearance) as needed to determine the CV risk category (see inclusion criteria).
^{[2]}Screening visit to re-evaluate inclusion/exclusion criteria for patients who were not eligible for participation based on data from Visit 1.
^{[3]}Inclusion/exclusion criteria were re-evaluated for selected study procedures that were performed on Visit 1.1 because patients failed to meet them at Visit 1.
^{[4]}Height at first screening visit only.
^{[5]}Vital signs, including systolic and diastolic blood pressure (mmHg), heart rate, respiratory rate and body temperature. Participants were seated for at least 5 minutes before assessments of vital signs.
^{[6]}For women of childbearing potential.
^{[7]}The patients fasted at least 10 hours before arriving at the Research Site, when all fasting blood samples were obtained. After blood samples were obtained, patients were given drug with food.
^{[8]}Review study drug compliance by unused capsule count, discussed with and counseled patients about compliance if needed; final study compliance at last visit.
^{[9]}Safety Laboratories - Complete Blood Count: Included RBC, Hgb, Hct, WBC and differential, and platelet count. Biochemistry includes total protein, albumin, alkaline phosphatase, ALT, AST, total bilirubin, glucose, calcium, electrolytes (sodium, potassium, chloride), blood urea nitrogen (BUN), serum creatinine, uric acid, creatine kinase, HbA1c. Safety labs were repeated as deemed necessary by the Investigator.
^{[10]}TG, TC, HDL-C, LDL-C, non-HDL-C, and VLDL-C.
^{[11]}Fasting blood sample were stored for future genetic testing at the discretion of the Sponsor. This sample was optional as local regulations may prohibit genetic samples to be collected or shipped outside the country, or patients may not have consented.
^{[12]} Used at the Sponsor's discretion to perform repeat analyses described in the protocol or to perform other tests related to cardiovascular health.
^{[13]}Site personnel contacted each patient by telephone in-between Visit 2 and Visit 3 and between Visit 3 and Visit 4. After Visit 4, contact was made every 3 months. The purpose of the contact was to collect information about efficacy events, adverse events, concomitant medications, confirm patient's current address and contact information, and remind patients about taking their study medication and logistics for the next visit.
^{[14]}Office visits continued at 360-day intervals and phone visits at 90-day intervals until study end date was determined.
^{[15]}The last visit (LV) could have occurred within 30 days after the study end date as determined by the DMC; the study end date is tentatively schedule for Day 2160 but the actual date was determined by the DMC may be different.

### Study Duration

Patients were randomized at different times during the enrollment period, but all ended the study at approximately the same date (i.e., at the study end date) and, therefore, the duration of follow-up differed based on date of randomization. It was planned that all randomized patients received study medication and were followed up until the study end date. It was expected that a minimum of approximately 1612 primary endpoint events were required during the study. 8179 patients were randomized at multiple Research Sites worldwide over a period of approximately 4.2 years. After randomization, patients were treated and followed up to an estimated maximum of 6.5 years. The study end date was determined to be when approximately 1612 primary efficacy events had been adjudicated. Table 2 shows the study milestones from the first patient screened to the last patient visit and subsequent database lock.

TABLE 2
Study Milestones

| Study Milestones | Date |
|---|---|
| First Patient Screened | 21 Nov. 2011 |
| First Patient Randomized | 28 Nov. 2011 |
| Last Patient Randomized | 4 Aug. 2016 |
| SAP Finalization | 8 Jul. 2016 |
| First DMC Interim Efficacy Review | 9 Sep. 2016 |
| Second DMC Interim Efficacy Review | 11 Aug. 2017 |
| First Patient Last Visit | 1 Mar. 2018 |
| Last Patient Last Visit | 31 May 2018 |
| Database Lock | 6 Sep. 2018 |

### Study Groups

At Visit 2 (Day 0), eligible study patients were randomly assigned to the following treatment groups:
Group 1: AMR101 (>96% E-EPA) 4 g daily (four 1000 mg capsules daily)
Group 2: placebo (four capsules daily)

The four AMR101 or placebo capsules daily were taken as two capsules in the morning and two capsules in the evening (twice-per-day dosing regimen).

### Number of Patients

This was an event-driven trial and it was expected that a minimum of 1612 primary efficacy endpoint events were required during the study. A total of approximately 8179 patients entered into the study to either receive AMR101 or placebo (approximately 4089 patients per treatment group) in order to observe an estimated 1612 events that made up the primary composite endpoint for efficacy.

### Randomization

On Day 0, eligible patients were randomized to one of the 2 study groups using a computer-generated randomization schema. Randomized treatment assignment to either AMR101 or placebo in a 1:1 ratio was provided using the internet (IWR).

### Blinding

This was a double-blind study. Patients, investigators, pharmacists and other supporting staff at the Research Sites; personnel and designees of the Sponsor, study administrators, and personnel at the organization(s); and vendors supporting the study were unaware of the randomization code (i.e., they did not know which study participants were receiving the experimental drug and which were receiving the placebo drug). The study medication AMR101 and placebo capsules were similar in size and appearance to maintain blinding.

During the double-blind treatment/follow-up period, everyone (patients, investigators, pharmacists and other supporting staff at the Research Sites; personnel and designees of the Sponsor, study administrators, and personnel at the organization(s); and vendors managing/supporting the study), with the exception of the laboratory personnel performing the analysis, were blinded to individual results of the efficacy laboratory measurements (including lipid values). Individual results from the lipid profile could be unblinded in the event of an emergency for a patient.

### Stratification

Participants were assigned to treatment groups stratified by CV risk category, use of ezetimibe, and by geographical region (e.g., Westernized, Eastern European, and Asian Pacific countries). There were two CV risk categories:
CV Risk Category 1: patients with established CVD defined in the inclusion criteria. Patients with diabetes and established CVD were included in this category. These patients are defined as the secondary prevention stratum, primary risk category, and/or secondary prevention cohort.
CV Risk Category 2: patients with diabetes and at least one additional risk factor for CVD, but no established CVD. These patients are defined as the primary prevention stratum, secondary risk category, and/or primary prevention cohort.

Stratification was recorded in the IWR at the time of enrollment. Approximately 70% of randomized patients were in the CV Risk Category 1 and approximately 30% of randomized patients were in the CV Risk Category 2. Enrollment with patients of a CV risk category was stopped when the planned number of patients in that risk category was reached.

### Study Population

Inclusion Criteria: A detailed list of the inclusion criteria for this study is provided in Tables 3-5. Specifically, Table 3 outlines the inclusion criteria for patients in this study whereas Tables 4 and 5 further outline the inclusion criteria based on whether that patient is part of the primary prevention risk category or the secondary prevention risk category of patients, respectively.

TABLE 3
Patient Inclusion Criteria
Study Inclusion Criteria

| | |
|---|---|
| 1 | Men or women ≥45 years of age with established CVD (i.e., |
| | Primary Prevention Risk Category; see Table 4) or ≥50 years |
| | of age with diabetes in combination with one additional risk |
| | factor for CVD (i.e., Secondary Prevention Risk Category; |
| | see Table 5). |
| 2 | Fasting TG levels ≥150 mg/dL (2.26 mmol/L) and <500 mg/dL |
| | (5.64 mmol/L). Due to the variability of triglycerides, a 10% |
| | allowance existed in the initial protocol, which permitted |
| | patients to be enrolled with qualifying triglyceride |
| | levels ≥135 mg/dL. Protocol amendment made in May of 2013 |
| | changed the lower limit of acceptable triglyceride levels |
| | from 150 mg/dL to 200 mg/dL, with no variability allowance. |
| 3 | LDL-C >40 mg/dL and ≤100 mg/dL and on stable statin therapy |
| | (±ezetimibe) for ≥4 weeks prior to the LDL-C and TG |
| | qualifying measurements for randomization. |
| 4 | Women who are not pregnant, not breastfeeding, not planning on |
| | becoming pregnant, and using an acceptable form of birth control |
| | during the study (if of child-bearing potential), unless their sexual |
| | partner(s) were surgically sterile or the woman was abstinent. Women |
| | of child bearing potential needed a negative urine pregnancy test |
| | prior to randomization. |
| 5 | Able to provide informed consent and adhere to study schedules. |
| 6 | Agree to follow and maintain a physician-recommended diet during |
| | the study. |

Stable therapy was defined as the same daily dose of the same statin for at least 28 days before the lipid qualification measurements (TG and LDL-C) and, if applicable, the same daily dose of ezetimibe for at least 28 days before the lipid qualification measurements (TG and LDL-C). Patients who had their statin therapy or use of ezetimibe initiated at Visit 1, or had their statin, statin dose, and/or ezetimibe dose changed at Visit 1, needed to go through a stabilization period of at least 28 days since initiation/change and had their qualifying lipid measurements measured (TG and LDL-C) after the washout period (at Visit 1.1). Statins may have been administered with or without ezetimibe.

If patients qualified at the first qualification visit (Visit 1) for TG and LDL-C and met all other inclusion/exclusion criteria, they were randomized at Visit 2. If patients did not qualify at the first qualifying visit (Visit 1), a second re-qualifying visit (Visit 1.1) was allowed. For some patients, because they needed to stabilize medications and/or needed to washout medications, the second re-qualifying visit (Visit 1.1) was needed after the stabilization/washout period.

Women were not considered to be of childbearing potential if they met one of the following criteria as documented by the investigator: they had a hysterectomy, tubal ligation or bilateral oophorectomy prior to signing the informed consent form; and/or they were post-menopausal, defined as year since their last menstrual period or had a follicle-stimulating hormone (FSH) level in a menopausal range.

Patients having established CVD (in CV Risk Category 1) were defined as detailed in Table 4.

TABLE 4
Inclusion Criteria for the Primary Prevention
Risk Category (CV Risk Category 1)
Primary Prevention Risk Category (i.e., Secondary Prevention Cohort)
Defined as men and women ≥45 years of
age with one or more of the following:

| | |
|---|---|
| 1 | Documented coronary artery disease (CAD; one or more of the |
| | following primary criteria must be satisfied): |

| | |
|---|---|
| a. | Documented multi vessel CAD (≥50% stenosis in |
| | at least two major epicardial coronary arteries - |
| | with or without antecedent revascularization). |
| b. | Documented prior MI. |
| c. | Hospitalization for high-risk non-ST-segment |
| | elevation acute coronary syndrome (NSTE-ACS) |
| | (with objective evidence of ischemia: ST-segment |
| | deviation or biomarker positivity). |

| | |
|---|---|
| 2 | Documented cerebrovascular or carotid disease (one of the following |
| | primary criteria must be satisfied): |

| a. | Documented prior ischemic stroke. |
|---|---|
| b. | Symptomatic carotid artery disease with ≥50% carotid |
| | arterial stenosis. |
| c. | Asymptomatic carotid artery disease with ≥70% carotid |
| | arterial stenosis per angiography or duplex ultrasound. |
| d. | History of carotid revascularization (catheter-based |
| | or surgical). |

| | |
|---|---|
| 3 | Documented peripheral arterial disease (PAD; one or more of the |
| | following primary criteria must be satisfied): |

| | |
|---|---|
| a. | Ankle-brachial index (ABI) <0.9 with symptoms of |
| | intermittent claudication. |
| b. | History of aorto-iliac or peripheral arterial intervention |
| | (catheter-based or surgical). |

Patients at high risk for CVD (in CV Risk Category 2) were defined as detailed in Table 5.

TABLE 5
Inclusion Criteria for the Secondary Prevention
Risk Category (CV Risk Category 2)
Secondary Prevention Risk Category (i.e., Primary Prevention Cohort)
Defined as having each of the following:

| | |
|---|---|
| 1 | Diabetes mellitus (Type 1 or Type 2) requiring treatment with |
| | medication. |
| 2 | Men and women ≥50 years of age. |
| 3 | One of the following at Visit 1 (additional risk factor for CVD): |

| | |
|---|---|
| a. | Men ≥55 years of age and Women ≥65 years of age. |
| b. | Cigarette smoker or stopped smoking within 3 months |
| | before Visit 1. |
| c. | Hypertension (blood pressure ≥140 mmHg systolic |
| | OR ≥90 mmHg diastolic) or on antihypertensive |
| | medication. |
| d. | HDL-C ≤40 mg/dL for men or ≤50 mg/dL for women. |
| e. | hs-CRP >3.00 mg/L (0.3 mg/dL). |
| f. | Renal dysfunction: Creatinine clearance (CrCL) >30 |
| | and <60 mL/min. |
| g. | Retinopathy, defined as any of the following: non- |
| | proliferative retinopathy, pre-proliferative retinopathy, |
| | proliferative retinopathy, maculopathy, advanced diabetic |
| | eye disease or a history of photocoagulation. |
| h. | Micro- or macroalbuminuria. Microalbuminuria is defined |
| | as either a positive micral or other strip test (may be |
| | obtained from medical records), an albumin/creatinine |
| | ratio ≥2.5 mg/mmol or an albumin excretion rate on timed |
| | collection ≥20 mg/min all on at least two successive |
| | occasions; macroalbuminuria, defined as Albustix or other |
| | dipstick evidence of gross proteinuria, an albumin/ |
| | creatinine ratio ≥25 mg/mmol or an albumin excretion rate |
| | on timed collection ≥200 mg/min all on at least two |
| | successive occasions. |
| i. | ABI <0.9 without symptoms of intermittent claudication |
| | (patients with ABI <0.9 with symptoms of intermittent |
| | claudication are counted under Secondary Prevention |
| | Risk Category). |

Patients with diabetes and CVD as defined above are eligible based on the CVD requirements and will be counted under CV Risk Stratum 1. Only patients with diabetes and no documented CVD as defined above needed at least one additional risk factor as listed, and were counted under Primary Prevention Risk Category.

Exclusion Criteria: Patients meeting the following exclusion criteria enumerated in Table 6 were not eligible for the study.

TABLE 6
Patient Exclusion Criteria
Study Exclusion Criteria

| | |
|---|---|
| 1 | Severe (New York Heart Association [NYHA] class IV) heart failure. |
| 2 | Any life-threatening disease expected to result in death within the next 2 years (other than CVD). |
| 3 | Diagnosis or laboratory evidence of active severe liver disease. |
| 4 | Hemoglobin A1c >10.0% (or 86 mmol/mol IFCC units) at screening (Visit 1). If patients failed this |
| | criterion (HbA1c >10.0% or 86 mmol/mol IFCC units) at Visit 1, they could have had their antidiabetic |
| | therapy optimized and be retested at Visit 1.1. |
| 5 | Poorly controlled hypertension: systolic blood pressure (SBP) ≥200 mmHg or diastolic blood pressure |
| | (DBP) ≥100 mmHg (despite antihypertensive therapy). |
| 6 | Planned coronary intervention or any non-cardiac major surgical procedure. |
| 7 | Known familial lipoprotein lipase deficiency (Fredrickson Type I), apolipoprotein C-II deficiency, or |
| | familial dysbetalipoproteinemia (Fredrickson Type III). |
| 8 | Participation in another clinical trial involving an investigational agent within 90 days prior to screening |
| | (Visit 1). Patients could not participate in any other investigational medication or medical device trial |
| | while participating in this study (participation in a registry or observational study without an additional |
| | therapeutic intervention was allowed). |
| 9 | Intolerance or hypersensitivity to statin therapy. |
| 10 | Known hypersensitivity to fish and/or shellfish, or ingredients of the study product or placebo. |
| 11 | History of acute or chronic pancreatitis. |
| 12 | Malabsorption syndrome and/or chronic diarrhea. (Note: patients who had undergone gastric/intestinal |
| | bypass surgery were considered to have malabsorption, hence were excluded; patients who had |
| | undergone gastric banding were allowed to enter the trial). |
| 13 | Use of non-study drug-related, non-statin, lipid-altering medications, dietary supplements, or foods |
| | during the screening period (after Visit 1) and/or plans for use during the treatment/follow-up period |
| | including: |

| | |
|---|---|
| a. | niacin >200 mg/day or fibrates during the screening period (after Visit 1) and/or planned to use |
| | during the study; patients who were taking niacin >200 mg/day or fibrates during the last 28 |
| | days before Visit 1 needed to go through washout of at least 28 days after their last use and |
| | have their qualifying lipids measured (TG and LDL-C) after the washout period (Visit 1.1). |
| b. | any omega-3 fatty acid medications (prescription medicines containing EPA and/or DHA) during |
| | the screening period (after Visit 1) and/or planned to use during the treatment/follow-up period |
| | of the study. To be eligible for participation in the study, patients who were taking omega-3 fatty |
| | acid medications during the last 28 days before Visit 1 (except patients in the Netherlands), |
| | needed to go through a washout period of at least 28 days after their last use and have their |
| | qualifying lipids measured (TG and LDL-C) after the washout period (at Visit 1.1). However, for |
| | patients in the Netherlands only being treated with omega-3 fatty acid medications containing |
| | EPA and/or DHA were excluded and no washout was allowed. |
| c. | dietary supplements containing omega-3 fatty acids (e.g., flaxseed, fish, krill, or algal oils) during |
| | the screening period (after Visit 1) and/or planned to use during the treatment/follow-up period |
| | of the study. To be eligible for participation in the study, patients who were taking >300 mg/day |
| | omega-3 fatty acids (combined amount of EPA and DHA) within 28 days before Visit 1 (except |
| | patients in the Netherlands), needed to go through a washout period of at least 28 days since |
| | their last use and have their qualifying lipid measurements measured (TG and LDL-C) after the |
| | washout period (at Visit 1.1). However, for patients in the Netherlands only being treated with |
| | dietary supplements containing omega-3 fatty acids of >300 mg/day EPA and/or DHA were |
| | excluded and no washout was allowed. |
| d. | bile acid sequestrants during the screening period (after Visit 1) and/or planned to use during |
| | the treatment/follow-up period of the study. To be eligible for participation in the study, patients |
| | who were taking bile acid sequestrants within 7 days before Visit 1 needed to go through a |
| | washout period of at least 7 days since their last use and have their qualifying lipid |
| | measurements measured (TG and LDL-C) after the washout period (at Visit 1.1). |
| e. | proprotein convertase subtilisin kexin 9 (PCSK9) inhibitors during the screening period (after |
| | Visit 1) and/or planned to use during the treatment/follow-up period of the study. To be eligible |
| | for participation in the study, patients could not have taken a PCSK9 inhibitor within 90 days |
| | prior to their screening visit. |

| | |
|---|---|
| 14 | Other medications (not indicated for lipid alteration): |

| a. | Tamoxifen, estrogens, progestins, thyroid hormone therapy, systemic corticosteroids (local, |
|---|---|
| | topical, inhalation, or nasal corticosteroids are allowed), HIV-protease inhibitors that have not |
| | been stable for ≥28 days prior to the qualifying lipid measurements (TG and LDL-C) during |
| | screening. To be eligible for participation in the study, patients who were not taking a stable |
| | dose of these medications within 28 days before Visit 1, needed to go through a stabilization |
| | period of at least 28 days since their last dose change and have their qualifying lipid |
| | measurements measured (TG and LDL-C) after the washout period (at Visit 1.1). |
| b. | Cyclophosphamide or systemic retinoids during the screening period (unless ≥28-day washout) |

and/or plans for use during the treatment/follow-up period. To be eligible for participation in the
study, patients who were taking these medications within 28 days before Visit 1, needed to go
through a washout period of at least 28 days since their last use and have their qualifying lipid
measurements measured (TG and LDL-C) after the washout period (at Visit 1.1).

| | |
|---|---|
| 15 | Known AIDS (HIV-positive patients without AIDS are allowed). |
| 16 | Requirement for peritoneal dialysis or hemodialysis for renal insufficiency or creatinine clearance <30 |
| | mL/min (0.50 mL/sec). |
| 17 | Unexplained elevated creatine kinase concentration >5 × ULN or elevation due to known muscle |
| | disease (e.g., polymyositis, mitochondrial dysfunction) at Visit 1. |
| 18 | Any condition or therapy which, in the opinion of the investigator, might pose a risk to the patient or |
| | make participation in the study not in the patient's best interest. |
| 19 | Drug or alcohol abuse within the past 6 months, and inability/unwillingness to abstain from drug abuse |
| | and excessive alcohol consumption during the study or drinking 5 units or more for men or 4 units or |
| | more for women in any one hour (episodic excessive drinking or binge drinking). Excessive alcohol |
| | consumption was on average >2 units of alcohol per day. A unit of alcohol was defined as a 12-ounce |
| | (350 mL) beer, 5-ounce (150 mL) wine, or 1.5-ounce (45 mL) of 80-proof alcohol for drinks. |
| 20 | Mental/psychological impairment or any other reason to expect patient difficulty in complying with the |
| | requirements of the study or understanding the goal and potential risks of participating in the study |
| | (evaluated at Visit 1). |

### Study Procedures

The Screening Period for this study included two visits, Visit 1 and Visit 1.1.

Screening Visit (Visit 1): During Visit 1, patients came to the Research Site and were instructed to fast for at least 10 hours before their visit. If patients qualified for randomization based on the procedures at Visit 1, they needed to be randomized within 42 days after Visit 1. The following procedures were performed at the screening Visit 1:
Obtained signed informed consent;
Assigned the patient a patient number;
Obtained medical, surgical, and family history;
Recorded demographics;
Obtained height, weight, and body mass index;
Obtained vital signs (systolic and diastolic blood pressure, heart rate, respiratory rate, and body temperature);
Obtained a 12-lead electrocardiogram;
Evaluated inclusion/exclusion criteria;
This included procedures and (fasting) blood samples (for example, hs-CRP, calculated creatinine clearance) as needed to determine the CV risk category (See inclusion criteria);
Obtained fasting blood samples for chemistry and hematology testing;
Obtained a fasting blood sample for the lipid profile (TG, TC, HDL-C, LDL-C, non-HDL-C, VLDL-C);
Performed a urine pregnancy test on women of childbearing potential;
Recorded concomitant medication(s); and
Instructed patient to fast for at least 10 hours prior to the next visit.

Screening Visit (Visit 1.1): Patients who qualified for study participation after Visit 1 because they met all inclusion criterion and none of the exclusion criteria, skipped Visit 1.1 and returned to the Research Site for Visit 2 to be randomized and to start the treatment/follow-up period of the study. For these patients, Visit 2 occurred soon after Visit 1. Patients who did not qualify at Visit 1 returned to the Research Site for a second qualifying visit (Visit 1.1) at the discretion of the investigator. At Visit 1.1, procedures that caused failure of eligibility at Visit 1 were repeated. Patients were eligible for randomization after Visit 1.1 if they met all inclusion criteria and if they no longer failed the exclusion criteria. If patients were evaluated at Visit 1.1 and qualified for randomization based on the repeated procedures at Visit 1.1, they needed to be randomized within 60 days after Visit 1. For some patients, Visit 1.1 was mandatory at least 28 days after Visit 1 in order to check eligibility. These were patients who at Visit 1 started treatment with a statin, changed their statin, changed the daily dose of their statin, started to washout prohibited medications or started a stabilization period with certain medications. (See inclusion/exclusion criteria above for details.) Any of these changes at Visit 1 may have affected the qualifying lipid levels and therefore, patients needed to have Visit 1.1 to determine whether they qualified based on lipid level requirements (TG and LDL-C) determined at Visit 1. Other procedures that caused failure of eligibility at Visit 1 were also repeated at Visit 1.1. The following procedures were performed at the screening Visit 1.1:
Obtained vital signs (systolic and diastolic blood pressure, heart rate, respiratory rate, and body temperature);
Evaluated inclusion/exclusion criteria; only those evaluations were repeated that deemed the patient not eligible on Visit 1;
Obtained fasting blood samples for chemistry and hematology testing. Only those samples were obtained that deemed the patient not eligible on Visit 1;
Obtained a fasting blood sample for the lipid profile (TG, TC, HDL-C, LDL-C, non-HDL-C, VLDL-C) if the patient was deemed not eligible on Visit 1. This included patients who at Visit 1 started treatment with a statin, changed their statin, changed the daily dose of their statin, started to washout prohibited medications or started a stabilization period with certain medications. (See inclusion/exclusion criteria for details.) These patients had a fasting blood sample collected at Visit 1.1 for the qualifying lipid values (TG and LDL-C), and the TG and LDL-C inclusion criteria were evaluated; and
Recorded concomitant medication(s).

The treatment/follow-up period for this study included Visit 2, Visit 3, and Visits 4-9. Every attempt was made to complete the follow-up visits during the defined window periods.

Randomization visit (Visit 2; Day 0): Qualified patients returned to the Research Site for Visit 2. The following procedures were performed at Visit 2:
Performed physical examination;
Obtained weight;
Obtained vital signs (systolic and diastolic blood pressure, heart rate, respiratory rate, and body temperature);
Measured waist circumference (one of the factors to diagnose metabolic syndrome);
Obtained a 12-lead electrocardiogram;
Evaluated inclusion/exclusion criteria;
Obtained fasting blood samples for:
   Chemistry and hematology testing;
   Lipid profile (baseline);
   Biomarker assays (baseline);
   Genetic testing (optional blood sample); and
   Archiving (in countries and at sites approved by international review board (IRB)/independent ethics committee (IEC) and dependent on country regulations).
Performed a urine pregnancy test on women of childbearing potential (must be negative for randomization);
Dispensed study drug and record randomization number;
Instructed patient on how to take study drug;
Administered study drug—Note: Study drug was taken orally with food following the collection of all fasting blood samples;
Assessed and recorded adverse events;
Recorded concomitant medication(s); and
Instructed patient:
   To bring all study supplies with them to the next visit;
   Not to take study drug on the morning of their next visit; and
   To fast for at least 10 hours prior to the next visit.

Visit 3 (Day 120; ˜4 Months): Patients returned to the Research Site for Visit 3 on Day 120±10 days. The following procedures were performed:
Physical examination;
Obtained weight;
Obtained vital signs (systolic and diastolic blood pressure, heart rate, respiratory rate, and body temperature);
Obtained fasting blood samples for:
   Chemistry and hematology testing; and
   Lipid profile.
Reviewed study drug compliance by unused capsule count; discussed with and counseled patients about compliance if needed;
Administered study drug—Note: Study drug should be taken orally with food following the collection of all fasting blood samples;
Assessed and recorded efficacy events;
Assessed and recorded adverse events;
Recorded concomitant medication(s);
Instructed patient:
   To bring all study supplies with them to the next visit;
   Not to take study drug on the morning of their next visit; and
   To fast for at least 10 hours prior to the next visit.

Visits 4, 5, 6, 7, 8, and 9: At Visit 4: Day 360±10; Visit 5: Day 720±10; Visit 6: Day 1080±10; and Visit 7: Day 1440±10: Visit 8: Day 1800±10, Visit 9: Day 2160±10, the following procedures were performed:
Physical examination;
Obtained weight;
Obtained vital signs (systolic and diastolic blood pressure, heart rate, respiratory rate, and body temperature);
Measured waist circumference (collected at Visit 5 only);
Obtained a 12-lead electrocardiogram;
Obtained fasting blood samples for:
   Chemistry and hematology testing;
   Lipid profile;
   Biomarker assays (collected at Visit 5 only); and
   Archiving (in countries and at sites approved by IRB/IEC and dependent on country regulations);
Reviewed study drug compliance by unused capsule count; discussed with and counseled patients about compliance if needed;
Administered study drug—Note: Study drug should be taken orally with food following the collection of all fasting blood samples;
Assessed and recorded efficacy events;
Assessed and recorded adverse events;
Recorded concomitant medication(s); and
Instructed patient:
   To bring all study supplies with them to the next visit;
   Not to take study drug on the morning of their next visit; and
   To fast for at least 10 hours prior to the next visit.

Additional Visits: The end date of the study was expected for Day 2160, but the actual end date was dependent on the determination of the study end date by the DMC and when approximately 1612 primary efficacy events had occurred. If the actual study end date was later than the expected end date, additional visits were planned between Visit 7 and the Last Visit with a maximum of 360±10 days between visits. If the actual study end date was sooner than the expected end date, fewer visits occurred, and the last visit (See below, section titled Last Visit—End of Study) occurred sooner. On additional visits, the same procedures were performed. Irrespective of the number of additional visits, after the DMC had established the end of the study date, there was a last visit with procedures as listed below in section titled Last Visit—End of Study.

Last Visit—End of Study: All patients completed the study at the same time (within a 30-day window after the study end date), irrespective of the date that they were randomized. The end date of the study was planned for Day 2160, but the actual end date was dependent on the determination of the study end date by the DMC when approximately 1612 primary efficacy events had occurred (event-driven trial). For each patient, the last visit may have occurred within 30 days after the actual study end date as determined by the DMC. However, for the efficacy endpoints based on CV events, only events occurring up to and including the scheduled actual study end date were included in the efficacy analyses. A final follow-up visit was required for all patients. In a rare case that a final follow-up visit did not occur within the 30-day timeframe following the study end date, any attempt to contact the patient was recorded on a special contact form, until/unless appropriate information was obtained. At the Last Visit, the following procedures were performed:
Physical examination;
Obtained weight;
Obtained vital signs (systolic and diastolic blood pressure, heart rate, respiratory rate, and body temperature);
Measured waist circumference;
Obtained a 12-lead electrocardiogram;
Obtained fasting blood samples for:
   Chemistry and hematology testing;
   Lipid profile;
   Biomarker assays; and
   Archiving (in countries and at sites approved by IRB/IEC and dependent on country regulations).
Determined study drug compliance by unused capsule count;
Assessed and recorded efficacy events;
Assessed and recorded adverse events; and
Recorded concomitant medication(s).

Telephoned Follow-up Contact: Site personnel contacted each patient by telephone on the following study days: Day 60±3 days; Day 180±5 days; Day 270±5 days; Day 450±5 days; Day 540±5 days; Day 630±5 days; Day 810±5 days; Day 900±5 days; Day 990±5 days; Day 1170±5 days; Day 1260±5 days; Day 1350±5 days; Day 1530±5 days; Day 1620±5 days; Day 1710±5 days; Day 1890±5 days; Day 1980±5 days; and Day 2070±5 days.

If the treatment/follow-up period of the study was extended beyond the expected end date (Day 2160), additional follow-up phone calls were made every 3 months in-between additional visits ±5 days. If the treatment/follow-up period of the study was shorter than the expected end date, less follow-up phone calls were needed. Every attempt was made to talk to each patient within this timeframe. The following information was collected from the patient:
Possible efficacy endpoints related to CV events. Patients were asked to return to the Research Site to assess for any endpoints or events identified;
Adverse events;
Concomitant medications; and
Current address and contact information.

Patients were reminded about the following items:
To take the study medication according to the dosing schedule assigned, with food;
When to return to the Research Center for the next visit;
To bring the unused study medication to the next visit;
To not take study drug on the morning of their next visit; and
To fast for at least 10 hours prior to the next visit.

### Laboratory Procedures

Clinical Laboratory Procedures and Evaluations: All clinical laboratory determinations for screening and safety were performed by a certified clinical laboratory under the supervision of the Sponsor or its designee. Whenever possible and appropriate, samples for the clinical laboratory procedures were collected after fasting for at least 10 hours. For the purposes of this study, fasting was defined as nothing by mouth except water (and any essential medications). The investigator reviewed and signed all laboratory test reports. At screening, patients who had laboratory values that were outside the exclusionary limits specified in the exclusion criteria were not enrolled in the study (patients would have been considered for the study if values were classified as not clinically significant by the investigator). After randomization, the investigator was notified if laboratory values were outside of their normal range. In this case, the investigator was required to conduct clinically appropriate follow-up procedures.

Safety Laboratory Tests: The safety parameters were analyzed by a certified clinical laboratory at screening (Visit 1 or Visit 1.1), Randomization visit (Visit 2; Day 0), Visit 3 (Day 120; ˜4 Months), and all other follow-up visits including the Last Visit. The safety laboratory tests included:
Hematology with complete blood count (CBC), including RBC, hemoglobin (Hgb), hematocrit (Hct), white cell blood count (WBC), white cell differential, and platelet count; and
Biochemistry panel including total protein, albumin, alkaline phosphatase, alanine am inotransferase (ALT/SGPT), aspartate am inotransferase (AST/SGOT), total bilirubin, glucose, calcium, electrolytes (sodium, potassium, chloride), blood urea nitrogen (BUN), serum creatinine, uric acid, creatine kinase, and HbA1c.

Each laboratory result was classified as low (L), normal (N), and high (H) at each visit according to the laboratory-supplied normal range. The shift from baseline was presented for each post-baseline visit and overall post-baseline visits. If multiple measurements for a test parameter were available for a post-baseline patient-visit, the most extreme value was included in the shift table. For shift from baseline to overall post-baseline visits, values from all visits (including unscheduled measurements) were included. The chemistry shift table included fasting lipid parameters. The continuous lipid values were presented as part of the efficacy analysis.

Fasting Lipid Profile: The fasting lipid panel included: TG, TC, LDL-C, HDL-C, non-HDL-C, and VLDL-C. At all visits, LDL-C was calculated using the Friedewald equation. At Visit 1 and Visit 1.1, direct LDL-C was used if at the same visit TG was greater than 400 mg/dL (4.52 mmol/L). These LDL-C values were used for the evaluation of the LDL-C inclusion criterion (LDL-C qualifying measurements for randomization) and for the assessment of changes in the statin therapy when LDL-C was not at goal. At all remaining visits (except Visit 2 and Visit 4), LDL-C was measured by direct LDL cholesterol or by preparative ultracentrifugation if at the same visit TG was greater than 400 mg/dL (4.52 mmol/L). In addition, irrespective of the TG levels, at Visit 2 (0 Months of Follow-up, baseline) and at Visit 4 (12 Months of Follow-up), LDL-C was measured by preparative ultracentrifugation. These preparative ultracentrifugation LDL-C measurements were used in the statistical analysis including the calculation of the percent change from baseline (1 year versus baseline). Hopkins LDL-C was calculated for each visit.

Genetic Testing: A fasting blood sample was stored for future genetic testing at the discretion of the Sponsor. The specifics of this test were determined at a later date. This sample was optional as local regulations may prohibit genetic samples to be collected or shipped outside the country, or patients may not have consented. Research on genetic testing looked for links between genes and certain diseases, including their treatment(s) such as medicines and medical care. The blood samples were collected in the study center with the regular protocol-required labs. Each patient tube with a sample for genetic testing was labeled with patient number only. The site maintained a Subject Code Identification List for cross-reference. The patient number did not contain any identifiable information (i.e., patient initials, date of birth, etc.). Un-analyzed samples were stored frozen by the Sponsor for a period of up to 2 years following the end of the study, at which time they were destroyed. If samples were tested, results were not reported to the patient, parents, relatives, or attending physician and were not recorded in the patient's medical records. There was no follow-up contact with the sites or patients regarding this sample. The subject could withdraw their consent for genetic testing at any time up to analysis, even after the sample had been obtained. The subject could notify the site in writing that they had withdrawn their consent for the genetic testing portion of the study, and it was documented by the site in the subject chart, as well as captured in the CRF. The lab was notified to pull the sample and destroy it. Potential genetic bioassays may have been performed and may have been as broad as a genome-wide association study (GWAS) or as limited as a single gene-target approach; potential target genes included but were not limited to the genes encoding: Apo C3, Apo A5, CETP, LPL, PCSK9, TNFα, TNFβ, ALOX5, COX2, FABP, haptoglobin 1, and haptoglobin 2.

Biomarkers Assays: The biomarker assays included: hs-CRP, Apo B, and hsTnT.

Additional laboratory tests: Additional laboratory tests were performed and included:
A urine pregnancy test was administered to women of childbearing potential at certain visits as listed in schedule of procedures (Table 1). The urine pregnancy tests were performed at the Research Site utilizing marketed test kits, or at a certified clinical laboratory;
A fasting blood sample (10 mL) for archiving. This sample was collected only at sites in countries where allowed by local regulations and at sites for which approved by the IRB or IEC. The plasma from the archiving sample was stored frozen in 2 separate equal aliquots, and was used at the Sponsor's discretion to perform repeat analyses described in the protocol or to perform other tests related to cardiovascular health; and
Potential non-genetic bioassays were performed, including but not limited to: Apo A1, Apo C3, Apo E, NMR lipid profile (particle size and number), oxidized LDL, Lp(a), Lp-PLA₂, serum fatty-acids concentrations, and gamma-glutamyltransferase (GGT).

Blinding of Laboratory Results: All efficacy laboratory results during the double-blind period of the trial were blinded (values not provided) to patients, investigators, pharmacists, and other supporting staff at the Research Sites; personnel and designees of the Sponsor; study administrators and personnel at the organization(s); and vendors managing and/or supporting the study, with the exception of the laboratory personnel conducting the assays. To ensure patient safety, hsTnT values were reported to the site.

Flagging of Critical Lab Values: Critical lab values are values that may have warranted medical intervention to avoid possible harm to a patient. Critical lab values were defined in the Laboratory Manual for the study, and the Research Site was notified of the occurrence of a critical lab value (critical high or critical low) by a special annotation (flag) in the laboratory reports provided to the Research Sites. Although laboratory values that were part of the efficacy endpoints during the double-blind period of the study were not provided to the Research Site, the sites were notified when the TG value of a patient sample was greater than 1000 mg/dL (11.29 mmol/L) (critical high TG value) or if the LDL-C values of a patient sample were greater than 130 mg/dL (3.37 mmol/L) (critical high LDL-C value). These critical high values were confirmed by a repeat measurement (new fasting blood sample) within 7 days. TG values of greater than 2000 mg/dL (22.58 mmol/L) were also flagged so that appropriate medical action could be taken by the investigator as soon as possible.

If TG values were confirmed critically high, patients could be discontinued from the study drug with the option to remain on study. The investigator used the best clinical judgment for each patient which included the use of approved TG-lowering medications after patients had discontinued from study drug. If LDL-C values were confirmed critically high, the investigator needed to take appropriate medical action which included: reinforcing/intensifying therapeutic lifestyle changes (including diet and physical activity), increasing the dose of the present statin therapy, adding ezetimibe, or prescribing a more potent statin to lower LDL-C. The investigator used the best clinical judgment for each patient.

### Medical Procedures

Medical, Surgical and Family History: Medical history, including family history and details regarding all illnesses and allergies, date(s) of onset, status of current condition, and smoking and alcohol use were collected on all patients.

Demographics: Demographic information including day, month, and year of birth, race, and gender were collected for all patients.

Vital Signs and Patient Measurements: Vital signs included systolic and diastolic blood pressure, heart rate, respiratory rate, and body temperature. Blood pressure was measured using a standardized process:
Patient sat for at least 5 minutes with feet flat on the floor and measurement arm supported so that the midpoint of the manometer cuff was at heart level; and
Used a mercury sphygmomanometer or automatic blood pressure device with an appropriately sized cuff with the bladder centered over the brachial artery.

Blood pressure was recorded to the nearest 2 mmHg mark on the manometer or to the nearest whole number on an automatic device. A blood pressure reading was repeated 1 to 2 minutes later, and the second reading recorded to the nearest 2 mmHg mark.

The baseline value categories and post-baseline endpoint value categories shown in Table 7 were measured and presented. Definitions for potentially clinically significant (PCS) vital signs treatment-emergent values are defined below in Table 8.

TABLE 7
Vital Signs Value Categories

| Vital Sign | Low | | Normal | High | |
|---|---|---|---|---|---|
| Systolic | ≤90 | mmHg | >90 mmHg | ≥160 | mmHg |
| Blood Pressure | | | to <160 mmHg | | |
| Diastolic | ≤50 | mmHg | >50 mmHg | ≥100 | mmHg |
| Blood Pressure | | | to <100 mmHg | | |
| Pulse | ≤50 | beats/min | >50 beats/min | ≥90 | beats/min |
| | | | to <90 beats/min | | |

TABLE 8
Potentially Clinically Significant Vial Signs Value Definitions

| Vital Sign | PCS Low | PCS High |
|---|---|---|
| Systolic | ≤90 mmHg AND decrease | ≥160 mmHg AND increase |
| Blood | of ≥20 mmHg; | of ≥20 mmHg; |
| Pressure | ≤90 mmHg; | ≥160 mmHg; |
| | decrease of ≥20 mmHg | increase of ≥20 mmHg |
| Diastolic | ≤50 mmHg AND decrease | ≥100 mmHg AND increase |
| Blood | of ≥10 mmHg; | of >10 mmHg; |
| Pressure | ≤50 mmHg; | ≥100 mmHg; |
| | decreased of >10 mmHg | increase of 10 mmHg |
| Pulse | ≤50 beats/min AND decrease | ≥90 beats/min AND increase |
| | of ≥15 beats/min; | of ≥15 beats/min; |
| | ≤50 beats/min; | ≥90 beats/min; |
| | decrease of ≥15 beats/min | increase of ≥15 beats/min |

Number (%) of patients with any post-baseline PCS vital sign values was summarized by treatment group. A listing of patients who meet the threshold criteria was provided.

Physical Examination: A physical examination included source documentation of general appearance, skin, and specific head and neck, heart, lung, abdomen, extremities, and neuromuscular assessments.

Height, Weight, and Body Mass Index: Height and weight were measured. Measurement of weight was performed with the patient dressed in indoor clothing, with shoes removed, and bladder empty.

Waist Circumference: Waist circumference was measured with a tape measure, as follows: Start at the top of the hip bone then bring the tape measure all the way around—level with the navel. Make sure the tape measure is snug, but without compressing the skin, and that it is parallel with the floor. Patients should not have held their breath while measuring waist circumference.

12-Lead Electrocardiogram (ECG): ECGs (standard 12-lead) were obtained annually. Site personnel made every attempt to perform a patient's ECG using the same equipment at each visit. ECGs were reviewed by the site for the detection of silent MI. Silent MIs were sent for event adjudication. All post-randomization ECGs (protocol-specified and other) were sent to the CEC for evaluation of silent MI. The 12-lead ECG parameters, including Heart Rate (bpm), PR Interval (msec), QRS Interval (msec), QT Interval (msec), and QTc Interval (msec), were measured, and Overall Interpretation and Silent MI (Yes/No) were summarized for all patients at Screening (Visit 1), Randomization visit (Visit 2; Day 0), and all other follow-up visits including the last visit of the study.

A treatment-emergent PCS high value at any time was defined as a change from a value less than or equal to the defined PCS value at baseline to a PCS high value at any post-baseline measurement. A treatment-emergent PCS low value at any time was defined as a change from a value greater than or equal to the lower PCS value at baseline to a PCS low value at any post-baseline measurement. Table 9 provides the PCS ECG values.

TABLE 9
Potentially Clinically Significant ECG Value Definitions

| ECG Parameter | PCS Low | PCS High |
|---|---|---|
| PR Interval | <120 msec | >120 msec and increase of >20 |
| | | msec from baseline |
| QRS Interval | N/A | >110 msec |
| QTc | N/A | >500 msec |

Number (%) of patients with post-baseline PCS ECG values were presented by treatment group. A listing of subjects with potentially clinically significant changes in ECG values was included.

### Treatment and Procedures

Treatment Regimen, Dosage, and Duration: Eligible study patients were randomly assigned on Day 0 to one of the 2 treatment groups. Patients in each group received either 4 g/day AMR101 or placebo for up to 6.5 years, depending on individual date of randomization and overall study stop date according to Table 10. The daily dose of study drug was 4 capsules per day taken as two capsules taken on two occasions per day (2 capsules were given twice daily).

TABLE 10
Dosing Schedule during the Treatment Period

| Treatment Group | Daily Dose | Number of Capsules per Day |
|---|---|---|
| 1 | 4 g | 4 capsules of 1000 mg AMR101 |
| 2 | Placebo | 4 capsules of matching placebo |

Patients were instructed to take the study drug with food (i.e., with or at the end of their morning and evening meals). On days that patients were scheduled for study visits, the daily dose of study drug was administered by site personnel with food provided by the site following collection of all fasting blood samples. For the purposes of this study, fasting was defined as nothing by mouth except water (and any essential medications) for at least 10 hours.

### Treatment Assignment

Identification Number: A unique patient identification number (patient number) was established for each patient at each site. The patient number was used to identify the patient throughout the study and was entered on all documentation. If a patient was not eligible to receive treatment, or if a patient discontinued from the study, the patient number could not be reassigned to another patient. The patient number was used to assign patients to one of the 2 treatment groups according to the randomization schedule.

Drug Randomization: Only qualified patients who meet all of the inclusion criteria and none of the exclusion criteria were randomized and received study medication starting at Visit 2 (Day 0). Eligible patients were randomly assigned to one of the 2 treatment groups. Randomization was stratified by CV risk category, use of ezetimibe, and by geographical region (Westernized, Eastern European, and Asian Pacific countries). Approximately 70% of randomized patients were in the CV Risk Category 1, including patients with established CVD, and approximately 30% of randomized patients were in the CV Risk Category 2, including patients with diabetes and at least one additional risk factor but no established CVD. Enrollment with patients of a CV risk category was stopped when the planned number of patients in that risk category was reached.

Emergency Unblinding: In an emergency, when knowledge of the patient's treatment assignment was essential for the clinical management or welfare of the patient, the investigator could request the patient's treatment assignment for unblinding. Prior to unblinding the patient's individual treatment assignment, the investigator assessed the relationship of an adverse event to the administration of the study drug (Yes or No). If the blind was broken for any reason, the investigator recorded the date and reason for breaking the blind on the appropriate Case Report Form (CRF) and source documents.

Compliance Control: Unless clear contraindications arise, patients were strongly encouraged to adhere to their treatment regimen with the study drug for the duration of the trial. Any interruptions of therapy were, if possible, brief (e.g., less than 4 weeks) and only for clinically indicated reasons, such as adverse events. Discontinuations were discouraged as much as possible. Any discontinuations were based on compelling clinical reasons. For every patient, an assessment of compliance to the study drug treatment regimen was obtained at each scheduled visit. Study medication was dispensed in amounts exceeding the amount required for the study. Patients were instructed to return all unused study medication at the next visit. Compliance to the study drug regimen was evaluated at each visit by counting unused capsules. Discrepancies were evaluated and discussed with each patient to assess compliance. If compliance was unsatisfactory, the patient was counseled about the importance of compliance to the dosing regimen. At the end of the study, the final study medication compliance was determined by unused capsule count.

### Study Restrictions

Concomitant Medications during Treatment/Follow-Up Period: Any medications administered during the study period were documented on the Concomitant Medication CRF. Patients had not taken any investigational agent within 90 days prior to screening. Patients could not participate in any other investigational medication trial while participating in this study. The following non-study drug related, non-statin, lipid-altering medications and supplements, and foods were prohibited during the study (from Visit 1 until after the Last Visit-End of Study), except for compelling medical reasons in Off Drug in Study (ODIS) patients:
niacin greater than 200 mg/day;
fibrates;
prescription omega-3 fatty acid medications;
dietary supplements containing omega-3 fatty acids (e.g., flaxseed, fish, krill, or algal oils);
bile acid sequestrants;
PCSK9 inhibitors;
cyclophosphamide; and
systemic retinoids.

If any of these products were used during the treatment/follow-up period of the study, it was for compelling medical reasons in ODIS patients and documented in the Concomitant Medication CRF. If the ODIS patient agreed to restart study medication, the use of excluded medication was discontinued. Foods enriched with omega-3 fatty acids were strongly discouraged after Visit 1 for the duration of the study. (This does not apply to the Netherlands or Canada only. Therefore, all centers in the Netherlands and Canada ignored this request.) The following products were allowed: statins, ezetimibe, and herbal products & dietary supplements not containing omega-3 fatty acids.

Statins:
The same statin at the same dose was continued until the end of the study, unless deemed medically necessary to change because of an adverse event or lack of efficacy (LOE). It was preferred that if LOE was the determining factor that ezetimibe was added to the present dose;
Switching between a brand name statin and the generic version of the same statin was allowed at any time during the study;
Statins were administered with or without ezetimibe;
Based on the FDA recommendation, simvastatin 80 mg was used only in patients who had been taking this dose for 12 months or more and had not experienced any muscle toxicity. (See reference: FDA Drug Safety Communication: Ongoing safety review of high-dose Zocor (simvastatin) and increased risk of muscle injury. (http://www.fda.gov/Drugs/DrugSafety/PostmarketDrugSafetylnformationforPatie ntsandProviders/ucm204882.htm)); and
Changing of the type of statin or the statin dose during the treatment/follow-up period of the study was only done for compelling medical reasons and was documented in the CRF. Maintaining statin therapy throughout the study was important and, in the rare circumstance that it became medically compelling to discontinue statin use, the patient could remain in the study and on study medication with approval from the Medical Monitor. Under such conditions, resumption of statin therapy was attempted when/if medically appropriate.
If the level of LDL-C exceeded 130 mg/dL (3.37 mmol/L) during the study (initial measurement and confirmed by a second determination at least 1 week later), the investigator either increased the dose of the present statin therapy or added ezetimibe to lower LDL-C. The investigator used the best clinical judgment for each patient.

LDL-C Rescue: If the level of LDL-C exceeded 130 mg/dL (3.37 mmol/L) during the study (initial measurement and confirmed by a second determination at least 1 week later), the investigator either increased the dose of the present statin therapy or added ezetimibe to lower LDL-C. The investigator used the best clinical judgment for each patient.

No data were available with regard to potential interactions between ethyl-EPA and oral contraceptives. There were no reports suggesting that omega-3 fatty acids, including ethyl-EPA, would decrease the efficacy of oral contraceptives.

Medications that were excluded if not at a stable dose for at least 28 days prior to screening could be initiated post-randomization if medically warranted (i.e., tamoxifen, estrogens, progestins, thyroid hormone therapy, systemic corticosteroids, and HIV-protease inhibitors).

Patient Restrictions: Beginning at the screening visit, all patients were instructed to refrain from excessive alcohol consumption, to follow a physician recommended diet, and to maintain it through the duration of the study. Excessive alcohol consumption is on average 2 units of alcohol per day or drinking 5 units or more for men or 4 units or more for women in any one hour (episodic excessive drinking or binge drinking). A unit of alcohol is defined as a 12-ounce (350 mL) beer, 5-ounce (150 mL) wine, or 1.5-ounce (45 mL) of 80-proof alcohol for drinks.

### Investigational Product

Clinical Trial Material: The following clinical materials were supplied by the Sponsor:
AMR101 1000 mg capsules
Placebo capsules (to match AMR101 1 g capsules)

The Sponsor supplied sufficient quantities of AMR101 1000 mg capsules and placebo capsules to allow for completion of the study. The lot numbers of the drugs supplied were recorded in the final study report. Records were maintained indicating the receipt and dispensation of all drug supplies. At the conclusion of the study, any unused study drug was destroyed.

Pharmaceutical Formulations: AMR101 1000 mg and placebo capsules (paraffin) were provided in liquid-filled, oblong, gelatin capsules. Each capsule was filled with a clear liquid (colorless to pale yellow in color). The capsules were approximately 25.5 mm in length with a diameter of approximately 9.5 mm.

Labeling and Packaging: Study medication was packaged in high-density polyethylene bottles. Labeling and packaging were performed according to GMP guidelines and all applicable country-specific requirements. The bottles were numbered for each patient based on the randomization schedule. The patient randomization number assigned by IWR or a designee of the Sponsor for the study (if no IWR system was used) corresponded to the number on the bottles. The bottle number for each patient was recorded in the Electronic Data Capture (EDC) system for the study.

### Dispensing Procedures and Storage Conditions

Dispensing Procedures: At Visit 2 (Day 0), patients were assigned a study drug according to their treatment group determined by the randomization schedule. Once assigned to a treatment group, patients received study drug supplies. At each visit, patients brought unused drug supplies dispensed to them earlier. From the drug supplies assigned to each patient, site personnel administered the drug while the patients were at the Research Site. The investigator or designee contacted the IWR system or a designee of the Sponsor for the study (if no IWR system is used) when any unscheduled replacements of study medication were needed. During the last visit of the treatment period, patients brought the unused drug supplies for site personnel to calculate the final study medication compliance by unused capsule count.

Storage Conditions: At the Research Sites, study drugs were stored at room temperature, 68° F. to 77° F. (20° C. to 25° C.). Storage temperature did not go below 59° F. (15° C.) or above 86° F. (30° C.), and the drug was stored in the original package. Study drugs were stored in a pharmacy or locked in a secure storage facility, accessible only to those individuals authorized by the investigator to dispense the drug. The investigator or designee kept accurate dispensing records. At the conclusion of the study, study site personnel accounted for all used and unused study drug. Any unused study drug was destroyed. The investigator agreed not to distribute the study drug to any patient, except those patients participating in the study.

### Efficacy Assessments

Specification of Variables and Procedures: The primary endpoint and the majority of the secondary and tertiary endpoints were based on clinical events related to CVD and mortality. All events occurring between randomization and the study end date (inclusive) were recorded. Only adjudicated events were included in the final analyses.

Primary Efficacy Endpoint: The primary efficacy endpoint was time from randomization to the first occurrence of the composite of the following clinical events: CV death; nonfatal MI (including silent MI; ECGs were performed annually for the detection of silent MIs); nonfatal stroke; coronary revascularization; and unstable angina determined to be caused by myocardial ischemia by invasive/non-invasive testing and requiring emergent hospitalization. The first occurrence of any of these major adverse vascular events during the follow-up period of the study was included in the incidence.

Secondary Efficacy Endpoints: The key secondary efficacy endpoint was the time from randomization to the first occurrence of the composite of CV death, nonfatal MI (including silent MI), or nonfatal stroke. Other secondary efficacy endpoints were time from randomization to the first occurrence of the individual or composite endpoints as follows (tested in the order listed):
The composite of CV death or nonfatal MI (including silent MI);
Fatal or nonfatal MI (including silent MI);
Non-elective coronary revascularization represented as the composite of emergent or urgent classifications;
CV death;
Unstable angina determined to be caused by myocardial ischemia by invasive/non-invasive testing and requiring emergent hospitalization;
Fatal and nonfatal stroke;
The composite of total mortality, nonfatal MI (including silent MI), or nonfatal stroke; and/or
Total mortality.

For the secondary endpoints that count a single event, the time from randomization to the first occurrence of this type of event was counted for each patient. For secondary efficacy endpoints that were composites of two or more types of events, the time from randomization to the first occurrence of any of the event types included in the composite were counted for each patient.

Tertiary Efficacy Endpoints: The following tertiary endpoints were evaluated as supporting efficacy and safety analyses. Where applicable and unless specified otherwise, endpoint analyses were conducted as time from randomization to the first occurrence of the individual or composite endpoint as follows:
Total CV events analysis defined as the time from randomization to occurrence of the first and all recurrent major CV events defined as CV death, nonfatal MI (including silent MI), nonfatal stroke, coronary revascularization, or unstable angina determined to be caused by myocardial ischemia by invasive/non-invasive testing and requiring emergent hospitalization;
Primary composite endpoint in subset of patients with diabetes mellitus at baseline;
Primary composite endpoint in the subset of patients with metabolic syndrome at baseline with waist circumference cut points specifically set at least 35 inches (88 cm) for all women and Asian, Hispanic, or Latino men, and at least 40 inches (102 cm) for all other men;
Primary composite endpoint in the subset of patients with impaired glucose metabolism at baseline (Visit 2 FBG of 100-125 mg/dL);
Key secondary composite endpoint in the subset of patients with impaired glucose metabolism at baseline (Visit 2 FBG 100-125 mg/dL);
The composite of CV death, nonfatal MI (including silent MI), nonfatal stroke, cardiac arrhythmia requiring hospitalization of at least 24 hours, or cardiac arrest;
The composite of CV death, nonfatal MI (including silent MI), non-elective coronary revascularizations (defined as emergent or urgent classifications), or unstable angina determined caused by myocardial ischemia by invasive/non-invasive testing and requiring emergent hospitalization;
The composite of CV death, nonfatal MI (including silent MI), non-elective coronary revascularizations (defined as emergent or urgent classifications), unstable angina determined caused by myocardial ischemia by invasive/non-invasive testing and requiring emergent hospitalization, nonfatal stroke, or PVD requiring intervention, such as angioplasty, bypass surgery, or aneurysm repair;
The composite of CV death, nonfatal MI (including silent MI), non-elective coronary revascularizations (defined as emergent or urgent classifications), unstable angina determined caused by myocardial ischemia by invasive/non-invasive testing and requiring emergent hospitalization, PVD requiring intervention, or cardiac arrhythmia requiring hospitalization of at least 24 hours;
New CHF;
New CHF as the primary cause of hospitalization;
Transient ischemic attack (TIA);
Amputation for PVD;
Carotid revascularization;
All coronary revascularizations defined as the composite of emergent, urgent, elective, or salvage;
Emergent coronary revascularizations;
Urgent coronary revascularizations;
Elective coronary revascularizations;
Salvage coronary revascularizations;
Cardiac arrhythmias requiring hospitalization of at least 24 hours;
Cardiac arrest;
Ischemic stroke;
Hemorrhagic stroke;
Fatal or nonfatal stroke in the subset of patients with a history of stroke prior to baseline;
New onset diabetes, defined as Type 2 diabetes newly diagnosed during the treatment/follow-up period;
New onset hypertension, defined as blood pressure of at least 140 mmHg systolic OR at least 90 mmHg diastolic newly diagnosed during the treatment/follow-up period;
Fasting TG, TC, LDL-C, HDL-C, non-HDL-C, VLDL-C, Apo B, hs-CRP (hs-CRP and log[hs-CRP]), hsTnT, and RLP-C (to be estimated from standard lipid panel, RLP-C=TC−HDL-C−LDL-C [Varbo 2014]), (based on ITT estimands):
   Assessment of the relationship between baseline biomarker values and treatment effects within the primary and key secondary composite endpoints;
   Assessment of the effect of AMR101 on each marker; and
   Assessment of the relationship between post-baseline biomarker values and treatment effects within the primary and key secondary composite endpoints by including post-baseline biomarker values (for example, at 4 months, or at 1 year) as a covariate.
Change in body weight; and
Change in waist circumference.

Where applicable and unless specified otherwise, for the tertiary endpoints that count a single event, the time from randomization to the first occurrence of this type of event was counted in each patient. Similarly, where applicable and unless specified otherwise, for tertiary endpoints that were composites of two or more types of events, the time from randomization to the first occurrence of any of the event types included in the composite was counted in each patient.

Other sensitivity, supportive, and exploratory analyses for the primary efficacy endpoint were carried out, namely, an on-treatment analysis which included primary event onset up to 0 and 30-days after the permanent discontinuation of the drug.

The following clinical events that were positively adjudicated by the Clinical Endpoint Committee were analyzed as tertiary endpoints for the intent-to-treat (ITT) population:
Composition of total mortality or congestive heart failure (CHF);
Composite of CV death or new CHF;
Sudden cardiac death;
Peripheral artery disease (PAD); and
Atrial fibrillation, or atrial flutter.

The above tertiary endpoints were analyzed similarly as the primary endpoint.

In addition, the following were analyzed as tertiary endpoints for the ITT population:
Relationship between on-treatment hs-CRP and the primary and key secondary endpoints; and
Relationship between on-treatment serum eicosapentaenoic acid (EPA) and the primary and key secondary endpoints.

To assess the relationship between on-treatment hs-CRP and the primary and key secondary endpoints, subgroup analyses were carried out as done for the ITT population for patients grouped according to values greater or equal to or less than 2 mg/dL at baseline and at 2 years. To assess the relationship between on-treatment serum EPA and the primary and key secondary endpoints, Kaplan-Meier (KM) curves were produced for AMR101 treated patients grouped into tertiles based on their values at year 1 and were compared with the placebo-treated patients.

### Safety Assessments

Specification of Variables and Procedures: Safety assessments included adverse events, clinical laboratory measurements (chemistry, hematology), 12-lead ECGs, vital signs (systolic and diastolic blood pressure, heart rate, respiratory rate, and body temperature), weight, waist circumference, and physical examinations as per Study Procedures in Table 1. A complete medical, surgical, and family history was completed at Visit 1. All laboratory test results were evaluated by the investigator as to their clinical significance. Any observation at physical examinations or laboratory values considered by the investigator to be clinically significant was considered an adverse event.

Adverse Events: An adverse event is defined as any untoward medical occurrence, which does not necessarily have a causal relationship with the medication under investigation. An adverse event can therefore be any unfavorable and/or unintended sign (including an abnormal laboratory finding), symptom, or disease temporally associated with the use of an investigational medication product, whether or not related to the investigational medication product. All adverse events, including observed or volunteered problems, complaints, or symptoms, were recorded on the appropriate CRF. Each adverse event was evaluated for duration, intensity, and causal relationship with the study medication or other factors.

Adverse events, which included clinical laboratory test variables, were monitored from the time of informed consent until study participation was complete. Patients were instructed to report any adverse event that they experienced to the investigator. Beginning with Visit 2, investigators assessed for adverse events at each visit and recorded the event on the appropriate adverse event CRF.

Wherever possible, a specific disease or syndrome rather than individual associated signs and symptoms was identified by the investigator and recorded on the CRF. However, if an observed or reported sign or symptom was not considered a component of a specific disease or syndrome by the investigator, it was recorded as a separate adverse event on the CRF.

Any medical condition that was present when a patient was screened or present at baseline that did not deteriorate was reported as an adverse event. However, medical conditions or signs or symptoms present at baseline and that changed in severity or seriousness at any time during the study were reported as an adverse event.

Clinically significant abnormal laboratory findings or other abnormal assessments that were detected during the study or were present at baseline and significantly worsened were reported as adverse events or serious adverse events (SAEs). The investigator exercised his or her medical and scientific judgment in deciding whether an abnormal laboratory finding, or other abnormal assessment, was clinically significant.

The investigator rated the severity (intensity) of each adverse event as mild, moderate, or severe, and also categorized each adverse event as to its potential relationship to study drug using the categories of Yes or No. The severity was defined as:
Mild—An event that is usually transient in nature and generally not interfering with normal activities.
Moderate—An event that is sufficiently discomforting to interfere with normal activities.
Severe—An event that is incapacitating with inability to work or do usual activity or inability to work or perform normal daily activity.

Causality Assessment: The relationship of an adverse event to the administration of the study drug was assessed according to the following definitions:
No (unrelated, not related, no relation)—The time course between the administration of study drug and the occurrence or worsening of the adverse event rules out a causal relationship and another cause (concomitant drugs, therapies, complications, etc.) is suspected.
Yes (related, probably related, possibly related)—The time course between the administration of study drug and the occurrence or worsening of the adverse event is consistent with a causal relationship and no other cause (concomitant drugs, therapies, complications, etc.) can be identified.

The following factors were also considered:
The temporal sequence from study medication administration;
The event occurred after the study medication was given. The length of time from study medication exposure to event was evaluated in the clinical context of the event;
Underlying, concomitant, intercurrent diseases;
Each report was evaluated in the context of the natural history and course of the disease being treated and any other disease the patient may have had;
Concomitant medication;
The other medications the patient was taking or the treatment the patient received were examined to determine whether any of them might have caused the event in question;
Known response pattern for this class of study medication;
Clinical and/or preclinical data may have indicated whether a particular response was likely to be a class effect;
Exposure to physical and/or mental stresses;
The exposure to stress might induce adverse changes in the patient and provide a logical and better explanation for the event;
The pharmacology and pharmacokinetics of the study medication; and
The known pharmacologic properties (absorption, distribution, metabolism, and excretion) of the study medication were considered.

Unexpected Adverse Events: An unexpected adverse event is an adverse event either not previously reported or where the nature, seriousness, severity, or outcome is not consistent with the current Investigator's Brochure.

Serious Adverse Events: A serious adverse event (SAE) is defined as an adverse event that meets any of the following criteria:
Results in death;
Is life-threatening. The term “life-threatening” in the definition of “serious” refers to an event in which the patient was at risk of death at the time of the event. It does not refer to an event, which hypothetically might have caused death, if it were more severe;
Requires hospitalization or prolongation of existing hospitalization. In general, hospitalization for treatment of a pre-existing condition(s) that did not worsen from baseline was not considered an adverse event and was not reported as SAE;
Results in disability/incapacity;
Is a congenital anomaly/birth defect; and
Is an important medical event. Important medical events that may not result in death, be life threatening, or require hospitalization were considered an SAE when, based upon appropriate medical judgment, they may have jeopardized the patient and may have required medical or surgical intervention to prevent one of the outcomes listed above. Examples of such medical events included allergic bronchospasm requiring intensive treatment in an emergency room or at home, blood dyscrasias, or convulsions that did not result in inpatient hospitalizations, or the development of drug dependency.

By design of this study, SAEs that were endpoint events were only recorded for the endpoint determination and not captured as SAEs. The intention was that the endpoint events were reported to IRBs as SAEs, unless the IRB required that these were reported. Investigators specifically informed their institution/IRB of this plan and confirmed whether or not they wanted the endpoint events reported. By agreement with the U.S. FDA, these endpoints were also not reported to the U.S. FDA as SAEs; rather they were reported as endpoint events. Following adjudication if the event was determined to not meet the criteria for an event, the event was evaluated as an SAE beginning with that day as Day 0.

Adverse Events of Special Interest: Bleeding-related adverse events, glucose control (fasting blood glucose and HbA1c), and indicators of hepatic disorders (e.g., ALT or AST increases >3×ULN, total bilirubin increases of ≥2×ULN) were summarized separately and compared between treatment groups.

### Serious Adverse Event Reporting—Procedure for Investigators

Initial Reports: All SAEs occurring from the time of informed consent until 28 days following the last administration of study medication were reported to the Sponsor or designee within 24 hours of the knowledge of the occurrence (this refers to any adverse event that meets any of the aforementioned serious criteria). SAEs that the investigator considered related to study medication occurring after the 28-day follow-up period were also reported to the Sponsor or designee. The investigator was required to submit SAE reports to the IRB or IEC in accordance with local requirements. All investigators involved in studies using the same investigational medicinal product (IMP) received any Suspected Unexpected Serious Adverse Reaction (SUSAR) reports for onward submission to their local IRB as required. All reports sent to investigators were blinded. In addition, regulatory agencies were notified of SAEs per the requirements of the specific regulatory jurisdiction regulations and laws.

Follow-Up Reports: The investigator followed the patient until the SAE subsided, or until the condition became chronic in nature, stabilized (in the case of persistent impairment), or the patient died. Within 24 hours of receipt of follow-up information, the investigator updated the SAE form electronically in the EDC system for the study and submitted any supporting documentation (e.g., laboratory test reports, patient discharge summary, or autopsy reports) to the Sponsor or designee via fax or email.

Reporting by the Sponsor: IRBs and IECs were informed of SUSARs according to local requirements. Cases were unblinded for reporting purposes as required.

Exposure In Utero During Clinical Trials: If a patient became pregnant during the study, the investigator reported the pregnancy to the Sponsor or designee within 24 hours of being notified. The Sponsor or designee then forwarded the Exposure In Utero form to the investigator for completion. The patient was followed by the investigator until completion of the pregnancy. If the pregnancy ended for any reason before the anticipated date, the investigator notified the Sponsor or designee. At the completion of the pregnancy, the investigator documented the outcome of the pregnancy. If the outcome of the pregnancy met the criteria for immediate classification as an SAE (i.e., postpartum complication, spontaneous abortion, stillbirth, neonatal death, or congenital anomaly), the investigator followed the procedures for reporting an SAE.

### Treatment Discontinuation/Patient Withdrawal

Patients could withdraw from the study at any time and for any reason. Study drug administration could also be discontinued at any time, at the discretion of the investigator. In any case, follow-up for efficacy and safety was continued in subjects that discontinued therapy, but remained in the study (i.e., ODIS patients).

Reasons for Early Study Drug Discontinuation: Study drug discontinuation was avoided as much as possible, but could have been done for any of the following reasons:
Patient withdrew consent or requested early discontinuation from the study for any reason. Patients were encouraged to continue to participate in the study for the entire duration of the study even if they choose not to take study medication any longer;
Occurrence of a clinical or laboratory adverse event, either serious or non-serious, at the discretion of the investigator. The Sponsor or designee was notified if a patient was discontinued because of an adverse event or laboratory abnormality. It was recommended that, unless clear contraindications arise, patients were strongly encouraged to adhere to their treatment regimen with the study drug for the duration of the trial. Any interruptions of therapy were, if possible, brief (e.g., <4 weeks) and only for clinically indicated reasons, such as adverse events. The following were considered a reason for discontinuation:
   ALT >3×ULN and bilirubin >1.5×ULN;
   ALT >5×ULN;
   ALT >3×ULN and appearance or worsening of hepatitis;
   ALT >3×ULN persisting for >4 weeks; and/or
   ALT >3×ULN and cannot be monitored weekly for 4 weeks.
Any medical condition or personal circumstance that, in the opinion of the investigator, exposed the patient to risk by continuing in the study or precluded adherence to the protocol;
Sponsor discontinued the study;
Investigative site closure, in the event that:
   Another investigative site cannot accommodate the patient, or
   The patient was unable or unwilling to travel to another investigative site; and/or
A TG value was flagged as critically high, i.e., greater than 1000 mg/dL (11.29 mmol/L), and confirmed as critically high by a repeat measurement (new fasting blood sample) within 7 days. In this case, a patient could be discontinued from study drug (with the option to remain ODIS) and other lipid-altering medications could be (re)initiated. If the TG value was flagged as greater than 2000 mg/dL (22.58 mmol/L) then appropriate medical action was taken by the investigator as soon as possible.

Occurrence of an outcome event according to the judgment of the investigator was not considered a valid reason for study drug discontinuation. Patients whose treatment with study medication was discontinued early, and had not withdrawn consent, stayed in the study and were monitored until the end of the study. Patients that continued in the study after at least 30 days cessation of therapy were characterized as ODIS. ODIS patients were asked to return to the study site for an interim visit once the patient had been off the study drug for more than 30 days. Procedures at this visit were consistent with those at Visit 5. If not contraindicated, patients also had the option to restart the study medication at any point once characterized as ODIS. For patients who discontinued study medication (e.g., for an AE that may or may not have been drug-related), a brief therapy interruption could have been followed with a re-challenge (re-initiating study medication) as soon as clinically appropriate, thereby allowing a causative role for study medication to be confirmed or ruled out and continuing a patient in the study and on the study drug if appropriate. The reason for study drug discontinuation or interruption was recorded on the CRF.

Follow-Up after Early Study Drug Discontinuation/Lost to Follow-Up

Patients who prematurely discontinued the study drug were not replaced. All randomized patients were followed up with until the study end date or death, regardless of whether they discontinued the study drug prematurely or not. Any event occurring after early study drug discontinuation was recorded up through the study end date. In order to follow the medical status of the patients, especially when they discontinued the study, investigators were encouraged to obtain information from the patient's primary care practitioner (physician or any other medical care provider). Investigators were also requested to try as much as possible to re-contact those patients at the end of the trial to obtain at least their vital status as well as their status with respect to the primary endpoint, and thus avoided lost to follow-up for the efficacy assessment. If patients were lost to follow-up, the CRF was completed up to the last visit or contact.

### Statistics

Randomized Population: The randomized population included all patients who signed the informed consent form and were assigned a randomization number at Visit 2 (Day 0).

Intent-to-Treat Population: The ITT population included all patients who were randomized via the IWRS (Interactive Web Response System). All efficacy analyses were performed on the ITT population. Patients were analyzed according to the randomized treatment.

Modified Intent-to-Treat Population: The Modified Intent-to-Treat (mITT) population included all randomized patients who had the study drug dispensed after randomization. Groups were defined based on the randomized treatment.

Per-Protocol Population: The per-protocol (PP) population included all mITT patients without any major protocol deviations, and who had at least 80% compliance while on treatment. To be included in the PP population the minimum time on therapy was 90 days.

Safety Population: All safety analyses were conducted based on the safety population, which is defined as all randomized patients. This was the same as the ITT population.

Statistical Methods: Safety and efficacy variables were analyzed using appropriate statistical methods that were described in detail in a separate Statistical Analysis Plan (SAP). The SAP was finalized before study unblinding.

Patient Disposition and Demographic/Baseline Characteristics: The number and percentage of patients was tabulated for each of the following categories for each treatment group:
Screened (total only);
Re-screened and reasons for re-screening (total only);
ITT overall and by stratification factors (CV risk, ezetimibe use, and geographical region);
mITT population; overall and by stratification factors (CV risk, ezetimibe use, and geographical region);
PP population; overall and by stratification factors (CV risk, ezetimibe use, and geographical region);
Safety population;
Patients who completed the study;
Patients who terminated from the trial early and the primary reason for early termination;
Patients who terminated the trial early prior to having a confirmed primary endpoint event;
Patients with complete follow-up, defined as those for whom all components of the primary endpoint have been ascertained during the entire observation period (or until death); and
Patients who, at the time of study completion, were discontinued from the study drug prematurely but continued within the study (i.e., ODIS patients), along with the primary reason.

For randomized patients who discontinued treatment with study drug, the primary reason for discontinuation was listed and summarized by treatment group. Demographic and baseline characteristics, including age, gender, ethnicity, race, height, body weight, BMI, diabetes, hypertension, metabolic syndrome, overweight/obese/normal according to BMI, and diabetes plus obesity were summarized using descriptive statistics by treatment group in the ITT population.

Demographic data and baseline characteristics were compared among treatment groups for the ITT and PP population. Differences in demographic and baseline characteristics were tested using a chi-square test (for categorical variables) or t-test (for continuous variables). The p-values used were considered descriptive, primarily as an assessment of the balance between the two groups. Age in years was calculated using the date of randomization (Visit 2) and the date of birth.

Study Medication Exposure and Compliance: Study drug exposure was summarized by treatment group using descriptive statistics for each time point and overall. Overall study drug compliance was calculated as the number of doses assumed to be taken relative to scheduled dosing period as follows:

Compliance (%)=(# Capsules of total dispensed−# Capsules of total returned)×100 (last dose date−first dose date+1)×4 capsules/day

Overall percent compliance was calculated per patient in the ITT and Modified ITT populations and summarized by treatment group using descriptive statistics.

Concomitant Therapies: Concomitant medication/therapy verbatim terms were coded using the latest available version, prior to database lock, of the World Health Organization Drug Dictionary and the Anatomical Therapeutic Chemical classification system. The numbers and percentages of patients in each treatment group taking concomitant medications were summarized. All verbatim descriptions and coded terms were listed for all non-study medications.

Analysis of Efficacy: For efficacy endpoints including CV events, only adjudicated events were included in the final statistical analyses.

Summary Statistics: Summary statistics (n, mean, standard deviation, median, minimum, and maximum) for the baseline and post-baseline measurements, the percent changes, or changes from baseline were presented by treatment group and by visit for all efficacy variables analyzed. The summary statistics included changes in body weight and body mass index from baseline by treatment group and by visit.

Primary Endpoint Analyses: The analysis of the primary efficacy endpoint was performed using the log-rank test comparing the 2 treatment groups (AMR101 and placebo) and including the stratification factor “CV risk category”, use of ezetimibe and geographical region (Westernized, Eastern European, and Asian Pacific countries) (each as recorded in the IWR at the time of enrollment) as covariates. The two-sided alpha level for the primary analysis was reduced from 0.05 to account for the interim analyses based on a group sequential design with O'Brien-Fleming boundaries generated using the Lan-DeMets alpha-spending function. The hazard ratio (HR) for treatment group (AMR101 vs. placebo) from a Cox proportional hazard model that included the stratification factor was also reported, along with the associated 95% confidence interval (CI). Kaplan-Meier estimates from randomization to the time to the primary efficacy endpoint were plotted.

The size and direction of the treatment effects of the individual components of the composite endpoint and their relative contribution to the composite endpoint were determined as well. All observed data that were positively adjudicated by the CEC, including data after discontinuation of study treatment for patients who discontinued study drug prematurely, were included in the primary analysis. Patients who did not experience a primary efficacy event prior to the end of the study or who withdrew from the study early without a preceding primary efficacy event were censored at the date of their last visit/phone contact. The longest prespecified interval between visits (onsite or phone) was 90 days. In view of the up to 90-day monitoring period for CV events, the primary endpoint for patients who had a non-CV death within 90 days of last contact without having had an earlier CV event was censored at the time of death. The primary endpoint for patients who had a non-CV death more than 90 days after last contact without having had an earlier CV event were censored at the time of last contact.

The primary analysis assumed that all silent MIs occurred on the date of the first tracing indicative of a silent MI; a second (sensitivity) analysis assumed that all silent MIs occurred on the day after the last prior normal ECG; and a third (sensitivity) analysis assumed that all silent MIs occurred at the mid-point between the last normal ECG and the ECG with the new MI. All deaths causally adjudicated as “undetermined” were combined with those adjudicated as “CV deaths” for the primary analysis. A sensitivity analysis of the CV death category was performed that excluded the “undetermined cause of death” cohort.

The primary efficacy analysis was performed on the ITT population. A sensitivity analysis was performed using the mITT and PP populations. As a sensitivity analysis, patients who discontinued the study drug prematurely were censored for the primary composite endpoint analysis on the date of drug discontinuation. The primary analysis was repeated using this censoring rule for the mITT population. As a supportive analysis, a multivariable, stratified Cox proportional hazards model was constructed for the primary endpoint to evaluate the treatment effect adjusting for important covariates.

Secondary Endpoint Analyses: The key secondary hypothesis was tested as part of the confirmatory process only if the primary analysis was statistically significant. For the analysis of secondary efficacy endpoints, the Type 1 error was controlled by testing each endpoint sequentially, starting with the key endpoint. Testing was done at a significance level consistent with that used for the primary endpoint and ceased when a secondary endpoint was found for which treatments did not significantly differ. P-values were presented for all analyses, but they were considered descriptive after the first non-significant result was obtained. Each of the secondary endpoints was analyzed by the same methods described for the primary efficacy endpoint. Kaplan-Meier estimated, the log-rank test stratified by stratification factors used at randomization, and the Cox proportional hazards model including the stratification factors as specified above for the primary efficacy endpoint were summarized by treatment group. In view of the 90-day monitoring period for CV events, the key secondary endpoint for patients who had a non-CV death within 90 days of last contact without having had an earlier CV event was censored at the time of death. The key secondary endpoint for patients who had a non-CV death more than 90 days after last contact without having had an earlier CV event was censored at the time of last contact. Kaplan-Meier curves stratified by each stratification factor were presented. These analyses were conducted for the ITT population.

Tertiary Endpoints Analyses: Time-to-event tertiary endpoints were analyzed by the same methods as described for the primary efficacy endpoint. Kaplan-Meier estimates, the log-rank test stratified by stratification factors used at randomization, and the Cox proportional hazards model as specified for the primary efficacy endpoint were summarized by treatment group. In view of the 90-day monitoring period for CV events, if applicable, tertiary endpoints for patients who had a non-CV death within 90 days of last contact without having had an earlier CV event were censored at the time of death. If applicable, tertiary endpoints for patients who had a non-CV death more than 90 days after last contact without having had an earlier CV event were censored at the time of last contact. Kaplan-Meier curves stratified by each of the stratification factors were presented.

The fasting lipid panel was tested at Screening (Visit 1 or Visit 1.1), Randomization visit (Visit 2; Day 0), Visit 3 (Day 120; ˜4 Months), and all other follow-up visits including the last visit. For change from baseline to 1 year, preparative ultracentrifugation measurements for LDL-C were analyzed, unless this value was missing. If the LDL-C preparative ultracentrifugation values were missing, then another LDL-C value was used, with prioritization of values obtained from LDL-C Direct measurements, followed by LDL-C derived by the Friedewald calculation (only for subjects with TG <400 mg/dL), and finally LDL-C derived using the calculation published by Hopkins University investigators (Martin S S, Blaha M J, Elshazly M B, et al. Comparison of a novel method versus the Friedewald equation for estimating LDL-C levels from the standard lipid profile. JAMA. 2013; 310:2061-8.). In addition, change from baseline to day 120 in LDL-C utilizing Friedewald's and Hopkins methods was analyzed, using the arithmetic mean of LDL-C obtained at Visit 2 (Day 0) and the preceding Visit 1 (or Visit 1.1). If one of these values was missing, the single available LDL-C value was used. LDL-C according to Hopkins was calculated at each visit.

The randomization visit was considered Baseline. If a baseline value was not available from the randomization visit, then the latest screening value was used. For measurements of lipids, lipoproteins and inflammatory markers, the change and the percent change were summarized at each visit. Since these biomarkers are typically not normally distributed, the Wilcoxon rank-sum test was used for treatment comparisons of the percent change from baseline, and medians and quartiles were provided for each treatment group. The medians of the differences between the treatment groups and 95% CIs were estimated with the Hodges-Lehmann method. In addition, shift tables were generated as appropriate.

As an additional exploratory analysis, the relationship between post-baseline biomarker values and treatment effects with the primary and key secondary endpoints were assessed by adding biomarker values (for example, at 4 months, or at 1 year, etc.) as time-dependent covariates in the Cox proportional hazards model. Diagnostic plots for the proportional hazards assumption were evaluated. Weight was measured at the screening visit and at all follow-up visits, including the last visit of the study. Waist circumference was measured at the randomization visit (Visit 2; Day 0), Visit 5 (Day 720), and the last visit of the study. Descriptive statistics were presented by visit and treatment group for baseline, post-treatment change from baseline, and the percent change from baseline. Analysis methods for repeated measurements were used to compare percent change from baseline between treatments.

Additional prespecified efficacy endpoints and analyses of this study are listed below. These endpoints and analyses were exploratory in nature and were not included in the original testing scheme:
Time-to-event analyses as done for the primary analysis were carried out at 1-year and 2-year landmarks for the ITT Population;
For the recurrent CV events analyses based on the 5-component MACE (CV death, nonfatal MI, nonfatal stroke, unstable angina requiring hospitalization, or coronary revascularization), a total CV event was performed using a Negative Binomial Model analysis;
An on-treatment sensitivity analysis was performed including primary events with onset up to 0 and 30 days after permanent discontinuation of study drug;
As done for the primary analysis, time-to-event analyses at 1-year and 2-year landmarks for the key secondary endpoints for the ITT Population;
An analysis of the following clinical events that are positively adjudicated as tertiary endpoints for the ITT Population:
   Composite of total mortality, or new CHF;
   Composite of CV death, or new CHF;
   Sudden cardiac death;
   Peripheral artery disease (PAD); and
   Atrial fibrillation, or atrial flutter.
An analysis of the following as tertiary endpoints for the ITT Population:
   Relationship between on-treatment hs-CRP and primary and key secondary endpoints; and
   Relationship between on-treatment serum EPA and primary and key secondary endpoints.
To assess relationships between on-treatment hs-CRP and primary and key secondary endpoints, subgroup analyses as done for the ITT population for patients grouped according to (1) values greater than or equal to or (2) less than 2 mg/dL at baseline and at 2 years;
To assess relationships between on-treatment serum EPA and primary and key secondary endpoints, Kaplan-Meier curves for AMR101 patients grouped into tertiles based on values at year 1 compared with placebo patients;
The following were added to the subgroup analyses:
   Baseline HbA1c value (<6.5%, ≥6.5%);
   Baseline PAD; and
   Baseline TG ≥150 mg/dL with HDL-C ≤40 mg/dL for males and ≤50 mg/dL for females.

The following list presents additional pre-specified exploratory efficacy analyses that are of particular interest to the general clinical and scientific community that were also explored in this study:
Nonfatal MI (including both clinical manifestation and silent MI categorizations) for the ITT population;
Evaluation of effect of time-weighted (or area under the curve [AUC]) EPA data on the primary and key secondary composite endpoints for the ITT population;
Sensitivity analyses on primary and key secondary composite endpoints by excluding elective coronary artery revascularizations if onset is less than 3 months post randomization; and also excluding peri-procedural MIs for the ITT population;
Two silent MI (SMI) sensitivity analyses on primary and key secondary composite endpoints—ITT Population:
   Counting all potential SMIs identified by CEC ECG reviewer, whether confirmed at final ECG or not; and
   Counting only potential SMIs that have at least one confirmatory ECG showing persistence of Q-waves (even if not present at final ECG).
Non-alcoholic fatty liver disease (NAFLD) analyses using NAFLD Fibrosis Score (NFS), assessing—ITT Population:
   Effect on primary and key secondary composite endpoints by baseline NFS category; and
   Treatment effect on change from baseline in NFS at 1 and 5 years.
Individual and combined on-treatment goal achievement of TG ≤150 mg/dL and hs-CRP ≤2 mg/L at 2 years, and end of study for the ITT population;
Additional renal function (eGFR) analyses—ITT population:
   Primary and key secondary composite endpoints for patients with baseline renal dysfunction [eGFR] ≥60 and <90 mL/min/1.73 m²; and
   Treatment effect on change from baseline in renal function (eGFR) at 1 and 5 years.
Sensitivity analyses on primary and key secondary composite endpoints by excluding patients with post-randomization LDL-C values greater than 100 mg/dL; and another for greater than 70 mg/dL for the ITT population;
Analyses of hospitalization data (pooled positively adjudicated unstable angina requiring hospitalization, congestive heart failure [CHF] requiring hospitalization, and cardiac arrhythmia requiring hospitalization) for the ITT population;
   Time from randomization to first hospitalization; and
   Recurrent event analysis on hospitalizations.
Additional subgroup analyses (U.S. versus Non-U.S.) on the primary and key secondary composite endpoints; also potentially other endpoints for the ITT population;
Additional subgroup analyses for patients with very high-risk CVD (defined as recurrent cardiovascular [CV] events or CV events in more than one vascular bed, i.e., polyvascular disease) on the primary and key secondary composite endpoints; also potentially other endpoints for the ITT population;
Sensitivity analyses for Apo B to assess whether subgroup(s) with Apo B reductions from baseline beyond certain threshold(s) have corresponding incremental reductions in clinical endpoint events;
Sensitivity analyses for myocardial infarctions excluding peri-procedural MIs (Type 4a);
   Additional analyses factoring for recency and number of prior MIs.
Sensitivity analyses for stroke, factoring for patients with history of stroke;
Sensitivity analyses for heart failure, factoring for patients with history of heart failure;
Sensitivity analyses for endpoints comprised of coronary revascularizations which exclude early elective revascularizations (e.g., within 30-90 days post-randomization);
Subgroup analyses of primary (and potentially key secondary) endpoint(s) among the following cohorts:
   High risk patients with “the hypertriglyceridemic waist” (obese patients at high CV risk);
   High risk subgroup defined by baseline hsTNT level (and potentially by NT-proBNP from archived frozen samples);
   High TG/low LDL-C phenotypes; and
   High-risk patients as defined by their atherothrombotic risk score.
Treatment effect on:
   Peripheral arterial events (e.g., major adverse limb events [MALE]); and
   Hypertension, using BP as a continuous variable.
Using archived frozen serum biosamples, additional analyses of fatty-acid levels (and ratios), including baseline and on-treatment effects on EPA, DHA, DPA, AA (and associated ratios) and relationships between fatty-acid levels and cardiovascular outcomes;
   Relationship between on-treatment fatty-acid levels;
   Baseline fatty-acid levels; and
   Study medication compliance.
Using archived frozen biosamples (e.g., serum and whole blood); potential analyses of treatment effects on biomarkers and genetic markers and associations with outcomes, including but not limited to the following:
   LDL-P;
   RLP-C (measured);
   LDL-TG;
   Ox-LDL;
   Galectin-3;
   Lp(a) at baseline, as a predictor of CVD benefit;
   LpPLA2;
   HDL2, HDL3, apo A-I, apo A-II, HDL-P, apo C-III (and apo C-III in apo-B containing proteins), apo A-V, Apo E subtypes (2, 3, 4), IL-6, lipoprotein lipase (LPL); and
   Analyses may include change (and percent change) from baseline, on-treatment comparisons between treatment groups with testing as predictors of CV risk.
Exploratory analyses of differential treatment effects for potential benefit (from adverse event reports) of:
   Ophthalmologic changes (e.g., incidence of age-related macular degeneration, progression of diabetic retinopathy);
   Cognitive impairment;
   Erectile dysfunction; and
   Ischemic cardiomyopathy (as indicated by hospitalization for CHF, ICD placement etc.).
Additional genetic bioassays including genes which may relate to triglyceride, lipid metabolism, and CVD; and
Effects of potential mediators identified post hoc on primary/key secondary outcome measures.

In this study, new onset diabetes was defined as Type 2 diabetes newly diagnosed during the treatment/follow-up period (i.e., patients with no history of diabetes at randomization). For purposes of this study, a diagnosis of diabetes was made based on the observation of:
HbA1c ≥6.5%. The test was performed in a laboratory using a method that is National Glycohemoglobin Standardization Program (NGSP) certified and standardized to the Diabetes Control and Complications Trial (DCCT) assay. In the absence of unequivocal hyperglycemia, HbA1c ≥6.5% was confirmed by repeat testing;
Fasting plasma glucose (FPG) ≥126 mg/dL (7.0 mmol/L). Fasting was defined as no caloric intake for at least 8 hours. In the absence of unequivocal hyperglycemia, FPG ≥126 mg/dL (7.0 mmol/L) was confirmed by repeat testing;
2-hr plasma glucose ≥200 mg/dL (11.1 mmol/L) during an Oral Glucose Tolerance Test (OGTT). The test was performed as described by the World Health Organization, using a glucose load containing the equivalent of 75 g anhydrous glucose dissolved in water. In the absence of unequivocal hyperglycemia, 2-hr plasma glucose ≥200 mg/dL (11.1 mmol/L) during an OGTT was confirmed by repeat testing; and/or
In a patient with classic symptoms of hyperglycemia or hyperglycemic crisis, a random plasma glucose ≥200 mg/dL (11.1 mmol/L).

In the absence of unequivocal hyperglycemia, the first three criteria were confirmed by repeat testing.

Exploratory Subgroup Analyses: Analyses of the effects that patients off the study drug and withdrawn from the study have on the primary endpoint were performed. Subgroup analyses of the primary and key secondary endpoints were performed as described for the primary endpoint. For each subgroup, Kaplan-Meier estimates, the log-rank test stratified by stratification factors used at randomization (except where the subgroup was a stratification factor), and HRs and CIs from the Cox proportional hazards model as specified for the primary efficacy endpoint were summarized by treatment group. Demographic, disease, treatment, and baseline lipid and lipoproteins parameters were explored.

Demographic parameters included: gender; age at baseline (<65 years and ≥65 years); race (white and nonwhite, or any other subset with at least 10% of the total number of patients); geographical region (Westernized, Eastern European, and Asian Pacific countries); and baseline ezetimibe use (yes/no).

Disease parameters included: CV risk category; the presence/absence of diabetes at baseline; and renal dysfunction at baseline (estimated glomerular filtration rate [eGFR]<60 mL/min/1.73 m²) using the Chronic Kidney Disease Epidemiology Collaboration (CKD-EPI) equation as follows:

eGFR=141×min (S_{cr}/κ,1)^{α}×max(S_{cr}/κ,1)⁻¹²⁰⁹×0.993^{Age}×1.018 [if female]×1.159 [if black]

Where:
S_{cr} is serum creatinine in mg/dL,
κ is 0.7 for females and 0.9 for males,
α is −0.329 for females and −0.411 for males,
min indicates the minimum of S_{cr}/κ or 1, and
max indicates the maximum of S_{cr}/κ or 1.

Treatment Parameters included: Statin intensity at baseline (statin type and regimen) and statin intensity categories as defined in ACC/AHA Cholesterol Guidelines (Stone 2013) and patient's 10-year CV Risk Score (Goff 2013).

Baseline Lipid and Lipoprotein Parameter included: LDL-C (by tertile); HDL-C (by tertile, and tertile by gender); TG (by tertile, and tertile by gender); RLP-C (by tertile); TG ≥150 mg/dL and TG <150 mg/dL; TG ≥200 mg/dL and TG <200 mg/dL; TG ≥median, TG <median; combined highest tertile for TG and lowest tertile for HDL-C; gender-specific highest tertile for TG and lowest tertile for HDL-C; TG ≥200 mg/dL with HDL-C ≤35 mg/dL; hs-CRP (≤3 mg/L and >3 mg/L) and by gender; hs-CRP (≤2 mg/L and >2 mg/L) and by gender; Apo B (by tertile); non-HDL-C (by tertile); baseline HbA1c value (<6.5%, ≥6.5%); baseline PAD; and baseline TG levels ≥150 mg/dL with HDL-C levels ≤40 mg/dL for males and ≤50 mg/dL for females.

A Cox proportional hazard (PH) model as mentioned above but additionally with baseline TG as a covariate were fitted to the data at each interim. Diagnostic plots for the PH assumption were evaluated. The consistency of the treatment effects in subgroups was assessed for the primary and key secondary efficacy endpoints. For each subgroup variable, a Cox PH model with terms for treatment, stratification factors (with the exception of those subgroup variables related to the stratification factors, e.g., CV risk category), subgroup, and treatment-by-subgroup interaction were performed. The main treatment effect was tested with this model. P-values for testing the interaction terms less than 0.15 were considered significant. Results were presented in a Forest plot.

Subgroup analyses of the primary and key secondary endpoints were performed as described for the primary endpoint. For each subgroup, Kaplan-Meier estimates, the log-rank test stratified by stratification factors used at randomization (except where the subgroup was a stratification factor), and HRs and CIs from the Cox proportional hazards model as specified for the primary efficacy endpoint were summarized by treatment group. All subgroup analyses were conducted for the ITT, mITT and PP populations.

Interim Efficacy Analysis: Two interim analyses were planned for the primary efficacy endpoint using adjudicated events when approximately 60% (967 events) and approximately 80% (1290 events) of the total number of primary endpoint events planned (1612) was reached. The planned interim analyses were based on a group-sequential design.

The interim results of the study were monitored by an independent Data Monitoring Committee (DMC). The analyses were performed by the independent statistical team who was unblinded to the treatment assignment and reported only to the DMC. If the study was terminated early following interim analysis, patients were notified promptly and brought in for their final close-out visit, and the final analyses of efficacy and safety included all data through their final visit. All suspected events were adjudicated in a blinded manner by the CEC. The time to event was calculated as the time from randomization to the onset date of the event (as determined by the CEC). Patients who did not experience any of the above events at the time of data cutoff for the interim but were still in the trial were considered censored at the time of their last regular contact before the interim data cutoff.

The alpha-levels for the two protocol prespecified interim analyses and the final analysis are based on a group sequential design (GSD) with O'Brien-Fleming boundaries generated using the Lan-DeMets alpha spending function. The one-sided alpha-levels and boundaries based on a Z-test and the achieved p-values for each of the two interim analyses and the final analysis are given in Table 11.

TABLE 11
Group Sequential P-Values Boundaries According to
Two Actual Interim Analyses Information Fractions

| | | | | Efficacy | Efficacy | |
|---|---|---|---|---|---|---|
| | | | | Boundary | Boundary | Achieved |
| | | No. of | Information | (1-sided | (2-sided | P-value |
| Look | Analysis | Events | Fraction | α-level) | α-level) | (2-sided) |
| 1 | IA#1 | 953 | 59.3% | 0.00356 | 0.0071 | 0.0000463 |
| 2 | IA#2 | 1218 | 75.8% | 0.00885 | 0.0177 | 0.00000082 |
| 3 | Final | 1606 | 100% | 0.02186 | 0.0437 | 0.00000001 |

Analysis of Safety: All analyses of safety were conducted on the safety population, which was defined as all randomized patients. The safety assessment was based on the frequency of adverse events, physical exams, vital signs, and safety laboratory tests. AEs with new onset during the study between the initiation of the study drug and 30 days after the last dose of the study drug for each patient was considered treatment-emergent (TEAEs). This included any AE with onset prior to initiation of study drug and increased severity after the treatment initiation.

Treatment-emergent adverse events were summarized by system organ class and preferred term, and by treatment. This included overall incidence rates (regardless of severity and relationship to study drug), and incidence rates for moderate or severe adverse events. A summary of SAEs and adverse events leading to early discontinuation (for 30 days) were presented through data listings. Patients who restarted the study drug were included in the summary of AEs leading to discontinuation. Safety laboratory tests and vital signs were summarized by post-treatment change from baseline for each of the parameters using descriptive statistics by treatment group. Those patients with significant laboratory abnormalities were identified in data listings. Additional safety parameters were summarized in data listings.

In addition to the treatment-emergent adverse events analyses, analyses on all AEs (serious and non-serious) and all serious AEs were performed.

All AEs included: TEAE by high level group term (HLGT); TEAE by high level term (HLT); and TEAE by system organ class (SOC), HLGT, HLT, and preferred term (PT) (4-level table).

All SAEs included: treatment emergent SAE by HLGT; treatment emergent SAE by HLT; and treatment emergent SAE by SOC, HLGT, HLT, and PT (4-level table).

### Clinical Laboratory Evaluation

The criteria for potentially clinically significant (PCS) laboratory values are provided in Tables 12 and 13. A treatment-emergent PCS high value at any time was defined as a change from a value less than or equal to the upper reference limit at baseline to a PCS high value at any post-baseline measurement. A treatment-emergent PCS low value at any time was defined as a change from a value greater than or equal to the lower reference limit at baseline to a PCS low value at any post-baseline measurement. Number (%) of patients with any post-baseline PCS laboratory values was summarized by treatment group. A listing of patients with PCS laboratory values at any time, i.e., baseline or at any post-baseline visit, were included.

TABLE 12
Potentially Clinically Significant Chemistry Values

| Parameter | PCS Low | | PCS High | |
|---|---|---|---|---|
| Albumin | ≤3.3 | g/dL | ≥5.8 | g/dL |
| Alkaline | Not Applicable (N/A) | | >1x ULN to 2x ULN | |
| Phosphate | | | >2x ULN to 3x ULN | |
| | | | >3x ULN | |
| ALT | N/A | | >1x ULN to 2x ULN | |
| | | | >2x ULN to 3x ULN | |
| | | | >3x ULN | |
| AST | N/A | | >1x ULN to 2x ULN | |
| | | | >2x ULN to 3x ULN | |
| | | | >3x ULN | |
| Bilirubin | N/A | | >1x ULN to 2x ULN | |
| | | | >2x ULN to 3x ULN | |
| | | | >3x ULN | |
| ALT + | N/A | | >3x ULN + 2x ULN | |
| Bilirubin | | | (Bilirubin) | |
| AST + | N/A | | >3x ULN + 2x ULN | |
| Bilirubin | | | (Bilirubin) | |
| Calcium | ≤7 | mg/dL | ≥11 | g/dL |
| | | | ≤12 | mg/dL |
| Chloride | <70 | mmol/L | >120 | mmol/L |
| Creatinine | <0.5 mg/dL | (Female) | >1.6 mg/dL | (Female) |
| | <0.65 mg/dL | (Male) | >2.0 mg/dL | (Male); |
| | | | ≥50% increase from baseline | |
| Creatine | <30 U/L | (Female) | >1x ULN to 5x ULN | |
| Kinase | <0.55 U/L | (Male) | >5x ULN to 10x ULN | |
| | | | >10x ULN | |
| Glucose | ≤36 | mg/dL; | ≥126 | mg/dL; |
| (fasting) | ≤70 | mg/dL | ≥130 | mg/dL |
| Potassium (K) | ≤3.0 | mEq/L | ≥150 | mEq/L |
| Total Protein | <5.0 | g/dL | ≥9.5 | g/dL |
| Urea Nitrogen | N/A | | ≥31 | mg/dL |
| (BUN) | | | | |
| Uric Acid | <1.9 mg/dL | (Female) | >7.5 mg/dL | (Female) |
| | <2.5 mg/dL | (Male) | >8 mg/dL | (Male) |

TABLE 13
Potentially Clinically Significant Hematology Values

| Parameter | PCS Low | | PCS High | |
|---|---|---|---|---|
| Red Blood | <3.5 × 10⁶/μL | (Female) | >3.5 × 10⁶/μL | (Female) |
| Cell (RBC) | <3.8 × 10⁶/μL | (Male) | >3.8 × 10⁶/μL | (Male) |

| | | | |
|---|---|---|---|
| Hemoglobin | <10.0 g/dL | (Female) | > |
| (Hgb) | <10.0 g/dL | (Male) | > |
| Hematocrit | <37% | (Female) | > |
| (Hct) | <42% | (Male) | > |

| | | |
|---|---|---|
| White Blood | <1.5 × 10³/μL | N/A |
| Cells (WBC) | | |
| White Cell | Segmented | Segmented |
| Differential | neutrophils <50% | neutrophils >70% |
| | Lymphocytes <30% | Lymphocytes >45% |
| | Monocytes N/A | Monocytes >6% |
| | Basophils N/A | Basophils >1% |
| | Eosinophils N/A | Eosinophils >3% |
| Platelet Count | <100 × 10³/μL | >500 × 10³/μL |

### Drug-Induced Liver Injury (DILI)

DILI cases were investigated through the following analyses:
A graph of distribution of peak values of alanine aminotransferase (ALT) versus peak values of total bilirubin (TBL) during the treatment period was prepared, using a logarithmic scale. In the graph, for each patient, the peak TBL times the Upper Limit of Normal (ULN) were plotted against the peak ALT times the ULN, where the peak TBL and peak ALT may or may not have happened on the same day of liver testing. The graph was divided into four quadrants with a vertical line corresponding to 3×ULN for ALT and a horizontal line corresponding to 2×ULN for TBL. The upper right quadrant was referred to as the potential Hy's Law quadrant, including potentially DILI cases.
A similar graph was plotted with respect to aspartate aminotransferase (AST).
The individual patient profile of liver function tests (ALT, AST, alkaline phosphatase [ALP] and TBL) over time was provided through a graph for all patients with peak value of ALT >3×ULN and peak value of TBL >2×ULN during the treatment period.
Number (%) of patients was provided for the following:
   ALT or AST >3×ULN;
   ALT or AST >3×ULN and TBL >2×ULN; and
   ALT or AST >3×ULN and TBL >2×ULN, and ALP <2×ULN.

### Study Design

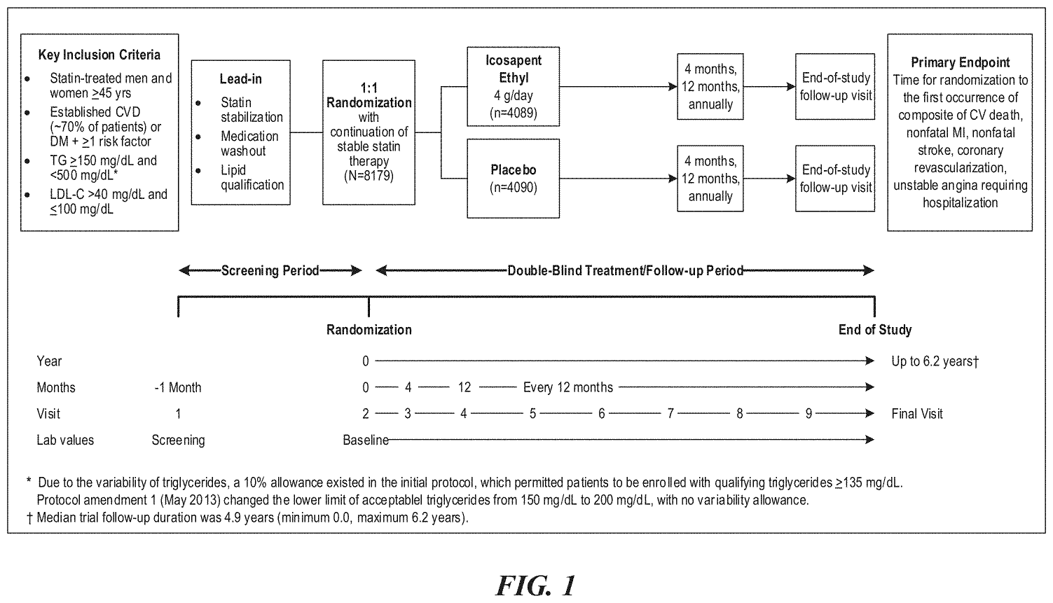

This was a Phase 3b, multi-center, multi-national, prospective, randomized, double-blind, placebo-controlled, parallel-group study. This was also an event-driven trial comparing the effect of AMR101 vs. placebo in terms of the composite endpoint listed above as the primary endpoint. The placebo contained mineral oil to mimic the color and consistency of icosapent ethyl in AMR101 and was administered in the same capsule fill volume and count as the AMR101. The study accrued a total of 1612 efficacy endpoint events with two planned interim analyses when approximately 967 (60%) and 1290 (80%) of the events had been adjudicated. The study included patients with established CVD (CV Risk Category 1) and patients at least 50 years old with diabetes and at least one additional risk factor for CVD but with CVD not established (CV Risk Category 2). Randomization was stratified by cardiovascular risk stratum which included the secondary-prevention cohort (i.e., CV Risk Category 1) or primary-prevention cohort (i.e., CV Risk Category 2), with the primary prevention cohort capped at 30% of enrollment, use or no use of ezetimibe, and by geographical region. Details of the study design are shown in FIG. 1.

Sample size calculation was based on the assumption of constant hazard, asymmetric recruitment rate overtime and without factoring for dropouts. A risk reduction corresponding to an HR of 0.85 (AMR101 vs. placebo) was assumed. 1612 events were required to detect this HR with approximately 90% power with one-sided alpha-level at 2.5% and with two interim analyses. The operating characteristics of this design were identical to those of a corresponding group sequential design with a two-sided alpha level of 0.05.

The recruitment period was assumed to be 4.2 years with 20% recruitment in the first year, 40% in the second year, 20% in the third year, 19% in the fourth year and the remaining 1% in the last 0.2 years. The estimated maximum study duration was 6.5 years unless the trial was terminated early for efficacy or safety issues. A one-year event rate of 5.2% (hazard=0.053) in the control arm was also assumed. Under these assumptions the number of patients enrolled was N=7990.

Since this was an events-driven trial, the ‘sample size’ was the number of events rather than the number of patients. The number of events that occurred depended primarily on three factors: how many patients were enrolled; the combined group event rate; and how long the patients were followed. Because of the difficulty in predicting the combined event rate, the Sponsor monitored the event rate as the trial progressed. If the combined event rate was less than anticipated, either increasing the number of patients, extending the length of follow-up, or a balance of adjusting both factors was necessary to achieve the sample size of 1612 events.

At completion of study enrollment, the actual number of patients randomized may have varied from the target number (either original or revised) as a result of the inherent lag between the date the last patient started screening and the date the last patient was randomized.

### Completion of Study

The end of the study was at the time the last patient-last visited during the follow-up period of the study. The IRB and IEC were notified about the end of the study according to country-specific regulatory requirements.

### Standardized Definitions for the Cardiovascular Trial Endpoint Events

In assessing patients in this clinical trial, the follow definitions were used:

Definition of Cardiovascular Death: Cardiovascular death includes death resulting from an acute myocardial infarction, sudden cardiac death, death due to CHF, death due to stroke, death due to CV procedures, death due to CV hemorrhage, and death due to other cardiovascular causes.

Death Due to Acute Myocardial Infarction: refers to a death by any mechanism (e.g., arrhythmia, CHF) within 30 days after an MI related to the immediate consequences of the MI, such as progressive CHF or recalcitrant arrhythmia. Mortal events that occur after a “break” (e.g., a CHF and arrhythmia-free period of at least a week) should be classified as CV or non-CV death, and if classified as a CV death, should be attributed to the immediate cause, even though the MI may have increased the risk of that event (e.g., the risk of arrhythmic death is increased for many months after an acute MI). Acute MI should be verified to the extent possible by the diagnostic criteria outlined for acute MI (see Definition of MI) or by autopsy findings showing recent MI or recent coronary thrombosis. Death resulting from a procedure to treat an MI (percutaneous coronary intervention (PCI), coronary artery bypass graft surgery (CABG)), or to treat a complication resulting from MI, should also be considered death due to acute MI. Death resulting from an elective coronary procedure to treat myocardial ischemia (i.e., chronic stable angina) or death due to an MI that occurs as a direct consequence of a CV investigation/procedure/operation should be considered as a death due to a CV procedure.

Sudden Cardiac Death: refers to a death that occurs unexpectedly, not within 30 days of an acute MI, and includes the following deaths: death witnessed and instantaneous without new or worsening symptoms; death witnessed within 60 minutes of the onset of new or worsening cardiac symptoms, unless the symptoms suggest an acute MI; death witnessed and attributed to an identified arrhythmia (e.g., captured on an electrocardiographic (ECG) recording, witnessed on a monitor, or unwitnessed but found on implantable cardioverter-defibrillator review); death after unsuccessful resuscitation from cardiac arrest; death after successful resuscitation from cardiac arrest and without identification of a non-cardiac etiology; and/or unwitnessed death without other cause of death (information regarding the patient's clinical status preceding death should be provided, if available).

General Considerations for Sudden Cardiac Death: A subject seen alive and clinically stable 12-24 hours prior to being found dead without any evidence or information of a specific cause of death should be classified as “sudden cardiac death.” Deaths for which there is no information beyond “patient found dead at home” are classified as “death due to other cardiovascular causes.” (See Definition of Undetermined Cause of Death, for full details below).

Death Due to Congestive Heart Failure: refers to a death in association with clinically worsening symptoms and/or signs of heart failure (See Definition of Heart Failure Event, for full details below). Deaths due to heart failure can have various etiologies, including single or recurrent myocardial infarctions, ischemic or non-ischemic cardiomyopathy, hypertension, or valvular disease.

Death Due to Stroke: refers to death after a stroke that is either a direct consequence of the stroke or a complication of the stroke. Acute stroke should be verified to the extent possible by the diagnostic criteria outlined for stroke.

Death Due to Cardiovascular Procedures: refers to death caused by the immediate complications of a cardiac procedure.

Death Due to Cardiovascular Hemorrhage: refers to death related to hemorrhage such as a non-stroke intracranial hemorrhage, non-procedural or non-traumatic vascular rupture (e.g., aortic aneurysm), or hemorrhage causing cardiac tamponade.

Death Due to Other Cardiovascular Causes: refers to a CV death not included in the above categories (e.g., pulmonary embolism or peripheral arterial disease).

Definition of Non-Cardiovascular Death: Non-cardiovascular death is defined as any death that is not thought to be due to a cardiovascular cause. The following is a suggested list of non-cardiovascular causes of death for this trial.
Non-malignant, Non-cardiovascular Death:
   Pulmonary;
   Renal;
   Gastrointestinal;
   Hepatobiliary;
   Pancreatic;
   Infection (includes sepsis)
   Non-infectious (e.g., systemic inflammatory response syndrome (SIRS));
   Hemorrhage that is neither cardiovascular bleeding nor a stroke;
   Accidental (e.g., physical accidents or drug overdoses) or trauma;
   Suicide;
   Prescription Drug Error (e.g., prescribed drug overdose, use of inappropriate drug, or drug-drug interaction); and
   Neurological process that is not a stroke or hemorrhage.
Malignancy: Malignancy is coded as cause of death, if:
   Death results directly from the cancer; or
   Death results from a concurrent illness that could be a consequence of a cancer; or
   Death results from withdrawal of other therapies because of concerns relating to the poor prognosis associated with the cancer; and
   Death results from an illness that is not a consequence of a cancer.

Cancer deaths may arise from cancers that were present prior to randomization or which developed subsequently. It may be helpful to distinguish these two scenarios (i.e., worsening of prior malignancy; new malignancy). Suggested categorization includes the following organ systems; Lung/larynx, breast, leukemia/lymphoma, upper GI, melanoma, central nervous system, colon/rectum, renal, bladder, prostate, other/unspecified, or unknown.

Definition of Undetermined Cause of Death: refers to a death not attributable to one of the above categories of cardiovascular death or to a non-cardiovascular cause. The inability to classify the cause of death is generally due to lack of information (e.g., the only available information is “patient died”) or when there is insufficient supporting information or detail to assign the cause of death. In this trial, when a cause of death was not readily apparent (e.g., found dead at home), the cause was assumed to be cardiovascular in origin, unless one of the following two scenarios occur: there was no information or data available regarding the circumstances of death other than that a death had occurred; or the available data are conflicting regarding whether the death was cardiovascular or non-cardiovascular.

Definition of Myocardial Infarction: The term myocardial infarction (MI) is used when there is evidence of myocardial necrosis in a clinical setting consistent with myocardial ischemia. In general, the diagnosis of MI requires the combination of evidence of myocardial necrosis (either changes in cardiac biomarkers or postmortem pathological findings); and supporting information derived from the clinical presentation, electrocardiographic changes, or the results of myocardial or coronary artery imaging.

The totality of the clinical, electrocardiographic, and cardiac biomarker information should be considered to determine whether or not an MI has occurred. Specifically, timing and trends in cardiac biomarkers and electrocardiographic information require careful analysis. The adjudication of MI should also take into account the clinical setting in which the event occurs. MI may be adjudicated for an event that has characteristics of a MI, but which does not meet the strict definition because biomarker or electrocardiographic results are not available.

The criteria for myocardial infarction include clinical presentation, biomarker evaluation, and ECG changes.

Clinical Presentation: The clinical presentation is consistent with diagnosis of myocardial ischemia and infarction. Other findings that might support the diagnosis of MI should be taken into account because a number of conditions are associated with elevations in cardiac biomarkers (e.g., trauma, surgery, pacing, ablation, congestive heart failure, hypertrophic cardiomyopathy, pulmonary embolism, severe pulmonary hypertension, stroke or subarachnoid hemorrhage, infiltrative and inflammatory disorders of cardiac muscle, drug toxicity, burns, critical illness, extreme exertion, and chronic kidney disease). Supporting information can also be considered from myocardial imaging and coronary imaging. The totality of the data may help differentiate acute MI from the background disease process.

Biomarker Evaluation: For cardiac biomarkers, laboratories should report an upper reference limit (URL). If the 99th percentile of the URL from the respective laboratory performing the assay is not available, then the URL for myocardial necrosis from the laboratory should be used. If the 99th percentile of the URL or the URL for myocardial necrosis is not available, the MI decision limit for the particular laboratory should be used as the URL. Laboratories can also report both the 99th percentile of the URL and the MI decision limit. Reference limits from the laboratory performing the assay are preferred over the manufacturer's listed reference limits in an assay's instructions for use. CK-MB and troponin are preferred, but CK may be used in the absence of CK-MB and troponin. For MI subtypes, different biomarker elevations for CK, CK-MB, or troponin were required. The specific criteria were referenced to the URL. In this study, patients may have presented acutely to hospitals which were not participating sites, it was not practical to stipulate the use of a single biomarker or assay, and the locally available results were to be used as the basis for adjudication. Since the prognostic significance of different types of myocardial infarctions (e.g., periprocedural myocardial infarction versus spontaneous myocardial infarction) may have been different, considerations evaluating outcomes for these subsets of patients separately were made.

ECG Changes: ECG changes can be used to support or confirm a MI.

Supporting evidence may be ischemic changes and confirmatory information may be new Q waves.

Criteria for acute myocardial ischemia (in absence of left ventricular hypertrophy (LVH) and left bundle branch block (LBBB)) include:
ST elevation: New ST elevation at the J point in two anatomically contiguous leads with the cut-off points: ≥0.2 mV in men (>0.25 mV in men <40 years) or ≥0.15 mV in women in leads V2-V3 and/or ≥0.1 mV in other leads.
ST depression and T-wave changes new horizontal or down-sloping ST depression ≥0.05 mV in two contiguous leads; and/or new T inversion ≥0.1 mV in two contiguous leads.

The above ECG criteria illustrate patterns consistent with myocardial ischemia. In patients with abnormal biomarkers, it is recognized that lesser ECG abnormalities may represent an ischemic response and may be accepted under the category of abnormal ECG findings.

Criteria for pathological Q-wave include: any Q-wave in leads V2-V3 ≥0.02 seconds or QS complex in leads V2 and V3; Q-wave ≥0.03 seconds and ≥0.1 mV deep or QS complex in leads I, II, aVL, aVF, or V4-V6 in any two leads of a contiguous lead grouping (I, aVL, V6; V4-V6; II, III, and aVF); and R-wave 0.04 s in V1-V2 and R/S ratio >1 with a concordant positive T-wave in the absence of a conduction defect.

The same criteria are used for supplemental leads V7-V9, and for the Cabrera frontal plane lead grouping.

Criteria for Prior Myocardial Infarction include: pathological Q-waves, as defined above; and R-wave ≥0.04 seconds in V1-V2 and R/S ≥1 with a concordant positive T-wave in the absence of a conduction defect.

Myocardial Infarction Subtypes: Several MI subtypes are commonly reported in clinical investigations, and each is defined below:

### 1. Spontaneous MI:

Detection of rise and/or fall of cardiac biomarkers with at least one value above the URL with at least one of the following:
   Clinical presentation consistent with ischemia;
   ECG evidence of acute myocardial ischemia;
   New pathological Q waves;
   Imaging evidence of new loss of viable myocardium or new regional wall motion abnormality; and/or
   Autopsy evidence of acute MI.
If biomarkers are elevated from a prior infarction, then a spontaneous myocardial infarction is defined as one of the following:
   Clinical presentation consistent with ischemia;
   ECG evidence of acute myocardial ischemia;
   New pathological Q waves;
   Imaging evidence of new loss of viable myocardium or new regional wall motion abnormality; and/or
   Autopsy evidence of acute MI; and
Both of the Following:
   Evidence that cardiac biomarker values were decreasing (e.g., two samples 3-6 hours apart) prior to the suspected MI (note: If biomarkers are increasing or peak is not reached, then a definite diagnosis of recurrent MI is generally not possible); and
   ≥20%, increase (and >URL) in troponin or CK-MB between a measurement made at the time of the initial presentation and a further sample taken 3-6 hours later.
      2. Percutaneous Coronary Intervention-Related Myocardial Infarction: is defined by any of the following criteria. MI associated with and occurring within 48 hours of PCI, with elevation of cardiac biomarker values to >5×99^{th} percentile of the URL in patients with normal baseline values (≤99^{th} percentile URL), or a rise of [cardiac biomarker] values ≥20% if baseline values are elevated and are stable or falling. This classification also requires at least one of the following:
Symptoms suggestive of myocardial ischemia (e.g., prolonged ischemia ≥20 min);
New ischemic changes on ECG or new LBBB;
Angiographic loss of patency of a major coronary artery or a side branch or persistent slow flow or no flow or embolization; and/or
Imaging evidence of new loss of viable myocardium or new regional wall motion abnormality.
   3. Coronary Artery Bypass Grafting-Related (CABG) Myocardial Infarction: is defined by the following criteria. Symptoms of cardiac ischemia were not required, and data was collected in such a way that analyses using ≥20% or ≥50% could both be performed.
Biomarker elevations within 48 hours of CABG:
   Troponin or CK-MB (preferred) >10×99^{th} percentile of the URL; and
   No evidence that cardiac biomarkers were elevated prior to the procedure; or
   Both of the following are true:
      ≥50% increase in the cardiac biomarker result; and
      Evidence that cardiac biomarker values were decreasing (e.g., two samples 3-6 hours apart) prior to the suspected MI; and
One of the following is true:
   New pathological Q-waves persistent through 30 days;
   New persistent non-rate-related LBBB;
   Angiographically documented new graft or native coronary artery occlusion;
   Other complication in the operating room resulting in loss of myocardium; or
   Imaging evidence of new loss of viable myocardium.
Autopsy evidence of acute MI.
   4. Silent Myocardial Infarction: is defined by the following:
No evidence of acute myocardial infarction; and
Any one of the following criteria:
   New pathological Q-waves. A confirmatory ECG is recommended if there have been no clinical symptoms or history of myocardial infarction;
   Imaging evidence of a region of loss of viable myocardium that is thinned and fails to contract, in the absence of a non-ischemic cause; and/or
   Autopsy evidence of a healed or healing MI.

In the case of evanescent Q waves, the last ECG determines whether a silent infarction has occurred.

Sub-classification of Myocardial Infarction: The universal MI definition includes clinical classification of different types of MI, electrocardiographic features, and by biomarker evaluation, with the definition of each provided below.

Clinical Classification of Different Types of Myocardial Infarction include the following:
Type 1: Spontaneous myocardial infarction related to ischemia due to a primary coronary event such as plaque erosion and/or rupture, fissuring, or dissection;
Type 2: Myocardial infarction secondary to ischemia due to either increased oxygen demand or decreased supply, e.g., coronary artery spasm, coronary embolism, anemia, arrhythmias, hypertension, or hypotension;
Type 3: Sudden unexpected cardiac death, including cardiac arrest, often with symptoms suggestive of myocardial ischemia, accompanied by presumably new ST elevation, or new LBBB, or evidence of fresh thrombus in a coronary artery by angiography and/or at autopsy, but death occurring before blood samples could be obtained, or at a time before the appearance of cardiac biomarkers in the blood;
Type 4a: Myocardial infarction associated with Percutaneous Coronary Intervention (PCI);
Type 4b: Myocardial infarction associated with stent thrombosis as documented by angiography or at autopsy;
Type 4c: Myocardial infarction associated with stent restenosis as detected by angiography or at autopsy; and
Type 5: Myocardial infarction associated with CABG.

By Electrocardiographic Features include:
ST-Elevation MI (STEMI). The additional categories of STEMI include: Q wave, non-Q-wave, or unknown (no ECG or ECG non-interpretable);
Non-ST-Elevation MI (NSTEMI). The additional categories of NSTEMI may include: Q wave, non-Q-wave, or unknown (no ECG or ECG non-interpretable); and
Unknown (no ECG or ECG not interpretable).

All events adjudicated as MI were classified as STEMI, NSTEMI, or Unknown; however, it is acknowledged that a significant proportion of periprocedural (PCI or CABG) events may have missing, inadequate, or uninterpretable ECG documentation.

By Biomarker Elevation (per universal MI definition): The magnitude of cardiac biomarker elevation can be calculated as a ratio of the peak biomarker value divided by the 99th percentile URL. The biomarker elevation can be provided for various MI subtypes.

Definition of Hospitalize of Unstable Angina: Unstable angina requiring hospitalization is defined as:
Ischemic discomfort (angina or symptoms thought to be equivalent) ≥10 minutes in duration occurring at rest or in an accelerating pattern with frequent episodes associated with progressively decreased exercise capacity;
Prompting an unscheduled hospitalization within 24 hours of the most recent symptoms. Hospitalization is defined as an admission to an inpatient unit or a visit to an emergency department that results in at least a 24-hour stay (or a date change if the time of admission/discharge is not available); and
At least one of the following:
   New or worsening ST or T wave changes on resting ECG (in absence of confounders, such as LBBB or LVH);
      Transient ST elevation (duration <20 minutes): New ST elevation at the J point in two anatomically contiguous leads with the cut-off points: ≥0.2 mV in men (>0.25 mV in men <40 years) or ≥0.15 mV in women in leads V2-V3 and/or ≥0.1 mV in other leads;
      ST depression and T-wave changes: New horizontal or down-sloping ST depression ≥0.05 mV in two contiguous leads; and/or new T inversion ≥0.1 mV in two contiguous leads.
   Definite evidence of inducible myocardial ischemia as demonstrated by:
      An early positive exercise stress test, defined as ST elevation or ≥2 mm ST depression prior to 5 mets; or at least one of the following: stress echocardiography (reversible wall motion abnormality); myocardial scintigraphy (reversible perfusion defect); or MRI (myocardial perfusion deficit under pharmacologic stress).
   Angiographic evidence of new or worse ≥70% lesion and/or thrombus in an epicardial coronary artery that is believed to be responsible for the myocardial ischemic symptoms/signs; and
   Need for coronary revascularization procedure (PCI or CABG) for the presumed culprit lesion(s). This criterion would be fulfilled if revascularization was undertaken during the unscheduled hospitalization, or subsequent to transfer to another institution without interceding home discharge;
Negative cardiac biomarkers and no evidence of acute MI.

General Considerations include:

Escalation of pharmacotherapy for ischemia, such as intravenous nitrates or increasing dosages of β-blockers, should be considered supportive of the diagnosis of unstable angina. However, a typical presentation and admission to the hospital with escalation of pharmacotherapy, without any of the additional findings listed under category 3, would be insufficient alone to support classification as hospitalization for unstable angina.

If subjects were admitted with suspected unstable angina, and subsequent testing revealed a noncardiac or non-ischemic etiology, this event should not have been recorded as hospitalization for unstable angina. Potential ischemic events meeting the criteria for myocardial infarction should not have been adjudicated as unstable angina.

Planned hospitalization or re-hospitalization for performance of an elective revascularization in patients who did not fulfill the criteria for unstable angina should not have been considered a hospitalization for unstable angina. For example: hospitalization of a patient with stable exertional angina for coronary angiography and PCI that is prompted by a positive outpatient stress test should not be considered hospitalization for unstable angina; or re-hospitalization of a patient meeting the criteria for unstable angina who was stabilized, discharged, and subsequently readmitted for revascularization, does not constitute a second hospitalization for unstable angina.

A patient who underwent an elective catheterization where incidental coronary artery disease was found and who subsequently underwent coronary revascularization was not to be considered as meeting the hospitalization for unstable angina endpoint.

Transient Ischemic Attack: Transient ischemic attack (TIA) is defined as a transient episode (<24 hours) of neurological dysfunction caused by focal brain, spinal cord, or retinal ischemia, without acute infarction.

Stroke: Stroke is defined as an acute episode of neurological dysfunction caused by focal or global brain, spinal cord, or retinal vascular injury.

Ischemic Stroke: Ischemic stroke is defined as an acute episode of focal cerebral, spinal, or retinal dysfunction caused by an infarction of central nervous system tissue. Hemorrhage may be a consequence of ischemic stroke. In this situation, the stroke is an ischemic stroke with hemorrhagic transformation and not a hemorrhagic stroke.

Hemorrhagic Stroke: Hemorrhagic stroke is defined as an acute episode of focal or global cerebral or spinal dysfunction caused by a nontraumatic intraparenchymal, intraventricular, or subarachnoid hemorrhage. However, microhemorrhages seen on T2-weighted MRI imaging, subdural, and epidural hemorrhages are not considered hemorrhagic strokes.

Undetermined Stroke: Undetermined stroke is defined as an acute episode of focal or global neurological dysfunction caused by presumed brain, spinal cord, or retinal vascular injury as a result of hemorrhage or infarction but with insufficient information to allow categorization as ischemic or hemorrhagic.

Stroke Disability: Stroke disability should be measured by a reliable and valid scale in all cases, typically at each visit and 90 days after the event. For example, the modified Rankin Scale shown below in Table 14 may be used to address this requirement:

TABLE 14
Rankin Scaled Used to Assess Stroke Disability in Patients

| Scale | Disability |
|---|---|
| 0 | No symptoms at all. |
| 1 | No significant disability despite symptoms; able to carry out all |
| | usual duties and activities. |
| 2 | Slight disability, unable to perform all previous activities but |
| | able to look after own affairs without assistance. |
| 3 | Moderate disability; requiring some help but able to walk without |
| | assistance. |
| 4 | Moderately severe disability, unable to walk without assistance |
| | and unable to attend to own bodily needs without assistance. |
| 5 | Severe disability, bedridden, incontinent, and requiring constant |
| | nursing and attention. |
| 6 | Dead. |

Additional Considerations: Evidence of vascular central nervous system injury without recognized neurological dysfunction may be observed. Examples include micro-hemorrhage, silent infarction, and silent hemorrhage. Subdural hematomas are intracranial hemorrhagic events and not strokes. The distinction between a transient ischemic attack and an ischemic stroke is the presence of infarction. Persistence of symptoms is an acceptable indicator of acute infarction.

Definition of Heart Failure Event: is defined as an event that meets all of the following criteria:
The patient is admitted to the hospital with a primary diagnosis of HF;
The patient's length-of-stay in the hospital extends for at least 24 hours (or a change in calendar date if the hospital admission and discharge times are unavailable);
The patient exhibits documented new or worsening symptoms due to HF on presentation, including at least one of the following: dyspnea (dyspnea with exertion, dyspnea at rest, orthopnea, paroxysmal nocturnal dyspnea), decreased exercise tolerance, fatigue, or other symptoms of worsened end-organ perfusion or volume overload (must be specified and described by the protocol);
The patient has objective evidence of new or worsening HF, consisting of at least two physical examination findings or one physical examination finding and at least one laboratory criterion, including:
   Physical examination findings considered to be due to heart failure, including new or worsened: Peripheral edema, increasing abdominal distention or ascites (in the absence of primary hepatic disease), S₃ gallop, clinically significant or rapid weight gain thought to be related to fluid retention; or
   Laboratory evidence of new or worsening HF, if obtained within 24 hours of presentation, including: increased B-type natriuretic peptide (BNP)/N-terminal pro-BNP (NT-proBNP) concentrations consistent with decompensation of heart failure (such as BNP >500 pg/mL or NT-proBNP >2,000 pg/mL). In patients with chronically elevated natriuretic peptides, a significant increase should be noted above baseline, radiological evidence of pulmonary congestion, or non-invasive or invasive diagnostic evidence of clinically significant elevated left- or right-sided ventricular filling pressure or low cardiac output. For example, echocardiographic criteria could include: E/e′>15 or D-dominant pulmonary venous inflow pattern, plethoric inferior vena cava with minimal collapse on inspiration, or decreased left ventricular outflow tract (LVOT) minute stroke distance (time velocity integral [TVI]) OR right heart catheterization showing a pulmonary capillary wedge pressure (pulmonary artery occlusion pressure) ≥18 mmHg, central venous pressure ≥12 mmHg, or a cardiac index <2.2 L/min/m².
The patient receives initiation or intensification of treatment specifically for HF, including at least one of the following: significant augmentation in oral diuretic therapy, intravenous diuretic, inotrope, or vasodilator therapy, or mechanical or surgical intervention. The mechanical or surgical intervention includes mechanical circulatory support (e.g., intra-aortic balloon pump, ventricular assist device) and/or mechanical fluid removal (e.g., ultrafiltration, hemofiltration, dialysis).

New Heart Failure/Heart Failure Not Requiring Hospitalization: is defined as an event that meets all of the following: the patient has an urgent, unscheduled office/practice or emergency department visit for a primary diagnosis of HF, but not meeting the criteria for a HF hospitalization; all signs and symptoms for HF hospitalization must be met; and the patient receives initiation or intensification of treatment specifically for HF, as detailed in the above section with the exception of oral diuretic therapy, which was not sufficient.

### Interventional Cardiology Definitions

### Clinical Definitions

Clinically Driven Target Lesion Revascularization: Revascularization is clinically driven if the target lesion diameter stenosis is less than 50% by quantitative coronary angiography (QCA) and the subject has clinical or functional ischemia which cannot be explained by another native coronary or bypass graft lesion. Clinical or functional ischemia includes any of the following: a history of angina pectoris, presumably related to the target vessel; objective signs of ischemia at rest (electrocardiographic changes) or during exercise test (or equivalent), presumably related to the target vessel; and abnormal results of any invasive functional diagnostic test (e.g., coronary flow reserve [CFR] or fractional flow reserve [FFR]).

Non-Target Lesion and Non-Target Lesion Revascularization: A lesion for which revascularization is not attempted or one in which revascularization is performed using a non-study device, respectively.

Non-Target Vessel and Non-Target Vessel Revascularization: A vessel for which revascularization is not attempted or one in which revascularization is performed using a non-study device, respectively.

Percutaneous Coronary Intervention (PCI) Status includes:
Elective: The procedure can be performed on an outpatient basis or during a subsequent hospitalization without significant risk of myocardial infarction (MI) or death. For stable in-patients, the procedure is being performed during this hospitalization for convenience and ease of scheduling and NOT because the patient's clinical situation demands the procedure prior to discharge.
Urgent: The procedure should be performed on an inpatient basis and prior to discharge because of significant concerns that there is risk of myocardial ischemia, MI, and/or death. Patients who are outpatients or in the emergency department at the time that the cardiac catheterization is requested would warrant hospital admission based on their clinical presentation.
Emergency: The procedure should be performed as soon as possible because of substantial concerns that ongoing myocardial ischemia and/or MI could lead to death. “As soon as possible” refers to a patient who is of sufficient acuity that one would cancel a scheduled case to perform this procedure immediately in the next available room during business hours, or one would activate the on-call team were this to occur during off-hours.
Salvage: The procedure is a last resort. The patient is in cardiogenic shock when the PCI begins (i.e., the time at which the first guide wire or intracoronary device is introduced into a coronary artery or bypass graft for the purpose of mechanical revascularization) or within the last ten minutes prior to the start of the case or during the diagnostic portion of the case, the patient has also received chest compressions or has been on unanticipated circulatory support (e.g., intra-aortic balloon pump, extracorporeal mechanical oxygenation, or cardiopulmonary support).

Percutaneous Coronary Intervention (PCI): Placement of an angioplasty guide wire, balloon, or other device (e.g., stent, atherectomy catheter, brachytherapy delivery device, or thrombectomy catheter) into a native coronary artery or coronary artery bypass graft for the purpose of mechanical coronary revascularization. In the assessment of the severity of coronary lesions with the use of intravascular ultrasound, CFR, or FFR, insertion of a guide wire was not considered PCI.

### Peripheral Vascular Intervention Definitions

Peripheral Vascular Intervention Definition: Peripheral vascular intervention is a catheter-based or open surgical procedure designed to improve peripheral arterial or venous blood flow or otherwise modify or revise vascular conduits. Procedures may include, but are not limited to, balloon angioplasty, stent placement, thrombectomy, embolectomy, atherectomy, dissection repair, aneurysm exclusion, treatment of dialysis conduits, placement of various devices, intravascular thrombolysis or other pharmacotherapies, and open surgical bypass or revision. In general, the intention to perform percutaneous peripheral vascular intervention is denoted by the insertion of a guide wire into a peripheral artery or vein. The target vessel(s) and the type of revascularization procedure (e.g., surgical bypass, thrombectomy, endarterectomy, percutaneous angioplasty, stent placement, thromboembolectomy, and thrombolysis) should be specified and recorded. For the sake of simplicity, this definition applies to the extracranial carotid artery and other non-cardiac arteries and veins, and it excludes the intracranial vessels and lymphatics.

Procedural Status includes:
Non-Elective: Non-elective procedures include emergent and urgent procedures. A non-elective procedure is a procedure that is performed without delay because there is clinical consensus that the procedure should occur imminently. Non-elective procedures imply a degree of instability of the patient, urgency of the medical condition, or instability of the threatening lesion.
   Emergent: A procedure that is performed immediately because of the acute nature of the medical condition (e.g., acute limb ischemia, acute aortic dissection) and the increased morbidity or mortality associated with a temporal delay in treatment.
   Urgent: An urgent procedure is one that is not emergent but is required to be performed on a timely basis (≥24 hrs) (e.g., a patient who has been stabilized following initial treatment of acute limb ischemia, and there is clinical consensus that a definitive procedure should occur within the next 24 hours).
Elective: An elective procedure is one that is scheduled and is performed on a patient with stable disease, or in whom there is no urgency and/or increased morbidity or mortality associated with a planned procedure.

Definition of Any Revascularization Procedure: Any revascularization includes any arterial vascular intervention done to treat ischemia or prevent major ischemic events, including percutaneous or surgical intervention of the coronary, peripheral, or carotid arteries. Aneurysm repairs, dissection repairs, arterial-venous fistula or graft placement or repairs, or renal arterial intervention for hypertension or renal dysfunction are not included.

Definition of Cardiac Arrhythmia Requiring Hospitalization: An arrhythmia that either results in hospitalization (≥24 hours) during or within 24 hours of the termination of the last episode for treatment or requires continued hospitalization for treatment, including any one of the following:
Atrial arrhythmia—atrial fibrillation, atrial flutter, supraventricular tachycardia that requires cardioversion, drug therapy, or is sustained for greater than 1 minute;
Ventricular arrhythmia—Ventricular tachycardia or ventricular fibrillation requiring cardioversion and/or intravenous antiarrhythmics; and/or
Bradyarrhythmia—High-level AV block (defined as third-degree AV block or second-degree AV block), junctional or ventricular escape rhythm, or severe sinus bradycardia (typically with heart rate <30 bpm). The bradycardia must require temporary or permanent pacing.

Definition of Cardiac Arrest (Sudden Cardiac Death): A sudden, unexpected death due to the cessation of cardiac mechanical activity, confirmed by the absence of a detectable pulse, unresponsiveness, and apnea (or agonal, gasping respirations) of presumed cardiac etiology. An arrest is presumed to be cardiac (i.e., related to heart disease) if this is likely, based on the available information, including hospital records and autopsy data. The cardiac arrest is further sub-classified into either: witnessed, occurring within 60 minutes from the onset of new symptoms, in the absence of a clear cause other than cardiovascular; or unwitnessed, within 24 hours of being observed alive, in the absence of pre-existing other non-cardiovascular causes of death.

Non-cardiac causes of cardiac arrest, such as drug overdose, suicide, drowning, hypoxia, exsanguination, cerebrovascular accident, subarachnoid hemorrhage, or trauma must not be present.

Definition of Resuscitated Cardiac Arrest: Resuscitated Cardiac Arrest is present when there is restoration of both organized electrical activity and organized mechanical activity resulting in restoration of spontaneous circulation (defined as the documented presence of a measurable pulse and blood pressure at any time after initiation of resuscitative efforts).

Criteria for the Diagnosis of Metabolic Syndrome: The diagnosis of metabolic syndrome requires the presence of three out of the following five specific components using the following criteria with cut points of parameters as defined in Table 1 and listed below, and waist circumference cut points further guided by the Table 15.
A waist circumference 35 inches (88 cm) for all women, and Asian, Hispanic, or Latino men, and waist circumference ≥40 inches (102 cm) for all other men;
Elevated TG (TG ≥150 mg/dL);
Reduced HDL-C(HDL-C <40 mg/dL if male; HDL-C <50 mg/dL if female);
Elevated blood pressure (systolic ≥130 mmHg and/or diastolic ≥85 mmHg) or an antihypertensive therapy with medical history of hypertension; and
Elevated fasting glucose (fasting glucose ≥100 mg/dL) or on drug therapy for elevated glucose.

TABLE 15
Current Recommended Waist Circumference Thresholds for
Abdominal Obesity by Organization and Population.

| | | Waist Circumference | |
|---|---|---|---|
| | | Threshold | |
| | Population | Men | Women |
| Organization | (Reference) | (cm) | (cm) |
| IDF (4) | Europid | ≥94 | ≥80 |
| WHO (7) | Caucasian | ≥94 | ≥80 |
| | | (increased | |
| | | risk) | |
| | | ≥102 | ≥88 |
| | | (still higher | |
| | | risk) | |
| AHA/NHLBI (ATP III)* | U.S. | ≥102 | ≥88 |
| Health Canada | Canada | ≥102 | ≥88 |
| European Cardiovascular | European | ≥102 | ≥88 |
| Societies | | | |
| IDF | Asian | ≥90 | ≥80 |
| | (including Japanese) | | |
| WHO | Asian | ≥90 | ≥80 |
| Japanese Obesity Society | Japanese | ≥85 | ≥90 |
| Cooperative Task Force | China | ≥85 | ≥80 |
| IDF | Middle East, | ≥94 | ≥80 |
| | Mediterranean | | |
| IDF | Sub-Saharan African | ≥94 | ≥80 |
| IDF | Ethnic Central & | ≥90 | ≥80 |
| | South American | | |

IDF = International Diabetes Federation; WHO = World Health Organization; AHA/NHLBI (ATP III) = American Heart Association/National Heart, Lung, and Blood Institute Adult Treatment Panel III;
*Recent AHA/NHLBI guidelines for metabolic syndrome recognize an increased risk for cardiovascular disease and diabetes at waist-circumference thresholds of ≥94 cm in men and ≥80 cm in women and identify these as optional cut points for individuals or populations with increased insulin resistance.

### Statistical Analysis

In this event-driven trial, it was estimated that approximately 1612 adjudicated primary endpoint events would be necessary to provide 90% power to detect a 15% lower risk of the primary composite endpoint in the AMR101 group than in the placebo group. This resulted in an estimated sample size of approximately 7990 patients to reach the number of primary endpoints. The primary efficacy analysis was based on the time from randomization to the first occurrence of any component of the primary composite endpoint. If the relative risk reduction with administration of AMR101 in the primary endpoint was significant (final two-sided alpha level=0.0437; determined from O'Brien-Fleming boundaries generated using the Lan-DeMets alpha-spending function after accounting for two protocol pre-specified interim efficacy analyses), in a hierarchical fashion, the key secondary endpoint and other prespecified secondary endpoints were to be tested at the same final alpha level of 0.0437. All primary efficacy analyses followed the intent-to-treat principle. HRs and 95% CI were generated using a Cox proportional hazard model with treatment as covariate and stratified by cardiovascular risk category, geographic region, and use of ezetimibe. Log-rank P values were reported from a Kaplan-Meier analysis, stratified by the three randomization factors, to evaluate the timing of events in the two treatment groups.

### Results

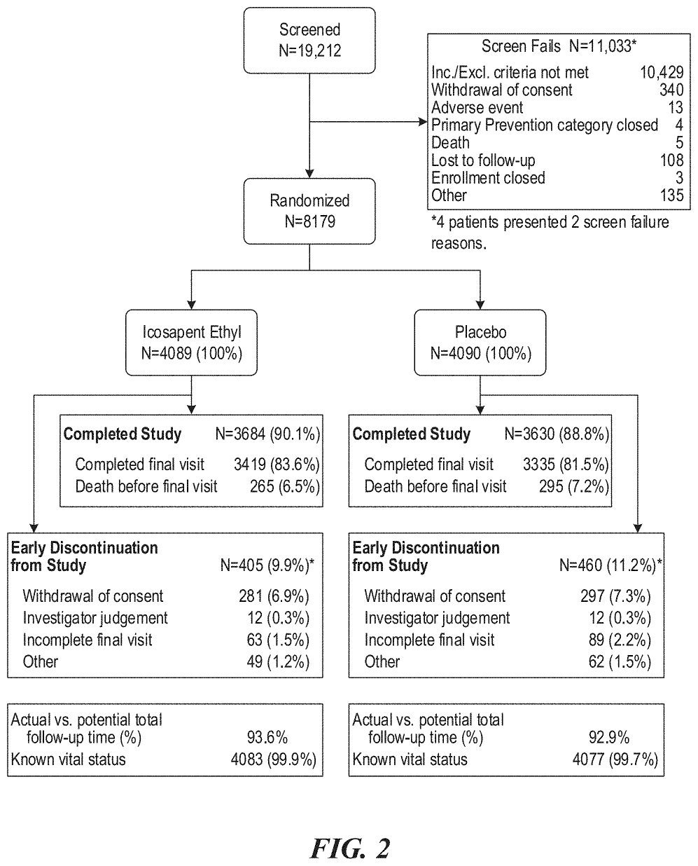

Subject Disposition: The subject disposition by treatment group is depicted in FIG. 2. A total of 19,212 patients were screened of whom 8,179 (43%) were randomized. At the time of database lock, vital status was available in 99.8%; 152 (1.9%) patients did not complete final study visits and 578 (7.1%) patients withdrew consent.

Demographic and Baseline Disease Characteristics: Among the patients who underwent randomization, 70.7% were enrolled on the basis of secondary prevention (i.e., patients had established cardiovascular disease) and 29.3% for primary prevention (i.e., patients had diabetes mellitus and at least one additional risk factor). The median age was 64 years, 28.8% were female, and 38.5% were from the United States. At baseline, the median LDL-cholesterol was 75.0 mg/dL, HDL-cholesterol was 40.0 mg/dL, and triglycerides were 216.0 mg/dL. The baseline characteristics of the patients are provided below in Table 16.

TABLE 16
Demographic and Randomization Stratification
Information of the ITT Population

| | Icosapent ethyl | | Placebo | |
|---|---|---|---|---|
| | (N = 4089) | | (N = 4090) | |
| Age (years), Median | 64.0 | (57.0-69.0) | 64.0 | (57.0-69.0) |
| (Q1-Q3) | | | | |
| Female, (n %) | 1162 | (28.4%) | 1195 | (29.2%) |
| Non-White, (n %) | 398 | (9.7%) | 401 | (9.8%) |
| Age ≥65 years, n (%) | 1857 | (45.4%) | 1906 | (46.6%) |
| Male, n (%) | 2927 | (71.6%) | 2895 | (70.8%) |
| White, n (%)^{[1]} | 3691 | (90.3%) | 3688 | (90.2%) |
| BMI (kg/m²), Median | 30.8 | (27.8-34.5) | 30.8 | (27.9-34.7) |
| (Q1-Q3) | | | | |
| BMI ≥30 (kg/M²), | 2331 | (57.0%) | 2362 | (57.8%) |
| n (%) | | | | |
| Geographic Region, | | | | |
| n (%) | | | | |
| Westernized ^{[2]} | 2906 | (71.1%) | 2905 | (71.0%) |
| Eastern Europe ^{[3]} | 1053 | (25.8%) | 1053 | (25.7%) |
| Asia Pacific ^{[4]} | 130 | (3.2%) | 132 | (3.2%) |
| CV Risk Category, | | | | |
| n (%) | | | | |
| Secondary Prevention | 2892 | (70.7%) | 2893 | (70.7%) |
| Primary Prevention | 1197 | (29.3%) | 1197 | (29.3%) |
| Ezetimibe Use, n (%) | 262 | (6.4%) | 262 | (6.4%) |
| Statin Intensity, n (%) | | | | |
| Low | 254 | (6.2%) | 267 | (6.5%) |
| Moderate | 2533 | (61.9%) | 2575 | (63.0%) |
| High | 1290 | (31.5%) | 1226 | (30.0%) |
| Missing | 12 | (0.3%) | 22 | (0.5%) |
| Diabetes, n (%) | | | | |
| Type I Diabetes | 27 | (0.7%) | 30 | (0.7%) |
| Type II Diabetes | 2367 | (57.9%) | 2363 | (57.8%) |
| No Diabetes at Baseline | 1695 | (41.5%) | 1694 | (41.4%) |
| Data Missing | 0 | | 3 | (0.1%) |
| hs-CRP (mg/L), Median | 2.2 | (1.1-4.5) | 2.1 | (1.1-4.5) |
| (Q1-Q3) | | | | |
| Triglycerides (mg/dL), | 216.5 | (176.5-272.0) | 216.0 | (175.5-274.0) |
| Median (Q1-Q3) | | | | |
| HDL-C (mg/dL), | 40.0 | (34.5-46.0) | 40.0 | (35.0-46.0) |
| Median (Q1-Q3) | | | | |
| LDL-C (mg/dL), | 74.0 | (61.5-88.0) | 76.0 | (63.0-89.0) |
| Median (Q1-Q3) | | | | |
| Triglycerides Category | | | | |
| <150 mg/dL | 412 | (10.1%) | 429 | (10.5%) |
| 150 to <200 mg/dL | 1193 | (29.2%) | 1191 | (29.1%) |
| ≥200 mg/dL | 2481 | (60.7%) | 2469 | (60.4%) |
| Triglycerides ≥200 | 823 | (20.1%) | 794 | (19.4%) |
| mg/dL and HDL-C ≤35 | | | | |
| mg/dL | | | | |
| EPA (μg/mL), Median | 26.1 | (17.1-40.1) | 26.1 | (17.1-39.9) |
| (Q1-Q3) | | | | |

In general, the baseline value is defined as the last non-missing measurement obtained prior to the randomization. The baseline LDL-C value obtained via preparative ultracentrifugation was used, unless this value was missing. If the LDL-C preparative ultracentrifugation value was missing, then another LDL-C value was be used, with prioritization of values obtained from LDL-C Direct measurements, followed by LDL-C derived by the Friedewald calculation (only for patients with TG <400 mg/dL), and finally LDL-C derived using the calculation published by Johns Hopkins University investigators. At Visit 1 and Visit 1.1, Direct LDL-C was used if at the same visit TG >400 mg/dL At all remaining visits, LDL-C was measured by Direct LDL-C or by preparative ultracentrifugation if at the same visit TG >400 mg/dL. For all other lipid and lipoprotein marker parameters, wherever possible, baseline was derived as the arithmetic mean of the Visit 2 (Day 0) value and the preceding Visit 1 (or Visit 1.1) value. If only one of these values was available, the single available value was used as baseline. The only significant baseline between group difference with p < 0.05 was LDL-C (p = 0.03).
^{[1]}Race as reported by the investigators.
^{[2]} Westernized region includes Australia, Canada, the Netherlands, New Zealand, United States, and South Africa.
^{[3]} Eastern European region includes Poland, Romania, Russian Federation, and Ukraine.
^{[4]} Asia Pacific region includes India.

The median trial follow-up duration was 4.9 years with a maximum of 6.2 years. The median change in triglycerides from baseline to one year was −18.3% (−39.0 mg/dL) in the AMR101 group and +2.2% (4.5 mg/dL) in the placebo group; the median reduction from baseline (as estimated with the use of the Hodges-Lehmann approach) was 19.7% greater in the AMR101 group than in the placebo group (a 44.5 mg/dL [0.50 mmol/L] greater reduction; P<0.001). The median change in LDL cholesterol level from baseline was an increase of 3.1% (2.0 mg/dL [0.05 mmol/L]) in the AMR101 group and an increase of 10.2% (7.0 mg/dL [0.18 mmol/L]) in the placebo group, a 6.6% (5.0 mg/dL [0.13 mmol/L]) lower increase with AMR101 than with placebo (P<0.001).

### Analyses of Primary Composite Endpoint

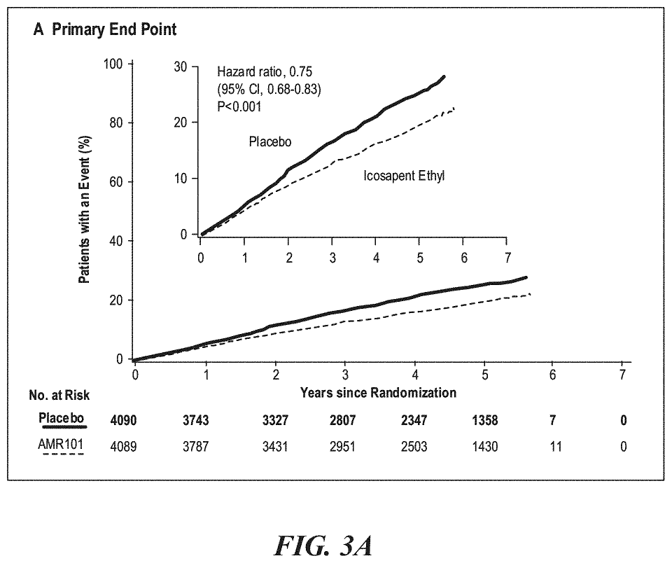

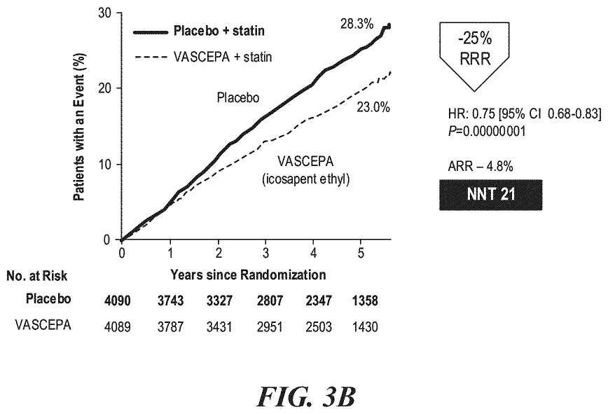

There were a total of 1606 adjudicated primary endpoint first events. FIG. 3A shows the Kaplan-Meier event curves for the primary efficacy endpoint of time to first occurrence of cardiovascular death, nonfatal myocardial infarction, nonfatal stroke, coronary revascularization, or unstable angina in the AMR101 and placebo groups with the inset showing the data on an expanded y axis. All patients were included in the analysis and patients experiencing more than one type of endpoint event were counted for their first occurrence in each event type. The primary endpoint as shown in FIG. 3A occurred in 17.2% of AMR101 patients versus in 22.0% of placebo patients (HR, 0.75; 95% CI, 0.68-0.83; P<0.001) for an absolute risk reduction (AAR) of 4.8% (95% CI, 3.1-6.5%) and number needed to treat (NNT) of 21 (95% CI, 15-33) over median follow-up of 4.9 years. Similarly, FIG. 3B shows the Kaplan-Meier estimates of the cumulative incidence of the primary composition endpoints over time. Significantly, FIG. 3B indicates a 25% relative risk reduction for the primary composite endpoint over the course of 5 years.

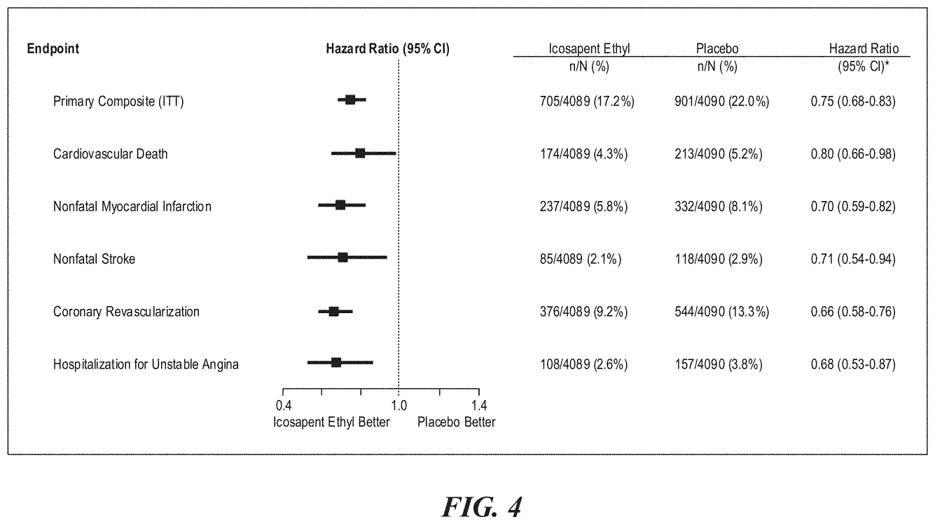

FIG. 4 lists the individual components of the primary endpoint analyzed as time to first event of each individual endpoint. Shown first in FIG. 4 is the HR and 95% CI for the primary composite endpoint event (time to first occurrence of either cardiovascular death, nonfatal myocardial infarction, nonfatal stroke, coronary revascularization, or unstable angina). Shown separately beneath FIG. 4 are HRs and 95% CIs for time to first occurrence of each type of individual primary endpoint component event, irrespective of whether contributing to the primary composite endpoint event or not.

### Analyses of Key Secondary Endpoints

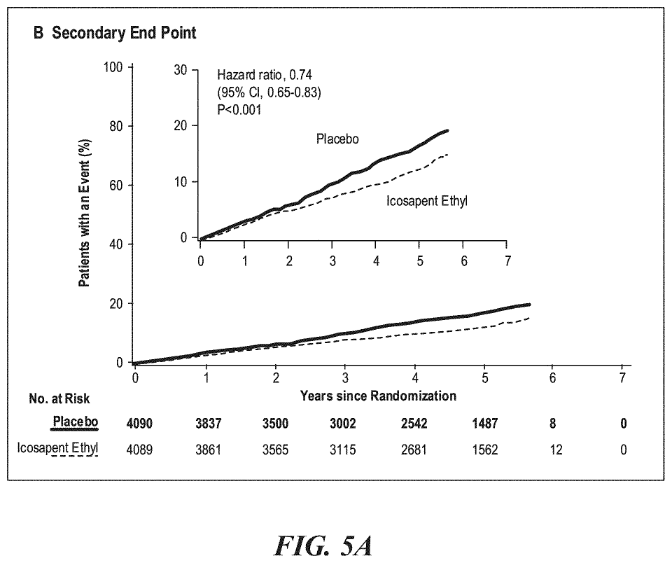

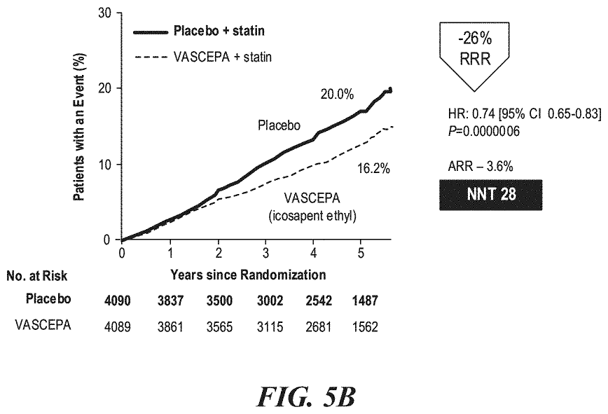

FIG. 5A shows the Kaplan-Meier event curves for the key secondary efficacy endpoint of time to first occurrence of cardiovascular death, nonfatal myocardial infarction, or nonfatal stroke in the AMR101 and placebo groups with the inset showing the data on an expanded y axis. All patients were included in the analysis, and patients experiencing more than one type of endpoint event were counted for their first occurrence in each event type. The key secondary efficacy endpoint as shown in FIG. 5A occurred in 11.2% of AMR101 patients versus 14.8% of placebo patients (HR, 0.74, 95% CI 0.65-0.83, P<0.001) for an absolute risk reduction of 3.6% (95% CI, 2.1-5.0%) and a number needed to treat of 28 (95% CI, 20-47) over median follow-up of 4.9 years. Similarly, FIG. 5B shows the Kaplan-Meier estimates of the cumulative incidence of the key secondary composition endpoints over time. Significantly, FIG. 5B indicates a 26% relative risk reduction for the key secondary composite endpoint over the course of 5 years.

### Analysis of Prespecified Subgroups

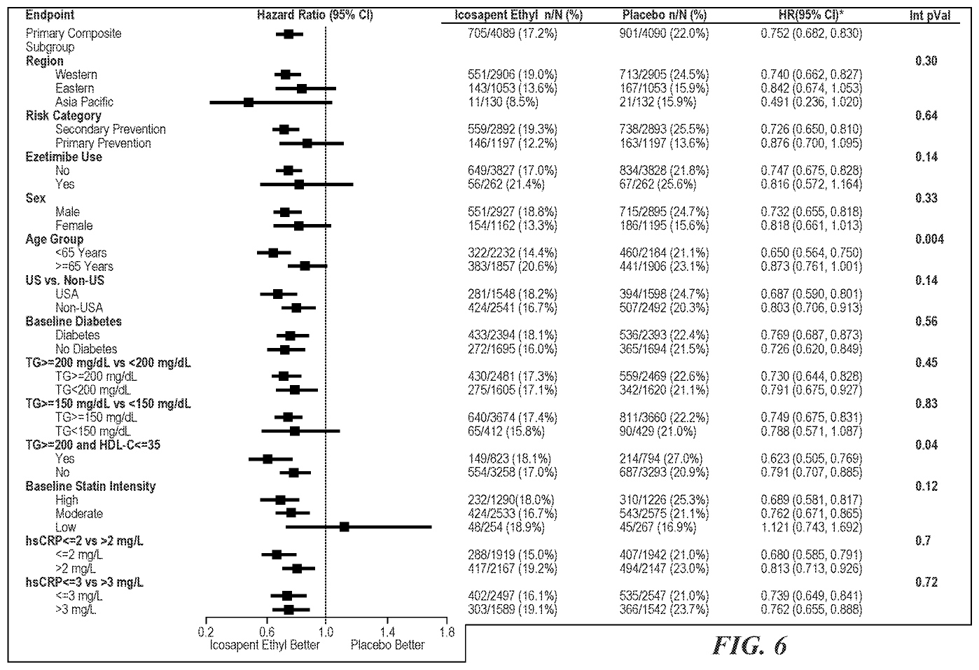

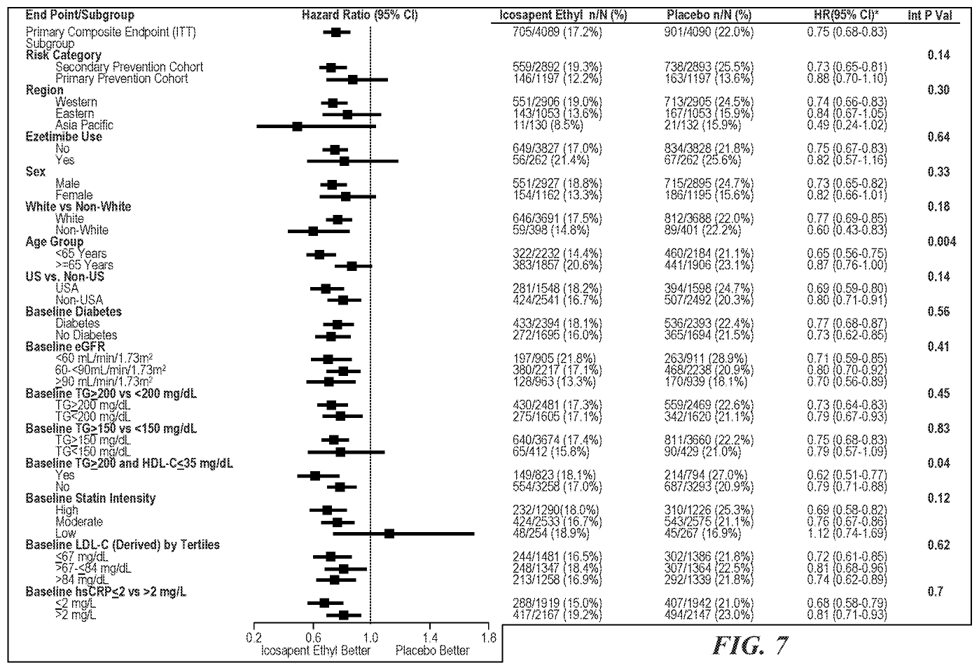

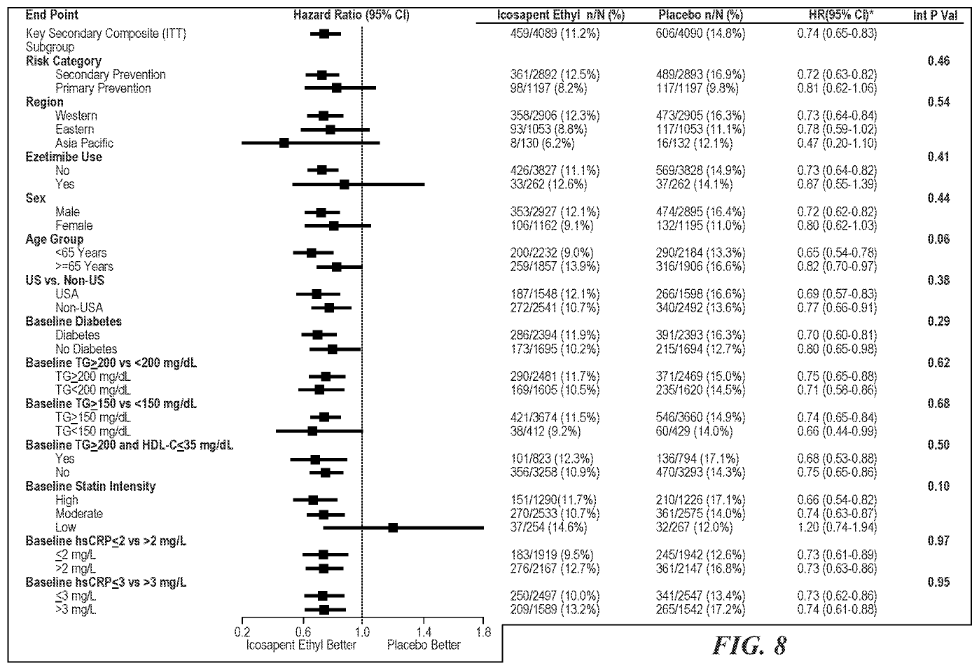

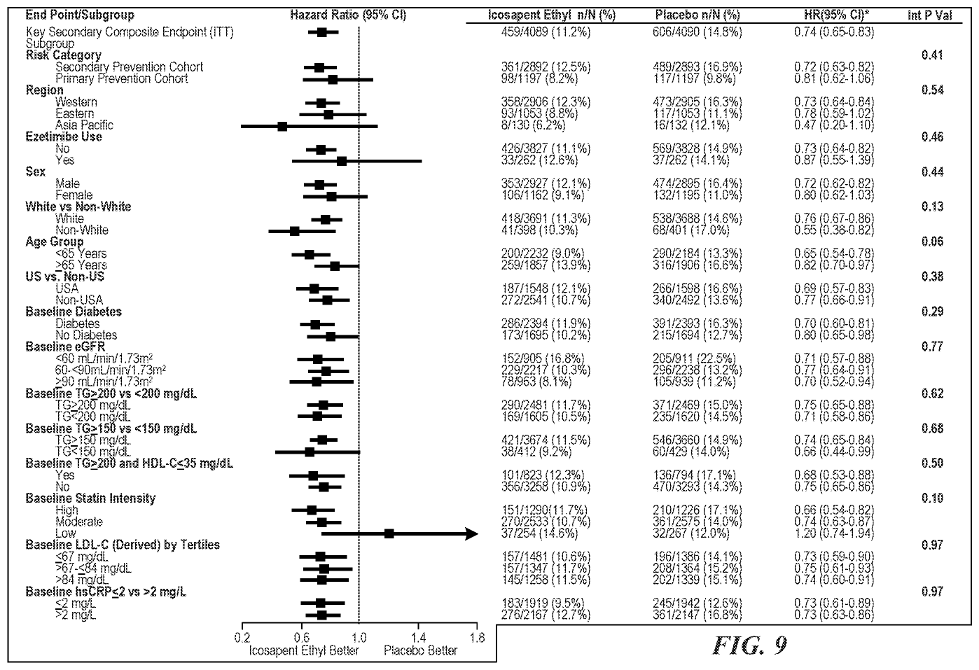

The primary efficacy outcomes in select prespecified subgroups are shown in FIGS. 6 and 7 with corresponding HRs and 95% CIs for the primary efficacy endpoint of time to first occurrence of cardiovascular death, nonfatal myocardial infarction, nonfatal stroke, coronary revascularization, or unstable angina from select prespecified subgroups in the AMR101 and placebo groups. The key secondary efficacy outcomes in select prespecified subgroups are shown in FIGS. 8 and 9 with corresponding HRs and 95% CIs for the key secondary efficacy endpoint of time to first occurrence of cardiovascular death, nonfatal myocardial infarction, nonfatal stroke, coronary revascularization, or unstable angina from select prespecified subgroups in the AMR101 and placebo groups. Significantly, FIGS. 6-9 indicate that a subject's baseline triglyceride levels (e.g., ≥150 vs. <150 mg/dL or ≥200 or <200 mg/dL) had no influence on the primary or key secondary efficacy endpoints.

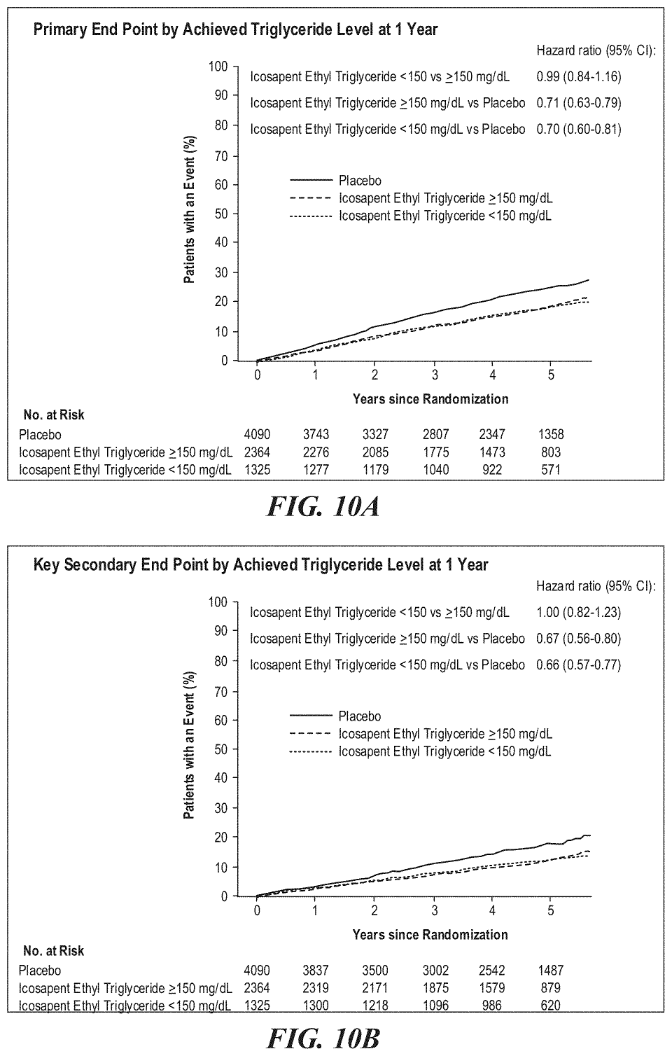

This conclusion is further substantiated by the combination of FIGS. 10A and 10B which show that achievement of on-treatment triglyceride levels above or below 150 mg/dL at one year did not influence the efficacy of AMR101 versus placebo. In particular, FIGS. 10A and 10B show the primary and key secondary endpoints by achieved triglyceride level (e.g., above or below 150 mg/dL) at 1 year (e.g., patients with a triglyceride level above or below 150 mg/dL after 1 year of having received the AMR101). FIG. 10A shows the Kaplan-Meier curves for the primary endpoint of time to first occurrence of cardiovascular death, nonfatal myocardial infarction, nonfatal stroke, coronary revascularization, or unstable angina in the AMR101 treatment group for patients with achieved triglycerides, and the placebo group at year 1. Conversely, FIG. 10B shows the Kaplan-Meier event curves for the key secondary endpoint of time to first occurrence of cardiovascular death, nonfatal myocardial infarction, or nonfatal stroke in the AMR101 treatment group for patients with achieved triglycerides, and the placebo group at year 1. Importantly, FIGS. 10A and 10B indicate that regardless of the subject's triglyceride levels at year 1, the subject experienced a statistically significant reduction in time to first occurrence of cardiovascular death, nonfatal myocardial infarction, nonfatal stroke, coronary revascularization, or unstable angina. The attainment of triglyceride levels of 150 mg/dL or higher or below 150 mg/dL at 1 year after randomization also had no influence on the efficacy of AMR101 as compared with placebo with respect to the primary or key secondary efficacy endpoint. In a post hoc analysis, no substantial difference in the benefit of AMR101 as compared with placebo was observed with respect to the primary endpoint according to whether the patients who received placebo had an increase in LDL cholesterol levels at 1 year or had no change or a decrease in LDL cholesterol levels.

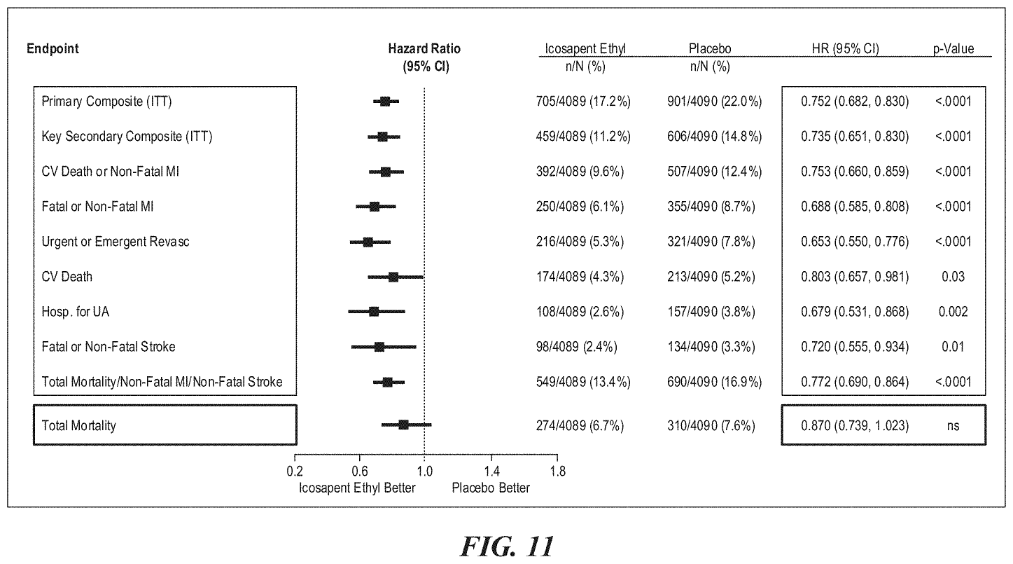

FIG. 11 depicts the prespecified hierarchical testing of the endpoints; except for the last hierarchical secondary endpoint of death from any cause (also referred to as total mortality), all other individual and composite ischemic endpoints were significantly reduced by AMR101, including cardiovascular death (4.3% versus 5.2%; HR, 0.80; 95% CI, 0.66-0.98; P=0.03). Total mortality was 6.7% versus 7.6% (HR, 0.87; 95% CI, 0.74-1.02; P=0.09) in the AMR101 and placebo groups, respectively. For each of the prespecified endpoints in FIG. 11, icosapent ethyl 4 g per day provided an RRR of 25% for the primary composite endpoint, 26% for the secondary composite endpoint, 25% for the composite of cardiovascular death or nonfatal myocardial infarction, 31% for fatal or nonfatal myocardial infarction, 35% for urgent or emergent revascularization, 20% for cardiovascular death, 32% for hospitalization for unstable angina, 28% for fatal or nonfatal stroke, 23% reduction in the composite of total mortality, nonfatal myocardial infarction, or nonfatal stroke, and lastly, a 13% reduction in total mortality.

Results for selected tertiary outcomes are shown in Table 17. A tertiary endpoint, adjudicated sudden cardiac death, was 2.1% versus 1.5% (HR, 0.69; 95% CI, 0.50-0.96).

TABLE 17
Selected Prespecified Adjudicated Tertiary Endpoints

| | Icosapent Ethyl | | Placebo | | HR | |
|---|---|---|---|---|---|---|
| Tertiary Endpoint | n/N (%) | | n/N (%) | | (95% CI) | |
| Primary Endpoint in | 433/2394 | (18.1%) | 536/2393 | (22.4%) | 0.77 | (0.68, 0.87) |
| Patients with Diabetes at | | | | | | |
| Baseline | | | | | | |
| New Heart Failure | 169/4089 | (4.1%) | 176/4090 | (4.3%) | 0.95 | (0.77, 1.17) |
| New Heart Failure | 141/4089 | (3.4%) | 144/4090 | (3.5%) | 0.97 | (0.77, 1.22) |
| Requiring Hospitalization | | | | | | |
| Transient Ischemic Attack | 64/4089 | (1.6%) | 48/4090 | (1.2%) | 1.32 | (0.91, 1.92) |
| Amputation for PVD | 22/4089 | (0.5%) | 21/4090 | (0.5%) | 1.04 | (0.57, 1.89) |
| Carotid Revascularization | 31/4089 | (0.8%) | 26/4090 | (0.6%) | 1.18 | (0.70, 1.98) |
| Coronary Revascularization | 376/4089 | (9.2%) | 544/4090 | (13.3%) | 0.66 | (0.58, 0.76) |
| Emergent Revascularization | 41/4089 | (1.0%) | 65/4090 | (1.6%) | 0.62 | (0.42, 0.92) |
| Urgent Revascularization | 181/4089 | (4.4%) | 268/4090 | (6.6%) | 0.66 | (0.54, 0.79) |
| Elective Revascularization | 194/4089 | (4.7%) | 278/4090 | (6.8%) | 0.68 | (0.57, 0.82) |
| Salvage Revascularization | 0/4089 | (0.0%) | 2/4090 | (0.0%) | 0.00 | (0.00, —) |
| Cardiac Arrhythmias | 188/4089 | (4.6%) | 154/4090 | (3.8%) | 1.21 | (0.97, 1.49) |
| Requiring Hospitalization | | | | | | |
| of ≥24 Hours | | | | | | |
| Cardiac Arrest | 22/4089 | (0.5%) | 42/4090 | (1.0%) | 0.52 | (0.31, 0.86) |
| Sudden Cardiac Death | 61/4089 | (1.5%) | 87/4090 | (2.1%) | 0.69 | (0.50, 0.96) |
| Ischemic Stroke | 80/4089 | (2.0%) | 122/4090 | (3.0%) | 0.64 | (0.49, 0.85) |
| Hemorrhagic Stroke | 13/4089 | (0.3%) | 10/4090 | (0.2%) | 1.28 | (0.56, 2.93) |
| New Onset of Diabetes^{[1]} | 65/1695 | (3.8%) | 63/1697 | (3.7%) | 1.04 | (0.73, 1.47) |

^{[1]}Patents with diabetes at baseline are excluded from this endpoint analysis.

Analysis of Additional Biomarker from Baseline

The effects on additional biomarkers to year 1 are shown in Table 18.

TABLE 18
Effect on Biomarkers from Baseline to Year 1

| | | | | | Median Between Group | | |
|---|---|---|---|---|---|---|---|
| | | | | | Difference at Year 1 | | |
| | Icosapent Ethyl | | Placebo | | Absolute | % | |
| | (N = 4089) | | (N = 4090) | | Change | Change | % |
| | Median | | Median | | from | from | Change |
| Biomarker | Baseline | Year 1 | Baseline | Year 1 | Baseline | Baseline | P-value |
| Triglycerides (mg/dL) | 216.5 | 175.0 | 216.0 | 221.0 | −44.5 | −19.7 | <0.0001 |
| Non-HDL-C (mg/dL) | 118.0 | 113.0 | 118.5 | 130.0 | −15.5 | −13.1 | <0.0001 |
| LDL-C (mg/dL) | 74.5 | 77.0 | 76.0 | 84.0 | −5.0 | −6.6 | <0.0001 |
| HDL-C (mg/dL) | 40.0 | 39.0 | 40.0 | 42.0 | −2.5 | −6.3 | <0.0001 |
| Apo B (mg/dL) | 82.0 | 80.0 | 83.0 | 89.0 | −8.0 | −9.7 | <0.0001 |
| hs-CRP (mg/L) | 2.2 | 1.8 | 2.1 | 2.8 | −0.9 | −39.9 | <0.0001 |
| EPA (μg/mL) | 26.1 | 144.0 | 26.1 | 23.3 | 114.9 | 358.8 | <0.0001 |

The effects on lipid, lipoprotein, and inflammatory markers over time for the ITT population are shown in Table 19.

TABLE 19
Lipid, Lipoprotein, and Inflammatory Marker Data over Time for the ITT Population

| | | Icosapent Ethyl (N = 4089) | | | | Placebo (N = 4090) | |
|---|---|---|---|---|---|---|---|
| | | | Median | | | | Median |
| | | | Absolute | Median % | | | Absolute |
| | | Median | Change | Change | Median % | Median | Change |
| | | Observed | from | from | Change | Observed | from |
| Biomarker | Visit | Value | Baseline | Baseline | P-value^{[1]} | Value | Baseline |
| Triglycerides | Baseline | 216.5 | | | | 216.0 | |
| (mg/dL) | Month 4 | 177.0 | −37.5 | −18.6 | <0.001 | 221.0 | 5.5 |
| | Year 1 | 175.0 | −39.0 | −18.3 | <0.001 | 221.0 | 4.5 |
| | Year 2 | 173.0 | −38.5 | −18.9 | <0.001 | 220.0 | 4.3 |
| | Year 3 | 167.0 | −44.0 | −21.7 | <0.001 | 212.0 | 1.0 |
| | Year 4 | 163.0 | −42.5 | −21.7 | <0.001 | 200.0 | −7.0 |
| | Year 5 | 158.0 | −38.0 | −20.0 | <0.001 | 193.0 | −3.0 |
| | Last Visit | 170.0 | −45.0 | −21.6 | <0.001 | 202.0 | −13.0 |
| Non-HDL-C | Baseline | 118.0 | | | | 118.5 | |
| (mg/dL) | Month 4 | 113.0 | −4.5 | −4.0 | <0.001 | 128.0 | 9.5 |
| | Year 1 | 113.0 | −4.0 | −3.6 | <0.001 | 130.0 | 12.0 |
| | Year 2 | 113.0 | −3.5 | −3.1 | 0.002 | 129.0 | 11.5 |
| | Year 3 | 112.0 | −4.8 | −4.2 | <0.001 | 128.0 | 10.5 |
| | Year 4 | 110.5 | −5.0 | −4.2 | <0.001 | 126.0 | 9.5 |
| | Year 5 | 109.0 | −5.0 | −4.4 | 0.004 | 123.0 | 7.0 |
| | Last Visit | 112.0 | −5.0 | −4.4 | <0.001 | 124.0 | 6.0 |
| LDL-C derived | Baseline | 74.0 | | | | 76.0 | |
| (mg/dL)^{[4]} | Year 1 | 77.0 | 2.0 | 3.1 | <0.001 | 84.0 | 7.0 |
| | Last Visit | 77.0 | 2.0 | 3.1 | <0.001 | 84.0 | 7.0 |
| LDL-C Hopkins | Baseline | 85.8 | | | | 86.7 | |
| (mg/dL) | Month 4 | 83.6 | −1.6 | −2.0 | 0.01 | 93.7 | 7.3 |
| | Year 1 | 85.3 | −1.1 | −1.2 | 0.06 | 95.8 | 9.3 |
| | Year 2 | 85.5 | −0.1 | −0.2 | <0.001 | 96.1 | 9.5 |
| | Year 3 | 84.6 | −1.0 | −1.2 | 0.01 | 95.7 | 9.0 |
| | Year 4 | 83.6 | −0.5 | −0.6 | 0.07 | 94.7 | 8.8 |
| | Year 5 | 82.2 | −0.8 | −0.7 | 0.23 | 91.6 | 6.2 |
| | Last Visit | 84.0 | −1.0 | −1.2 | 0.14 | 92.1 | 5.7 |
| HDL-C | Baseline | 40.0 | | | | 40.0 | |
| (mg/dL) | Month 4 | 39.0 | −1.0 | −2.8 | <0.001 | 42.0 | 2.0 |
| | Year 1 | 39.0 | −1.0 | −2.6 | <0.001 | 42.0 | 1.5 |
| | Year 2 | 40.0 | 0.0 | 0.0 | 0.21 | 42.0 | 1.5 |
| | Year 3 | 40.0 | 0.0 | 0.0 | 0.006 | 42.0 | 1.5 |
| | Year 4 | 40.5 | 0.5 | 1.0 | <0.001 | 43.0 | 2.0 |
| | Year 5 | 41.0 | 0.0 | 0.0 | 0.02 | 43.0 | 1.5 |
| | Last Visit | 41.0 | 1.0 | 2.5 | <0.001 | 42.0 | 2.0 |
| Apo B | Baseline | 82.0 | | | | 83.0 | |
| (mg/dL) | Year 2 | 80.0 | −2.0 | | 0.05 | 89.0 | 6.0 |
| | Last Visit | 80.0 | −2.0 | | 0.06 | 86.0 | 4.0 |
| hs-CRP | Baseline | 2.2 | | | | 2.1 | |
| (mg/L) | Year 2 | 1.8 | −0.2 | −13.9 | 0.04 | 2.8 | 0.5 |
| | Last Visit | 1.8 | −0.2 | −12.6 | 0.75 | 2.8 | 0.4 |
| Log hs-CRP | Baseline | 0.8 | | | | 0.8 | |
| (mg/L) | Year 2 | 0.6 | −0.1 | −21.8 | <.0001 | 1.0 | 0.3 |
| | Last Visit | 0.6 | −0.1 | −23.1 | <.0001 | 1.0 | 0.3 |
| EPA | Baseline | 26.1 | | | | 26.1 | |
| (μg/mL)^{[5]} | Year 1 | 144.0 | 112.6 | 393.5 | <0.001 | 23.3 | −2.9 |

| | | | | Between Group Difference | | |
|---|---|---|---|---|---|---|
| | | Placebo (N = 4090) | | Median | | |
| | | Median % | | Absolute | Median % | |
| | | Change | Median % | Change | Change | Median % |
| | | from | Change | from | from | Change |
| Biomarker | Visit | Baseline | P-value^{[1]} | Baseline^{[2]} | Baseline^{[2]} | P value^{[3]} |
| Triglycerides | Baseline | | | | | |
| (mg/dL) | Month 4 | 2.7 | <0.001 | −45.5 | −20.1 | <0.001 |
| | Year 1 | 2.2 | <0.001 | −44.5 | −19.7 | <0.001 |
| | Year 2 | 2.1 | <0.001 | −43.8 | −19.7 | <0.001 |
| | Year 3 | 0.4 | <0.001 | −45.5 | −20.3 | <0.001 |
| | Year 4 | −3.7 | >0.99 | −38.0 | −17.4 | <0.001 |
| | Year 5 | −1.5 | 0.23 | −33.5 | −16.7 | <0.001 |
| | Last Visit | −6.5 | <0.001 | −32.0 | −14.1 | <0.001 |
| Non-HDL-C | Baseline | | | | | |
| (mg/dL) | Month 4 | 8.2 | <0.001 | −14.3 | −12.2 | <0.001 |
| | Year 1 | 10.4 | <0.001 | −15.5 | −13.1 | <0.001 |
| | Year 2 | 9.8 | <0.001 | −14.5 | −12.5 | <0.001 |
| | Year 3 | 9.2 | <0.001 | −14.5 | −12.4 | <0.001 |
| | Year 4 | 8.1 | <0.001 | −14.0 | −12.0 | <0.001 |
| | Year 5 | 6.1 | <0.001 | −11.0 | −9.9 | <0.001 |
| | Last Visit | 5.1 | <0.001 | −10.0 | −8.6 | <0.001 |
| LDL-C derived | Baseline | | | | | |
| (mg/dL)^{[4]} | Year 1 | 10.2 | <0.001 | −5.0 | −6.6 | <0.001 |
| | Last Visit | 10.2 | <0.001 | −5.0 | −6.6 | <0.001 |
| LDL-C Hopkins | Baseline | | | | | |
| (mg/dL) | Month 4 | 8.7 | <0.001 | −8.7 | −10.3 | <0.001 |
| | Year 1 | 10.9 | <0.001 | −9.6 | −11.4 | <0.001 |
| | Year 2 | 11.4 | <0.001 | −9.4 | −11.1 | <0.001 |
| | Year 3 | 10.5 | <0.001 | −8.7 | −10.4 | <0.001 |
| | Year 4 | 10.1 | <0.001 | −8.9 | −10.6 | <0.001 |
| | Year 5 | 6.9 | <0.001 | −6.6 | −8.0 | <0.001 |
| | Last Visit | 6.5 | <0.001 | −6.2 | −7.4 | <0.001 |
| HDL-C | Baseline | | | | | |
| (mg/dL) | Month 4 | 4.7 | <0.001 | −3.0 | −7.2 | <0.001 |
| | Year 1 | 3.8 | <0.001 | −2.5 | −6.3 | <0.001 |
| | Year 2 | 4.2 | <0.001 | −2.0 | −4.6 | <0.001 |
| | Year 3 | 4.0 | <0.001 | −1.5 | −3.8 | <0.001 |
| | Year 4 | 4.8 | <0.001 | −1.5 | −3.9 | <0.001 |
| | Year 5 | 3.0 | <0.001 | −1.5 | −3.0 | <0.001 |
| | Last Visit | 5.7 | <0.001 | −1.0 | −3.0 | <0.001 |
| Apo B | Baseline | | | | | |
| (mg/dL) | Year 2 | 7.8 | <0.001 | −8.0 | −9.7 | <0.001 |
| | Last Visit | 4.5 | <0.001 | −5.0 | −6.7 | <0.001 |
| hs-CRP | Baseline | | | | | |
| (mg/L) | Year 2 | 32.3 | <0.001 | −0.9 | −39.9 | <0.001 |
| | Last Visit | 29.9 | <0.001 | −0.8 | −37.6 | <0.001 |
| Log hs-CRP | Baseline | | | | | |
| (mg/L) | Year 2 | 0.0 | 0.9203 | −0.4 | −22.5 | <0.001 |
| | Last Visit | −4.0 | 0.0481 | −0.4 | −21.2 | <0.001 |
| EPA | Baseline | | | | | |
| (μg/mL)^{[5]} | Year 1 | −12.8 | <0.001 | 114.9 | 358.8 | <0.001 |

### Safety Results

The results from this study showed no new or unexpected important AEs were observed in the safety population for this study as shown below in Tables 20 and 21. These conclusions are consistent with the independent DMC review conclusions and with quarterly safety review conclusions.

TABLE 20
Overview of Treatment-Emergent Adverse
Events of the Safety Population

| | AMR101 | | Placebo | | P- |
|---|---|---|---|---|---|
| | (N = 4089) | | (N = 4090) | | value^{[1]} |
| Subjects with at Least One | 3343 | (81.8%) | 3326 | (81.3%) | 0.63 |
| TEAE ^{[2]}, n(%) | | | | | |
| Serious TEAE | 1252 | (30.6%) | 1254 | (30.7%) | 0.98 |
| TEAE Leading to Withdrawal | 321 | (7.9%) | 335 | (8.2%) | 0.60 |
| of Study Drug ^{[3]} | | | | | |
| Serious TEAE Leading to | 88 | (2.2%) | 88 | (2.2%) | 1.00 |
| Withdrawal of Study Drug ^{[3]} | | | | | |
| Serious TEAE Leading to | 94 | (2.3%) | 102 | (2.5%) | 0.61 |
| Death^{[4]} | | | | | |

Note:
A treatment-emergent adverse event (TEAE) is defined as an event that first occurs or worsens in severity on or after the date of dispensing study drug and within 30 days after the completion or withdrawal from study. Percentages are based on the number of patients randomized to each treatment group in the Safety population (N). Events that were positively adjudicated as clinical endpoints are not included.
^{[1]}P-value from Fisher's Exact test.
^{[2]} All adverse events are coded using the Medical Dictionary for Regulatory Activities (MedDRA Version 20.1).
^{[3]} Withdrawal of study drug excludes patients who were off drug in study (ODIS) for 30 days or more, and restarted study drug.
^{[4]}The most common serious TEAEs leading to death by system organ class were neoplasms (1.1%); infections and infestations (0.4%); respiratory, thoracic, and mediastinal disorders (0.2%); cardiac disorders (0.2%); and vascular disorders (0.1%). No serious TEAEs leading to death by system organ class were statistically significant across treatment groups except for cardiac disorders, which occurred in 3 (0.1%) of VASCEPA ® patients and 15 (0.4%) of placebo patents (p = 0.008).

TABLE 21
Serious Bleeding Treatment-Emergent
Adverse Events by Preferred Term

| | Icosapent Ethyl | Placebo | |
|---|---|---|---|
| Preferred Term | (N = 4089) | (N = 4090) | P-value ^{[1]} |
| Bleeding related disorders | 111 (2.7%) | 85 (2.1%) | 0.06 |
| Gastrointestinal bleeding | 62 (1.5%) | 47 (1.1%) | 0.15 |
| Central nervous system | 14 (0.3%) | 10 (0.2%) | 0.42 |
| bleeding | | | |
| Other bleeding | 41 (1.0%) | 30 (0.7%) | 0.19 |

Note:
A treatment-emergent adverse event (TEAE) is defined as an event that first occurs or worsens in severity on or after the date of dispensing study drug and within 30 days after the completion or withdrawal from study. Percentages are based on the number of subjects randomized to each treatment group in the Safety population (N). Events that were positively adjudicated as clinical endpoints are not included. All adverse events are coded using the Medical Dictionary for Regulatory Activities (MedDRA Version 20.1).
^{[1]} Fishers Exact test.

Adverse events occurring in at least 5% of the patients are reported in Table 22. Compared with placebo, AMR101 was associated with a significantly higher rate of atrial fibrillation (5.3% versus 3.9%) and peripheral edema (6.5% vs 5%), but a lower rate of diarrhea (9% vs 11.1%), anemia (4.7% vs 5.8%), and gastrointestinal adverse events (33.0% to 35.1%). There was no significant difference in the prespecified adjudicated tertiary endpoint of heart failure (4.1% vs 4.3%). The prespecified adjudicated tertiary endpoint of atrial fibrillation or flutter requiring hospitalization was more common with the AMR101 group than the placebo group (3.1% vs 2.1%; P=0.004).

TABLE 22
Number (%) Patients with Most Frequent
Treatment-Emergent Adverse Events
(≥5%) in Either Treatment Group by Preferred
Term for the Safety Population

| | Icosapent Ethyl | Placebo | |
|---|---|---|---|
| Preferred Term | (N = 4089) | (N = 4090) | P-value ^{[1]} |
| Diarrhea | 367 (9.0%) | 453 (11.1%) | 0.002 |
| Back pain | 335 (8.2%) | 309 (7.6%) | 0.29 |
| Hypertension | 320 (7.8%) | 344 (8.4%) | 0.35 |
| Nasopharyngitis | 314 (7.7%) | 300 (7.3%) | 0.56 |
| Arthralgia | 313 (7.7%) | 310 (7.6%) | 0.90 |
| Upper respiratory tract | 312 (7.6%) | 320 (7.8%) | 0.77 |
| infection | | | |
| Bronchitis | 306 (7.5%) | 300 (7.3%) | 0.80 |
| Chest pain | 273 (6.7%) | 290 (7.1%) | 0.48 |
| Peripheral edema | 267 (6.5%) | 203 (5.0%) | 0.002 |
| Pneumonia | 263 (6.4%) | 277 (6.8%) | 0.56 |
| Influenza | 263 (6.4%) | 271 (6.6%) | 0.75 |
| Dyspnea | 254 (6.2%) | 240 (5.9%) | 0.52 |
| Urinary tract infection | 253 (6.2%) | 261 (6.4%) | 0.75 |
| Cough | 241 (5.9%) | 241 (5.9%) | 1.00 |
| Osteoarthritis | 241 (5.9%) | 218 (5.3%) | 0.27 |
| Dizziness | 235 (5.7%) | 246 (6.0%) | 0.64 |
| Pain in extremity | 235 (5.7%) | 241 (5.9%) | 0.81 |
| Cataract | 233 (5.7%) | 208 (5.1%) | 0.22 |
| Fatigue | 228 (5.6%) | 196 (4.8%) | 0.11 |
| Constipation | 221 (5.4%) | 149 (3.6%) | <0.001 |
| Atrial fibrillation | 215 (5.3%) | 159 (3.9%) | 0.003 |
| Angina pectoris | 200 (4.9%) | 205 (5.0%) | 0.84 |
| Anemia | 191 (4.7%) | 236 (5.8%) | 0.03 |

Note:
A treatment-emergent adverse event (TEAE) is defined as an event that first occurs or worsens in severity on or after the date of dispensing study drug and within 30 days after the completion or withdrawal from study. Percentages are based on the number of patients randomized to each treatment group in the Safety population (N). Events that were positively adjudicated as clinical endpoints are not included. All adverse events are coded using the Medical Dictionary for Regulatory Activities (MedDRA Version 20.1).
^{[1]} P-value from Fishers Exact test.

Serious treatment-emergent events occurring in at least 2% of the patients are reported in Table 23.

TABLE 23
Number (%) Patients with Serious Treatment-Emergent Adverse
Events (≥2%) in Either Treatment Group by Preferred Term

| | Icosapent Ethyl | Placebo | |
|---|---|---|---|
| Preferred Term | (N = 4089) | (N = 4090) | P-value ^{[1]} |
| Pneumonia | 105 (2.6%) | 118 (2.9%) | 0.42 |

Note:
A treatment-emergent adverse event (TEAE) is defined as an event that first occurs or worsens in severity on or after the date of dispensing study drug and within 30 days after the completion or withdrawal from study. Percentages are based on the number of subjects randomized to each treatment group in the Safety population (N). Events that were positively adjudicated as clinical endpoints are not included. All adverse events are coded using the Medical Dictionary for Regulatory Activities (MedDRA Version 20.1).
^{[1]} Fishers Exact test.

Adjudicated events from hospitalization for atrial fibrillation or atrial flutter are reported in Table 24.

TABLE 24
Number (%) Patients with Serious Treatment-Emergent Adverse
Events (≥2%) in Either Treatment Group by Preferred Term

| | Icosapent Ethyl | Placebo | |
|---|---|---|---|
| Preferred Term | (N = 4089) | (N = 4090) | P-value ^{[1]} |
| Positively Adjudicated Atrial | 127 (3.1%) | 84 (2.1%) | 0.0037 |
| Fibrillation/Flutter ^{[1]} | | | |

Note:
A treatment-emergent adverse event (TEAE) is defined as an event that first occurs or worsens in severity on or after the date of dispensing study drug and within 30 days after the completion or withdrawal from study. Percentages are based on the number of subjects randomized to each treatment group in the Safety population (N). Events that were positively adjudicated as clinical endpoints are not included.
All adverse events are coded using the Medical Dictionary for Regulatory Activities (MedDRA Version 20.1).
^{[1]} Fishers Exact test.

Tolerability of gastrointestinal TEAEs in either treatment group are reported in Table 25.

TABLE 25
Tolerability of gastrointestinal TEAEs

| Primary System Organ | Icosapent Ethyl | Placebo | |
|---|---|---|---|
| Class Preferred Term | (N = 4089) | (N = 4090) | P-value ^{[1]} |
| Gastrointestinal | 1350 (33.0%) | 1437 (35.1%) | 0.04 |
| disorders | | | |
| Diarrhea | 367 (9.0%) | 453 (11.1%) | 0.002 |
| Constipation | 221 (5.4%) | 149 (3.6%) | <0.001 |
| Nausea | 190 (4.6%) | 197 (4.8%) | 0.75 |
| Gastroesophageal | 124 (3.0%) | 118 (2.9%) | 0.70 |
| Reflux Disease | | | |

Note:
A treatment-emergent adverse event (TEAE) is defined as an event that first occurs or worsens in severity on or after the date of dispensing study drug and within 30 days after the completion or withdrawal from study. Percentages are based on the number of patients randomized to each treatment group in the Safety population (N). Events that were positively adjudicated as clinical endpoints are not included.
All adverse events are coded using the Medical Dictionary for Regulatory Activities (MedDRA Version 20.1).
^{[1]} P value from Fisher's Exact test.

When grouping treatment-emergent serious adverse events for bleeding, the rate was 2.7% in the AMR101 group versus 2.1% in the placebo group (P=0.06), although there were no fatal bleeding events in either group, and no significant increases in adjudicated hemorrhagic stroke (0.3% vs 0.2%; P=0.55), serious central nervous system bleeding (0.3% versus 0.2%; P=0.42), or gastrointestinal bleeding (1.5% versus 1.1%; P=0.15). Table 26 enumerates the serious bleeding treatment-emergent adverse events by preferred term.

TABLE 26
Assessment of Serious Bleeding Treatment-Emergent
Adverse Events by Category and by Preferred Term.

| | Icosapent Ethyl | | Placebo | | |
|---|---|---|---|---|---|
| | (N = 4089) | | (N = 4090) | | P-value^{[1]} |
| Patients with Bleeding- | 111 | (2.7%) | 85 | (2.1%) | 0.06 |
| Related Disorders^{[2]} | | | | | |
| By Category | | | | | |
| Gastrointestinal Bleeding^{[3]} | 62 | (1.5%) | 47 | (1.1%) | 0.15 |
| Central Nervous System Bleeding^{[4]} | 14 | (0.3%) | 10 | (0.2%) | 0.42 |
| Other Bleeding^{[5]} | 41 | (1.0%) | 30 | (0.7%) | 0.19 |
| By Preferred Term | | | | | |
| Gastrointestinal Hemorrhage | 26 | (0.6%) | 20 | (0.5%) | 0.38 |
| Rectal Hemorrhage | 10 | (0.2%) | 6 | (0.1%) | 0.33 |
| Subdural Hematoma | 9 | (0.2%) | 5 | (0.1%) | 0.30 |
| Hematuria | 8 | (0.2%) | 4 | (0.1%) | 0.27 |
| Epistaxis | 7 | (0.2%) | 4 | (0.1%) | 0.39 |
| Lower Gastrointestinal Hemorrhage | 5 | (0.1%) | 4 | (0.1%) | 0.75 |
| Post Procedural Hemorrhage | 5 | (0.1%) | 3 | (0.1%) | 0.51 |
| Hemorrhagic Anemia | 4 | (0.1%) | 1 | (0.0%) | 0.22 |
| Gastric Ulcer Hemorrhage | 3 | (0.1%) | 1 | (0.0%) | 0.37 |
| Hematemesis | 3 | (0.1%) | 0 | (0.0%) | 0.12 |
| Hemorrhoidal Hemorrhage | 3 | (0.1%) | 1 | (0.0%) | 0.37 |
| Melaena | 3 | (0.1%) | 4 | (0.1%) | >0.99 |
| Upper Gastrointestinal Hemorrhage | 3 | (0.1%) | 3 | (0.1%) | >0.99 |
| Diverticulum Intestinal Hemorrhagic | 3 | (0.1%) | 3 | (0.1%) | >0.99 |
| Shock Hemorrhagic | 2 | (0.0%) | 0 | (0.0%) | 0.25 |
| Cystitis Hemorrhagic | 2 | (0.0%) | 0 | (0.0%) | 0.25 |
| Subarachnoid Hemorrhage | 2 | (0.0%) | 1 | (0.0%) | 0.62 |
| Subdural Hemorrhage | 2 | (0.0%) | 1 | (0.0%) | 0.62 |
| Traumatic Hematoma | 2 | (0.0%) | 1 | (0.0%) | 0.62 |
| Duodenal Ulcer Hemorrhage | 2 | (0.0%) | 0 | (0.0%) | 0.25 |
| Aortic Aneurysm Rupture | 1 | (0.0%) | 1 | (0.0%) | >0.99 |
| Ecchymosis | 1 | (0.0%) | 0 | (0.0%) | 0.50 |
| Extravasation Blood | 1 | (0.0%) | 0 | (0.0%) | 0.50 |
| Gastric Hemorrhage | 1 | (0.0%) | 3 | (0.1%) | 0.62 |
| Gastrointestinal Angiodysplasia Hemorrhagic | 1 | (0.0%) | 0 | (0.0%) | 0.50 |
| Genital Hemorrhage | 1 | (0.0%) | 0 | (0.0%) | 0.50 |
| Hematochezia | 1 | (0.0%) | 2 | (0.0%) | >0.99 |
| Hematoma | 1 | (0.0%) | 1 | (0.0%) | >0.99 |
| Hemoptysis | 1 | (0.0%) | 0 | (0.0%) | 0.50 |
| Hemorrhagic Transformation Stroke | 1 | (0.0%) | 0 | (0.0%) | 0.50 |
| Hemothorax | 1 | (0.0%) | 1 | (0.0%) | >0.99 |
| Intra-Abdominal Hemorrhage | 1 | (0.0%) | 0 | (0.0%) | 0.50 |
| Large Intestinal Hemorrhage | 1 | (0.0%) | 1 | (0.0%) | >0.99 |
| Mallory-Weiss Syndrome | 1 | (0.0%) | 0 | (0.0%) | 0.50 |
| Menorrhagia | 1 | (0.0%) | 0 | (0.0%) | 0.50 |
| Pancreatitis Hemorrhagic | 1 | (0.0%) | 0 | (0.0%) | 0.50 |
| Peptic Ulcer Hemorrhage | 1 | (0.0%) | 0 | (0.0%) | 0.50 |
| Post Procedural Hematoma | 1 | (0.0%) | 1 | (0.0%) | >0.99 |
| Retinal Hemorrhage | 1 | (0.0%) | 1 | (0.0%) | >0.99 |
| Retroperitoneal Hemorrhage | 1 | (0.0%) | 0 | (0.0%) | 0.50 |
| Ulcer Hemorrhage | 1 | (0.0%) | 0 | (0.0%) | 0.50 |
| Urinary Bladder Hemorrhage | 1 | (0.0%) | 1 | (0.0%) | >0.99 |
| Hemarthrosis | 0 | (0.0%) | 1 | (0.0%) | >0.99 |
| Brain Contusion | 0 | (0.0%) | 2 | (0.0%) | 0.50 |
| Intracranial Hemorrhage | 0 | (0.0%) | 1 | (0.0%) | >0.99 |
| Immune Thrombocytopenic Purpura | 0 | (0.0%) | 1 | (0.0%) | >0.99 |
| Catheter Site Hemorrhage | 0 | (0.0%) | 1 | (0.0%) | >0.99 |
| Mouth Hemorrhage | 0 | (0.0%) | 1 | (0.0%) | >0.99 |
| Esophageal Hemorrhage | 0 | (0.0%) | 1 | (0.0%) | >0.99 |
| Cerebral Hemorrhage | 0 | (0.0%) | 2 | (0.0%) | 0.50 |
| Pericardial Hemorrhage | 0 | (0.0%) | 1 | (0.0%) | >0.99 |
| Post Procedural Hematuria | 0 | (0.0%) | 1 | (0.0%) | >0.99 |
| Renal Hemorrhage | 0 | (0.0%) | 1 | (0.0%) | >0.99 |
| Retroperitoneal Hematoma | 0 | (0.0%) | 1 | (0.0%) | >0.99 |
| Traumatic Intracranial Hemorrhage | 0 | (0.0%) | 1 | (0.0%) | >0.99 |
| Diverticulitis Intestinal Hemorrhagic | 0 | (0.0%) | 1 | (0.0%) | >0.99 |
| Hemorrhagic Duodenitis | 0 | (0.0%) | 1 | (0.0%) | >0.99 |

Note:
A treatment-emergent adverse event (TEAE) is defined as an event that first occurs or worsens in severity on or after the date of dispensing study drug and within 30 days after the completion or withdrawal from study. Percentages are based on the number of patients randomized to each treatment group in the Safety population (N). Events that were positively adjudicated as clinical endpoints are not included.
All adverse events are coded using the Medical Dictionary for Regulatory Activities (MedDRA Version 20.1).
^{[1]}P value from Fisher's Exact test.
^{[2]}Bleeding related events are identified using the Hemorrhage terms (excl laboratory terms), a Standard MedDRA Query (SMQ).
^{[3]}Gastrointestinal (Gl) related bleeding events are identified using the Gastrointestinal hemorrhage SMQ.
^{[4]}Central nervous system (CNS) related bleeding events are identified using the Central Nervous System hemorrhages and cerebrovascular conditions SMQs.
^{[5]}Other bleeding events are identified from the Hemorrhage terms (excluding laboratory terms) SMQ excluding Gl bleeding and CNS bleeding.

Among the 8,179 patients (70.7% secondary prevention) followed for a median 4.9 years, the primary endpoint occurred in 17.2% of AMR101 patients versus 22.0% of placebo (HR, 0.75; 95% CI, 0.68-0.83; P<0.001) and the key secondary endpoint in 11.2% versus 14.8% (HR, 0.74; 95% CI, 0.65-0.83; P<0.001). Additional ischemic endpoints, assessed according to a prespecified hierarchical schema, were significantly reduced, including cardiovascular death (4.3% versus 5.2%; HR, 0.80; 95% CI, 0.66-0.98; P=0.03). Atrial fibrillation or flutter hospitalization was more common with the AMR101 patients than the placebo patients (3.1% versus 2.1%; P=0.004); serious bleeding occurred in 2.7% of the AMR101 patients versus 2.1% in the placebo patients (P=0.06). There were no significant differences between treatments in the overall rate of treatment emergent adverse events or serious adverse events leading to withdrawal of the study drug as shown in Table 20. The only serious adverse event occurring at a frequency greater than or equal to 2% was pneumonia at 2.6% in the AMR101 group versus 2.9% in the placebo group (P=0.42).

### Conclusion

In this study, the risk of the primary composite endpoint of cardiovascular death, nonfatal myocardial infarction, nonfatal stroke, coronary revascularization, or unstable angina, assessed in a time-to-event analysis, was significantly lower, by 25%, among the patients who received 2 g of icosapent ethyl twice daily than among those who received the placebo, corresponding to an absolute between-group difference of 4.8 percentage points in the rate of the endpoint and a number needed to treat of 21. The risk of the key secondary composite endpoint of cardiovascular death, nonfatal myocardial infarction, or nonfatal stroke in a time-to-event analysis was also significantly lower, by 26%, in patients who received 2 g of icosapent ethyl twice daily than among those who received the placebo, corresponding to an absolute between-group difference of 3.6 percentage points in the rate of the endpoint and a number needed to treat of 28. Prespecified hierarchical testing of other secondary endpoints revealed that the risks of a variety of fatal and nonfatal ischemic events were lower in the AMR101 group than in the placebo group, including a 20% lower risk of cardiovascular death. The benefits were observed against a background of appropriate statin use among patients who had a median LDL cholesterol level of 75.0 mg/dL at baseline.

Overall adverse event rates were similar across treatment groups. There were numerically more serious adverse events related to bleeding, though overall rates were low, with no fatal bleeding observed in either group and no significant increase in adjudicated hemorrhagic stroke or serious central nervous system or gastrointestinal bleeding. There was a significantly higher rate of hospitalization for atrial fibrillation or flutter, though rates were low in those patients who received 2 g of icosapent ethyl twice daily. Adverse event and serious adverse event rates leading to study drug discontinuation were similar to placebo. The rates of adverse events and serious adverse events leading to discontinuation of the trial drug were similar in the two groups.

The results from this study stand apart from the negative findings of several recent trials of other agents that also lower triglyceride levels, such as other omega-3 fatty acids, extended-release niacin, fenofibrate, and cholesteryl ester transfer protein-inhibitors. It is not known whether the lack of benefit of omega-3 fatty acids in previous trials might be attributable to the low dose or the low ratio of EPA to DHA. Both the formulation (a highly purified and stable EPA acid ethyl ester) and dose (4 grams daily) used in this study are different from all prior omega-3 outcome trials. Despite utilizing a standard PROBE design limitation of those previous trials which included an open label design without placebo, used a low-intensity statin, and were conducted in a single country, in contrast to the present report, patients in those trials had higher baseline LDL-C levels (182 mg/dL prior to statin initiation) and lower triglyceride values (151 mg/dL). In contrast, the present study provides robust, multinational data showing significant reductions in ischemic events with administration of icosapent ethyl in patients with well-controlled LDL-C. Metabolic data support that icosapent ethyl does not raise LDL cholesterol levels, which DHA containing formulations do.

A triglyceride level of at least 150 mg/dL was required for inclusion in this study; however, owing to initial allowance for variability in these levels and differences between qualifying and randomization measurements, 10.3% of enrolled patients had triglycerides less than 150 mg/dL on study entry. Cardiovascular benefits appeared similar across baseline levels of triglycerides (e.g., 135-149, 150 to 199, and 200 mg/dL or greater). Additionally, the robust reduction in major adverse cardiovascular events with administration of icosapent ethyl appeared to occur irrespective of an achieved triglyceride level above or below 150 mg/dL at one year, suggesting that the cardiovascular risk reduction was not tied to achieving a more normal (i.e., <150 mg/dL) triglyceride level. These observations suggest that at least some of the impact of icosapent ethyl on the reduction in ischemic events may be explained by metabolic effects other than triglyceride lowering.

Mechanisms responsible for the benefit in the present study are currently not known. The timing of divergence of the Kaplan-Meier event curves suggests a delayed onset to benefit, which may reflect the time to benefit from triglyceride reduction or other mechanisms. The modestly higher rate of bleeding suggests that there might be an anti-thrombotic mechanism of action. However, it is unlikely that an anti-thrombotic effect would reduce elective revascularization. Also, if the full explanation were an antiplatelet or anticoagulant effect, one might expect a large increase in major bleeding, which was not seen. Potentially, membrane-stabilizing effects could explain part of the benefit. Stabilization and/or regression of coronary plaque may also play a part. The observation in the present study of a lower rate of sudden cardiac death might support that mechanism, though this finding should be viewed as exploratory. It is also possible that the 40% reduction in hs-CRP observed in patients from this trial may contribute to benefit. Samples (e.g., serum and plasma) from patients who participated in this trial have been banked for biomarker and genetic analyses, which may provide more information regarding mechanisms of action.

Regarding higher rates of diarrhea in the mineral oil placebo group, a post hoc analysis excluding patients with diarrhea still resulted in a significant risk reduction of 25% in the primary endpoint. Also, there were no differences in the primary or key secondary endpoints for placebo patients with an increase in LDL-C compared to those with no change or a decrease in LDL-C.

In conclusion, AMR101 4 grams daily demonstrated similar overall adverse event rates as placebo, and reduced important ischemic events, including cardiovascular death, in statin-treated patients with elevated triglycerides. Compared with placebo, icosapent ethyl 4 g per day significantly reduced cardiovascular events by 25% including: a 31% reduction in heart attack, 28% reduction in stroke, 31% reduction in myocardial infarction, and a 20% reduction in death due to cardiovascular events.

The following are key conclusions obtained from this trial that indicate a very favorable risk-benefit profile with (1) significant reduction in primary endpoint with an RRR of 24.8%, ARR of 4.8%, NNT of 21, and a p-value of 0.00000001; (2) significant reduction in key secondary endpoint with an RRR of 26.5%, ARR of 3.6%, NNT of 28, and a p-value of 0.0000062; (3) consistent results across subgroups to include triglycerides and secondary and primary prevention; (4) consistent results across hierarchical secondary endpoints to include cardiovascular death; (5) consistent results across recurrent events; and (6) safety with a small but insignificant increase in atrial fibrillation/flutter with low event rates and non-significant increase in serious bleeding with low event rates.

### Example 2: The Impact of Icosapent Ethyl on Recurrent Events and Total Ischemic Events in Statin-Treated Patients

Despite statin therapy, patients with established cardiovascular disease or diabetes remain at high risk for, not only first but also, recurrent ischemic events. The study results described in Example 1 demonstrated that icosapent ethyl reduces the first occurrence of the composite of cardiovascular death, nonfatal myocardial infarction, nonfatal stroke, coronary revascularization, or unstable angina, with a 25% relative risk reduction and a 4.8% absolute risk reduction. The time to first occurrence of the composite of cardiovascular death, nonfatal myocardial infarction, and nonfatal stroke was also reduced with icosapent ethyl, with a 26% relative risk reduction and a 3.6% absolute risk reduction.

The objective of the following study was to assess the impact of icosapent ethyl on recurrent events and total ischemic events. With a greater number of events, it was contemplated that there might be sufficient statistical power to examine the effect of icosapent ethyl in the two separate cardiovascular risk strata in the trial: patients with established atherosclerosis or patients with diabetes plus at least one other cardiovascular risk factor. Accordingly, the goal of the following study was to determine if icosapent ethyl administered at 4 g per day (e.g., 2 g twice daily) reduced total major adverse cardiovascular events in patients with fasting triglycerides ≥150 and <500 mg/dL and LDL-cholesterol >40 and ≤100 mg/dL who are at increased cardiovascular risk despite statin therapy.

### Study Design

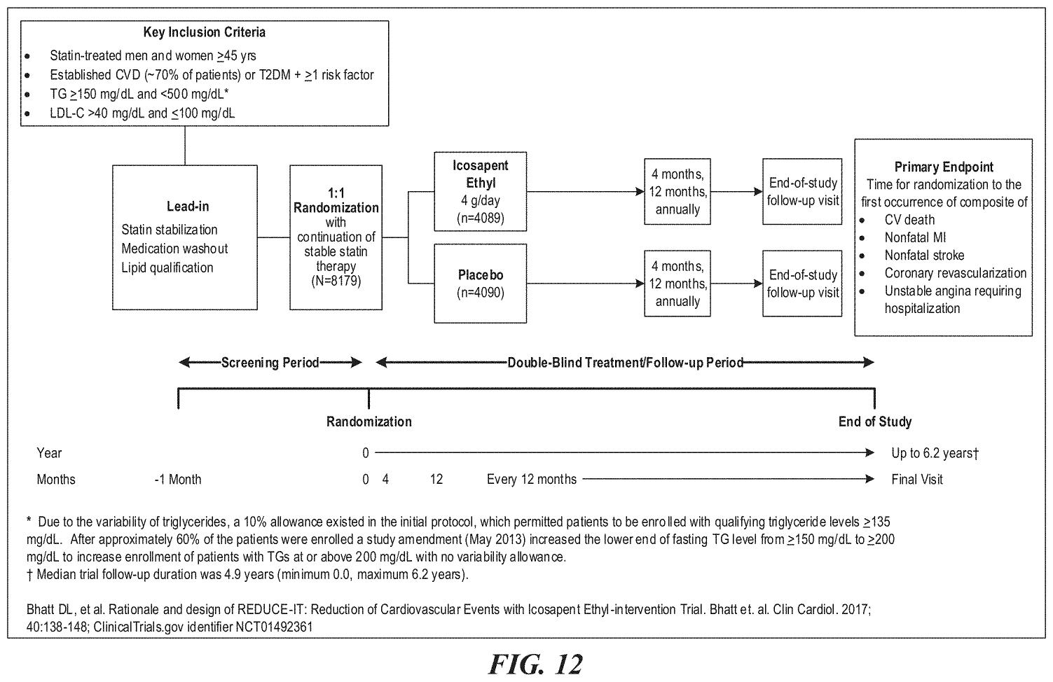

The following study was a multi-center, placebo-controlled clinical trial the details of which are described above in Example 1, the REDUCE-IT design. As shown in FIG. 12, patients were randomized in a double-blind manner to icosapent ethyl 4 g per day (2 g twice daily with food) versus placebo. Randomization was stratified by cardiovascular risk cohort (i.e., secondary or primary prevention), use of ezetimibe, and by geographic region.

### Study Population

The study participants included patients with a history of atherosclerosis or diabetes who were on statins and had fasting triglycerides ≥150 and <500 mg/dL and LDL-cholesterol >40 and ≤100 mg/dL. Of the study participants, 71% of the patients had a history of atherosclerosis and 29% had a history of diabetes. In order to be eligible for the trial, patients had to be at least 45 years of age with either established cardiovascular disease (i.e., secondary prevention stratum) or at least 50 years old with Type 2 or Type 1 diabetes mellitus requiring treatment with medication and at least one additional risk factor (i.e., primary prevention stratum).

The secondary prevention stratum consisted of patients with documented coronary artery disease (≥50% stenosis in at least two major epicardial coronary arteries with or without prior revascularization; prior MI; hospitalization for non-ST-segment elevation acute coronary syndrome with ST-segment deviation or positive biomarkers); documented cerebrovascular disease (prior ischemic stroke; symptomatic ≥50% carotid stenosis; asymptomatic carotid disease with ≥70% stenosis; history of carotid revascularization); or documented peripheral artery disease (ankle-brachial index <0.9 with symptoms of intermittent claudication; history of aorto-iliac or peripheral surgery or intervention).

The primary prevention stratum consisted of patients with no documented cardiovascular disease as defined above, with diabetes, and with at least one of the following cardiovascular risk factors: men at least 55 years of age or women at least 65 years of age; cigarette smoker or stopped smoking within 3 months before first visit; blood pressure at least 140 mmHg systolic or at least 90 mmHg diastolic or on antihypertensive medication; HDL-cholesterol not greater than 40 mg/dL for men or not greater than 50 mg/dL for women; hs-CRP greater than 3 mg/L; creatinine clearance greater than 30 and less than 60 mL/min; non-proliferative retinopathy, pre-proliferative retinopathy, proliferative retinopathy, maculopathy, advanced diabetic eye disease or a history of photocoagulation; micro- or macro-album inuria; or asymptomatic ankle-brachial index less than 0.9.

The participants were required to have fasting triglycerides between ≥150 mg/dL and <500 mg/dL and LDL-cholesterol >40 mg/dL and ≤100 mg/dL. In the initial version of the clinical trial protocol, a 10% allowance in qualifying triglyceride levels was allowed, and therefore patients with triglycerides of at least 135 mg/dL were randomized. The study included 841 (10.3%) patients with baseline triglyceride levels less than 150 mg/dL. After approximately 60% of the patients were enrolled, an amendment changed the lower limit of allowed triglyceride levels to 200 mg/dL with no variability allowance. Patients were required to be on stable statin therapy for at least four weeks.

Exclusion criteria for the study participants included severe heart failure or liver disease, hemoglobin A1c levels greater than 10.0%, planned coronary intervention, familial lipoprotein lipase deficiency, intolerance or hypersensitivity to statins, history of acute or chronic pancreatitis, and hypersensitivity to fish, shellfish, or ingredients of icosapent ethyl or placebo.

### Main Outcomes and Measures

The primary outcome for the study was total recurrent events consisting of the composite of cardiovascular death, nonfatal myocardial infarction, nonfatal stroke, coronary revascularization, or hospitalization for unstable angina. Recurrent event analyses were also performed for the key secondary endpoint, a composite of cardiovascular death, nonfatal myocardial infarction, or nonfatal stroke. For each of these composite endpoints, the effects of icosapent ethyl in the secondary and primary prevention strata were examined separately.

### Statistical Considerations

Demographic and baseline disease characteristics are presented using frequencies and percentages for categorical variables and medians with interquartile ranges for continuous variables. Between treatment group comparisons were derived using the chi-square test for categorical variables and Wilcoxon rank test for continuous variables. All clinical endpoint events used in the efficacy analyses were adjudicated by an independent Clinical Endpoint Committee (CEC) who were blinded to the treatment assignment. Since the primary efficacy endpoint was the time from randomization to the first occurrence of any component of the composite endpoint, and recurrence of such events within each patient is possible, a pre-specified analysis using a Cox proportional-hazard with the counting-process formulation of Andersen and Gill was performed to model the first and all recurrent cardiovascular events. HRs and corresponding 95% CIs are reported from this model. In addition, as a marginal model and an extension of survival models based on the Cox proportional hazard model, the modified Wei-Lin-Weissfeld (WLW) method for analysis of recurrent events in the presence of deaths was carried out as a supportive analysis. In addition, as pre-specified, a recurrent event analysis using the Andersen-Gill and Wei-Lin-Weissfeld methods was carried out for the individual primary event components other than CV death. Though not pre-specified, additional recurrent event analyses were performed for the key secondary endpoint, which is a composite of CV death, nonfatal MI, or nonfatal stroke, and for the primary endpoint and the key secondary endpoint in the primary and secondary prevention strata to explore further the consistency of clinical benefit of icosapent ethyl. In subgroup analyses of the two cardiovascular risk strata (i.e., primary and secondary prevention), site-level discrepancies in cardiovascular risk group assignment occurring at entry and detected during the study (1.8%) were adjusted to conform with documented medical history data prior to randomization. All efficacy analyses were performed according to the intention-to-treat principle. All tests were based on a 2-sided nominal significance level of 5% with no adjustments for multiple comparisons.

### Results

### Baseline Characteristics

A total of 8,179 patients were randomized and followed for a median of 4.9 years. The patients were well matched in the icosapent ethyl and placebo groups as shown in Table 16 (see Example 1). The secondary and primary prevention according to the adjusted stratification for this study are shown in Table 27.

TABLE 27
Secondary and Primary Prevention Per Adjusted Stratification
for Patients Randomized to Placebo or Icosapent Ethyl.

| | | |
|---|---|---|
| Icosapent ethyl | Placebo | |
| (N = 4089) | (N = 4090) | P-value ^{[1]} |

Stratification Factors

| | | | |
|---|---|---|---|
| | | | 0.7367 |
| Secondary Prevention per | 2933 (71.7%) | 2920 (71.4%) | |
| Adjusted Stratification | | | |
| Primary Prevention per | 1156 (28.3%) | 1170 (28.6%) | |
| Adjusted | | | |
| Stratification | | | |

^{[1]} P-value is from a Wilcoxon rank-sum test for continuous variables and a chi-square test for categorical variables.

At baseline, the patient's median triglyceride levels were 216 mg/dL and median LDL-C levels were 75 mg/dL. Additional baseline characteristics of the patients with no events, a single event, and multiple recurrent events are shown in Table 28.

TABLE 28
Baseline Characteristics of Patients with No Events, a Single Event, or Multiple Events.
Baseline Characteristics in Patients with No Events, a Single Event, or Multiple Events

| | | | |
|---|---|---|---|
| No Events | 1 Event | Multiple Events | |
| (N = 6573) | (N = 844) | (N = 762) | P-value ^{[1]} |

Demographics

| | | | | | | | |
|---|---|---|---|---|---|---|---|
| Age (years), Median | 63.0 | (57.0-69.0) | 65.0 | (59.0-71.0) | 64.0 | (58.0-70.0) | <.0001 |
| (Q1 − Q3) | | | | | | | |
| Age ≥65 years, n(%) | 2939 | (44.7%) | 456 | (54.0%) | 368 | (48.3%) | <.0001 |
| Male, n(%) | 4556 | (69.3%) | 661 | (78.3%) | 605 | (79.4%) | <.0001 |
| White, n(%)^{[2]} | 5921 | (90.1%) | 765 | (90.6%) | 693 | (90.9%) | 0.6908 |
| BMI (kg/m²), Median | 30.8 | (27.8-34.6) | 31.1 | (27.8-34.7) | 30.8 | (28.0-34.2) | 0.5124 |
| (Q1 − Q3) | | | | | | | |
| BMI ≥30, n(%)^{[3]} | 3762 | (57.2%) | 499 | (59.1%) | 432 | (56.7%) | 0.7771 |

Stratification Factors

| | | | | | | | |
|---|---|---|---|---|---|---|---|
| Geographic Region, | | | | | | | <.0001 |
| n(%) | | | | | | | |
| Westernized ^{[4]} | 4547 | (69.2%) | 639 | (75.7%) | 625 | (82.0%) | |
| Eastern Europe ^{[5]} | 1796 | (27.3%) | 185 | (21.9%) | 125 | (16.4%) | |
| Asian Pacific ^{[6]} | 230 | (3.5%) | 20 | (2.4%) | 12 | (1.6%) | |
| CV Risk Category as | | | | | | | <.0001 |
| Randomized, n(%) | | | | | | | |
| Secondary Prevention | 4488 | (68.3%) | 640 | (75.8%) | 657 | (86.2%) | |
| per Randomization | | | | | | | |
| Primary Prevention per | 2085 | (31.7%) | 204 | (24.2%) | 105 | (13.8%) | |
| Randomization | | | | | | | |
| CV Risk Category | | | | | | | <.0001 |
| Actual, n(%) | | | | | | | |
| Secondary Prevention | 4537 | (69.0%) | 652 | (77.3%) | 664 | (87.1%) | |
| per Adjusted | | | | | | | |
| Stratification | | | | | | | |
| Primary Prevention per | 2036 | (31.0%) | 192 | (22.7%) | 98 | (12.9%) | |
| Adjusted Stratification | | | | | | | |
| Ezetimibe Use, n(%) | 401 | (6.1%) | 59 | (7.0%) | 64 | (8.4%) | 0.0378 |

Statin Intensity and Diabetes Status

| Statin Intensity, n(%) | | | | | | | 0.0819 |
|---|---|---|---|---|---|---|---|
| Low | 428 | (6.5%) | 49 | (5.8%) | 44 | (5.8%) | |
| Moderate | 4141 | (63.0%) | 519 | (61.5%) | 448 | (58.8%) | |
| High | 1974 | (30.0%) | 274 | (32.5%) | 268 | (35.2%) | |
| Missing | 30 | (0.5%) | 2 | (0.2%) | 2 | (0.3%) | |
| Diabetes, n(%) | | | | | | | 0.5535 |
| Type I Diabetes | 44 | (0.7%) | 5 | (0.6%) | 8 | (7.0%) | |
| Type II Diabetes | 3773 | (57.4%) | 511 | (60.5%) | 445 | (58.4%) | |
| Both Type I and Type | 1 | (0.0%) | 0 | | 0 | | |
| II Diabetes | | | | | | | |
| No Diabetes at | 2752 | (41.9%) | 328 | (38.9%) | 309 | (40.6%) | |
| Baseline | | | | | | | |

| | | | | |
|---|---|---|---|---|
| Missing | 3 | (0.0%) | 0 | 0 |

Laboratory Measurements

| | | | | | | | |
|---|---|---|---|---|---|---|---|
| hs-CRP (mg/L), Median | 2.1 | (1.1-4.4) | 2.4 | (1.2-5.3) | 2.4 | (1.2-4.6) | 0.0004 |
| (Q1 − Q3) | | | | | | | |
| Triglycerides (mg/dL), | 215.5 | (176.0-272.0) | 215.5 | (175.0-270.3) | 223.0 | (178.5-285.5) | 0.0539 |
| Median (Q1 − Q3) | | | | | | | |
| HDL-C (mg/dL), Median | 40.0 | (35.0-46.0) | 39.5 | (34.4-45.5) | 38.8 | (33.5-44.5) | <.0001 |
| (Q1 − Q3) | | | | | | | |
| LDL-C (mg/dL), Median | 75.0 | (62.0-89.0) | 75.0 | (63.0-88.0) | 75.0 | (63.0-89.0) | 0.9903 |
| (Q1 − Q3) | | | | | | | |
| Triglycerides Category | | | | | | | 0.3523 |
| <150 mg/dL | 686 | (10.4%) | 79 | (9.4%) | 76 | (10.0%) | |
| 150 to <200 mg/dL | 1922 | (29.2%) | 259 | (30.7%) | 203 | (26.6%) | |
| ≥200 mg/dL | 3961 | (60.3%) | 506 | (60.0%) | 483 | (63.4%) | |
| Triglycerides ≥200 | 1254 | (19.1%) | 173 | (20.5%) | 190 | (24.9%) | 0.0005 |
| mg/dL and HDL-C ≤35 | | | | | | | |
| mg/dL | | | | | | | |
| EPA (μg/mL), Median | 26.2 | (17.2-40.3) | 24.6 | (15.9-36.7) | 26.9 | (17.7-40.2) | 0.0141 |
| (Q1 − Q3) | | | | | | | |

In general, the baseline value is defined as the last non-missing measurement obtained prior to the randomization.
The baseline LDL-C value obtained via preparative ultracentrifugation was used, unless this value was missing. If the LDL-C preparative ultracentrifugation value was missing, then another LDL-C value was be used, with prioritization of values obtained from LDL-C Direct measurements, followed by LDL-C derived by the Friedewald calculation (only for subjects with TG <400 mg/dL), and finally LDL-C derived using the calculation published by Johns Hopkins University investigators.
For all other lipid and lipoprotein marker parameters, wherever possible, baseline was derived as the arithmetic mean of the Visit 2 (Day 0) value and the preceding Visit 1 (or Visit 1.1) value. If only one of these values was available, the single available value was used as baseline.
^{[1]} P-value is from a Wilcoxon rank-sum test for continuous variables and a chi-square test for categorical variables.
^{[2]}Race as reported by the investigators.
^{[3]} Percentages are based on the number of randomized subjects.
^{[4]} Westernized region includes Australia, Canada, the Netherlands, New Zealand, United States, and South Africa.
^{[5]} Eastern European region includes Poland, Romania, Russian Federation, and Ukraine.
^{[6]} Asia Pacific region includes India.

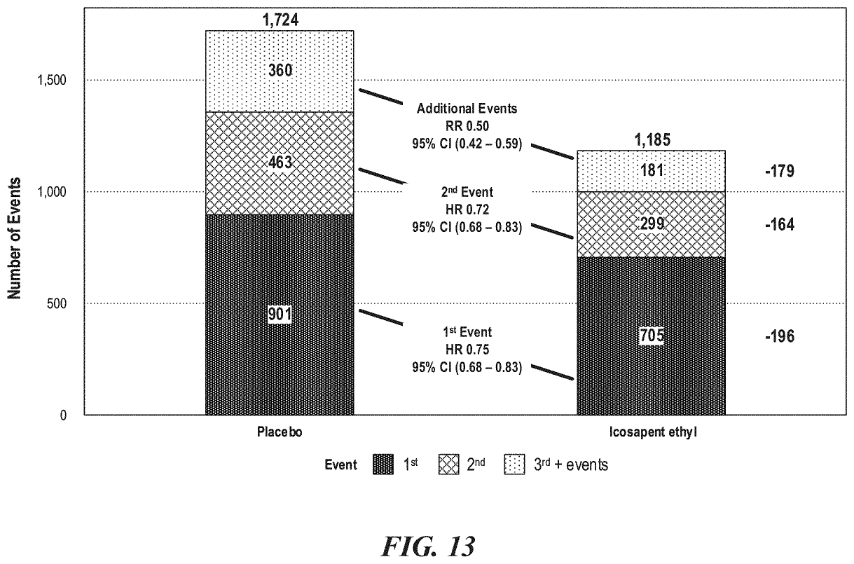

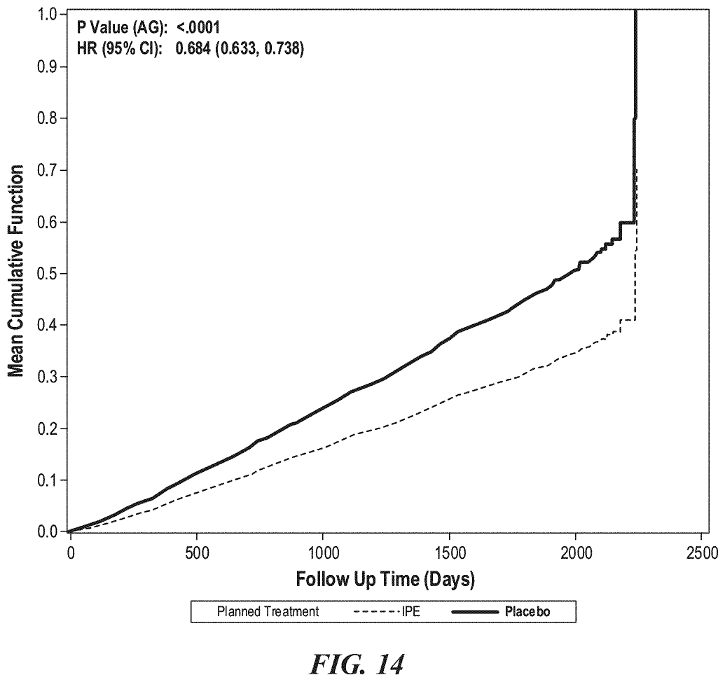

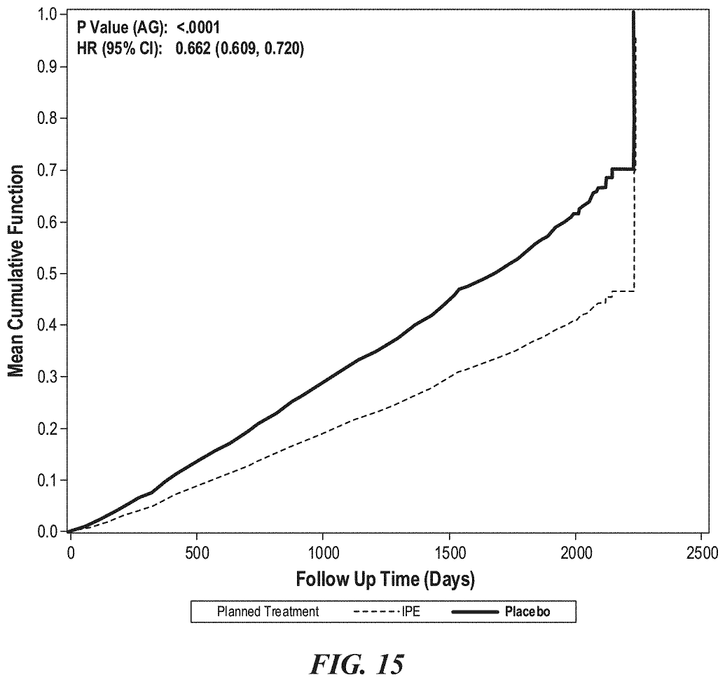

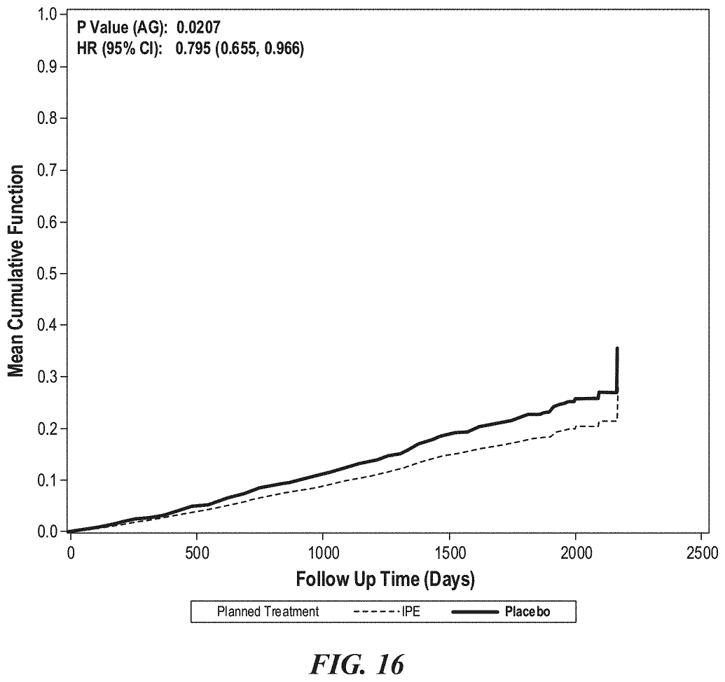

Total Events for Primary Efficacy Endpoint: The total events for the primary efficacy endpoint showed that of 8,179 patients, there were 1,606 (i.e., 55.2% of the endpoints) first primary endpoints and 1,303 (i.e., 44.8% of the endpoints) additional primary endpoints, for a total of 2,909 endpoint events among the 1,606 patients. There were 762 second events, 272 third events, and 269 fourth or more events. FIG. 13 shows a distribution of first and recurrent events in the patients randomized to icosapent ethyl or placebo before and after the trial. In the overall trial, total primary endpoints were reduced from 1,724 to 1,185 (HR 0.68, 95% CI 0.63-0.74, P<0.0001) with icosapent ethyl as shown in FIG. 13. Within the primary endpoint reductions, first events were reduced from 901 to 705 (i.e., a total reduction of 196), second events were reduced from 463 to 299 (i.e., a total reduction of 164), and additional endpoints were reduced from 360 to 131 (i.e., a total reduction of 179) with icosapent ethyl (see FIG. 13). Using the Wei-Lin-Weissfeld model, the first occurrence of a primary composite endpoint was reduced with icosapent ethyl versus placebo (HR 0.75, 95% CI 0.68-0.83, P<0.0001) as was the second occurrence (HR 0.72, 95% CI 0.62-0.83, P<0.0001). FIGS. 14-16 depict the overall cumulative event curves from the primary endpoint of cardiovascular death, nonfatal myocardial infarction, nonfatal stroke, coronary revascularization, and unstable angina. The overall cumulative events are shown in FIG. 14, the secondary prevention stratum events are shown in FIG. 15, and the primary prevention stratum events are shown in FIG. 16.

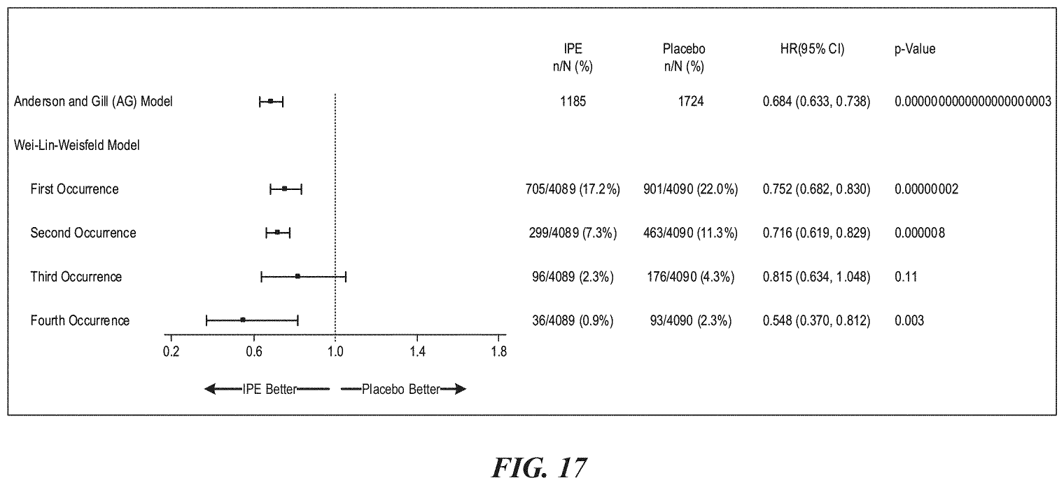

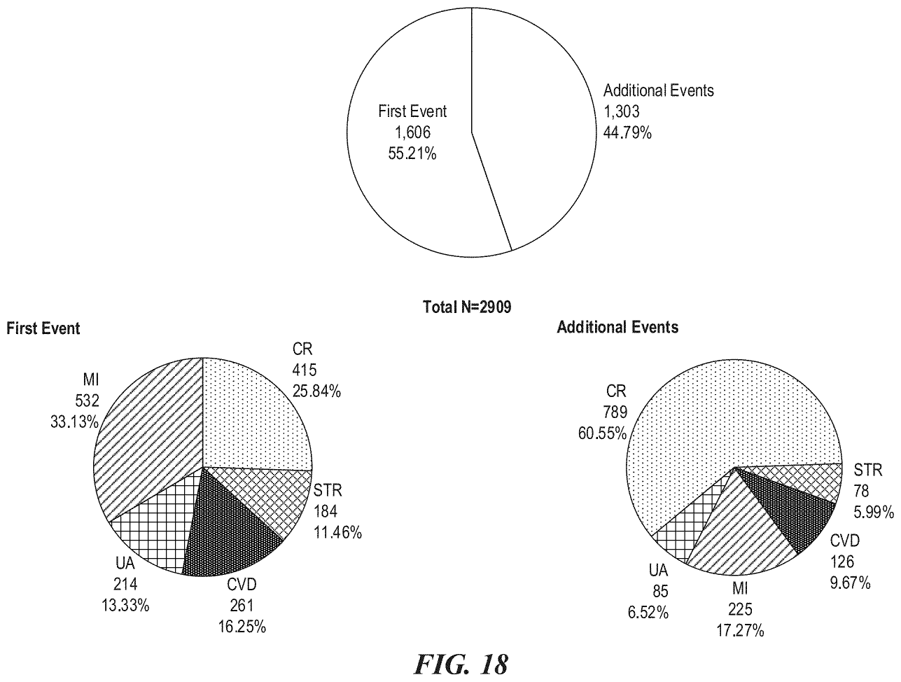

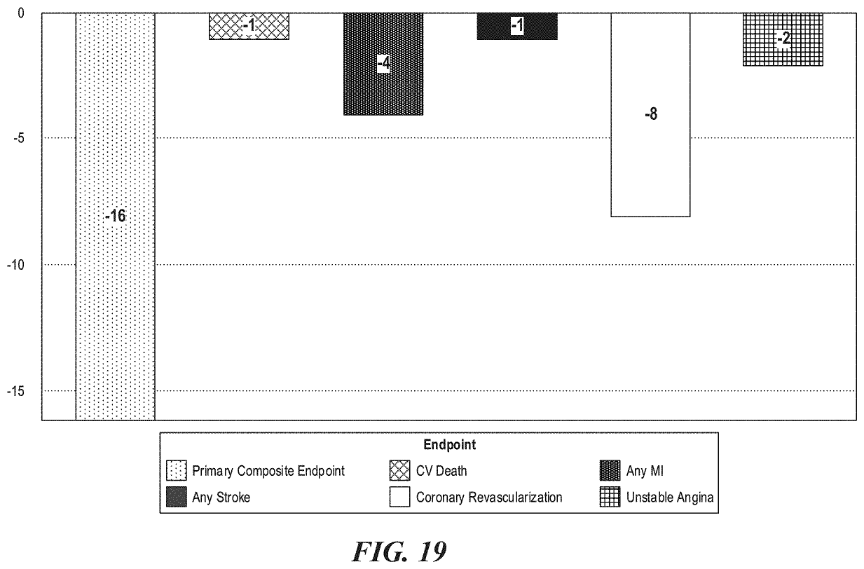

The total events for each occurrence of the primary endpoint, inclusive of the first and all subsequent occurrences of primary endpoint components (i.e., cardiovascular death, nonfatal myocardial infarction, nonfatal stroke, coronary revascularization, and unstable angina) are shown in FIG. 17. Importantly, FIG. 17 shows that the times to first occurrence, second occurrence, third occurrence, or fourth occurrence of the primary composite endpoint were consistently reduced in the icosapent ethyl group as compared to the placebo control group. The proportions of first and subsequent primary endpoint events, overall and by component, are depicted in FIG. 18. The risk differences for every 100 patients treated for five years with icosapent ethyl versus placebo control for the components of the composite primary endpoint are shown in FIG. 19.

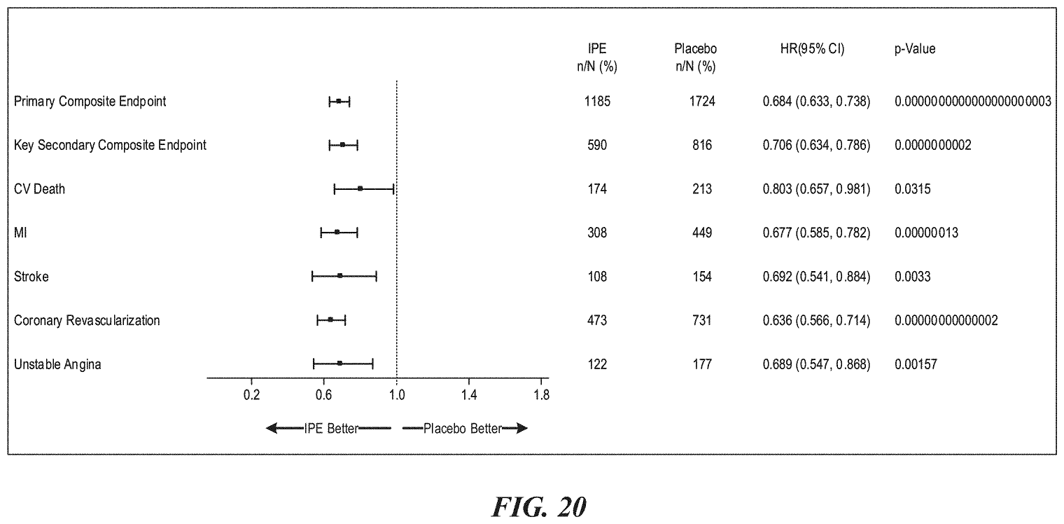

The total events for each component of the primary and key secondary efficacy endpoints inclusive of the first and all subsequent occurrences of the primary endpoint components (i.e., cardiovascular death, nonfatal myocardial infarction, nonfatal stroke, coronary revascularization, and unstable angina) and key secondary endpoint components (i.e., nonfatal myocardial infarction, nonfatal stroke, and cardiovascular death) are shown in FIG. 20. Importantly, FIG. 20 shows that total events for each component of the primary endpoint were also significantly reduced. In the secondary prevention stratum, total primary endpoint events were reduced from 1,468 to 988 (HR 0.66, 95% CI 0.61-0.72, P<0.0001), and in the primary prevention stratum, from 256 to 197 (HR 0.79, 95% CI 0.65-0.96, P=0.018; P_{interaction}=0.098). Without adjusting for stratification differences, total primary endpoint events in the secondary prevention stratum were reduced from 1,461 to 964 (HR 0.65, 95% CI 0.60-0.71, P<0.0001) and from 263 to 221 (HR 0.86, 95% CI 0.71-1.03, P=0.105) in the primary prevention stratum; P_{interaction}=0.009.

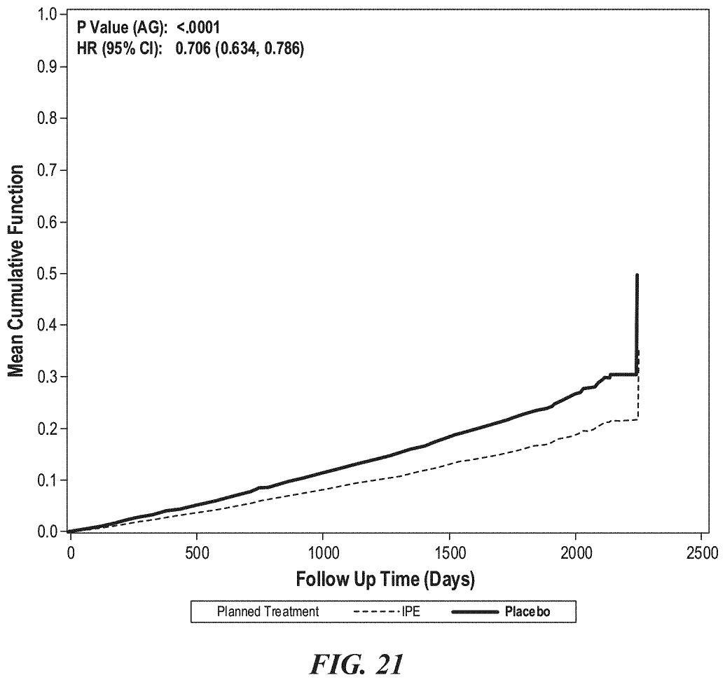

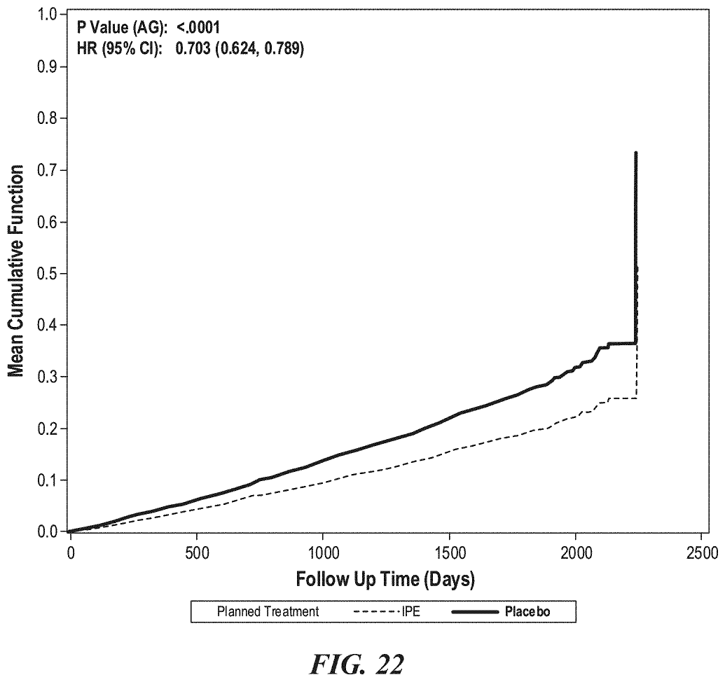

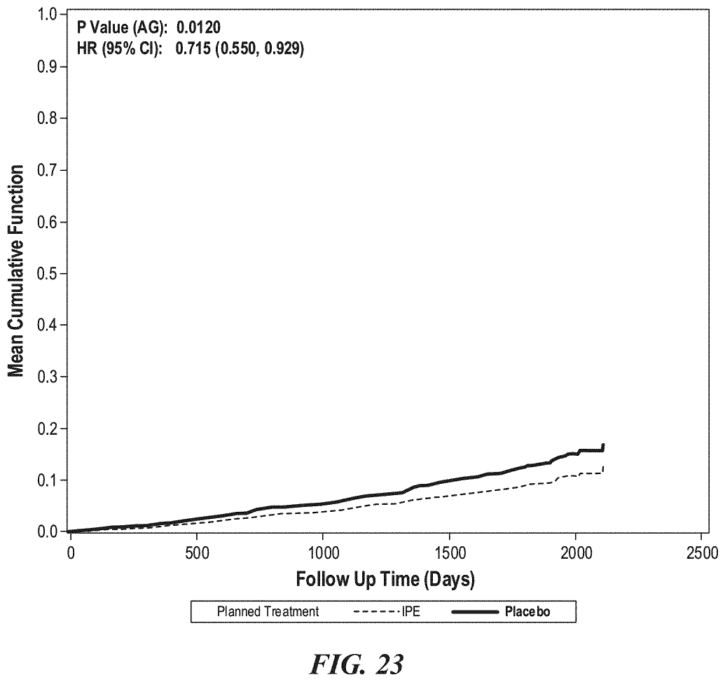

Total Events for the Key Secondary Efficacy Endpoint: FIGS. 21-23 depict the cumulative event curves from the key secondary endpoint of cardiovascular death, nonfatal myocardial infarction, and nonfatal stroke. The overall cumulative events are shown in FIG. 21, the secondary prevention stratum events are shown in FIG. 22, and the primary prevention stratum events are shown in FIG. 23. Total key secondary endpoints were significantly reduced from 861 to 590 (HR 0.71, 95% CI 0.63-0.79, P<0.0001) with icosapent ethyl versus placebo as shown in FIG. 21. Similar patterns were seen for the key secondary endpoint, both in the secondary prevention (HR 0.70, 95% CI 0.63-0.79, P<0.0001) and primary prevention (HR 0.71, 95% CI 0.55-0.93, P=0.011) strata as shown in FIGS. 22 and 23, respectively, P_{interaction}=0.90. Without adjustment for stratification differences, total key secondary endpoint events in the secondary prevention stratum were reduced from 671 to 478 (HR 0.69, 95% CI 0.61-0.78, P<0.0001) and from 142 to 112 (HR 0.78, 95% CI 0.60-1.00, P=0.047) in primary prevention; P_{interaction}=0.39.

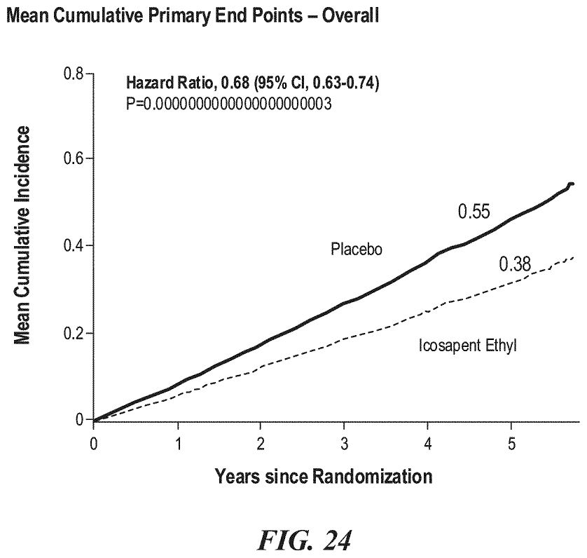

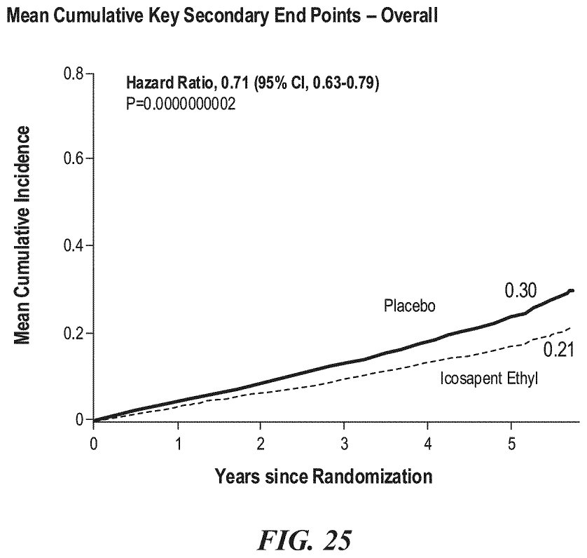

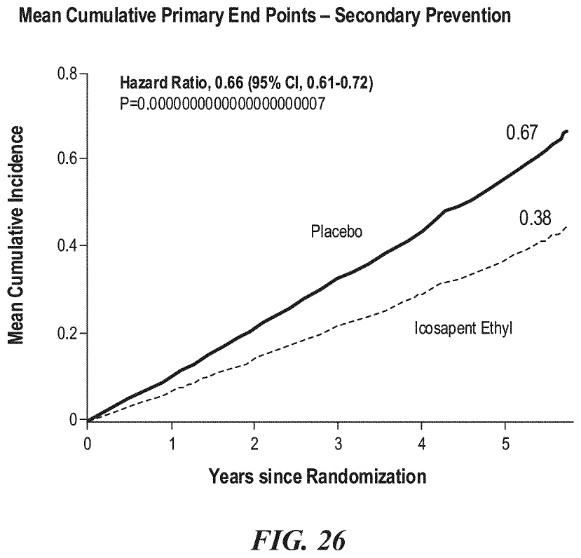

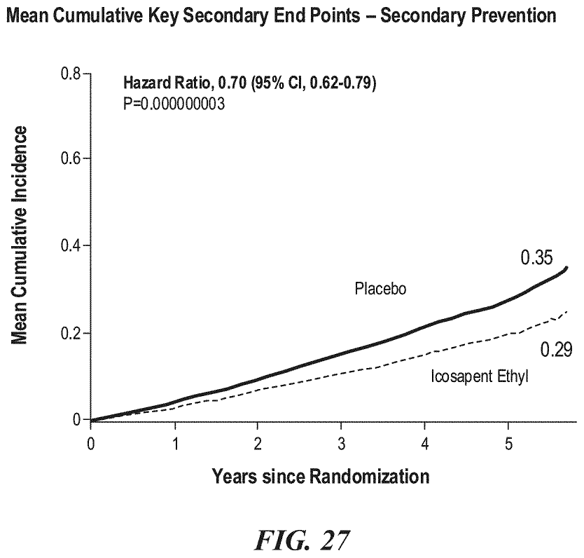

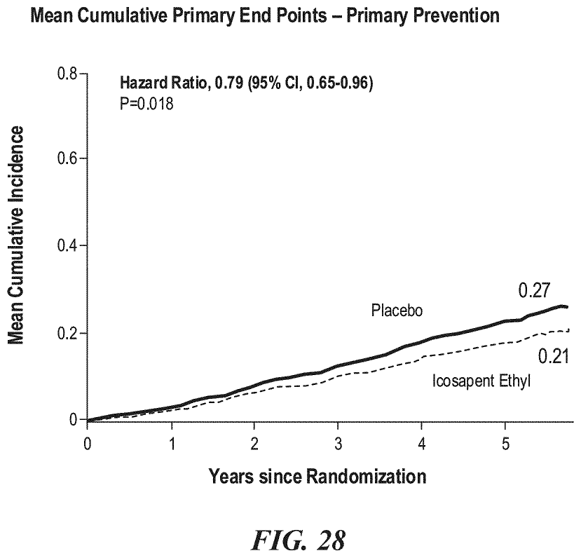

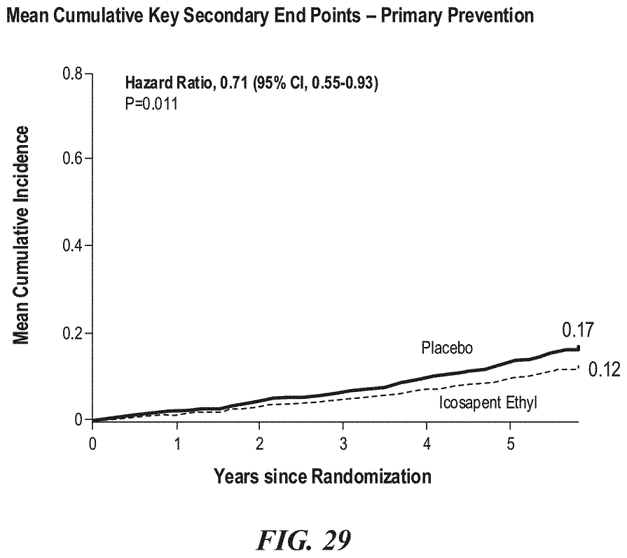

Similarly, the total events for the primary and key secondary efficacy endpoints are further depicted in FIGS. 24-29 as a function of the total cumulative incidence versus years since randomization. This contrasts FIGS. 14-16 and FIGS. 21-23 which report the total events for the primary and key secondary efficacy endpoints as a function of the mean cumulative function versus follow-up time in days from randomization. FIGS. 24 and 25 show the overall mean cumulative recurrent events of the primary composite endpoint and key secondary endpoint, respectively. FIGS. 26 and 27 depict the recurrent events of primary and key secondary endpoints for the secondary prevention stratum, respectively. Lastly, FIGS. 28 and 29 further depict the recurrent events of primary and key secondary endpoints for the primary prevention stratum, respectively.

Overall, the results of this study indicated that the use of icosapent ethyl was superior as compared to a placebo in reducing total ischemic events, with a consistent benefit in secondary as well as primary prevention.

### Conclusion

This study, an analysis of the total events in the REDUCE-IT trial as outlined above in Example 1, indicated a significant reduction in ischemic events with icosapent ethyl versus placebo. More specifically, the results from this study show that there was a 32% relative risk reduction and in total events for the primary composite efficacy outcome. In addition, first events were reduced by 25%, second events were reduced by 28%, and third or more events were reduced by 50%. For every 100 patients treated with icosapent ethyl for five years, approximately 16 total primary endpoint events could be prevented: 1 cardiovascular death, 4 myocardial infarctions, 1 stroke, 8 coronary revascularizations, and 2 episodes of unstable angina. An examination of total events for the key secondary endpoint corroborated the significant reduction in important ischemic events seen with the primary endpoint. There was a consistent benefit in both the secondary prevention and primary prevention strata.

There were significant reductions in the number of total events for each individual component of the composite primary endpoint. This benefit of icosapent ethyl across a variety of different endpoints (i.e., coronary, cerebral, fatal, nonfatal, ischemic events, revascularizations) suggests that the drug benefit is not likely to be explained by triglyceride lowering alone but rather, it strongly suggests that there are multiple mechanisms of action of the drug beyond triglyceride lowering that work together to achieve the observed benefits. Basic investigations support this contention. Icosapent ethyl was well tolerated with no significant difference in rates of serious adverse events versus placebo. Although overall rates were low in both treatment groups, and none of the events were fatal, there was a trend towards increased serious bleeding with no significant increases in adjudicated hemorrhagic stroke, serious central nervous system bleeding, or gastrointestinal bleeding. There was a small, but statistically significant, increase in hospitalization for atrial fibrillation or flutter noted in the REDUCE-IT study as described in Example 1. Nevertheless, the large number of important ischemic events averted, including a significant reduction in cardiovascular death, provides a very favorable risk-benefit profile. Given the broad inclusion criteria and relatively few exclusion criteria, these results are likely generalizable to a large proportion of statin-treated patients with atherosclerosis or diabetes.

In conclusion, icosapent ethyl 4 g per day (i.e., 2 g twice per day) significantly reduces total ischemic events in patients with established atherosclerosis or with diabetes and additional cardiovascular risk factors already being treated with statin therapy, with consistent benefits across a variety of individual ischemic endpoints. In patients with elevated triglycerides with cardiovascular disease or diabetes, icosapent ethyl reduces total ischemic events in both secondary and primary prevention. In such patients with fasting triglycerides 135 mg/dL and above, icosapent ethyl should be considered in order to reduce the total burden of atherosclerotic events.

### Example 3: The Impact of Icosapent Ethyl on Treating and/or Preventing Patients Infected with SARS-CoV-2

The objective of the following study is to assess the impact of icosapent ethyl on treating and/or preventing infection with SARS-CoV-2, development of COVID-19, and/or symptoms thereof. This study provides substantial preclinical and clinical data to suggest that EPA has anti-inflammatory, anti-thrombotic, and potentially anti-viral properties, which ultimately may result in beneficial effects within those infected patients.

Methods

Study Design

The following study includes (A) a multi-center, placebo-controlled clinical trial the details of which are described above in Example 1, the REDUCE-IT (R-I) design; and (B) a multi-center, placebo-controlled clinical trial the details of which are described in U.S. Patent Application Publication No. 2007/0148643, filed on Nov. 26, 2003, entitled “Treatment of Huntington's disease with EPA;” U.S. Patent Application Publication No. 2009/0270504, filed on Apr. 24, 2009, entitled “Treatment of Huntington's disease with EPA;” WIPO Publication No. WO 2000/044360, filed on Jan. 20, 2000, entitled “Drugs for Treatment of Psychiatric and Brain Disorders;” WIPO Publication No. WO 2000/0044361, Filed Jan. 21, 2000, entitled “Highly Purified Ethyl EPA and Other EPA Derivatives for Psychiatric and Neurological Disorders;” U.S. Pat. No. 6,384,077, filed Jan. 27, 2000, and entitled “Highly Purified EPA for Treatment of Schizophrenia and Related Disorders;” U.S. Patent Application Publication No. US 2002/0169209, filed Apr. 30, 2002, entitled “Potentiation of Therapeutic Effects of Fatty Acids,” all of which are incorporated by reference herein in their entirety. For (A), as shown in FIG. 12, patients were randomized in a double-blind manner to icosapent ethyl 4 g per day (2 grams twice daily with food) versus placebo.

### Results

### Adverse Event Outcomes

Tables 29A-29C tabulate the adverse events (AEs) across the studied patient population. As demonstrated in Tables 29A-29C, in comparing the AEs across the patient populations, there were a number of events related to the symptoms associated with patients infected with SARS-CoV-2 that have symptoms of COVID-19 and may or may not have COVID-19 that were reduced in patients administered icosapent ethyl as compared to those patients who received a placebo control.

For example, the data demonstrates that those patients who received icosapent ethyl exhibit fewer coughs and fewer nasopharyngitis incidents as compared to those who received the placebo control (Table 29A (respiratory)). There was further evidence for a reduction in wheezing as well as fewer cases of systemic inflammatory response syndrome (SIRS, a precursor to sepsis) (Table 29B (respiratory and immunological, respectively)). The reduction in systemic inflammatory response was particularly significant. In specific, systemic inflammatory response was associated with a risk ratio (RR) of 0.143 and an p-value of 0.0338. Accordingly, the data suggest that icosapent ethyl benefits patients suffering from coughs and/or mucosal infections (e.g., nasopharyngitis) and reduces SIRS.

TABLE 29A
Tabulated Binary/Discretized Events for Patients in the Study Population
Present Groupwise Comparisons for Other Candidates
Tabulate Binary/Discretized Events for Other Candidates
Other Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Cardiovascular

| | | | | |
|---|---|---|---|---|
| R-I | Erectile dysfunction (AE table) | 0.784 (0.535 to 1.151) 2 | 1.8%/−0.4%/232 3 | p = 0.2135 2 |

Musculoskeletal

| | | | | |
|---|---|---|---|---|
| R-I | Blood CPK increased (AE table) | 0.684 (0.485 to 0.964) 2 | 1,6%/−0.6%/164 3 | p = 0.0289 5 |
| R-I | Muscle spasms (AE table) | 0.579 (0.421 to 0.795) 3 | 2.0%/−1.1%/95 4 | p = 0.0006 5 |
| ISS | Muscle spasms, Related (AE table) | 0.741 (0.046 to 11.826) 2 | 0.2%/−0.0%/2007 1 | p = 0.8317 1 |

Respiratory

| | | | | |
|---|---|---|---|---|
| R-I | Cough (AE table) | 0.526 (0.245 to 1.131) 3 | 0.4%/−0.2%/455 2 | p = 0.0942 4 |
| ISS | Cough (AE table) | 1.928 (0.692 to 5.373) −3 | 1.5%/0.9%/−112 −3 | p = 0.2008 −2 |
| ISS | Nasopharyngitis, Any Severity (AE table) | 0.588 (0.344 to 1.004) 3 | 4.3%/−2.3%/43 5 | p = 0.0492 5 |
| ISS | Nasopharyngitis, Mild Severity (AE table) | 0.494 (0.265 to 0.921) 3 | 3.3%/−2.3%/43 5 | p = 0.0234 5 |

Dermatological

| | | | | |
|---|---|---|---|---|
| R-I | Dermatitis, allergic (AE table) | 0.250 (0.071 to 0.885) 4 | 0.2%/−0.2%/454 2 | p = 0.0200 5 |
| ISS | Hot Flush (AE table) | 0.556 (0.125 to 2.474) 3 | 0.6%/−0.3%/292 2 | P = 0.4344 2 |

Gastrointestinal

| | | | | |
|---|---|---|---|---|
| ISS | Constipation, Related (AE table) | 0.148 (0.017 to 1.265) 5 | 0.5%/−0.8%/122 3 | p = 0.0430 5 |
| R-I | Decreased Appetite (AE table) | 2.584 (1.329 to 5.024) −4 | 0.5%/0.5%/−215 −3 | p = 0.0037 −5 |
| ISS | Diarrhea, Any Severity (AE table) | 0.847 (0.603 to 1.191) 1 | 9.8%/−1.6%/61 4 | p = 0.3398 2 |
| ISS | Diarrhea, Mild Severity (AE table) | 0.741 (0.498 to 1.103) 2 | 7.4%/−2.2%/45 5 | p = 0.1388 3 |
| ISS | Diarrhea, Related (AE table) | 0.834 (0.575 to 1.210) 1 | 8.4%/−1.5%/65 4 | p = 0.3388 2 |
| ISS | Flatulence, Any Severity (AE table) | 0.463 (0.152 to 1.408) 3 | 1.1%/−0.8%/121 3 | p = 0.1645 3 |
| ISS | Flatulence, Mild Severity (AE table) | 0.424 (0.125 to 1.440) 3 | 0.9%/−0.8%/129 3 | p = 0.1559 3 |
| ISS | Flatulence, Related (AE table) | 0.463 (0.152 to 1.408) 3 | 1.1%/−0.8%/121 3 | p = 0.1645 3 |
| ISS | Frequent Bowel Movements, Any | [0 drug vs 3 PBO] | 0.2%/−0.6%/173 3 | p = 0.0440 5 |
| | Severity (AE table) | | | |
| ISS | Frequent Bowel Movements, Mild | [0 drug vs 2 PBO] | 0.2%/−0.4%/259 2 | p = 0.1002 3 |
| | Severity (AE table) | | | |
| ISS | Frequent Bowel Movements, Related | [0 drug vs 3 PBO] | 0.2%/−0.6%/173 3 | p = 0.0440 5 |
| | (AE table) | | | |
| R-I | Gastroesophageal reflux (AE table) | 0.333 (0.090 to 1.231) 4 | 0.1%/−0.1%/682 1 | p = 0.0831 4 |
| R-I | Large intestine polyps (AE table) | 0.385 (0.137 to 1.078) 4 | 0.2%/−0.2%/511 1 | p = 0.0591 4 |
| R-I | NASH (AE table) | 0.167 (0.020 to 1.384) 5 | 0.1%/−0.1%/818 1 | p = 0.0587 4 |
| R-I | NASH for b/l NFS >0.675 (AE table) | 0.252 (0.028 to 2.251) 4 | 0.3%/−0.4%/273 2 | p = 0.1822 3 |
| R-I | NASH+Hepatic Steatosis for | 0.740 (0.342 to 1.601) 2 | 1.6%/−0.5%/209 3 | p = 0.4421 2 |
| | NFS >0.675 (AE table) | | | |
| ISS | Weight loss, Related (AE table) | 0.247 (0.026 to 2.369) 4 | 0.3%/−0.4%/230 3 | p = 0.1890 3 |

Neurological

| | | | | |
|---|---|---|---|---|
| ISS | Chorea, Related (AE table) | 0.445 (0.107 to 1.853) 3 | 0.7%/−0.5%/187 3 | p = 0.2528 2 |
| ISS | Huntington's Chorea, Any Severity (AE | 0.247 (0.067 to 0.908) 4 | 1.0%/−1.3%/77 4 | p = 0.0224 5 |
| | table) | | | |
| ISS | Huntington's Chorea, Mild Severity (AE | 0.318 (0.083 to 1.223)4 | 0.8%/−0.9%/109 3 | p = 0.0782 4 |
| | table) | | | |

Gynecological

| | | | | |
|---|---|---|---|---|
| ISS | Menorrhagia (AE table) | 0.185 (0.021 to 1.653) 5 | 0.4%/−0.6%/159 3 | p = 0.0899 4 |

TABLE 29B
Tabulated Binary/Discretized Events for Patients in the Study Population
Present Groupwise Comparisons for Retrospective Candidates
Tabulate Binary/Discretized Events for Retrospective Candidates
Retrospective Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Cardiovascular

| | | | | |
|---|---|---|---|---|
| R-I | Deep Venous Thromboembolism (AE table) | 0.625 (0.205 to 1.909) 2 | 0.2%/−0.1%/1364 1 | p = 0.4053 2 |
| R-I | Embolism and Thrombosis (AE table) | 1.064 (0.716 to 1.581) −1 | 1.2%/0.1%/−1358 −1 | p = 0.7584 −1 |

Respiratory

| | | | | |
|---|---|---|---|---|
| R-I | Asthma (AE table) | 1.076 (0.742 to 1.560) −1 | 1.3%/0.1%/−1019 −1 | p = 0.7000 −1 |
| R-I | COPD (AE table) | 1.084 (0.837 to 1.405) −1 | 2.7%/0.2%/−453 −2 | p = 0.5399 −1 |
| R-I | Cystic Fibrosis (AE table) | [No cases in study] | | |
| R-I | Wheezing (AE table) | 0.639 (0.379 to 1.076) 2 | 0.7%/−0.3%/315 2 | p = 0.0896 4 |

Nephrological

| | | | | |
|---|---|---|---|---|
| R-I | CKD (AE table) | 1.123 (0.766 to 1.646) −1 | 1.3%/0.1%/−680 −1 | p = 0.5529 −1 |
| R-I | Diabetic Nephropathy (AE table) | 0.909 (0.387 to 2.139) 1 | 0.3%/−0.0%/4100 1 | p = 0.8275 1 |
| R-I | ESRD (AE table) | 0.667 (0.111 to 3.989) 2 | 0.1%/−0.0%/4092 1 | p = 0.6548 1 |
| R-I | Glomerulonephritis and Nephrotic | [0 drug vs 4 PBO] | 0.0%/−0.1%/1023 1 | p = 0.0455 5 |
| | Syndrome (AE table) | | | |
| R-I | IgA Nephropathy (AE table) | [No cases in study] | | |
| R-I | Nephrolithiasis (AE table) | 1.050 (0.777 to 1.419) −1 | 2.0%/0.1%/−1017 −1 | p = 0.7526 −1 |
| R-I | Nephropathies (AE table) | 0.679 (0.380 to 1.213)2 | 0.6%/−0.2%/455 2 | p = 0.1883 3 |
| R-I | Nephrotic Syndrome (AE table) | [0 drug vs 1 PBO] | 0.0%/−0.0%/4090 1 | p = 03173 2 |
| R-I | Renal Transplant (AE table) | [No cases in study] | | |

Gastrointestinal

| | | | | |
|---|---|---|---|---|
| R-I | Chrohn's Disease (AE table) | 0.333 (0.035 to 3.204) 4 | 0.0%/−0.0%/2045 1 | p = 0.3173 2 |
| R-I | Hepatitis C (AE table) | [1 drug vs 0 PBO] | 0.0%/0.0%/−4089 −1 | p = 0.3172 −2 |
| R-I | Ulcerative Colitis (AE table) | [0 drug vs 1 PBO] | 0.0%/−0.0%/4090 1 | p = 0.3173 2 |

Hematologic

| | | |
|---|---|---|
| R-I | Sickle Cell Disease (AE table) | [No cases in study] |

Musculoskeletal

| | | | | |
|---|---|---|---|---|
| R-I | Bone Fractures (AE table) | 2.000 (0.181 to 22.053) −3 | 0.0%/0.0%/−4088 −1 | p = 0.5635 −1 |
| R-I | Dermatitis psoriasiform (AE table) | [1 drug vs 0 PBO] | 0.0%/0.0%/−4089 −1 | p = 0.3172 −2 |
| R-I | Guttate psoriasis (AE table) | [1 drug vs 0 PBO] | 0.0%/0.0%/−4089 −1 | p = 0.3172 −2 |
| R-I | Limb Fractures and Dislocations (AE table) | 0.883 (0.541 to 1.439) 1 | 0.8%/−0.1%/1024 1 | p = 0.6164 1 |
| R-I | Lupus (AE table) | [0 drug vs 1 PBO] | 0.0%/−0.0%/4090 1 | p = 0.3173 2 |
| R-I | Psoriasis (AE table) | 1.750 (0.949 to 3.230) −3 | 0.5%/0.3%/−341 −2 | p = 0.0696 −4 |
| R-I | Psoriatic Conditions (AE table) | 1.875 (1.024 to 3.435) −3 | 0.6%/0.3%/−292 −2 | p = 0.0384 −5 |
| R-I | Rheumatoid Arthritis (AE table) | [0 drug vs 3 PBO] | 0.0%/−0.1%/1363 1 | p = 0.0832 4 |

Immunologic

| | | | | |
|---|---|---|---|---|
| R-I | Sepsis (AE table) | 0.807 (0.477 to 1.364) 1 | 0.7%/−0.1%/682 1 | p = 0.4216 2 |
| R-I | Systemic Inflammatory Response | 0.143 (0.018 to 1.161) 5 | 0.1%/−0.1%/682 1 | p = 0.0338 5 |
| | Syndrome (AE table) | | | |

Gynecological

| | | |
|---|---|---|
| R-I | PMS/Dysmenorrhea (AE table) | [No cases in study] |
| R-I | Polycystic Ovarian Syndrome (AE table) | [No cases in study] |

Genitourinary

| | | |
|---|---|---|
| R-I | Male Infertility (AE table) | [No cases in study] |

Psychiatric

| | | | | |
|---|---|---|---|---|
| R-I | Bipolar Disease (AE table) | 0.333 (0.035 to 3.204) 4 | 0.0%/−0.0%/2045 1 | p = 0.3173 2 |
| R-I | Depression: Depressed mood | 1.106 (0.861 to 1.422) −1 | 2.9%/0.3%/−340 −2 | p = 0.4288 −2 |
| | disorders and disturbances (AE table) | | | |
| R-I | Depression: Depressive disorders | 1.123 (0.867 to 1.454) −1 | 2.8%/0.3%/−314 −2 | p = 0.3785 −2 |
| | (AE table) | | | |
| R-I | Depression: Depressive disorders- | [2 drug vs 0 PBO] | 0.0%/0.0%/−2044 −1 | p = 0.1572 −3 |
| | Agitated depression (AE table) | | | |

TABLE 29C
Tabulated Binary/Discretized Events for Patients in the Study Population
Retrospective Candidates by Risk Ratio

| | | Adjusted % | | |
|---|---|---|---|---|
| | | Change | | |
| | | Risk | | |
| Source | Candidate Outcome | Ratio | 95% CI | P-Value |
| set | Candidate Outcome | (95% CI) | AR/ARR/NNT | Value |
| R-I | Depression: Depressive | 1.097 (0.843 to 1.428) −1 | 2.6%/0.2%/−408 −2 | p = 0.4893 −2 |
| | disorders - Depression (AE table) | | | |
| R-I | Depression: Depressive | 3.001 (0.312 to 28.836) −4 | 0.0%/0.0%/−2044 −1 | p = 0.3171 −2 |
| | disorders - Major depression (AE table) | | | |
| R-I | Depression: Depressive | 2.000 (0.181 to 22.053) −3 | 0.0%/0.0%/−4088 −1 | p = 0.5635 −1 |
| | disorders - Persistent depressive | | | |
| | disorder (AE table) | | | |
| R-I | Depression: Mood alterations - | 1.000 (0.290 to 3.452) −1 | 0.1%/0.0%/−3344802 −1 | p = 0.9997 −1 |
| | Depressed mood (AE table) | | | |
| R-I | Depression: Mood alterations - | 0.500 (0.045 to 5.513) 3 | 0.0%/−0.0%/4091 1 | p = 0.5638 1 |
| | Depressive symptom (AE table) | | | |
| R-I | Depression: Mood alterations | 0.857 (0.288 to 2.549) 1 | 0.2%/−0.0%/4096 1 | p = 0.7817 1 |
| | with depressive symptoms (AE table) | | | |
| R-I | PTSD (AE table) | 0.667 (0.111 to 3.989) 2 | 0.1%/−0.0%/4092 1 | p = 0.6548 1 |
| R-I | Psychosis: Psychotic disorder | 0.500 (0.045 to 5.513) 3 | 0.0%/−0.0%/4091 1 | p = 0.5638 1 |
| | NEC (AE table) | | | |
| R-I | Schizophrenia NEC (AE table) | 0.500 (0.045 to 5.513) 3 | 0.0%/−0.0%/4091 1 | p = 0.5638 1 |

Neurological

| | | | | |
|---|---|---|---|---|
| R-I | ADHD (AE table) | 1.000 (0.141 to 7.097) −1 | 0.0%/0.0%/−8362005 −1 | p = 0.9998 −1 |
| R-I | Alzheimer's Disease (AE table) | 1.000 (0.290 to 3.452) −1 | 0.1%/0.0%/−3344802 −1 | p = 0.9997 −1 |
| R-I | Cognitive Decline: Attention | 1.000 (0.141 to 7.097) −1 | 0.0%/0.0%/−8362005 −1 | p = 0.9998 −1 |
| | disorders and disturbances (AE table) | | | |
| R-I | Cognitive Decline: Cognitive | 1.000 (0.141 to 7.097) −1 | 0.0%/0.0%/−8362005 −1 | p = 0.9998 −1 |
| | and attention disorders and | | | |
| | disturbances (AE table) | | | |
| R-I | Cognitive Decline: Cognitive | [No cases in study] | | |
| | disturbances (AE table) | | | |
| R-I | Epilepsy (AE table) | 1.000 (0.063 to 15.986)−1 | 0.0%/0.0%/−1.67e+07 −1 | p = 0.9999 −1 |
| R-I | Migraine (AE table) | 2.751 (0.877 to 8.631) −4 | 0.2%/0.2%/−584 −1 | p = 0.0704 −4 |
| R-I | Multiple Sclerosis (AE table) | [0 drug vs 1 PBO] | 0.0%/−0.0%/4090 1 | p = 0.3173 2 |

Oncology

| | | | | |
|---|---|---|---|---|
| R-I | Acute Myeloid Leukaemia (AE table) | 1.000 (0.141 to 7.097) −1 | 0.0%/0.0%/−8362005 −1 | p = 0.9998 −1 |
| R-I | Breast Cancer (AE table) | 1.000 (0.351 to 2.849) −1 | 0.2%/0.0%/−2389144 −1 | p = 0.9996 −1 |
| R-I | Chronic Lymphycytic Leukaemia | [3 drug vs 0 PBO] | 0.0%/0.1%/−1363 −1 | p = 0.0832 −4 |
| | (AE table) | | | |
| R-I | Colon Cancer (AE table) | 2.000 (0.603 to 6.638) −3 | 0.1%/0.1%/−1022 −1 | p = 0.2477 −2 |
| R-I | Gastric Cancer (AE table) | 2.000 (0.181 to 22.053) −3 | 0.0%/0.0%/−4088 −1 | p = 0.5635 −1 |
| R-I | Leukaemia (AE table) | 1.000 (0.063 to 15.986) −1 | 0.0%/0.0%/−1.67e+07 −1 | p = 0.9999 −1 |
| R-I | Liver Cancer (AE table) | 2.000 (0.181 to 22.053) −3 | 0.0%/0.0%/−4088 −1 | p = 0.5635 −1 |
| R-I | Lung Cancer (AE table) | 0.637 (0.247 to 1.640) 2 | 0.2%/−0.1%/1023 1 | p = 0.3455 2 |
| R-I | Mucositis (AE table) | [No cases in study] | | |
| R-I | Pancreatic Cancer (AE table) | 5.001 (0.585 to 42.789) −5 | 0.1%/0.1%/−1022 −1 | p = 0.1023 −3 |
| R-I | Squamous Cell Carcinoma of | [0 drug vs 1 PBO] | 0.0%/−0.0%/4090 1 | p = 0.3173 2 |
| | Head and Neck (AE table) | | | |

Ophthalmologic

| | | | | |
|---|---|---|---|---|
| R-I | Age-Related Macular | 2.000 (0.181 to 22.053) −3 | 0.0%/0.0%/−4088 −1 | p = 0.5635 −1 |
| | Degeneration (AE table) | | | |
| R-I | Dry Eye (AE table) | 1.129 (0.698 to 1.828) −1 | 0.8%/0.1%/−1020 −1 | p = 0.6203 −1 |
| R-I | Macular Degeneration (AE table) | 1.188 (0.612 to 2.306) −1 | 0.4%/0.1%/−1361 −1 | p = 0.6108 −1 |

Dental

| | | | | |
|---|---|---|---|---|
| R-I | Periodontitis (AE table) | 1.667 (0.399 to 6.971) −2 | 0.1%/0.0%/−2044 −1 | p = 0.4791 −2 |

Leukocyte Physiology. Tables 30-31 tabulate the leukocyte physiology across the studied patient population. As demonstrated in Tables 30-31, in comparing the leukocyte physiology across the patient populations, patients administered icosapent ethyl exhibited a decrease in the neutrophil/leukocyte ratio as compared to those patients who received a placebo control, suggesting that administration of icosapent ethyl creates a less pro-inflammatory state in the patients administered icosapent ethyl (Table 30).

The data also show that there as a tendency for a left shift on the WBC differential, meaning the mix of WBCs was more likely to favor lymphocytes, and less prone to favoring neutrophils (Table 31). This further suggests a less pro-inflammatory state in patients administered icosapent ethyl, to the extent that a right shift often associates with more inflammation.

TABLE 30
Tabulated Binary/Discretized Events for Leukocyte Physiology in the Study Population

Present Groupwise Comparisons for Leukocyte Physiology Candidates
Tabulate Binary/Discretized Events for Leukocyte Physiology Candidates
Leukocyte Physiology Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Lymphocytes
Low Lymphocytes

| | | | | |
|---|---|---|---|---|
| R-I | Lymphocytes: PCS Low <30% | 0.924 (0.904 to 0.945) 1 | 80.3%/−6.3%/16 5 | p = 0.0000 5 |

High Lymphocytes

| | | | | |
|---|---|---|---|---|
| R-I | Lymphocytes: PCS High >45% | 1.543 (1.296 to 1.838) −2 | 6.2%/2.7%/−38 −5 | p = 0.0000 −5 |

Tabulate Adjusted Percent Change for Leukocyte Physiology Candidates
Leukocyte Physiology Candidates by Placebo-Adjusted Percent Change

| | | | | |
|---|---|---|---|---|
| | Candidate | Adjusted % | | |
| Source | Outcome | Change | 95% CI | P-Value |
| studyabbrS | octileX | ttxDPX | ttciDPX | ttpDPX |

Neutrophils

| | | | | |
|---|---|---|---|---|
| R-I | Neutrophils (Day 120) | +1.11% | (−.436 to +2.66%) | p = 0.1594 |
| R-I | Neutrophils (Day 360) | −3% | (−4.71 to −1.29%) | p = 0.0006 |
| R-I | Neutrophils (Day 720) | −4.7% | (−6.39 to −3.01%) | p = 0.0000 |
| R-I | Neutrophils (Day 1080) | −4.52% | (−6.41 to −2.63%) | p = 0.0000 |
| R-I | Neutrophils (Day 1440) | −3.06% | (−5.19 to −.926%) | p = 0.0050 |
| R-I | Neutrophils/Leukocytes (Ratio) (Day 120) | −2.06% | (−2.64 to −1.48%) | p = 0.0000 |
| R-I | Neutrophils/Leukocytes (Ratio) (Day 360) | −3.87% | (−4.49 to −3.25%) | p = 0.0000 |
| R-I | Neutrophils/Leukocytes (Ratio) (Day 720) | −4.61% | (−5.28 to −3.94%) | p = 0.0000 |
| R-I | Neutrophils/Leukocytes (Ratio) (Day 1080) | −4.21% | (−5.04 to −3.38%) | p = 0.0000 |
| R-I | Neutrophils/Leukocytes (Ratio) (Day 1440) | −4.1% | (−4.98 to −3.22%) | p = 0.0000 |

TABLE 31
Number of Subjects with Treatment
Emergent Potentially Significant Laboratory
Exams: Hematology Population
Number (%) of Subjects with Treatment-
Emergent Potentially Clinically Significant
Laboratory Exams: Hematology Safety Population

| | AMR101 | Placebo |
|---|---|---|
| Visit, n(%) | (N = 4089) | (N = 4090) |
| WBC Differential (Monocytes) | 3974 | 3973 |
| Monocytes: PCS High >6% | 3101 (78.0%) | 3191 (80.3%) |
| Platelet Count | 3968 | 3972 |
| PCS High >500 × 10\hat{ }3/uL | 16 (0.4%) | 18 (0.5%) |
| PCS Low <100 × 10\hat{ }3/uL | 19 (0.5%) | 43 (1.1%) |

Erythrocyte Physiology. The erythrocyte physiology for the patient population was determined as enumerated in Tables 32A-32B. Table 33 further enumerates tabulated adjusted change for erythrocyte physiology for the patient population.

TABLE 32A
Tabulated Binary/Discretized Events for Erythrocyte Candidates
Present Groupwise Comparisons for Erythrocyte Physiology Candidates
Tabulate Binary/Discretized Events for Erythrocyte Physiology Candidates
Erythrocyte Physiology Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Clinical Anemia

| | | | | |
|---|---|---|---|---|
| R-I | Anemia (AE table) | 0.810 (0.672 to 0.975) 1 | 5.2%/−1.1%/91 4 | p = 0.0255 5 |
| ISS | Hemoglobin Decreased (AE table) | [0 drug vs 2 PBO] | 0.2%/−0.4%/259 2 | p = 0.1002 3 |

RBC Mass

| | | | | |
|---|---|---|---|---|
| R-I | Erythrocytes low (Day 120) | 0.814 (0.685 to 0.968) 1 | 6.3%/−1.3%/77 4 | p = 0.0198 5 |
| R-I | Erythrocytes low (Day 360) | 0.932 (0.781 to 1.113) 1 | 6.4%/−0.4%/223 3 | p = 0.4371 2 |
| R-I | Erythrocytes low (Day 720) | 0.940 (0.779 to 1.133) 1 | 6.4%/−0.4%/251 2 | p = 0.5130 1 |
| R-I | Erythrocytes low (Day 1080) | 0.781 (0.642 to 0.950) 2 | 6.8%/−1.7%/60 4 | p = 0.0131 5 |
| R-I | Erythrocytes low (Day 1440) | 0.888 (0.720 to 1.093) 1 | 7.0%/−0.8%/120 3 | p = 0.2620 2 |
| R-I | Erythrocytes low (Day 1800) | 0.975 (0.756 to 1.258) 1 | 8.3%/−0.2%/485 2 | P = 0.8479 1 |

Hemoglobin
Low Hemoglobin

| | | | | |
|---|---|---|---|---|
| R-I | Hemoglobin shift: normal to low | 1.013 (0.787 to 1.303) −1 | 3.3%/0.0%/−2370 −1 | p = 0.9211 −1 |
| | (Day 120) | | | |
| R-I | Hemoglobin shift: normal to low | 0.828 (0.652 to 1.051) 1 | 3.9%/−0.7%/134 3 | p = 0.1200 3 |
| | (Day 360) | | | |
| R-I | Hemoglobin shift: normal to low | 0.815 (0.657 to 1.011) 1 | 5.3%/−1.1%/93 4 | p = 0.0626 4 |
| | (Day 720) | | | |
| R-I | Hemoglobin shift: normal to low | 0.703 (0.570 to 0.868) 2 | 6.5%/−2.3%/44 5 | p = 0.0010 5 |
| | (Day 1080) | | | |
| R-I | Hemoglobin shift: normal to low | 0.685 (0.553 to 0.849) 2 | 7.3%/−2.7%/37 5 | p = 0.0005 5 |
| | (Day 1440) | | | |
| R-I | Hemoglobin shift: normal to low | 0.827 (0.630 to 1.085) 1 | 7.8%/−1.5%/67 4 | p = 0.1690 3 |
| | (Day 1800) | | | |

High Hemoglobin

| | | | | |
|---|---|---|---|---|
| R-I | Hemoglobin shift: normal to high | 1.668 (0.915 to 3.042) −2 | 0.6%/0.3%/−313 −2 | p = 0.0914 −4 |
| | (Day 120) | | | |
| R-I | Hemoglobin shift: normal to high | 0.908 (0.571 to 1.442) 1 | 1.1%/−0.1%/961 1 | p = 0.6814 1 |
| | (Day 360) | | | |
| R-I | Hemoglobin shift: normal to high | 1.144 (0.760 to 1.721) −1 | 1.5%/0.2%/−491 −2 | p = 0.5202 −1 |
| | (Day 720) | | | |
| R-I | Hemoglobin shift: normal to high | 2.241 (1.330 to 3.778) −3 | 1.3%/1.0%/−103 −3 | p = 0.0018 −5 |
| | (Day 1080) | | | |
| R-I | Hemoglobin shift: normal to high | 1.829 (1.036 to 3.229) −3 | 1.2%/0.7%/−145 −3 | p = 0.0344 −5 |
| | (Day 1440) | | | |
| R-I | Hemoglobin shift: normal to high | 1.383 (0.686 to 2.788) −2 | 1.3%/0.4%/−240 −3 | p = 0.3625 −2 |
| | (Day 1800) | | | |

Hematocrit
Low Hematocrit

| | | | | |
|---|---|---|---|---|
| R-I | Hematocrit shift: normal to low | 0.828 (0.612 to 1.120) 1 | 2.3%/−0.4%/228 3 | p = 0.2197 2 |
| | (Day 120) | | | |
| R-I | Hematocrit shift: normal to low | 0.784 (0.583 to 1.054) 2 | 2.6%/−0.6%/158 3 | p = 0.1056 3 |
| | (Day 360) | | | |
| R-I | Hematocrit shift: normal to low | 0.747 (0.579 to 0.964) 2 | 3.8%/−1.1%/90 4 | p = 0.0246 5 |
| | (Day 720) | | | |
| R-I | Hematocrit shift: normal to low | 0.607 (0.469 to 0.785) 2 | 4.5%/−2.2%/45 5 | p = 0.0001 5 |
| | (Day 1080) | | | |
| R-I | Hematocrit shift: normal to low | 0.650 (0.500 to 0.845) 2 | 5.0%/−2.1%/47 5 | p = 0.0012 5 |
| | (Day 1440) | | | |
| R-I | Hematocrit shift: normal to low | 0.859 (0.627 to 1.179) 1 | 5.9%/−0.9%/111 3 | p = 0.3473 2 |
| | (Day 1800) | | | |

High Hematocrit

| | | | | |
|---|---|---|---|---|
| R-I | Hematocrit shift: normal to high | 1.507 (0.952 to 2.386) −2 | 1.1%/0.4%/−234 −3 | p = 0.0783 −4 |
| | (Day 120) | | | |
| R-I | Hematocrit shift: normal to high | 1.273 (0.811 to 1.999) −2 | 1.1%/0.3%/−364 −2 | p = 0.2928 −2 |
| | (Day 360) | | | |
| R-I | Hematocrit shift: normal to high | 1.181 (0.724 to 1.927) −1 | 1.1%/0.2%/−566 −1 | p = 0.5047 −1 |
| | (Day 720) | | | |
| R-I | Hematocrit shift: normal to high | 1.433 (0.826 to 2.487) −2 | 1.0%/0.4%/−282 −2 | p = 0.1983 −3 |
| | (Day 1080) | | | |
| R-I | Hematocrit shift: normal to high | 2.208 (1.155 to 4.221) −3 | 1.0%/0.7%/−138 −3 | p = 0.0139 −5 |
| | (Day 1440) | | | |
| R-I | Hematocrit shift: normal to high | 1.420 (0.582 to 3.462) −2 | 0.8%/0.3%/−354 −2 | p = 0.4381 −2 |
| | (Day 1800) | | | |

Heme Derivative (Antioxidant/CHD Risk Marker)

| | | | | |
|---|---|---|---|---|
| R-I | Bilirubin >1x ULN to 2x ULN | 3.157 (2.392 to 4.168) −4 | 3.3%/3.5%/−29 −5 | p = 0.0000 −5 |
| R-I | Bilirubin >2x ULN to 3x ULN | 3.668 (1.024 to 13.136) −4 | 0.2%/0.2%/−497 −2 | p = 0.0323 −5 |
| R-I | Bilirubin high (Day 120) | 4.288 (2.849 to 6.454) −4 | 1,9%/2.4%/−42 −5 | p = 0.0000 −5 |
| R-I | Bilirubin high (Day 360) | 3.263 (2.203 to 4.832) −4 | 1.9%/2.0%/−50 −5 | p = 0.0000 −5 |
| R-I | Bilirubin high (Day 720) | 5.052 (3.174 to 8.039) −5 | 2.0%/2.6%/−38 −5 | p = 0.0000 −5 |
| R-I | Bilirubin high (Day 1080) | 2.430 (1.683 to 3.508) −3 | 2.4%/2.0%/−50 −5 | p = 0.0000 −5 |
| R-I | Bilirubin high (Day 1440) | 3.543 (2.243 to 5.596) −4 | 2.3%/2.5%/−40 −5 | p = 0.0000 −5 |
| R-I | Bilirubin high (Day 1800) | 2.670 (1.569 to 4.545) −4 | 2.6%/2.3%/−43 −5 | p = 0.0002 −5 |
| R-I | Bilirubin shift: normal to high | 5.106 (2.754 to 9.467) −5 | 1.0%/1.3%/−77 −4 | p = 0.0000 −5 |
| | (Day 120) | | | |

TABLE 32B
Tabulated Binary/Discretized Events for Erythrocyte Candidates
Erythrocyte Physiology Candidates by Risk Ratio

| | | Adjusted % | | |
|---|---|---|---|---|
| | Candidate | Change | | |
| | Outcome | Risk | | |
| Source | Candidate | Ratio | 95% CI | P-Value |
| set | Outcome | (95% CI) | AR/ARR/NNT | Value |
| R-I | Bilirubin shift: normal to high | 5.431 (2.773 to 10.636) −5 | 0.9%/1.3%/−80 −4 | p = 0.0000 −5 |
| | (Day 360) | | | |
| R-I | Bilirubin shift: normal to high | 4.803 (2.516 to 9.168) −4 | 1.0%/1.3%/−76 −4 | p = 0.0000 −5 |
| | (Day 720) | | | |
| R-I | Bilirubin shift: normal to high | 2.235 (1.374 to 3.636) −3 | 1.4%/1.0%/−95 −4 | p = 0.0009 −5 |
| | (Day 1080) | | | |
| R-I | Bilirubin shift: normal to high | 5.445 (2.687 to 11.034) −5 | 1.3%/1.8%/−57 −4 | p = 0.0000 −5 |
| | (Day 1440) | | | |
| R-I | Bilirubin shift: normal to high | 2.556 (1.242 to 5.259) −4 | 1.4%/1.2%/−81 −4 | p = 0.0081 −5 |
| | (Day 1800) | | | |

TABLE 33
Tabulated Adjusted Percent Change for Erythrocyte Physiology Candidates
Tabulate Adjusted Percent Change for Erythrocyte Physiology Candidates
Erythrocyte Physiology Candidates by Placebo-Adjusted Percent Change

| | | | | |
|---|---|---|---|---|
| | Candidate | Adjusted % | | |
| Source | Outcome | Change | 95% CI | P-Value |
| studyabbrS | octitleX | ttxDPX | ttciDPX | ttpDPX |

RBC Mass

| | | | | |
|---|---|---|---|---|
| R-I | Erythrocytes (Day 120) | +.72% | (+.452 to +.988%) | p = 0.0000 |
| R-I | Erythrocytes (Day 360) | +.26% | (−.045 to +.565%) | p = 0.0943 |
| R-I | Erythrocytes (Day 720) | +.29% | (−.049 to +.629%) | p = 0.0937 |
| R-I | Erythrocytes (Day 1080) | +.43% | (+.047 to +.813%) | p = 0.0278 |
| R-I | Erythrocytes (Day 1440) | +.66% | (+.215 to +1.11%) | p = 0.0037 |

Hemoglobin

| | | | | |
|---|---|---|---|---|
| R-I | Hemoglobin (Day 120) | +.53% | (+.259 to +.801%) | p = 0.0001 |
| R-I | Hemoglobin (Day 360) | +1% | (+.665 to +1.33%) | p = 0.0000 |
| R-I | Hemoglobin (Day 720) | +1.51% | (+1.13 to +1.89%) | p = 0.0000 |
| R-I | Hemoglobin (Day 1080) | +2.09% | (+1.66 to +2.52%) | p = 0.0000 |
| R-I | Hemoglobin (Day 1440) | +2.36% | (+1.88 to +2.84%) | p = 0.0000 |

Hematocrit

| | | | | |
|---|---|---|---|---|
| R-I | Hematocrit (Day 120) | +.42% | (+.113 to +.727%) | p = 0.0074 |
| R-I | Hematocrit (Day 360) | +.78% | (+.441 to +1.12%) | p = 0.0000 |
| R-I | Hematocrit (Day 720) | +1.28% | (+.906 to +1.65%) | p = 0.0000 |
| R-I | Hematocrit (Day 1080) | +1.59% | (+1.16 to +2.02%) | p = 0.0000 |
| R-I | Hematocrit (Day 1440) | +1.86% | (+1.39 to +2.33%) | p = 0.0000 |

Antioxidants. The antioxidative capacity of icosapent ethyl was evaluated across the patient population. Tables 34A-34B enumerate the changes in the uric acid levels in the patient population upon administration of icosapent ethyl, and Tables 35A-35B enumerate the endogenous extracellular antioxidants candidates by risk ratio in the patient population upon administration of icosapent ethyl.

As shown in Table 35A, patients administered icosapent ethyl exhibited an increase in bilirubin levels as compared to patients who received a placebo control. The increase in bilirubin levels is significant as bilirubin is a potent endogenous antioxidant. It is contemplated that EPA can activate the heme oxidase pathway which functions to promote conversion of heme to biliverdin, a very potent endogenous antioxidant. Biliverdin is then reduced to bilirubin by biliverdin reductase. Bilirubin is also a potent endogenous antioxidant. Both biliverdin and bilirubin reduce oxidative stress (e.g., H₂O₂-induced oxidative stress in HUVEC), and bilirubin can be converted back into biliverdin, perpetuating antioxidant effects.

TABLE 34A
Tabulated Binary/Discretized Events for Uric Acid Candidates
Present Groupwise Comparisons for Uric Acid Candidates
Tabulate Binary/Discretized Events for Uric Acid Candidates
Uric Acid Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Clinical Hyperuricemia

| | | | | |
|---|---|---|---|---|
| R-I | Blood uric acid increased (AE table) | 1.154 (0.753 to 1.768) −1 | 1.0%/0.1%/−680 −1 | p = 0.5098 −1 |
| R-I | Gout (AE table) | 1.347 (1.075 to 1.687) −2 | 3.6%/1.1%/−93 −4 | p = 0.0094 −5 |
| R-I | Hyperuricemia (AE table) | 1.737 (0.990 to 3.050) −3 | 0.6%/0.3%/−292 −2 | p = 0.0514 −4 |

Uric Acid Level (Antioxidant)
Low Uric Acid Level (Antioxidant)

| | | | | |
|---|---|---|---|---|
| R-I | Urate shift: normal to low (Day 120) | 1.654 (0.396 to 6.915) −2 | 0.1%/0.1%/−1647 −1 | p = 0.4860 −2 |
| R-I | Urate shift: normal to low (Day 360) | 0.733 (0.255 to 2.110) 2 | 0.2%/−0.1%/1418 1 | p = 0.5629 1 |
| R-I | Urate shift: normal to low (Day 720) | 0.361 (0.096 to 1.360) 4 | 0.2%/−0.2%/533 1 | p = 0.1161 3 |
| R-I | Urate shift: normal to low (Day 1080) | 0.474 (0.119 to 1.893) 3 | 0.2%/−0.1%/740 1 | p = 0.2795 2 |
| R-I | Urate shift: normal to low (Day 1440) | 0.210 (0.046 to 0.973) 4 | 0.3%/−0.4%/280 2 | p = 0.0275 5 |
| R-I | Uric acid PCS Low <1.9 mg/dL (Women) | 1.000 (0.063 to 15.986) −1 | 0.0%/0.0%/−1.58e+07 −1 | p = 0.9999-1 |
| R-I | Uric acid PCS Low <2.5 mg/dL (Men) | 0.650 (0.324 to 1.305) 2 | 0.4%/−0.2%/569 1 | p = 0.2223 2 |

High Uric Acid Level (Antioxidant)

| | | | | |
|---|---|---|---|---|
| R-I | Urate high (Day 120) | 1.398 (1.246 to 1.568) −2 | 13.4%/4.4%/−22 −5 | p = 0.0000 −5 |
| R-I | Urate high (Day 360) | 1.350 (1.197 to 1.523) −2 | 13.0%/3.9%/−26 −5 | p = 0.0000 −5 |
| R-I | Urate high (Day 720) | 1.323 (1.162 to 1.506) −2 | 12.4%/3.4%/−29 −5 | p = 0.0000 −5 |
| R-I | Urate high (Day 1080) | 1.102 (0.961 to 1.263) −1 | 12.7%/1.2%/−81 −4 | p = 0.1654 −3 |
| R-I | Urate high (Day 1440) | 1.109 (0.956 to 1.286) −1 | 12.7%/1.3%/−76 −4 | p = 0.1714 −3 |
| R-I | Urate high (Day 1800) | 1.084 (0.883 to 1.331) −1 | 12.1%/1.0%/−103 −3 | p = 0.4413 −2 |
| R-I | Urate shift: normal to high (Day 120) | 1.689 (1.369 to 2.084) −3 | 5.5%/2.8%/−36 −5 | p = 0.0000 −5 |
| R-I | Urate shift: normal to high (Day 360) | 1.674 (1.363 to 2.056) −3 | 6.0%/3.0%/−33 −5 | p = 0.0000 −5 |
| R-I | Urate shift: normal to high (Day 720) | 1.513 (1.227 to 1.865) −2 | 6.2%/2.5%/−39 −5 | p = 0.0001 −5 |
| R-I | Urate shift: normal to high (Day 1080) | 1.118 (0.910 to 1.374) −1 | 7.1%/0.8%/−127 −3 | p = 0.2866 −2 |
| R-I | Urate shift: normal to high (Day 1440) | 1.079 (0.879 to 1.324) −1 | 8.2%/0.6%/−160 −3 | p = 0.4685 −2 |
| R-I | Urate shift: normal to high (Day 1800) | 1.029 (0.782 to 1.355) −1 | 8.2%/0.2%/−422 −2 | p = 0.8364 −1 |
| R-I | Uric acid PCS High >7.5 mg/dL (Women) | 1.180 (0.974 to 1.429) −1 | 5.0%/0.8%/−120 −3 | p = 0.0903 −4 |
| R-I | Uric acid PCS High >8.0 mg/dL (Men) | 1.313 (1.183 to 1.458) −2 | 15.3%/4.2%/−24 −5 | p = 0.0000 −5 |

TABLE 34B
Tabulated Adjusted Percent Change for Uric Acid Candidates
Tabulate Adjusted Percent Change for Uric Acid Candidates
Uric Acid Candidates by Placebo-Adjusted Percent Change

| | | | | |
|---|---|---|---|---|
| | Candidate | Adjusted % | | |
| Source | Outcome | Change | 95% CI | P-Value |
| studyabbrS | octitleX | ttxDPX | ttciDPX | ttpDPX |

Uric Acid Level (Antioxidant)

| | | | | |
|---|---|---|---|---|
| R-I | Urate (Day 120) | +4.31% | (+3.59 to +5.03%) | p = 0.0000 |
| R-I | Urate (Day 360) | +4.07% | (+3.2 to +4.94%) | p = 0.0000 |
| R-I | Urate (Day 720) | +3.66% | (+2.69 to +4.63%) | p = 0.0000 |
| R-I | Urate (Day 1080) | +2.94% | (+1.83 to +4.05%) | p = 0.0000 |
| R-I | Urate (Day 1440) | +3.23% | (+1.95 to +4.51%) | p = 0.0000 |

TABLE 35A
Endogenous Extracellular Antioxidants Candidates by Risk Ratio
Endogenous Extracellular Antioxidants Candidates by Risk Ratio
Endogenous Extracellular Antioxidants Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Heme Derivative (Antioxidant/CHD Risk Marker)

| | | | | |
|---|---|---|---|---|
| R-I | Bilirubin >1x ULN to 2x ULN | 3.157 (2.392 to 4.168) −4 | 3.3%/3.5%/−29 −5 | p = 0.0000 −5 |
| R-I | Bilirubin >2x ULN to 3x ULN | 3.668 (1.024 to 13.136 −4 | 0.2%/0.2%/−497 −2 | p = 0.0323 −5 |
| R-I | Bilirubin high (Day 120) | 4.288 (2.849 to 6.454) −4 | 1.9%/2.4%/−42 −5 | p = 0.0000 −5 |
| R-I | Bilirubin high (Day 360) | 3.263 (2.203 to 4.832) −4 | 1.9%/2.0%/−50 −5 | p = 0.0000 −5 |
| R-I | Bilirubin high (Day 720) | 5.052 (3.174 to 8.039) −5 | 2.0%/2.6%/−38 −5 | p = 0.0000 −5 |
| R-I | Bilirubin high (Day 1080) | 2.430 (1.683 to 3.508) −3 | 2.4%/2.0%/−50 −5 | p = 0.0000 −5 |
| R-I | Bilirubin high (Day 1440) | 3.543 (2.243 to 5.596) −4 | 2.3%/2.5%/−40 −5 | p = 0.0000 −5 |
| R-I | Bilirubin high (Day 1800) | 2.670 (1.569 to 4.545) −4 | 2.6%/2.3%/−43 −5 | p = 0.0002 −5 |
| R-I | Bilirubin shift: normal to high | 5.106 (2.754 to 9.467) −5 | 1.0%/1.3%/−77 −4 | p = 0.0000 −5 |
| | (Day 120) | | | |
| R-I | Bilirubin shift: normal to high | 5.431 (2.773 to 10.636) −5 | 0.9%/1.3%/−80 −4 | p = 0.0000 −5 |
| | (Day 360) | | | |
| R-I | Bilirubin shift: normal to high | 4.803 (2.516 to 9.168) −4 | 1.0%/1.3%/−76 −4 | p = 0.0000 −5 |
| | (Day 720) | | | |
| R-I | Bilirubin shift: normal to high | 2.235 (1.374 to 3.636) −3 | 1.4%/1.0%/−95 −4 | p = 0.0009 −5 |
| | (Day 1080) | | | |
| R-I | Bilirubin shift: normal to high | 5.445 (2.687 to 11.034) −5 | 1.3%/1.8%−57 −4 | p = 0.0000 −5 |
| | (Day 1440) | | | |
| R-I | Bilirubin shift: normal to high | 2.556 (1.242 to 5.259) −4 | 1.4%/1.2%/−81 −4 | p = 0.0081 −5 |
| | (Day 1800) | | | |

Albumin (Antioxidant/Inverse CHD Risk Marker)

| | | | | |
|---|---|---|---|---|
| R-I | Albumin high (Day 120) | 2.263 (1.388 to 3.689) −3 | 1.0%/0.8%/−133 −3 | p = 0.0008 −5 |
| R-I | Albumin high (Day 360) | 1.901 (1.217 to 2.969) −3 | 1.2%/0.7%/−138 −3 | p = 0.0041 −5 |
| R-I | Albumin high (Day 720) | 1.365 (0.975 to 1.911) −2 | 2.1%/0.6%/−156 −3 | p = 0.0684 −4 |
| R-I | Albumin high (Day 1080) | 1.449 (0.993 to 2.115) −2 | 1.9%/0.7%/−140 −3 | p = 0.0528 −4 |
| R-I | Albumin high (Day 1440) | 1.540 (1.001 to 2.369) −2 | 1.8%/0.8%/−132 −3 | p = 0.0477 −5 |
| R-I | Albumin shift: normal to high | 3.055 (1.709 to 5.461) −4 | 0.8%/0.8%/−123 −3 | p = 0.0001 −5 |
| | (Day 120) | | | |
| R-I | Albumin shift: normal to high | 2.364 (1.388 to 4.027) −3 | 0.9%/0.7%/−137 −3 | p = 0.0011 −5 |
| | (Day 360) | | | |
| R-I | Albumin shift: normal to high | 1.475 (1.028 to 2.116) −2 | 1.9%/0.7%/−140 −3 | p = 0.0337 −5 |
| | (Day 720) | | | |
| R-I | Albumin shift: normal to high | 1.257 (0.848 to 1.864) −2 | 1.8%/0.4%/−248 −3 | p = 0.2542 −2 |
| | (Day 1080) | | | |
| R-I | Albumin shift: normal to high | 1.531 (0.974 to 2.408) −2 | 1.6%/0.7%/−145 −3 | p = 0.0629 −4 |
| | (Day 1440) | | | |

Clinical Hyperuricemia

| | | | | |
|---|---|---|---|---|
| R-I | Blood uric acid increased (AE | 1.154 (0.753 to 1.768) −1 | 1.0%/0.1%/−680 −1 | p = 0.5098 −1 |
| | table) | | | |
| R-I | Hyperuricemia (AE table) | 1.737 (0.990 to 3.050) −3 | 0.6%/0.3%/−292 −2 | p = 0.0514 −4 |

Uric Acid Level (Antioxidant)
Low Uric Acid Level (Antioxidant)

| | | | | |
|---|---|---|---|---|
| R-I | Urate shift: normal to low (Day | 1.654 (0.396 to 6.915) −2 | 0.1%/0.1%/−1647 −1 | p = 0.4860 −2 |
| | 120) | | | |
| R-I | Urate shift: normal to low (Day | 0.733 (0.255 to 2.110) 2 | 0.2%/−0.1%/1418 1 | p = 0.5629 1 |
| | 360) | | | |
| R-I | Urate shift: normal to low (Day | 0.361 (0.096 to 1.360) 4 | 0.2%/−0.2%/533 1 | p = 0.1161 3 |
| | 720) | | | |
| R-I | Urate shift: normal to low (Day | 0.474 (0.119 to 1.893) 3 | 0.2%/−0.1%/740 1 | p = 0.2795 2 |
| | 1080) | | | |
| R-I | Urate shift: normal to low (Day | 0.210 (0.046 to 0.973) 4 | 0.3%/−0.4%/280 2 | p = 0.0275 5 |
| | 1440) | | | |
| R-I | Uric Acid PCS Low <1.9 mg/dL | 1.000 (0.063 to 15.986) −1 | 0.0%/0.0%/−1.58e+07 −1 | p = 0.9999 −1 |
| | (Women) | | | |
| R-I | Uric Acid PCS Low <2.5 mg/dL | 0.650 (0.324 to 1.305) 2 | 0.4%/−0.2%/569 1 | p = 0.2223 2 |
| | (Men) | | | |

High Uric Acid Level (Antioxidant)

| | | | | |
|---|---|---|---|---|
| R-I | Urate high (Day 120) | 1.398 (1.246 to 1.568) −2 | 13.4%/4.4%/−22 −5 | p = 0.0000 −5 |
| R-I | Urate high (Day 360) | 1.350 (1.197 to 1.523) −2 | 13.0%/3.9%/−26 −5 | p = 0.0000 −5 |
| R-I | Urate high (Day 720) | 1.323 (1.162 to 1.506) −2 | 12.4%/3.4%/−29 −5 | p = 0.0000 −5 |
| R-I | Urate high (Day 1080) | 1.102 (0.961 to 1.263) −1 | 12.7%/1.2%/−81 −4 | p = 0.1654 −3 |
| R-I | Urate high (Day 1440) | 1.109 (0.956 to 1.286) −1 | 12.7%/1.3%/−76 −4 | p = 0.1714 −3 |
| R-I | Urate high (Day 1800) | 1.084 (0.883 to 1.331) −1 | 12.1%/1.0%/−103 −3 | p = 0.4413 −2 |
| R-I | Urate shift: normal to high (Day | 1.689 (1.369 to 2.084) −3 | 5.5%/2.8%/−36 −5 | p = 0.0000 −5 |
| | 120) | | | |
| R-I | Urate shift: normal to high (Day | 1.674 (1.363 to 2.056) −3 | 6.0%/3.0%/−33 −5 | p = 0.0000 −5 |
| | 360) | | | |
| R-I | Urate shift: normal to high (Day | 1.513 (1.227 to 1.865) −2 | 6.2%/2.5%/−39 −5 | p = 0.0001 −5 |
| | 720) | | | |
| R-I | Urate shift: normal to high (Day | 1.118 (0.910 to 1.374) −1 | 7.1%/0.8%/−127 −3 | p = 0.2866 −2 |
| | 1080) | | | |
| R-I | Urate shift: normal to high (Day | 1.079 (0.879 to 1.324) −1 | 8.2%/0.6%/−160 −3 | p = 0.4685 −2 |
| | 1440) | | | |

TABLE 35B
Endogenous Extracellular Antioxidants Candidates by Risk Ratio
Endogenous Extracellular Antioxidants Candidates by Risk Ratio

| | | Risk | | |
|---|---|---|---|---|
| | Candidate | Ratio | | |
| set | Outcome | (95% CI) | AR/ARR/NNT | Value |
| R-I | Urate shift: normal to high (Day 1800) | 1.029 (0.782 to 1.355) −1 | 8.2%/0.2%/−422 −2 | p = 0.8364 −1 |
| R-I | Uric acid PCS High >7.5 mg/dL (Women) | 1.180 (0.974 to 1.429) −1 | 5.0%/0.8%/−120 −3 | p = 0.0903 −4 |
| R-I | Uric acid PCS High >8.0 mg/dL (Men) | 1.313 (1.183 to 1.458) −2 | 15.3%/4.2%/−24 −5 | p = 0.0000 −5 |

Cardiovascular Candidates. The cardiovascular risk events for the patient population were determined as enumerated in Tables 36A-36B.

TABLE 36A
Tabulated Binary/Discretized Events for Cardiovascular Candidates
Tabulate Binary/Discretized Events for Cardiovascular Candidates
Cardiovascular Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Cardiovascular

| | | | | |
|---|---|---|---|---|
| R-I | Deep Venous Thromboembolism (AE table) | 0.625 (0.205 to 1.909) 2 | 0.2%/−0.1%/1364 1 | p = 0.4053 2 |
| R-I | Embolism and Thrombosis (AE table) | 1.064 (0.716 to 1.581) −1 | 1.2%/0.1%−1358 −1 | p = 0.7584 −1 |
| R-I | Erectile dysfunction (AE table) | 0.784 (0.535 to 1.151) 2 | 1.8%/−0.4%/232 3 | p = 0.2135 2 |
| R-I | Carotid bruit (AE table) | 0.435 (0.207 to 0.913) 3 | 0.4%/−0.3%/315 2 | p = 0.0234 5 |
| R-I | Microalbuminuria (AE table) | 0.214 (0.062 to 0.745) 4 | 0.2%/−0.3%/372 2 | p = 0.0076 5 |
| R-I | Proteinuria (AE table) | 0.609 (0.314 to 1.181) 2 | 0.5%/−0.2%/455 2 | p = 0.1383 3 |
| ISS | Urine Protein Present (AE table) | [0 drug vs 1 PBO] | 0.1%/−0.2%/519 1 | p = 0.2453 2 |

Hypertension

| | | | | |
|---|---|---|---|---|
| R-I | Hypertension (AE table) | 0.930 (0.804 to 1.077) 1 | 8.1%/−0.6%/171 3 | p = 0.3329 2 |
| R-I | Hypertensive Crisis (AE Table) | 0.698 (0.439 to 1.110) 2 | 0.9%/−0.3%/315 2 | p = 0.1267 3 |
| R-I | Vascular hypertensive disorder (AE table) | 0.914 (0.796 to 1.050) 1 | 9.0%/−0.8%/124 3 | p = 0.2027 2 |

Diastolic Blood Pressure
Low Diastolic Blood Pressure

| | | | | |
|---|---|---|---|---|
| R-I | DBP Low (≤50 mmHG) | 1.136 (0.732 to 1.763) −1 | 1.0%/0.1%/−794 −1 | p = 0.5703 −1 |
| R-I | DBP Low and dropped ≥10 mmHg | 1.030 (0.644 to 1.648) −1 | 0.9%/0.0%/−3916 −1 | p = 0.9021 −1 |
| R-I | DBP dropped ≥10 mmHG | 1.087 (1.028 to 1.150) −1 | 37.8%/3.2%/−32 −5 | p = 0.0037 −5 |

High Diastolic Blood Pressure

| | | | | |
|---|---|---|---|---|
| R-I | DBP High (≥100 mmHg) | 1.121 (0.931 to 1.350) −1 | 5.3%/0.6%/−165 −3 | p = 0.2279 −2 |
| R-I | DBP High and Rose ≥10 mmHg | 1.092 (0.888 to 1.344) −1 | 4.3%/0.4%/−264 −2 | p = 0.4050 −2 |
| R-I | DBP rose ≥10 mmHg | 0.983 (0.927 to 1.041) 1 | 36.5%/−0.6%/158 3 | p = 0.5566 1 |

Systolic Blood Pressure
Low Systolic Blood Pressure

| | | | | |
|---|---|---|---|---|
| R-I | SBP Low (≤90 mmHg) | 1.819 (1.093 to 3.055) −3 | 0.8%/0.5%/−221 −3 | p = 0.0216 −5 |
| R-I | SBP dropped ≥20 mmHg | 1.117 (1.033 to 1.208) −1 | 24.0%/2.6%/−38 −5 | p = 0.0057 −5 |
| R-I | SBP low and dropped ≥20 mmHg | 1.422 (0.792 to 2.553) −2 | 0.6%/0.2%/−497 −2 | p = 0.2362 −2 |

High Systolic Blood Pressure

| | | | | |
|---|---|---|---|---|
| R-I | SBP High (≥160 mmHg) | 0.912 (0.833 to 1.000) 1 | 18.8%/−1.7%/58 4 | p = 0.0492 5 |
| R-I | SBP High and rose ≥20 mmHg | 0.908 (0.805 to 1.023) 1 | 11.9%/−1.1%/87 4 | p = 0.1130 3 |
| R-I | SBP rose ≥20 mmHg | 0.976 (0.912 to 1.045) 1 | 29.6%/−0.7%/140 3 | p = 0.4855 2 |

Lipids

| | | | | |
|---|---|---|---|---|
| ISS | Cholesterol Increased (AE table) | 0.165 (0.036 to 0.759) 5 | 0.9%/−1.4%/69 4 | p = 0.0082 5 |

TABLE 36B
Cardiovascular Percent Change for Cardiovascular Candidates
Tabulate Adjusted Percent Change for Cardiovascular Candidates
Cardiovascular Candidates by Placebo-Adjusted Percent Change

| | | | | |
|---|---|---|---|---|
| | Candidate | Adjusted % | | |
| Source | Outcome | Change | 95% CI | P-Value |
| studyabbrS | octitleX | ttxDPX | ttciDPX | ttpDPX |

Blood Pressure

| | | | | |
|---|---|---|---|---|
| R-I | DBP (Day 120) | −.9% | (−1.4 to −.4%) | p = 0.0004 |
| R-I | DBP (Day 360) | −.8% | (−1.34 to −.259%) | p = 0.0038 |
| R-I | DBP (Day 720) | −.6% | (−1.19 to −5.8e−03%) | p = 0.0478 |
| R-I | DBP (Day 1080) | −.5% | (−1.16 to +.16%) | p = 0.1378 |
| R-I | DBP (Day 1440) | −.4% | (−1.15 to +.352%) | p = 0.2969 |

Blood Pressure

| | | | | |
|---|---|---|---|---|
| R-I | SBP (Day 120) | −1% | (−1.47 to −.53%) | p = 0.0000 |
| R-I | SBP (Day 360) | −1.1% | (−1.61 to −.586%) | p = 0.0000 |
| R-I | SBP (Day 720) | −1% | (−1.57 to −.429%) | p = 0.0006 |
| R-I | SBP (Day 1080) | −1.1% | (−1.95 to −.25%) | p = 0.0112 |
| R-I | SBP (Day 1440) | −.9% | (−1.85 to +.047%) | p = 0.0625 |

Dermatological Candidates. The dermatological conditions for the patient population were determined as enumerated in Table 37.

TABLE 37
Tabulated Binary/Discretized Events for Dermatological Candidates
Present Groupwise Comparisons for Dermatological Candidates
Tabulate Binary/Discretized Events for Dermatological Candidates
Dermatological Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Dermatological

| | | | | |
|---|---|---|---|---|
| R-I | Dermatitis, allergic (AE table) | 0.250 (0.071 to 0.885) 4 | 0.2%/−0.2%/454 2 | p = 0.0200 5 |
| ISS | Hot Flush (AE table) | 0.556 (0.125 to 2.474) 3 | 0.6%/−0.3%/292 2 | P = 0.4344 2 |
| R-I | Severe dermatitis, allergic | 0.250 (0.028 to 2.236) 4 | 0.1%/−0.1%/1363 1 | p = 0.1797 3 |
| | (AE table) | | | |

Gastrointestinal Candidates. The gastrointestinal events for the patient population were determined as enumerated in Table 38.

TABLE 38
Tabulated Binary/Discretized Events for Gastrointestinal Candidates
Present Groupwise Comparisons for Gastrointestinal Candidates
Tabulate Binary/Discretized Events for Gastrointestinal Candidates
Gastrointestinal Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Intestinal Tract

| | | | | |
|---|---|---|---|---|
| R-I | Chrohn's Disease (AE table) | 0.333 (0.035 to 3.204) 4 | 0.0%/−0.0%/2045 1 | p = 0.3173 2 |
| ISS | Constipation, Related (AE table) | 0.148 (0.017 to 1.265) 5 | 0.5%/−0.8%/122 3 | p = 0.0430 5 |
| R-I | Decreased Appetite (AE table) | 2.584 (1.329 to 5.024) −4 | 0.5%/0.5%/−215 −3 | p = 0.0037 −5 |
| ISS | Diarrhea, Any Severity (AE table) | 0.847 (0.603 to 1.191) 1 | 9.8%/−1.6%/61 4 | p = 0.3398 2 |
| ISS | Diarrhea, Mild Severity (AE table) | 0.741 (0.498 to 1.103) 2 | 7.4%/−2.2%/45 5 | p = 0.1388 3 |
| ISS | Diarrhea, Related (AE table) | 0.834 (0.575 to 1.210) 1 | 8.4%/−1.5%/65 4 | p = 0.3388 2 |
| ISS | Flatulence, Any Severity (AE table) | 0.463 (0.152 to 1.408) 3 | 1.1%/−0.8%/121 3 | p = 0.1645 3 |
| ISS | Flatulence, Mild Severity (AE table) | 0.424 (0.125 to 1.440) 3 | 0.9%/−0.8%/129 3 | p = 0.1559 3 |
| ISS | Flatulence, Related (AE table) | 0.463 (0.152 to 1.408) 3 | 1.1%/−0.8%/121 3 | p = 0.1645 3 |
| ISS | Frequent Bowel Movements, Any | [0 drug vs 3 PBO] | 0.2%/−0.6%/173 3 | p = 0.0440 5 |
| | Severity (AE table) | | | |
| ISS | Frequent Bowel Movements, Mild | [0 drug vs 2 PBO] | 0.2%/−0.4%/259 2 | p = 0.1002 3 |
| | Severity (AE table) | | | |
| ISS | Frequent Bowel Movements, Related | [0 drug vs 3 PBO] | 0.2%/−0.6%/173 3 | p = 0.0440 5 |
| | (AE table) | | | |
| R-I | Gastroesophageal reflux (AE table) | 0.333 (0.090 to 1.231) 4 | 0.1%/−0.1%/682 1 | p = 0.0831 4 |
| R-I | Large intestine polyps (AE table) | 0.385 (0.137 to 1.078) 4 | 0.2%/−0.2%/511 1 | p = 0.0591 4 |
| R-I | Ulcerative Colitis (AE table) | [0 drug vs 1 PBO] | 0.0%/−0.0%/4090 1 | p = 0.3173 2 |

Hepatic

| | | | | |
|---|---|---|---|---|
| R-I | Cholestasis (AE table) | 1.000 (0.063 to 15.986) −1 | 0.0%/0.0%/−1.67e+07 −1 | p = 0.9999 −1 |
| R-I | Hepatitis C (AE table) | [1 drug vs 0 PBO] | 0.0%/0.0%/−4089 −1 | p = 0.3172 −2 |
| R-I | Jaundice cholestatic (AE table) | [4 drug vs 0 PBO] | 0.0%/0.1%/−1022 −1 | p = 0.0454 −5 |
| R-I | NASH (AE table) | 0.167 (0.020 to 1.384) 5 | 0.1%/−0.1%/818 1 | p = 0.0587 4 |
| R-I | NASH for b/l NFS >0.675 (AE table) | 0.252 (0.028 to 2.251) 4 | 0.3%/−0.4%/273 2 | p = 0.1822 3 |
| R-I | NASH+Hepatic Steatosis for NFS >0.675 | 0.740 (0.342 to 1.601) 2 | 1,6%/−0.5%/209 3 | p = 0.4421 2 |
| | (AE table) | | | |

General

| | | | | |
|---|---|---|---|---|
| ISS | Weight loss, Related (AE table) | 0.247 (0.026 to 2.369) 4 | 0.3%/−0.4%/230 3 | p = 0.1890 3 |

Tabulate Adjusted Percent Change for Gastrointestinal Candidates
Gastrointestinal Candidates by Placebo-Adjusted Percent Change

| | | | | |
|---|---|---|---|---|
| | Candidate | Adjusted % | | |
| Source | Outcome | Change | 95% CI | P-Value |
| studyabbrS | octitleX | ttxDPX | ttciDPX | ttpDPX |

Alkaline Phosphatase (CHD Risk Marker)

| | | | | |
|---|---|---|---|---|
| R-I | Alkaline phosphatase (Day 120) | −10.4% | (−11.1 to −9.6%) | p = 0.0000 |
| R-I | Alkaline phosphatase (Day 360) | −12.5% | (−13.4 to −11.6%) | p = 0.0000 |
| R-I | Alkaline phosphatase (Day 720) | −12.6% | (−13.8 to −11.5%) | p = 0.0000 |
| R-I | Alkaline phosphatase (Day 1080) | −14.1% | (−16.2 to −12.1%) | p = 0.0000 |
| R-I | Alkaline phosphatase (Day 1440) | −13.1% | (−14.6 to −11.7%) | p = 0.0000 |

Genitourinary Candidates. Table 39 enumerates the genitourinary events in the patient population.

TABLE 39
Tabulated Binary/Discretized Events for Genitourinary Candidates
Present Groupwise Comparisons for Genitourinary Candidates
Tabulate Binary/Discretized Events for Genitourinary Candidates
Genitourinary Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Genitourinary

| | | |
|---|---|---|
| R-I | Male Infertility (AE table) | [No cases in study] |

Gynecological Candidates. Table 40 enumerates the gynecological events in the patient population.

TABLE 40
Tabulated Binary/Discretized Events for Gynecological Candidates
Present Groupwise Comparisons for Gynecological Candidates
Tabulate Binary/Discretized Events for Gynecological Candidates
Gynecological Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Gynecological

| | | | | |
|---|---|---|---|---|
| ISS | Menorrhagia (AE table) | 0.185 (0.021 to 1.653) 5 | 0.4%/−0.6%/159 3 | p = 0.0899 4 |
| R-I | PMS/Dysmenorrhea (AE table) | [No cases in study] | | |
| R-I | Polycystic Ovarian Syndrome (AE table) | [No cases in study] | | |

Hematological Candidates. Table 41 enumerates the hematological risks in the patient population.

TABLE 41
Tabulated Binary/Discretized Events for Hematological Candidates
Present Groupwise Comparisons for Hematologic Candidates
Tabulate Binary/Discretized Events for Hematologic Candidates
Hematologic Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Hematologic

| | | | | |
|---|---|---|---|---|
| R-I | Pancytopenia (AE table) | 0.556 (0.186 to 1.657) 3 | 0.2%/−0.1%/1023 1 | p = 0.2848 2 |
| R-I | Sickle Cell Disease (AE table) | [No cases in study] | | |

Infectious Disease Candidates. Table 42 enumerates the infectious diseases in the patient population. Several of the infectious disease events as shown below have a risk ratio (RR) less than 0.66 and some have an RR of no more than 0.25. Infectious disease events are particular significance include furuncle, gingivitis, mucosal inflammation, severe systemic inflammatory response, systemic inflammatory response syndrome (SIRS), tooth infection, and vulvovaginal mycotic infection.

Further, while not all of the infected tissues are directly associated with SARS-CoV-2 and/or COVID-19, diminished infection on one tissue can infer diminished potential in SARS-CoV-2 and/or COVID-19 targeted tissues. For example, an infection in the furuncle, while not life threatening, if the infection is treated and/or diminished, can impart beneficial effects in tissues specifically targeted by SARS-CoV-2 and/or COVID-19.

The data further show an impressive RR for severe SIRS of approximately 0.17 and an associated p value of 0.06. SIRS is also a precursor to both sepsis and acute respiratory distress syndrome (ARDS). SIRS, sepsis, and ARDS can each lead to death from COVID-19. Therefore, the reduction in SIRS as evidenced by the data enumerated in Table 42 suggests that icosapent ethyl can impart beneficial effects in reducing SIRS, sepsis, and ARDS and reduce death by infection of COVID-19.

TABLE 42
Tabulated Binary/Discretized Events for Infectious Disease Candidates
Present Groupwise Comparisons for Infectious Disease Candidates
Tabulate Binary/Discretized Events for Infectious Disease Candidates
Infectious Disease Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Infectious Disease

| | | | | |
|---|---|---|---|---|
| R-I | Cough (AE table) | 1.000 (0.841 to 1.189) −1 | 5.9%/0.0%/−69394 −1 | p = 0.9978 −1 |
| R-I | Ear infection (AE table) | 0.821 (0.515 to 1.307) 1 | 0.9%/−0.2%/585 1 | p = 0.4047 2 |
| R-I | Furuncle (AE table) | 0.500 (0.171 to 1.462) 3 | 0.2%/−0.1%/818 1 | p = 0.1965 3 |
| R-I | Gastroenteritis, viral (AE table) | 0.789 (0.525 to 1.185) 2 | 1.1%/−0.3%/372 2 | p = 0.2518 2 |
| R-I | Gingivitis (AE table) | 0.500 (0.171 to 1.462) 3 | 0.2%/−0.1%/818 1 | p = 0.1965 3 |
| R-I | Helicobacter infection (AE table) | 0.556 (0.186 to 1.657) 3 | 0.2%/−0.1%/1023 1 | p = 0.2848 2 |
| R-I | Herpes simplex (AE table) | 0.625 (0.205 to 1.909) 2 | 0.2%/−0.1%/1364 1 | p = 0.4053 2 |
| R-I | Herpes Zoster (AE table) | 0.960 (0.695 to 1.325) 1 | 1.8%/−0.1%/1371 1 | p = 0.8027 1 |
| R-I | Infected bite (AE table) | 0.714 (0.227 to 2.249) 2 | 0.1%/−0.0%/2046 1 | p = 0.5637 1 |
| R-I | Influenza (AE table) | 0.971 (0.824 to 1.144) 1 | 6.5%/−0.2%/515 1 | p = 0.7225 1 |
| R-I | Influenza like illness (AE table) | 0.852 (0.489 to 1.483) 1 | 0.6%/−0.1%/1024 1 | p = 0.5710 1 |
| R-I | Mucosal inflammation (AE table) | 0.600 (0.144 to 2.510) 2 | 0.1%/−0.0%/2046 1 | p = 0.4795 2 |
| R-I | Nasopharyngitis, Mild Severity (AE table) | 0.975 (0.840 to 1.133) 1 | 7.7%/−0.2%/516 1 | p = 0.7429 1 |
| R-I | Pneumonia (AE table) | 0.950 (0.807 to 1.118) 1 | 6.6%/−0.3%/293 2 | p = 0.5350 1 |
| R-I | Respiratory tract infection (AE table) | 0.939 (0.629 to 1.401) 1 | 1,2%/−0.1%/1368 1 | p = 0.7578 1 |
| R-I | Rhinitis (AE table) | 0.727 (0.383 to 1.383) 2 | 0.5%/−0.1%/682 1 | p = 0.3296 2 |
| R-I | Sepsis (AE table) | 0.786 (0.499 to 1.237) 2 | 0.9%/−0.2%/455 2 | p = 0.2970 2 |
| R-I | Severe gastroenteritis (AE table) | 0.667 (0.273 to 1.630) 2 | 0.2%/−0.1%/1023 1 | p = 0.3708 2 |
| R-I | Severe sepsis (AE table) | 0.759 (0.437 to 1.318) 2 | 0.6%/−0.2%/585 1 | p = 0.3259 2 |
| R-I | Severe systemic inflammatory | 0.167 (0.020 to 1.384) 5 | 0.1%/−0.1%/818 1 | p = 0.0587 4 |
| | response syndrome (AE table) | | | |
| R-I | Severe urinary tract infection (AE table) | 0.650 (0.324 to 1.305) 2 | 0.4%/−0.2%/585 1 | p = 0.2223 2 |
| R-I | Systemic inflammatory response | 0.250 (0.053 to 1.177) 4 | 0.1%/−0.1%/682 1 | p = 0.0577 4 |
| | syndrome (AE table) | | | |
| R-I | Tooth infection (AE table) | 0.690 (0.391 to 1.217) 2 | 0.6%/−0.2%/455 2 | p = 0.1975 3 |
| R-I | Upper respiratory tract infection (AE table) | 0.975 (0.840 to 1.133) 1 | 7.7%/−0.2%/516 1 | p = 0.7429 1 |
| R-I | Viral infection (AE table) | 0.792 (0.434 to 1.443) 2 | 0.5%/−0.1%/819 1 | p = 0.4450 2 |
| R-I | Vulvovaginal mycotic infection (AE table) | 0.455 (0.158 to 1.307) 3 | 0.2%/−0.1%/682 1 | p = 0.1334 3 |

Musculoskeletal Candidates. Table 43 enumerates the musculoskeletal risks in the patient population.

TABLE 43
Tabulated Binary/Discretized Events for Musculoskeletal Candidates
Present Groupwise Comparisons for Musculoskeletal Candidates
Tabulate Binary/Discretized Events for Musculoskeletal Candidates
Musculoskeletal Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Musculoskeletal

| | | | | |
|---|---|---|---|---|
| R-I | Blood CPK increased (AE table) | 0.684 (0.485 to 0.964) 2 | 1.6%/−0.6%/164 3 | p = 0.0289 5 |
| R-I | Bone Fractures (AE table) | 2.000 (0.181 to 22.053) −3 | 0.0%/0.0%/−4088 −1 | p = 0.5635 −1 |
| R-I | Dermatitis psoriasiform (AE table) | [1 drug vs 0 PBO] | 0.0%/0.0%/−4089 −1 | p = 0.3172 −2 |
| R-I | Guttate psoriasis (AE table) | [1 drug vs 0 PBO) | 0.0%/0.0%/−4089 −1 | p = 0.3172 −2 |
| R-I | Limb Fractures and Dislocations (AE table) | 0.883 (0.541 to 1.439) 1 | 0.8%/−0.1%/1024 1 | p = 0.6164 1 |
| R-I | Lupus (AE table) | [0 drugs vs 1 PBO] | 0.0%/−0.0%/4090 1 | p = 0.3173 2 |
| R-I | Muscle spasms (AE table) | 0.579 (0.421 to 0.795) 3 | 2.0%/−1.1%/95 4 | p = 0.0006 5 |
| ISS | Muscle spasms, Related (AE table) | 0.741 (0.046 to 11.826) 2 | 0.2%/−0.0%/2007 1 | p = 0.8317 1 |
| R-I | Psoriasis (AE table) | 1.750 (0.949 to 3.230) −3 | 0.5%/0.3%/−341 −2 | p = 0.0696 −4 |
| R-I | Psoriatic Conditions (AE table) | 1.875 (1.024 to 3.435) −3 | 0.6%/0.3%/−292 −2 | p = 0.0384 −5 |
| R-I | Rheumatoid Arthritis (AE table) | [0 drug vs 3 PBO] | 0.0%/−0.1%/1363 1 | p = 0.0832 4 |

Neuropsychiatric Candidates. Table 44 enumerates the neuropsychiatric events in the patient population.

TABLE 44
Tabulated Binary/Discretized Events for Neuropsychiatric Candidates
Present Groupwise Comparisons for Neuropsychiatric Candidates
Tabulate Binary/Discretized Events for Neuropsychiatric Candidates
Neuropsychiatric Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Psychiatric

| | | | | |
|---|---|---|---|---|
| R-I | Bipolar Disease (AE table) | 0.333 (0.035 to 3.204) 4 | 0.0%/−0.0%/2045 1 | p = 0.3173 2 |
| R-I | Depression: Depressed mood | 1.106 (0.861 to 1.422) −1 | 2.9%/0.3%/−340 −2 | p = 0.4288 −2 |
| | disorders and disturbances (AE | | | |
| | table) | | | |
| R-I | Depression: Depressive | 1.123 (0.867 to 1.454) −1 | 2.8%/0.3%/−314 −2 | p = 0.3785 −2 |
| | disorders (AE table) | | | |
| R-I | Depression: Depressive | [2 drugs vs 0 PBO] | 0.0%/0.0%/−2044 −1 | p = 0.1572 −3 |
| | disorders - Agitated depression | | | |
| | (AE table) | | | |
| R-I | Depression: Depressive | 1.097 (0.843 to 1.428) −1 | 2.6%%/0.2%/−408 −2 | p = 0.4893 −2 |
| | disorders - Depression (AE table) | | | |
| R-I | Depression: Depressive | 3.001 (0.312 to 28.836) −4 | 0.0%/0.0%/−2044 −1 | p = 0.3171 −2 |
| | disorders - Major depression (AE | | | |
| | table) | | | |
| R-I | Depression: Depressive | 2.000 (0.181 to 22.053) −3 | 0.0%/0.0%/−4088 −1 | p = 0.5635 −1 |
| | disorders - Persistent depressive | | | |
| | disorder (AE table) | | | |
| R-I | Depression: Mood alterations - | 1.000 (0.290 to 3.452) −1 | 0.1%/0.0%/−3344802 −1 | p = 0.9997 −1 |
| | Depressed mood (AE table) | | | |
| R-I | Depression: Mood alterations - | 0.500 (0.045 to 5.513) 3 | 0.0%/−0.0%/4091 1 | p = 0.5638 1 |
| | Depressive symptom (AE table) | | | |
| R-I | Depression: Mood alterations | 0.857 (0.288 to 2.549) 1 | 0.2%/−0.0%/4096 1 | p = 0.7817 1 |
| | with depressive symptoms (AE | | | |
| | table) | | | |
| R-I | PTSD (AE table) | 0.667 (0.111 to 3.989) 2 | 0.1%/−0.0%/4092 1 | p = 0.6548 1 |
| R-I | Psychosis: Psychotic disorder | 0.500 (0.045 to 5.513) 3 | 0.0%/−0.0%/4091 1 | p = 0.5638 1 |
| | NEC (AE table) | | | |
| R-I | Schizophrenia NEC (AE table) | 0.500 (0.045 to 5.513) 3 | 0.0%/−0.0%/4091 1 | p = 0.5638 1 |

Neurological

| | | | | |
|---|---|---|---|---|
| R-I | ADHD (AE table) | 1.000 (0.141 to 7.097) −1 | 0.0%/0.0%/−8362005 −1 | p = 0.9998 −1 |
| R-I | Alzheimer's Disease (AE table) | 1.000 (0.290 to 3.452) −1 | 0.1%/0.0%/−3344802 −1 | p = 0.9997 −1 |
| ISS | Chorea, Related (AE table) | 0.445(0.107 to 1.853) 3 | 0.7%/−0.5%/187 3 | p = 0.2528 2 |
| R-I | Cognitive Decline: Attention | 1.000 (0.141 to 7.097) −1 | 0.0%/0.0%/−8362005 −1 | p = 0.9998 −1 |
| | disorders and disturbances (AE | | | |
| | table) | | | |
| R-I | Cognitive Decline: Cognitive and | 1.000 (0.141 to 7.097) −1 | 0.0%/0.0%/−8362005 −1 | p = 0.9998 −1 |
| | attention disorders and | | | |
| | disturbances (AE table) | | | |
| R-I | Cognitive Decline: Cognitive | [No cases in study] | | |
| | disturbances (AE table) | | | |
| R-I | Epilepsy (AE table) | 1.000 (0.063 to 15.986) −1 | 0.0%/0.0%/−1.67e+07 −1 | p = 0.9999 −1 |
| ISS | Huntington's Chorea, Any | 0.247 (0.067 to 0.908) 4 | 1.0%/−1.3%/77 4 | p = 0.0224 5 |
| | Severity (AE table) | | | |
| ISS | Huntington's Chorea, Mild | 0.318 (0.083 to 1.223) 4 | 0.8%/−0.9%/109 3 | p = 0.0782 4 |
| | Severity (AE table) | | | |
| R-I | Migraine (AE table) | 2.751 (0.877 to 8.631) −4 | 0.2%/0.2%/−584 −1 | p = 0.0704 −4 |
| R-I | Multiple Sclerosis (AE table) | [0 drugs vs 1 PBO] | 0.0%/−0.0%/4090 1 | p = 0.3173 2 |

Nephrological Candidates. Table 45 enumerates the nephrological events in the patient population.

TABLE 45
Tabulated Binary/Discretized Events for Nephrological Candidates
Present Groupwise Comparisons for Nephrological Candidates
Tabulate Binary/Discretized Events for Nephrological Candidates
Nephrological Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Nephrological

| | | | | |
|---|---|---|---|---|
| R-I | CKD (AE table) | 1.123 (0.766 to 1.646) −1 | 1.3%/0.1%/−680 −1 | p = 0.5529 −1 |
| R-I | Diabetic Nephropathy (AE table) | 0.909 (0.387 to 2.139) 1 | 0.3%/−0.0%/4100 1 | p = 0.8275 1 |
| R-I | ESRD (AE table) | 0.667 (0.111 to 3.989) 2 | 0.1%/−0.0%/4092 1 | p = 0.6548 1 |
| R-I | Glomerulonephritis and Nephrotic | [0 drug vs 4 PBO] | 0.0%/−0.1%/1023 1 | p = 0.0455 5 |
| | Syndrome (AE table) | | | |
| R-I | IgA Nephropathy (AE table) | [No cases in study] | | |
| R-I | Nephrolithiasis (AE table) | 1.050 (0.777 to 1.419) −1 | 2.0%/0.1%/−1017 −1 | p = 0.7526 −1 |
| R-I | Nephropathies (AE table) | 0.679 (0.380 to 1.213) 2 | 0.6%/−0.2%/455 2 | p = 0.1883 3 |
| R-I | Nephrotic Syndrome (AE table) | [0 drug vs 1 PBO] | 0.0%/−0.0%/4090 1 | p = 0.3173 2 |
| R-I | Rental Transplant (AE table) | [No cases in study] | | |

Respiratory Candidates. Table 46 enumerates the respiratory conditions in the patient population. As shown in Table 46, a number of the AEs have an RR of less than 0.66 and some, have a p-value of less than 0.05. Notable respiratory conditions with low RR and p-values include atelectasis, bronchiectasis, cough, emphysema, nasopharyngitis, orthopnea, pulmonary edema, and wheezing. Of particular interest in relation to conditions that lead to death from COVID-19 are pulmonary edema, wheezing, cough, orthopnea, nasopharyngitis, and atelectasis.

TABLE 46
Tabulated Binary/Discretized Events for Respiratory Candidates
Present Groupwise Comparisons for Respiratory Candidates
Tabulate Binary/Discretized Events for Respiratory Candidates
Respiratory Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Respiratory

| | | | | |
|---|---|---|---|---|
| R-I | Allergic sinusitis (AE table) | 0.500 (0.092 to 2.729) 3 | 0.1%/−0.0%/2046 1 | p = 0.4142 2 |
| R-I | Asthma (AE table) | 1.076 (0.742 to 1.560) −1 | 1.3%/0.1%/−1019 −1 | p = 0.7000 −1 |
| R-I | Atelectasis (AE table) | 0.500 (0.243 to 1.030) 3 | 0.4%/−0.3%/372 2 | p = 0.0551 4 |
| R-I | Bronchiectasis (AE table) | 0.500 (0.125 to 1.998) 3 | 0.1%/−0.1%/1364 1 | p = 0.3172 2 |
| R-I | Bronchitis chronic (AE table) | 0.667 (0.273 to 1.630) 2 | 0.2%/−0.1%/1023 1 | p = 0.3708 2 |
| R-I | COPD (AE table) | 1.084 (0.837 to 1.405) −1 | 2.7%/0.2%/−453 −2 | p = 0.5399 −1 |
| R-I | Cough (AE table) | 1.928 (0.692 to 5.373) −3 | 1,5%/0.9%/−112 −3 | p = 0.2008 −2 |
| R-I | Cough (AE table) | 0.526 (0.245 to 1.131) 3 | 0.4%/−0.2%/455 2 | p = 0.0942 4 |
| R-I | Cough (AE table) | 1.000 (0.841 to 1.189) −1 | 5.9%/0.0%/−69394 −1 | p = 0.9978 −1 |
| R-I | Cystic Fibrosis (AE table) | [No cases in study] | | |
| R-I | Emphysema (AE table) | 0.600 (0.294 to 1.266) 2 | 0.4%/−0.2%/511 1 | p = 0.1567 3 |
| R-I | Influenza (AE table) | 0.971 (0.824 to 1.144) 1 | 6.5%/−0.2%/515 1 | p = 0.7225 1 |
| ISS | Nasopharyngitis, Any Severity (AE | 0.588 (0.344 to 1.004) 3 | 4.3%/−2.3%/43 5 | p = 0.0492 5 |
| | table) | | | |
| ISS | Nasopharyngitis, Mild Severity (AE | 0.494 (0.265 to 0.921) 3 | 3.3%/−2.3%/43 5 | p = 0.0234 5 |
| | table) | | | |
| R-I | Nasopharyngitis, Mild Severity (AE | 0.975 (0.840 to 1.133) 1 | 7.7%/−0.2%/516 1 | p = 0.7429 1 |
| | table) | | | |
| R-I | Orthopnea (AE table) | 0.500 (0.151 to 1.660) 3 | 0.1%/−0.1%/1023 1 | p = 0.2480 2 |
| R-I | Pneumonia (AE table) | 0.950 (0.807 to 1.118) 1 | 6.6%/−0.3%/293 2 | p = 0.5350 1 |
| R-I | Pulmonary Edema (AE table) | 0.556 (0.257 to 1.202) 3 | 0.3%/−0.2%/511 1 | p = 0.1301 3 |
| R-I | Respiratory Failure (AE table) | 0.909 (0.497 to 1.664) 1 | 0.5%/−0.0%/2050 1 | p = 0.7576 1 |
| R-I | Respiratory tract infection (AE | 0.939 (0.629 to 1.401) 1 | 1.2%/−0.1%/1368 1 | p = 0.7578 1 |
| | table) | | | |
| R-I | Severe chronic obstructive | 0.857 (0.458 to 1.607) 1 | 0.5%/−0.1%/1365 1 | p = 0.6307 1 |
| | pulmonary disease (AE table) | | | |
| R-I | Upper respiratory tract infection | 0.975 (0.840 to 1.133) 1 | 7.7%/−0.2%/516 1 | p = 0.7429 1 |
| | (AE table) | | | |
| R-I | Wheezing (AE table) | 0.639 (0.379 to 1.076) 2 | 0.7%/−0.3%/315 2 | p = 0.0896 4 |

Endocrinologic Candidates. Table 47 enumerates the endocrinologic conditions in the patient population.

TABLE 47
Tabulated Binary/Discretized Events for Endocrinologic Candidates
Present Groupwise Comparisons for Endocrinologic Candidates
Tabulate Binary/Discretized Events for Endocrinologic Candidates
Endocrinologic Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Carbohydrate Metabolism
Low Carbohydrate Metabolism

| | | | | |
|---|---|---|---|---|
| R-I | Blood glucose increased (AE table) | 0.400 (0.126 to 1.275) 3 | 0.2%/−0.1%/682 1 | p = 0.1086 3 |

High Carbohydrate Metabolism

| | | | | |
|---|---|---|---|---|
| R-I | Hyperglycemia (AE table) | 0.764 (0.562 to 1.037) 2 | 2.0%/−0.5%/186 3 | p = 0.0830 4 |

Calcium
Low Calcium

| | | | | |
|---|---|---|---|---|
| R-I | Calcium low (Day 120) | 1.399 (0.444 to 4.404) −2 | 0.2%/0.1%/−1944 −1 | p = 0.5643 −1 |
| R-I | Calcium low (Day 360) | 9.856 (1.262 to 76.953) −5 | 0.2%/0.2%/−410 −2 | p = 0.0071 −5 |
| R-I | Calcium low (Day 720) | 2.911 (0.789 to 10.744) −4 | 0.2%/0.2%/−567 −1 | p = 0.0926 −4 |

High Calcium

| | | | | |
|---|---|---|---|---|
| R-I | Calcium high (Day 120) | 0.799 (0.517 to 1.236) 2 | 1.0%/−0.2%/430 2 | p = 0.3131 2 |
| R-I | Calcium high (Day 360) | 0.915 (0.593 to 1.412) 1 | 1.1%/−0.1%/1019 1 | p = 0.6885 1 |
| R-I | Calcium high (Day 720) | 0.767 (0.491 to 1.200) 2 | 1.2%/−0.3%/325 2 | p = 0.2443 2 |

Potassium
Low Potassium

| | | | | |
|---|---|---|---|---|
| R-I | Hypokalemia (AE table) | 1.064 (0.784 to 1.445) −1 | 2.0%/0.1%/−815 −1 | p = 0.6895 −1 |

High Potassium

| | | | | |
|---|---|---|---|---|
| R-I | Hyperkalemia (AE table) | 0.582 (0.377 to 0.898) 3 | 1.1%/−0.6%/178 3 | p = 0.0132 5 |

Magnesium

| | | | | |
|---|---|---|---|---|
| R-I | Hypomagnesemia (AE table) | 0.675 (0.422 to 1.078) 2 | 0.9%/−0.3%/292 2 | p = 0.0977 4 |

Liver-Related CHD Risk Factor Candidates. Tables 48A-48B enumerate the liver-related risk factors in the patient population.

TABLE 48A
Tabulated Binary/Discretized Events for Liver-Related CHD Candidates
Present Groupwise Comparisons for Liver-Related CHD Risk Factor Candidates
Tabulate Binary/Discretized Events for Liver-Related CHD Risk Factor Candidates
Liver-Related CHD Risk Factor Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

Alkaline Phosphatase (CHD Risk Marker)
Low Alkaline Phosphatase (CHD Risk Marker)

| | | | | |
|---|---|---|---|---|
| R-I | ALP shift: normal to low (Day 120) | 3.341 (1.343 to 8.311) −4 | 0.3%/0.4%/−267 −2 | p = 0.0058 −5 |
| R-I | ALP shift: normal to low (Day 360) | 2.394 (0.994 to 5.767) −3 | 0.3%/0.3%/−359 −2 | p = 0.0444 −5 |
| R-I | ALP shift: normal to low (Day 720) | 2.783 (1.178 to 6.572) −4 | 0.4%/0.4%/−252 −2 | p = 0.0147 −5 |
| R-I | ALP shift: normal to low (Day 1080) | 1.938 (0.831 to 4.522) −3 | 0.4%/0.3%/−360 −2 | p = 0.1188 −3 |
| R-I | ALP shift: normal to low (Day 1440) | 1.199 (0.562 to 2.556) −1 | 0.6%/0.1%/−956 −1 | p = 0.6378 −1 |

High Alkaline Phosphatase (CHD Risk Marker)

| | | | | |
|---|---|---|---|---|
| R-I | ALP >2x ULN (AE table) | 0.600 (0.335 to 1.075) 2 | 0.6%/−0.3%/341 2 | p = 0.0825 4 |
| R-I | ALP > ULN (AE table) | 0.624 (0.545 to 0.714) 2 | 9.8%/−4.5%/22 5 | p = 0.0000 5 |
| R-I | ALP high (Day 120) | 0.623 (0.485 to 0.801) 2 | 3.2%/−1.5%/67 4 | p = 0.0002 5 |
| R-I | ALP high (Day 360) | 0.542 (0.420 to 0.700) 3 | 3.4%/−2.0%/50 5 | p = 0.0000 5 |
| R-I | ALP high (Day 720) | 0.568 (0.449 to 0.717) 3 | 4.4%/−2.4%/41 5 | p = 0.0000 5 |
| R-I | ALP high (Day 1080) | 0.486 (0.375 to 0.630) 3 | 4.4%/−3.0%/33 5 | p = 0.0000 5 |
| R-I | ALP high (Day 1440) | 0.483 (0.362 to 0.644) 3 | 4.2%/−2.9%/34 5 | p = 0.0000 5 |
| R-I | ALP high (Day 1800) | 0.527 (0.362 to 0.767) 3 | 4.3%/−2.7%/37 5 | p = 0.0006 5 |
| R-I | ALP shift: normal to high (Day 120) | 0.401 (0.267 to 0.603) 3 | 1.5%/−1.3%/78 4 | p = 0.0000 5 |
| R-I | ALP shift: normal to high (Day 360) | 0.335 (0.227 to 0.497) 4 | 1.8%/−1.8%/54 4 | p = 0.0000 5 |
| R-I | ALP shift: normal to high (Day 720) | 0.397 (0.291 to 0.541) 4 | 3.0%/−2.6%/39 5 | p = 0.0000 5 |
| R-I | ALP shift: normal to high (Day 1080) | 0.395 (0.280 to 0.528) 4 | 3.3%/−3.0%/34 5 | p = 0.0000 5 |
| R-I | ALP shift: normal to high (Day 1440) | 0.406 (0.287 to 0.575) 3 | 3.2%/−2.7%/37 5 | p = 0.0000 5 |
| R-I | ALP shift: normal to high (Day 1800) | 0.439 (0.280 to 0.688) 3 | 3.3%/−2.6%/39 5 | p = 0.0002 5 |

Albumin (Antioxidant/Inverse CHD Risk Marker)

| | | | | |
|---|---|---|---|---|
| R-I | Albumin high (Day 120) | 2.263 (1.388 to 3.689) −3 | 1.0%/0.8%/−133 −3 | p = 0.0008 −5 |
| R-I | Albumin high (Day 360) | 1.901 (1.217 to 2.969) −3 | 1.2%/0.7%/−138 −3 | p = 0.0041 −5 |
| R-I | Albumin high (Day 720) | 1.365 (0.975 to 1.911) −2 | 2.1%/0.6%/−156 −3 | p = 0.0684 −4 |
| R-I | Albumin high (Day 1080) | 1.449 (0.993 to 2.115) −2 | 1.9%/0.7%/−140 −3 | p = 0.0528 −4 |
| R-I | Albumin high (Day 1440) | 1.540 (1.001 to 2.369) −2 | 1.8%/0.8%/−132 −3 | p = 0.0477 −5 |
| R-I | Albumin shift: normal to high (Day 120) | 3.055 (1.709 to 5.461) −4 | 0.8%/0.8%/−123 −3 | p = 0.0001 −5 |
| R-I | Albumin shift: normal to high (Day 360) | 2.364 (1.388 to 4.027) −3 | 0.9%/.07%/−137 −3 | p = 0.0011 −5 |
| R-I | Albumin shift: normal to high (Day 720) | 1.475 (1.028 to 2.116) −2 | 1.9%/0.7%/−140 −3 | p = 0.0337 −5 |
| R-I | Albumin shift: normal to high (Day | 1.257 (0.848 to 1.864) −2 | 1.8%/0.4%/−248 −3 | p = 0.2542 −2 |
| | 1080) | | | |
| R-I | Albumin shift: normal to high (Day | 1.531 (0.974 to 2.408) −2 | 1.6%/0.7%/−145 −3 | p = 0.0629 −4 |
| | 1440) | | | |

TABLE 48B
Liver-Related CHD Risk Factor Candidates by Placebo-Adjusted Percent Change
Tabulate Adjusted Percent Change for Liver-Related CHD Risk Factor Candidates
Liver-Related CHD Risk Factor Candidates by Placebo-Adjusted Percent Change

| | | | | |
|---|---|---|---|---|
| | Candidate | Adjusted % | | |
| Source | Outcome | Change | 95% CI | P-Value |
| studyabbrS | octitleX | ttxDPX | ttciDPX | ttpDPX |

Alkaline Phosphatase (CHD Risk Marker)

| | | | | |
|---|---|---|---|---|
| R-I | Alkaline phosphatase (Day 120) | −10.4% | (−11.1 to −9.6%) | p = 0.0000 |
| R-I | Alkaline phosphatase (Day 360) | −12.5% | (−13.4 to −11.6%) | p = 0.0000 |
| R-I | Alkaline phosphatase (Day 720) | −12.6% | (−13.8 to −11.5%) | p = 0.0000 |
| R-I | Alkaline phosphatase (Day 1080) | −14.1% | (−16.2 to −12.1%) | p = 0.0000 |
| R-I | Alkaline phosphatase (Day 1440) | −13.1% | (−14.6 to −11.7%) | p = 0.0000 |

Platelet/RES Risk Factor Candidates. Table 49 enumerates the platelet and RES exam conditions in the patient population.

TABLE 49
Tabulated Binary/Discretized Events for Platelet/RES Candidates
Present Groupwise Comparisons for Platelet/RES Candidates
Tabulate Binary/Discretized Events for Platelet/RES Candidates
Platelet/RES Candidates by Risk Ratio

| | | | | |
|---|---|---|---|---|
| | | Risk | | |
| Data- | Candidate | Ratio | | |
| set | Outcome | (95% CI) | AR/ARR/NNT | P Value |

RES/Exam

| | | | | |
|---|---|---|---|---|
| R-I | Hepatomegaly (AE table) | 0.304 (0.131 to 0.709) 4 | 0.4%/−0.4%/256 2 | p = 0.0034 5 |
| R-I | Spleen disorders (AE table) | 0.353 (0.139 to 0.894) 4 | 0.3%/−0.3%/372 2 | p = 0.0217 5 |
| R-I | Splenomegaly (AE table) | 0.400 (0.126 to 1.275) 3 | 0.2%/−0.1%/682 1 | p = 0.1086 3 |

Platelet Levels
Low Platelet Levels

| | | | | |
|---|---|---|---|---|
| R-I | Low platelets (Day 360) | 0.366 (0.253 to 0.530) 4 | 2.0%/−1.8%/54 4 | p = 0.0000 5 |
| R-I | Low platelets (Day 720) | 0.799 (0.608 to 1.051) 2 | 3.1%/−0.7%/144 3 | p = 0.1085 3 |
| R-I | Low platelets (Day 1080) | 0.688 (0.512 to 0.925) 2 | 3.2%/−1.2%/85 4 | p = 0.0126 5 |
| R-I | Platelet count: PCS Low <100 × | 0.442 (0.258 to 0.758) 3 | 0.8%/−0.6%/166 3 | p = 0.0022 5 |
| | 10\hat{ }3/uL | | | |

High Platelet Levels

| | | | | |
|---|---|---|---|---|
| R-I | High platelets (Day 720) | 1.503 (1.007 to 2.242) −2 | 1.5%/0.6%/−162 −3 | p = 0.0444 −5 |

To further investigate the effects of icosapent ethyl on managing infection and/or tissue injury, a sub-analysis of the AEs was evaluated (Tables 50 and 51). The sub-analysis was aimed at investigating the effects of AEs related to imparting resilience to the integumentary system. The AEs related to infection (including the integument, broadly including the skin and mucosa), “downstream” sequelae of infection (e.g., hepatosplenomegaly, SIRS, and sepsis), related laboratory findings (e.g., evidences for the acute phase response, such as thrombocytosis, leukocytosis), and AEs affecting the respiratory system were included in this analysis. This analysis eliminates AEs that are unrelated to infectious diseases and allows an analysis to specifically target AEs related to infectious disease and related responses.

TABLE 50
Number (%) of Subjects with Treatment-Emergent Adverse Events of Infectious
Diseases Terms of Interest by System Organ Class and Preferred Term
ITT Population

| | AMR101 | | Placebo | | Fisher's |
|---|---|---|---|---|---|
| | (N = 4089) | | (N = 4090) | | Exact |
| | n (%) | | n (%) | | p-value |
| Subjects with at least one Infectious | 812 | (19.9) | 883 | (21.6) | 0.0562 |
| Disease TEAE of Interest | | | | | |
| Infections and infestations | 563 | (13.8) | 599 | (14.6) | 0.2676 |
| Pneumonia | 263 | (6.4) | 277 | (6.8) | 0.5627 |
| Influenza | 263 | (6.4) | 271 | (6.6) | 0.7541 |
| Sepsis | 33 | (0.8) | 42 | (1.0) | 0.3535 |
| Tooth infection | 20 | (0.5) | 29 | (0.7) | 0.2514 |
| Rhinitis | 16 | (0.4) | 22 | (0.5) | 0.4166 |
| Vulvovaginal mycotic infection | 5 | (0.1) | 11 | (0.3) | 0.2097 |
| Gingivitis | 5 | (0.1) | 10 | (0.2) | 0.3013 |
| Helicobacter infection | 5 | (0.1) | 9 | (0.2) | 0.4235 |
| Herpes simplex | 5 | (0.1) | 8 | (0.2) | 0.5807 |
| Respiratory, thoracic and mediastinal | 287 | (7.0) | 318 | (7.8) | 0.2050 |
| disorders | | | | | |
| Cough | 241 | (5.9) | 241 | (5.9) | 1.0000 |
| Wheezing | 23 | (0.6) | 36 | (0.9) | 0.1162 |
| Emphysema | 12 | (0.3) | 20 | (0.5) | 0.2144 |
| Atelectasis | 11 | (0.3) | 22 | (0.5) | 0.0795 |
| Bronchitis chronic | 8 | (0.2) | 12 | (0.3) | 0.5029 |
| Orthopnea | 4 | (0.1) | 8 | (0.2) | 0.3873 |
| Bronchiectasis | 3 | (0.1) | 6 | (0.1) | 0.5076 |
| Allergic sinusitis | 2 | (0.0) | 4 | (0.1) | 0.6874 |
| Blood and lymphatic system disorders | 28 | (0.7) | 48 | (1.2) | 0.0279 |
| Thrombocytopenia | 23 | (0.6) | 31 | (0.8) | 0.3393 |
| Splenomegaly | 4 | (0.1) | 10 | (0.2) | 0.1792 |
| Neutropenia | 3 | (0.1) | 8 | (0.2) | 0.2262 |
| General disorders and administration | 28 | (0.7) | 40 | (1.0) | 0.1800 |
| site conditions | | | | | |
| Influenza like illness | 23 | (0.6) | 27 | (0.7) | 0.6709 |
| Mucosal inflammation | 3 | (0.1) | 5 | (0.1) | 0.7264 |
| Systemic inflammatory response | 2 | (0.0) | 8 | (0.2) | 0.1092 |
| syndrome | | | | | |
| Hepatobiliary disorders | 6 | (0.1) | 22 | (0.5) | 0.0037 |
| Hepatomegaly | 5 | (0.1) | 19 | (0.5) | 0.0065 |
| Hepatosplenomegaly | 1 | (0.0) | 3 | (0.1) | 0.6249 |

Note:
A treatment-emergent adverse event (TEAE) is defined as an event that first occurs or worsens in severity on or after the date of dispensing study drug and within 30 days after the completion or withdrawal from study. For each subject, multiple TEAE TEAEs are listed in descending order of AMR101 frequency. Percentages are based on the number of subjects randomized to each treatment group in the Safety population (N). Events that were positively adjudicated as clinical endpoints are not included.
[1] All adverse events are coded using the Medical Dictionary for Regulatory Activities (MedDRA Version 20.1).

TABLE 51
Number (%) of Subjects with Treatment-Emergent Adverse Events Terms and
Labs of Interest by System Organ Class and Preferred Term
ITT Population

| | AMR101 | | Placebo | | Fisher's |
|---|---|---|---|---|---|
| | (N = 4089) | | (N = 4090) | | Exact |
| | n (%) | | n (%) | | p-value |
| Subjects with at least one Infectious | 3394 | (83.0) | 3549 | (86.8) | <.0001 |
| Disease Events of Interest | | | | | |
| Lab Reported Events | 3269 | (79.9) | 3427 | (83.8) | <.0001 |
| WBC Differential (Lymphocytes): | 3067 | (75.0) | 3317 | (81.1) | <.0001 |
| PCS Low <30% | | | | | |
| WBC Differential (Lymphocytes): | 301 | (7.4) | 195 | (4.8) | <.0001 |
| PCS High >45% | | | | | |
| Platelet Count: PCS Low <100 × | 19 | (0.5) | 43 | (1.1) | 0.0030 |
| 10\hat{ }3/uL | | | | | |
| Platelet Count: PCS High >500 × | 16 | (0.4) | 18 | (0.4) | 0.8639 |
| 10\hat{ }3/uL | | | | | |
| Infections and infestations | 563 | (13.8) | 599 | (14.6) | 0.2676 |
| Pneumonia | 263 | (6.4) | 277 | (6.8) | 0.5627 |
| Influenza | 263 | (6.4) | 271 | (6.6) | 0.7541 |
| Sepsis | 33 | (0.8) | 42 | (1.0) | 0.3535 |
| Tooth infection | 20 | (0.5) | 29 | (0.7) | 0.2514 |
| Rhinitis | 16 | (0.4) | 22 | (0.5) | 0.4166 |
| Vulvovaginal mycotic infection | 5 | (0.1) | 11 | (0.3) | 0.2097 |
| Gingivitis | 5 | (0.1) | 10 | (0.2) | 0.3013 |
| Helicobacter infection | 5 | (0.1) | 9 | (0.2) | 0.4235 |
| Herpes simplex | 5 | (0.1) | 8 | (0.2) | 0.5807 |
| Respiratory, thoracic and mediastinal | 287 | (7.0) | 318 | (7.8) | 0.2050 |
| disorders | | | | | |
| Cough | 241 | (5.9) | 241 | (5.9) | 1.0000 |
| Wheezing | 23 | (0.6) | 36 | (0.9) | 0.1162 |
| Emphysema | 12 | (0.3) | 20 | (0.5) | 0.2144 |
| Atelectasis | 11 | (0.3) | 22 | (0.5) | 0.0795 |
| Bronchitis chronic | 8 | (0.2) | 12 | (0.3) | 0.5029 |
| Orthopnea | 4 | (0.1) | 8 | (0.2) | 0.3873 |
| Bronchiectasis | 3 | (0.1) | 6 | (0.1) | 0.5076 |
| Allergic sinusitis | 2 | (0.0) | 4 | (0.1) | 0.6874 |
| Blood and lymphatic system disorders | 28 | (0.7) | 48 | (1.2) | 0.0279 |
| Thrombocytopenia | 23 | (0.6) | 31 | (0.8) | 0.3393 |
| Splenomegaly | 4 | (0.1) | 10 | (0.2) | 0.1792 |
| Neutropenia | 3 | (0.1) | 8 | (0.2) | 0.2262 |
| General disorders and administration | 28 | (0.7) | 40 | (1.0) | 0.1800 |
| site conditions | | | | | |
| Influenza like illness | 23 | (0.6) | 27 | (0.7) | 0.6709 |
| Mucosal inflammation | 3 | (0.1) | 5 | (0.1) | 0.7264 |
| Systemic inflammatory response | 2 | (0.0) | 8 | (0.2) | 0.1092 |
| syndrome | | | | | |
| Hepatobiliary disorders | 6 | (0.1) | 22 | (0.5) | 0.0037 |
| Hepatomegaly | 5 | (0.1) | 19 | (0.5) | 0.0065 |
| Hepatosplenomegaly | 1 | (0.0) | 3 | (0.1) | 0.6249 |

Note:
A treatment-emergent adverse event (TEAE) is defined as an event that first occurs or worsens in severity on or after the date of dispensing study drug and within 30 days after the completion or withdrawal from study. For each subject, multiple TEAE TEAEs are listed in descending order of AMR101 frequency. Percentages are based on the number of subjects randomized to each treatment group in the Safety population (N). Events that were positively adjudicated as clinical endpoints are not included.
[1] All adverse events are coded using the Medical Dictionary for Regulatory Activities (MedDRA Version 20.1).

The infectious disease and related responses selected for this sub-analysis were as follows: dermatitis, allergic; spleen disorders; splenomegaly; hepatomegaly; cough; low platelets; lymphocytes: pcs high >45%; lymphocytes: pcs low <30%; platelet count: pcs low <100×10\hat{ }3/ul; nasopharyngitis, mild severity; nasopharyngitis, any severity; systemic inflammatory response syndrome; wheezing; pneumonia; influenza; gastroenteritis, viral; sepsis; tooth infection; rhinitis; vulvovaginal mycotic infection; furuncle; gingivitis; helicobacter infection; herpes simplex; influenza like illness; mucosal inflammation; emphysema; atelectasis; chronic bronchitis; orthopnea; allergic sinusitis; thrombocytopenia; neutropenia; hepatomegaly; hepatosplenomegaly; severe sepsis; severe urinary tract infection; severe gastroenteritis; severe dermatitis, allergic; and severe systemic inflammatory response syndrome.

### Conclusion

Taken together, these data suggest that icosapent ethyl has beneficial effects in patients suffering from coughs and mucosal infections (e.g., nasopharyngitis), symptoms associated with COVID-19. Moreover, coughs are mediated by the leukotrienes, including LTB4 and the LTC4, LTD4, and LTE4 series (collectively, the slow reacting substance of anaphylaxis/sepsis). It is contemplated that EPA can moderate these leukotrienes, similar to how it moderates the eicosanoids, which can reduce the incidents of symptoms associated with COVID-19 (e.g., coughing).

EPA's ability to moderate inflammation in the mucosal tissue is also contemplated to impart a beneficial effect on patients suffering from SARS-CoV-2 infection and/or COVID-19. For example, in moderating the eicosanoid response (e.g., PGD3 vs. PGD2, PGE3 vs. PGE2, TXA3 vs. TXA2, PGI3 vs. PGI2), EPA can reduce the inflammatory response in the mucosal tissue and relieve some of the symptoms and/or discomfort associated with SARS-CoV-2 infection and/or COVID-19 disease.

In moderating both the leukotrienes and eicosanoids response in patients, administration of icosapent ethyl could make tissues more resilient to tissue injury and inflammatory changes and, therefore, better equipped to fight viral infections. So, while icosapent ethyl is not an anti-viral agent per se, administration of icosapent ethyl to patients can nevertheless impart beneficial effects that can make the patient more able to fight the viral infection.

It is further contemplated that differences in the biomarkers of patients administered icosapent ethyl versus placebo is explained by the promotion by EPA of the heme oxidase pathway. For example, patients administered icosapent ethyl exhibited reductions in blood pressure, SIRS (the precursor to sepsis), and increases in bilirubin levels—biomarkers frequently associated with the activation of the heme oxidase pathway. Significantly, by activating the heme oxidase pathway, EPA might impart beneficial effects that can fight infectious diseases such as COVID-19. Moreover, activation of the heme oxidase pathway may help reduce and/or prevent symptoms associated with COVID-19 by inhibiting sepsis, acute lung injury, hypertension, renal injury, and/or pain.

### Example 4: The Impact of Icosapent Ethyl on Treating and/or Preventing Patients Infected with SARS-CoV-2

The objective of the following study is to implement a clinical trial to evaluate the impact of icosapent ethyl on treating and/or preventing infection with SARS-CoV-2, development of COVID-19, and/or symptoms thereof. This study was designed with the understanding of the urgency and need for prevention and treatment therapies for patients infected with SARS-CoV-2, development of COVID-19, and/or symptoms thereof. Given the medical demand, the study includes the use of supplies, including both clinical and commercial, that are currently available.

In the recent REDUCE-IT cardiovascular outcomes trial described in Examples 1-2, icosapent ethyl at 4 grams daily demonstrated robust and consistent reductions in cardiovascular risk in statin-treated patients across multiple endpoints and subgroups. Icosapent ethyl also demonstrated an attractive safety profile. Example 3 provides evidence that suggests that icosapent ethyl has beneficial effects in patients suffering symptoms associated with COVID-19.

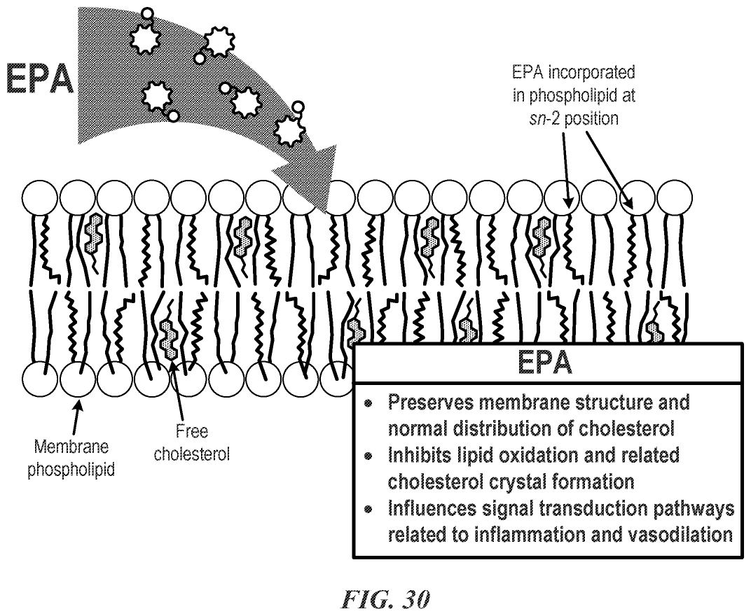

While the mechanistic effects of EPA are not fully understood, they cannot be explained solely by mechanisms such as triglyceride lowering. Clinical and preclinical data support that efficacy is due at least in part to the role of EPA as a bioactive lipid that preserves membrane structure and normal distribution of cholesterol, inhibits lipid oxidation and cholesterol crystal formation, influences signal transduction pathways associated with inflammation and vasodilation, and transcriptional regulation of multiple related genetic pathways (FIG. 30).

Relevant to the acute needs and rapid therapeutic timing in the care of SARS-CoV-2 infected patients and those having COVID-19, and/or signs or symptoms thereof, PK studies showed that following icosapent ethyl dosing, maximum concentration of total EPA was reached in approximately 5-6 hours in plasma and approximately 8-24 hours in red blood cells (a marker of peripheral tissue uptake). The mean terminal half-life of total EPA in plasma was long, ranging between 70 and 89 hours.

### Methods

### Study Design

This study will include the use of currently available icosapent ethyl 1 g or 0.5 g capsules to be administered at 4 g/day (taken as either two 1 g capsules twice daily with food or four 0.5 g capsules taken twice daily with food).

While the peak single dose plasma concentrations are rapidly reached after administration of icosapent ethyl, given the time needed for EPA to reach full plasma steady state (e.g., 28 days), in-hospital loading doses will be increased, e.g., 8 g/day or 10 g/day while hospitalized and/or 4 g/day at discharge. In patients who are nil per os (NPO), dosing by feeding tube can be used.

If possible, patients will be followed beyond their hospitalization stays and infection with SARS-CoV-2 and/or having COVID-19 disease, signs, or symptoms. For example, patients will be followed to observe potentially long-term cardiovascular effects, changes in inflammatory conditions, and/or other benefits imparted by the administration of icosapent ethyl.

### Data Collections

For each patient, prior medical history, as available, e.g., medical history, prior medications, including omega-3 use, prior respiratory disease, date of COVID-19 symptom onset, presenting symptoms, admission diagnoses, and allergies will be determined.

### Clinical Outcomes

The following clinical outcomes will be determined: length of hospital stay; concomitant medications; ICU admission and length of ICU stay (or other intensive observation settings if ICU care distributed to overflow wards)); use and length of oxygen support; use and length of ventilation support; development and duration of SIRS, sepsis, or ARDS and component criteria; all clinical laboratory tests, including blood chemistry, hematology, coagulation studies, blood gases, COVID-19 or other microbiology, urine tests, radiologic tests, ECGs, pulmonary function tests, and vital signs; other COVID-19 complications; and cardiovascular outcomes.

### Biomarkers

The following biomarkers will be determined at the time of admission, discharge, and during the follow-up phase: high-sensitivity C-reactive protein (hs-CRP), lipoprotein-associated phospholipase A2, oxidized LDL-C levels, the AA-to-EPA ratio, soluble intercellular adhesion molecule-1 (ICAM-1), vascular cell adhesion molecule 1 (VCAM-1), and interleukin 10 (IL-10). These biomarkers will provide information regarding the anti-inflammatory effects of the administration of icosapent ethyl. For example, IL-10 is an anti-inflammatory cytokine involved in macrophage recruitment that contributes to reducing inflammation in the body.

### Cytokines

The following cytokines will be determined at the time of admission, discharge, and during the follow-up phase: adiponectin and adipocytokine. The changes in cytokine levels will further provide information regarding the inflammatory response of the administration of icosapent ethyl. For example, it is contemplated that EPA will increase adiponectin, which is an adipocytokine with anti-inflammatory and anti-atherogenic properties.

### Inflammatory Mediators

The following inflammatory mediators will be determined at the time of admission, discharge, and during the follow-up phase: tumor necrosis factor alpha (TNF-α) and interleukin 1 beta (IL-1β). It is contemplated that EPA reduces the production of inflammatory mediators such as TNF-α and IL-1β compared to another long chain polyunsaturated fatty acid docosahexaenoic acid (DHA).

### Research and Post-Discharge Outcomes

Plasma, serum, and if possible, red cell pellet for future testing will determined at the time of admission and discharge as well as during the out-patient follow-up phase.

The following assays will be performed both in real-time and for future testing: biomarkers of inflammation; hs-CRP and cytokines; eicosanoid derivatives panel (e.g., prostaglandins, thromboxanes, prostacyclins, leukotrienes, resveratrol); acute phase reactants from real time assays during hospitalization or collected during outpatient follow-up phase; clinical chemistry (including pre-albumin), hematology, coagulation studies, and bleeding time during outpatient follow-up; troponin I (CTI) and D-dimer; EPA exposure; EPA in acellular and cellular blood fractions to correlate clinical response with EPA exposure from the different dose-loading groups.

The following post-discharge outcomes will be performed: serial lab studies listed above; vital signs; weight/BMI; concomitant medications, adverse events, and general health status (e.g., EQ-5D).

### Conclusion

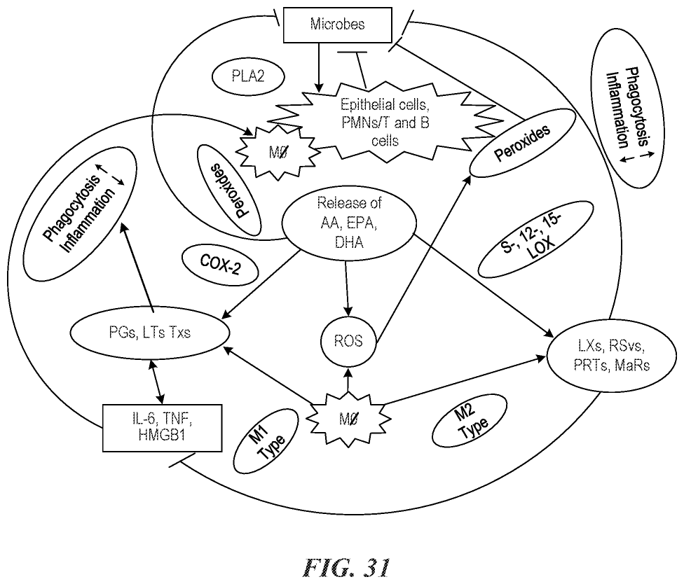

It is contemplated that this study will provide evidence that oral or IV administration of icosapent ethyl may aid prevention of, or recovery from, infections and, when present in adequate amounts in immunocytes and body fluids (especially in the alveolar fluid), may decrease morbidity and mortality of the current pandemic (FIG. 31).

### Example 5: The Effects of Icosapent Ethyl on Inflammatory Biomarkers in Ambulatory Patients with COVID-19

A 14-day long, prospective, multi-site, two-armed, randomized, open-label study on adult outpatients who had received a positive SARS-CoV-2 test result within the preceding 72 hours was initiated (VASCEPA-COVID-19, NCT04412018) to investigate the effects of icosapent ethyl (Vascepa™) on inflammatory biomarkers in individuals with COVID-19.

### Study Design and Conduct

### Overview

The coronavirus disease 2019 (COVID-19) pandemic remains a source of considerable morbidity and mortality throughout the world, with few safe and effective treatments currently available. Furthermore, the therapies that have demonstrated efficacy (e.g., corticosteroids) studied hospitalized and/or markedly ill patients. For the majority of patients in the community who develop symptomatic COVID-19, therapeutic options to reduce symptoms, inflammatory response, or disease progression are extremely limited.

This was a 14-day long, prospective, multi-site, two-armed, randomized, open-label study of approximately 100 individuals in Canada with a COVID-19-positive diagnosis or who have recently undergone testing for SARS-CoV-2. Participants were randomized (1:1) to receive either icosapent ethyl (IPE) (4 g twice daily (BID)/8 g daily for 3 days, then 2 g BID/4 g daily for the subsequent 11 days) or usual care. Blood samples are collected to determine if IPE use lowers circulating pro-inflammatory biomarkers. It was found that IPE significantly reduced high-sensitivity C-reactive protein (hs-CRP) and improved symptomatology compared with patients assigned to usual care.

Eligible patients were randomly allocated to the IPE group or the usual care group. Randomization was performed using envelope randomization via stratification using random permuted blocks. Patients allocated to the IPE group received a loading dose of 4 g IPE twice daily for 3 days followed by 2 g twice daily for 11 days. The usual care group received no intervention. Participant characteristics were obtained at baseline, while blood samples and clinical outcomes were obtained/measured at baseline and at follow-up.

### Study Objective

The study object was to document the short-term (14 days) effect of IPE (4 g BID for 3 days, then 2 g BID for the subsequent 11 days) versus usual care on inflammatory biomarkers in adults with a COVID-19-positive diagnosis or who had recently undergone testing for SARS-CoV-2.

### Study Endpoints

The primary biomarker endpoint was the change in high-sensitivity C-reactive protein (hs-CRP) levels from baseline (day 1) to follow-up (day 14+3) within-groups. D-dimer, erythrocyte sedimentation rate (ESR), complete blood count, differential count, serum albumin levels, the neutrophil-to-lymphocyte ratio (NLR), and the systemic immune-inflammation index (defined by platelet count multiplied by NLR) from baseline to follow-up were secondary biomarker endpoints. The clinical endpoint was the change in InFLUenza Patient-Reported Outcome (FLU-PRO©) diary scores (total and domain breakdown). The FLU-PRO© score is designed to evaluate the presence, severity, and duration of influenza symptoms in clinical trials and standardize symptom assessment with respect to viral infections. The diary provides a comprehensive evaluation of symptomology over 32 distinct questions. Respondents answer on an ordinal scale from 0-4 across six domains: Body/Systemic, Chest/Respiratory, Eyes, Gastrointestinal, Nose, and Throat. The validity and reliability of the FLU-PRO© diary has been investigated. The ability to successfully adapt the measure for COVID-19 experimentation has also been explained (www.evidera.com/flu-pro/). Two COVID-19-related questions were added (“Do you have a loss of taste?”, “Do you have a loss of smell?”) to the master questionnaire and treated as independent domains. Additional clinical endpoints included measuring FLU-PRO© symptom prevalence and investigating correlations between improvements in FLU-PRO© and decreases in hs-CRP levels.

### Screening and Consent

Adults between the ages of 18 and 75 years were eligible to participate in the trial if they met the following inclusion and exclusion criteria. Adult outpatients who had received a positive COVID-19 diagnosis within the preceding 72 hours were contacted via telephone by an individual within the circle of care. A member of the research team then introduced the potential participant to the study and invited them to join the study. After verbal informed consent had been obtained, the participant's World Health Organization Symptom Severity rating was determined, and the participant was asked to complete a FLU-PRO© diary.

### Withdrawal

Individuals who provided informed consent could withdraw at any point during the study. They did not need to provide a reason for their decision to withdraw, and they were not asked to participate in any further follow-up visits or procedures. If these individuals had already provided (a) blood sample(s), these were destroyed and any data that had been generated from these samples were excluded from the final analyses.

### Discontinuation Criteria

Development of a condition that prevents or precludes oral intake. Study drug discontinuation or withdrawal of participants was able to be done at any time on recommendation of the participant's primary care physician or if deemed by the investigator to be in best interest of the participant. Women who had a positive pregnancy test at any point following entry into study were withdrawn from the study and replaced. All other withdrawn participants were not replaced since the attrition rate was expected to be very low because of the short duration of the study.

Any participant who discontinued the study medication was requested to complete the final study visit at 14 (±3) days.

### Inclusion Criteria

1. Male or female outpatients who are 18 to 75 years old and who have received a positive local SARS-CoV-2 test result within the preceding 72 hours;
2. At least one of the following symptoms:
   a. Fever
   b. Cough
   c. Sore throat
   d. Shortness of breath
   e. Myalgia

### Exclusion Criteria

1. Individuals currently participating in another interventional trial that will or may interfere with the primary outcome;
2. Hospitalized individuals;
3. Individuals who have a current medical condition for which life expectancy is less than 3 months;
4. Individuals with a history of acute end-organ injury (e.g., myocardial infarction, stroke, hospitalization for acute lung, liver or kidney disease) within the last month;
5. Individuals with active severe liver disease;
6. Individuals with a history of acute or chronic pancreatitis;
7. Women who are pregnant, may be pregnant, are planning on becoming pregnant, or are lactating;
8. Women of child-bearing potential who are not using at least one form of highly effective (hormonal contraceptives [e.g., combined oral contraceptives, patch, vaginal ring, injectables, and implants]; intrauterine device or intrauterine system; tubal ligation or whose partner has had a vasectomy) and one effective (barrier methods such male condom, female condom, cervical cap, diaphragm, or contraceptive sponge) method of contraception;
9. Individuals with a history of hemodynamic instability within past 72 hours including a systolic blood pressure of <95 mmHg and/or a diastolic blood pressure of <50 mmHg;
10. Individuals with known hypersensitivity to fish and/or shellfish, or ingredients of IPE;
11. Individuals with any other condition which, in the opinion of the Investigator, would place the participant at increased risk, preclude obtaining voluntary consent or confound the objectives of study; and
12. Individuals who are unable to swallow IPE capsules whole.

### Study Visits

After informed consent had been obtained, arrangements were made for a qualified research staff to visit the participants at their residences. The research staff was made aware of the randomization result.

The research staff was equipped with the mandatory personal protective equipment on Day 1 (Visit 2). All research staff had previously undergone training on how to appropriately use their personal protective equipment. Key clinical information was collected, and a baseline blood sample drawn at this visit. Participants were advised at the end of the visit which study group they had been assigned to; those allocated to the active drug group were provided with a supply of IPE.

Participants received another visit from a research staff on Day 14(+3) (Visit 3). The participant was asked to complete another FLU-PRO© diary, and a second blood sample was drawn. The bottles that contained the IPE capsules were collected for study agent reconciliation.

The visit schedule is summarized in the table below:

TABLE 52
Visit Schedule

| | | | End of Study |
|---|---|---|---|
| | | Randomization | Visit 3 |
| | Screening | Visit 2 | Study Day |
| | Visit 1 | Study Day 1 | 14(+3) |
| COVID-19 Status | X | X^{a} | X^{a} |
| Inclusion/Exclusion Criteria | X | | |
| Informed Consent | X (verbal) | X (signed) | |
| Demographics | X | | |
| Clinical and Medical History | X | | |
| Physical Characteristics^{b} | X | | |
| Vital Signs Measurements | | X | |
| FLU-PRO Score | X | | X |
| WHO Symptom Severity rating | X | | X |
| Venous Blood Sample | | X^{c} | X^{c} |
| Urine Pregnancy Test^{d} | | X | |
| Randomization | | X | |
| Dispensation of IP | | X | |
| Reconciliation of IP | | | X |

^{a}if participant had been included under the “screened for SARS-CoV-2” criterion;
^{b}patient- reported values;
^{c}may include results for clinical purposes;
^{d}only for women who are of childbearing potential.

### Biobanking

The blood samples that were collected were divided into two samples. One was processed immediately for assessment of the primary, secondary, and tertiary outcomes. The other was stored for future analyses of other markers that emerge as important modulators of COVID-19.

### Concomitant and Prohibited Medications

Study participants were discouraged from initiating new dietary fish oils or fish oil supplements. They could, however, continue to receive all other usual medications, rehabilitation, procedures, and interventions as prescribed or recommended by their healthcare providers.

All non-study medications were recorded in the CRF as concomitant medications.

### Safety Monitoring and Adverse Events

Safety monitoring was undertaken at each study visit in accordance with Health Canada regulations and local reporting guidelines. All serious, unexpected adverse drug reactions (ADRs) were reported to Health Canada in accordance with Division 5 of Part C of the Food and Drug Regulations.

### Adverse Event

An adverse event (AE) is defined as any untoward medical occurrence, including an exacerbation of a pre-existing condition, in a patient in a clinical investigation who received a pharmaceutical product. The event does not necessarily have to have a causal relationship with this treatment.

An AE can therefore be any unfavorable and unintended sign (including an abnormal laboratory finding), symptom, or disease temporally associated with the use of a medicinal product, whether or not considered related to the medicinal product.

### Serious Adverse Event

A serious adverse event (SAE) is defined as any AE, which fulfills at least one of the following criteria:
a) results in death, is life-threatening, or which refers to an event in which the patient was at risk of death at the time of the event; it does not refer to an event that hypothetically might have caused death if more severe;
b) requires inpatient hospitalization or prolongation of existing hospitalization;
c) results in persistent or significant disability or incapacity;
d) is a congenital anomaly/birth defect; or
e) is deemed serious for any other reason if it is an important medical event when based on appropriate medical judgment which may jeopardize the patient and may require medical or surgical intervention to prevent one of the other outcomes listed in the above definitions.

### Intensity of Adverse Event

The intensity of the AE should be judged based on the following:
a) Mild: Awareness of sign(s) or symptom(s) that is/are easily tolerated;
b) Moderate: Sufficient discomfort to cause interference with usual activity; or
c) Severe: Incapacitating or causing inability to work or to perform usual activities.

### Causal Relationship of Adverse Event

Medical judgment should be used to determine the relationship, considering all relevant factors, including pattern of reaction, temporal relationship, de-challenge or re-challenge, confounding factors such as concomitant medication, concomitant diseases, and relevant history. Assessment of causal relationship should be recorded in the case report forms.

Yes: There is a reasonable causal relationship between the investigational product dispensed and the AE.

Arguments that may suggest that there is a reasonable possibility of a causal relationship could be:
a) The event is consistent with the known pharmacology of the drug;
b) The event is known to be caused by or attributed to the drug class;
c) A plausible time to onset of the event relative to the time of drug exposure;
d) Evidence that the event is reproducible when the drug is re-introduced;
e) No medically sound alternative etiologies that could explain the event (e.g., pre-existing or concomitant diseases, or co-medications);
f) The event is typically drug-related and infrequent in the general population not exposed to drugs (e.g., Stevens-Johnson syndrome); or
g) An indication of dose-response (i.e., greater effect size if the dose is increased, smaller effect size if dose is reduced).

No: There is no reasonable causal relationship between the investigational product dispensed and the AE.

Arguments that may suggest that there is no reasonable possibility of a causal relationship could be:
a) No plausible time to onset of the event relative to the time of drug exposure is evident (e.g., pre-treatment cases, diagnosis of cancer or chronic disease within days/weeks of drug administration; an allergic reaction weeks after discontinuation of the drug concerned);
b) Continuation of the event despite the withdrawal of the medication, taking into account the pharmacological properties of the compound (e.g., after five half-lives);
c) Additional arguments amongst those stated before, like an alternative explanation (e.g., situations where other drugs or underlying diseases appear to provide a more likely explanation for the observed event than the drug concerned); or
d) Disappearance of the event even though the trial drug treatment continues or remains unchanged.

### Responsibilities for SAE Reporting

Sites must report all SAEs within one business day of becoming aware of the event. For each such adverse event, the investigator will provide the onset date, end date, intensity, treatment required, outcome, seriousness, and action taken with the investigational drug. The investigator will determine the expectedness of the investigational drug to the event.

The investigator does not need to actively monitor participants for adverse events once the clinical trial has ended. There is no protocol specified follow-up period after the final study visit.

### Sponsor Obligation to Report Serious Unexpected Adverse Drug Reactions (SU-ADRs) to Health Canada

All adverse drug reactions (ADR) and the reporting of unusual failures in efficacy of the study drug are to be reported to HLS within five (5) days of the ADR being identified by the study Sponsor. The study Sponsor is required to inform Health Canada, in an expedited manner, of any serious unexpected adverse drug reaction, in respect of the study drug:
a) Where it is neither fatal nor life-threatening, within fifteen (15) days after becoming aware of the information; or
b) Where it is fatal or life-threatening, within seven (7) days after becoming aware of the information. Within eight (8) days after having initially informed Health Canada of the fatal or life-threatening ADR, submit as complete a report as possible. Follow-up reports of fatal or life-threatening reactions must include an assessment of the importance and implication of the findings, including relevant previous experience with the same or similar drugs.

Each ADR which is subject to expedited reporting to Health Canada should be reported individually in accordance with the data element(s) specified in the Health Canada/ICH Guidance Document E2A: “Clinical Safety Data Management: Definitions and Standards for Expedited Reporting.”

Expedited reports are required for events that meet all these three criteria: serious, unexpected, and a suspected causal relationship.
1. Serious
   a) Any untoward medical occurrence that at any dose:
   b) results in death,
   c) is life-threatening,
   d) requires inpatient hospitalization or prolongation of existing hospitalization,
   e) results in persistent or significant disability/incapacity, or
   f) is a congenital anomaly/birth defect.
2. Expectedness
An “unexpected” adverse reaction is one in which the nature or severity is not consistent with information in the relevant source document(s), such as the IB or Product Monograph.
3. Causality
Causality assessment is required for clinical investigation cases:
   a) All cases judged by either the reporting healthcare professional or the sponsor as having a reasonable suspected causal relationship to the medicinal product qualify as ADRs and should be reported.
   b) Concomitantly, adverse reactions that are considered to be unrelated to the study drug by both the investigator and the sponsor should not be reported.

### Statistical Analyses

Baseline characteristics, biomarkers, and clinical endpoints were described as frequencies and percentages (for categorical data) or medians with interquartile ranges (for continuous variables). Cohort comparisons were performed with the Wilcoxon Signed Rank (within-group), Mann-Whitney U (between group, continuous variables) or Fisher's Exact (between group, categorical variables) statistical tests. Within- and between-group p-values describing clinical (FLU-PRO©) score changes were conducted using a Wald test on least squares-mean estimated treatment difference, from an analysis of variance (ANOVA) model. Within- and between-group comparisons for the primary biomarker endpoint included median change from baseline. hs-CRP analyses were conducted with unadjusted and adjusted data (for sex, age [men <45 versus ≥45 years and women <55 versus ≥55 years], and baseline cardiovascular risk [absence or presence of cardiovascular comorbidities]). The model adjustments for baseline covariates were warranted based on current literature. Remaining unadjusted secondary biomarker data were evaluated via two-tailed within-group Wilcoxon Signed Rank Tests and between-group Mann-Whitney Tests. The prevalence and change from baseline total and individual domain FLU-PRO© scores were calculated as measures of alterations in symptom severity. Correlations between FLU-PRO© score improvement and hs-CRP reduction were calculated using the Spearman correlation coefficient. A p-value of less than 0.05 was considered significant without multiplicity adjustment, and all analyses were conducted using a modified intention-to-treat model.

### Results

With ethical committee approval and informed consent, patients within the Greater Toronto Area, Canada, were recruited if they received a positive local SARS-CoV-2 polymerase chain reaction (PCR) test result within the preceding 72 hours of enrollment and at least one of the following symptoms: fever, cough, sore throat, shortness of breath, or myalgia. Individuals were excluded if they were hospitalized, pregnant, had a history of acute (<1 month) end-organ injury (e.g., myocardial infarction, stroke, hospitalization for acute lung, liver, or kidney disease), history of acute or chronic pancreatitis, active severe liver disease, hypersensitive to fish, shellfish, or ingredients of IPE, history of hemodynamic instability within the past 72 hours or other situations that reduced the likelihood of completing the study protocol. There was no requirement for having hypertriglyceridemia at entry.

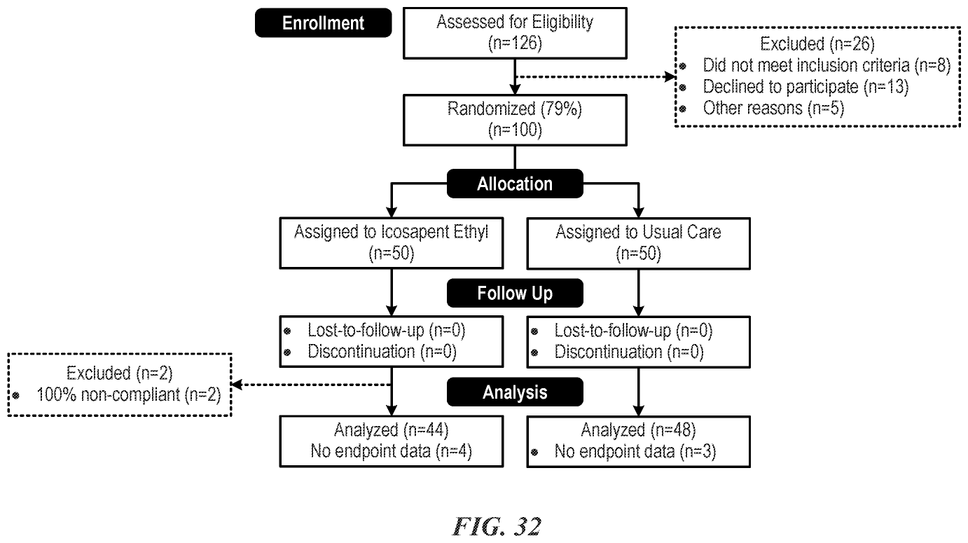

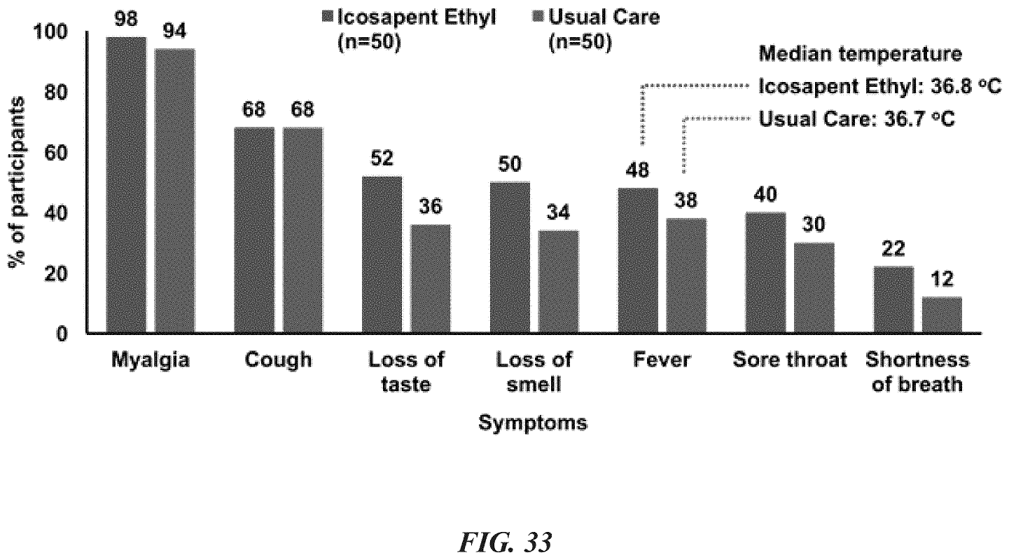

Among 126 individuals assessed for eligibility, 79.4% (n=100) were randomized in a 1:1 ratio to either IPE (a loading dose of 4 g twice daily taken orally for 3 days, followed by 2 g twice daily for 11 days) or usual care (FIG. 32). Enrollment began on Jun. 4, 2020, with 14+3-day follow-up through Nov. 6, 2020. Baseline characteristics were similar between groups, and all patients were symptomatic, with myalgia, cough, and loss of taste/smell being among the most common symptoms (Table 53, FIG. 33).

TABLE 53
Participant Baseline Characteristics and Associated Between-Group Comparisons

| Baseline | Icosapent Ethyl | | Usual Care | | |
|---|---|---|---|---|---|
| Characteristics | (n = 50) | | (n = 50) | | P-value* |
| Age, Years | | | | | |
| Median (IQR) | 46.0 | (32.0, 54.0) | 40.0 | (29.0, 53.0) | 0.27 |
| Sex, no. (%) | | | | | |
| Female | 26 | (52.0) | 29 | (58.0) | 0.69 |
| Male | 24 | (48.0) | 21 | (42.0) | 0.69 |
| Body Mass Index¹, kg/m² | | | | | |
| Median (IQR) | 24.8 | (22.6, 27.1) | 24.2 | (21.6, 27.3) | 0.44 |
| Any comorbidity, no. (%) | 18 | (36.0) | 21 | (42.0) | 0.68 |
| CAD | 1 | (2.0) | 1 | (2.0) | 1.00 |
| Cerebrovascular Disease | 0 | (0) | 2 | (4.0) | 0.49 |
| Diabetes | 4 | (8.0) | 5 | (10.0) | 1.00 |
| Dyslipidemia | 3 | (6.0) | 7 | (14.0) | 0.29 |
| Heart Failure | 0 | (0) | 1 | (2.0) | 1.00 |
| Hypertension | 10 | (20.0) | 7 | (14.0) | 0.21 |
| Nephropathy | 2 | (4.0) | 0 | (0) | 0.21 |
| Neuropathy | 1 | (2.0) | 0 | (0) | 0.46 |
| Peripheral Artery Disease | 0 | (0) | 0 | (0) | — |
| Stroke/TIA | 0 | (0) | 0 | (0) | — |
| Unstable angina | 0 | (0) | 0 | (0) | — |
| Smoker | | | | | |
| Current | 0 | (0) | 3 | (6.0) | 0.23 |
| Past | 0 | (0) | 1 | (2.0) | 1.00 |
| Family History of CAD | 6 | (12.0) | 3 | (6.0) | 0.26 |
| Other | 2 | (4.0) | 2 | (4.0) | 1.00 |
| Temperature, ° C., median (IQR)² | 36.8 | (36.6, 37.0) | 36.7 | (36.6, 36.9) | 0.07 |
| Heart Rate, bpm, median (IQR)³ | 80.0 | (71.0, 87.0) | 80.0 | (74.0, 88.0) | 0.54 |
| Sitting Blood Pressure, mmHg³ | | | | | |
| Systolic, median (IQR) | 121.0 | (115.0, 129.0) | 118.0 | (113.0, 131.0) | 0.80 |
| Diastolic, median (IQR) | 80.0 | (77.0, 84.0) | 79.0 | (75.0, 84.0) | 0.21 |
| Lipid Assessment, mmol/L, | | | | | |
| median (IQR): | | | | | |
| Total Cholesterol⁴ | 4.2 | (3.7, 4.6) | 4.3 | (3.7, 5.2) | 0.17 |
| HDL-C⁴ | 1.1 | (0.9, 1.3) | 1.2 | (1.0, 1.4) | 0.13 |
| LDL-C⁵ | 2.2 | (1.8, 2.6) | 2.3 | (1.8, 3.0) | 0.33 |
| Non-HDL-C⁴ | 3.2 | (2.4, 3.4) | 3.0 | (2.4, 4.1) | 0.45 |
| Triglycerides⁴ | 1.5 | (1.0, 2.4) | 1.4 | (1.1, 2.3) | 1.00 |

*Between-group p-value, calculated via two-tailed Mann-Whitney U Test for continuous variables, Fisher's Exact test for categorical variables. Excluded subjects with missing values:
¹n = 49 in IPE Cohort, n = 49 in Usual Care Cohort.
²n = 46 in IPE Cohort, n = 49 in Usual Care Cohort.
³n = 45 in IPE Cohort, n = 49 in Usual Care Cohort.
⁴n = 49 in IPE Cohort, n = 48 in Usual Care Cohort.
⁵n = 48 in IPE Cohort, n = 46 in Usual Care Cohort.
CAD, coronary artery disease; HDL-C, high-density lipoprotein cholesterol; IQR, interquartile range; LDL-C, low-density lipoprotein cholesterol; TIA, transient ischemic attack.

As shown in Table 53, no significant difference in characteristics appeared between the two groups at baseline. The total number of women (n=55) was slightly higher than men (n=45). Comorbidities existed in less than half of the total population. No notable differences in other characteristics such as vital sign measurements and lipid assessments were present.

hs-CRP level is hypothesized to assist in predicting COVID-19-induced respiratory decline. The prespecified primary biomarker outcome was change in hs-CRP from baseline (day 1) to follow-up (day 14+3) within each arm. Median between-group baseline levels of hs-CRP were not statistically different (IPE group, 3.2 mg/L [interquartile range, IQR 0.9, 11.6]; usual care group, 2.3 mg/L [IQR 0.7, 6.5], P=0.16). Among patients randomized to receive usual care, the primary biomarker endpoint of median hs-CRP change at follow-up was −0.1 mg/L (IQR [−3.2, 1.7], P=0.51). Among those assigned to receive IPE, median hs-CRP change at follow-up was −0.5 mg/L (IQR [−6.9, 0.4], P=0.011) which corresponds to a 25% significant reduction from baseline levels (Table 54). A non-significant p-value of 0.082 was obtained when comparing the two groups for unadjusted values. However, within and between (P=0.043) group differences were significant after adjustment for age, sex, and baseline predicted cardiovascular risk (Table 54). Analyses of secondary biomarkers revealed a reduction in D-dimer levels from baseline to follow-up within the IPE group (Table 55). Secondary laboratory parameters are shown in Table 56.

TABLE 54
hs-CRP Changes from Baseline to Day 14 Follow-up

| | | | Median | Median | |
|---|---|---|---|---|---|
| hs-CRP | | 14 + 3 | Percent | Change from | |
| Median | Baseline | Days | Change from | Baseline | |
| (IQR) | (mg/L) | (mg/L) | Baseline | (mg/L) | P-value* |
| Icosapent Ethyl | 3.2 | 1.6 | −25.0 | −0.5 | 0.011 |
| (n = 44) | (0.9, 11.6) | (0.6, 4.4) | (−80.1, 26.7) | (−6.9, 0.4) | |
| Usual Care | 2.3 | 2.1 | −5.6 | −0.1 | 0.51 |
| (n = 47) | (0.7, 6.5) | (0.5, 5.8) | (−57.1, 84.2) | (−3.2, 1.7) | |

*P-value (within-group median change from baseline)
P-value (between-group, unadjusted) = 0.082
P-value (between-group, adjusted) = 0.043
IQR, interquartile range.

Table 54 shows within-group comparisons of hs-CRP levels for the IPE and usual care cohorts. A significant relative reduction of 25% was observed in hs-CRP level (median change from baseline of −0.5, P=0.011) in the IPE cohort while there were no significant changes in the usual care cohort. The between-group difference was not significant for the unadjusted values (P=0.082 for change from baseline hs-CRP), but after adjustment for age, sex, and baseline cardiovascular risk, the between-group p-value was significant (P=0.043 for change from baseline hs-CRP). Sex and age adjustments: men <45 versus years and women <55 versus 55 years. Baseline cardiovascular risk is described as the absence or presence of cardiovascular comorbidities.

TABLE 55
D-dimer and Erythrocyte Sedimentation Rate Biomarker Endpoints

| | | Day | P-value |
|---|---|---|---|
| | Baseline | 14 + 3 | (within- |
| D-dimer | (μg/L) | (μg/L) | group) |
| Icosapent Ethyl (n = 44) | 324.0 | 286.5 | 0.048 |
| Usual Care (n = 47) | 292.5 | 270.0 | 0.53 |
| | | Day | P-value |
| Erythrocyte | Baseline | 14 + 3 | (within- |
| Sedimentation Rate | (mm/hour) | (mm/hour) | group) |
| Icosapent Ethyl (n = 44) | 18.0 | 15.5 | 0.20 |
| Usual Care (n = 47) | 14.0 | 15.0 | 0.96 |

P-value (between-groups) 0.30
P-value (between-groups) 0.28

Table 55 shows median changes in D-dimer and ESR from baseline to Day 14 follow-up. A significant within-group D-dimer difference occurred in the IPE cohort (P=0.048) but not within the usual care cohort (P=0.53). The between-group D-dimer comparison was not significant. There were no significant within-group or between-group changes in ESR. Data are presented as median values.

TABLE 56
Secondary Biomarker Endpoints

| | Icosapent Ethyl | | | Usual Care | | |
|---|---|---|---|---|---|---|
| | (n = 44) | | | (n = 47) | | |
| Secondary Biomarker | | 14 + 3 | P- | | 14 + 3 | P- |
| Endpoints (median) | Baseline | Days | value* | Baseline | Days | value* |
| ESR, mm/hr | 18.0 | 15.5 | 0.20 | 14.0 | 15.0 | 0.96 |
| D-dimer, μg/L | 324.0 | 286.5 | <0.05 | 292.5 | 270.0 | 0.53 |
| Complete Blood Count | | | | | | |
| Hemoglobin, g/L | 139.5 | 130.5 | <0.01 | 144.0 | 137.0 | <0.01 |
| Hematocrit, L/L | 0.4 | 0.4 | <0.01 | 0.4 | 0.4 | <0.01 |
| MCV, fl | 86.5 | 87.5 | 0.03 | 89.0 | 89.0 | 0.32 |
| MCH, pg | 30.0 | 29.4 | 0.40 | 30.0 | 30.0 | 0.28 |
| MCHC, g/L | 338.0 | 339.5 | 0.22 | 339.0 | 338.0 | 0.31 |
| RDW, % CV | 12.3 | 12.5 | 0.14 | 12.2 | 12.2 | 0.62 |
| Platelet, *10⁹/L | 227.0 | 261.5 | <0.01 | 235.0 | 253.0 | 0.13 |
| RBC Count, *10¹²/L | 4.9 | 4.7 | <0.01 | 4.7 | 4.5 | <0.01 |
| WBC Count, *10⁹/L | 6.1 | 7.3 | <0.01 | 6.0 | 6.8 | 0.01 |
| WBC Differential, *10⁹/L | | | | | | |
| Neutrophils | 3.0 | 3.9 | <0.01 | 3.4 | 3.6 | 0.10 |
| Lymphocytes | 1.9 | 2.4 | 0.01 | 2.1 | 2.1 | 0.06 |
| Monocytes | 0.5 | 0.6 | 0.08 | 0.5 | 0.5 | 0.23 |
| Eosinophils | 0.1 | 0.1 | 0.07 | 0.1 | 0.1 | <0.01 |
| Basophils | 0.0 | 0.0 | 0.07 | 0.0 | 0.0 | 0.69 |
| NLR | 1.7 | 1.6 | 0.07 | 1.6 | 1.6 | 0.45 |
| SII, *10⁹/L | 339.8 | 431.9 | <0.01 | 427.0 | 411.7 | 0.26 |
| HbA1C, % | 5.2 | 5.1 | <0.01 | 5.2 | 5.2 | 0.01 |
| Albumin, g/L | 44.0 | 43.0 | 0.88 | 45.0 | 44.0 | <0.01 |
| Creatinine, umol/L | 74.0 | 73.5 | 0.21 | 67.0 | 71.0 | 0.46 |
| eGFR, mL/min/1.73 m² | 86.0 | 87.0 | 0.17 | 92.0 | 90.0 | 0.30 |
| Lipid Assessment, mmol/L | | | | | | |
| Total Cholesterol | 4.3 | 4.7 | <0.01 | 4.3 | 4.7 | 0.17 |
| HDL-C | 1.1 | 1.2 | <0.01 | 1.2 | 1.3 | 0.14 |
| LDL-C | 2.2 | 2.5 | <0.01 | 2.3 | 2.5 | 0.04 |
| Non-HDL-C | 3.2 | 3.3 | <0.01 | 3.0 | 3.3 | 0.29 |
| Triglycerides | 1.4 | 1.5 | 0.19 | 1.4 | 1.2 | 0.04 |

*Within-group p-value, calculated via two-tailed Wilcoxon Sign Rank Test; eGFR, estimated glomerular filtration rate; ESR, erythrocyte sedimentation rate; HbA1c, hemoglobin A1c; HDL-C, high-density lipoprotein cholesterol; LDL-C, low-density lipoprotein cholesterol; MCH, mean corpuscular hemoglobin; MCHC, mean corpuscular hemoglobin concentration; MCV, mean corpuscular volume; NLR, neutrophil-lymphocyte ratio; Non-HDL-C, non-high-density lipoprotein cholesterol; RBC, red blood cell; RDW, red cell distribution width; WBC, white blood cell.

Table 56 shows within-group comparisons of secondary biomarker endpoints. D-dimer was significantly reduced within the IPE group but not within the usual care group. The IPE group experienced a rise in platelets to a greater extent than the usual care group, leading to a significant increase in the systemic immune-inflammation index (SII) within the respective groups. SII was calculated as the product of NLR*platelets. Participants with paired blood samples were included in the analyses.

The prespecified clinical outcome for the trial was the change in symptomatology as assessed by FLU-PRO© score, a validated patient-reported outcome measure used to evaluate the presence, severity, and duration of flu-like symptoms in clinical trials. The 32-item score provides a comprehensive evaluation of the full range of symptoms across six symptom domains including nose, throat, eyes, chest/respiratory, gastrointestinal, and body/systemic, and was adapted to capture COVID-19-specific symptoms such as loss of taste/smell. Patients were asked to rate each domain on a 5-point ordinal scale that ranged from 0 (no symptoms) to 4 (very frequent symptoms).

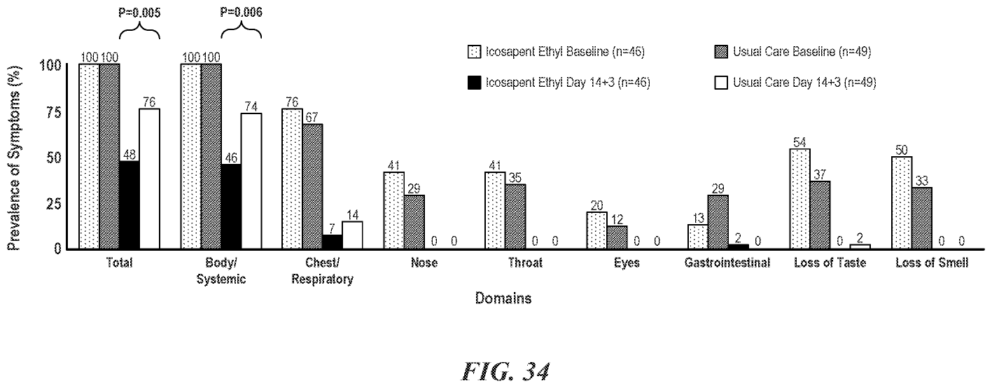

By design, at entry, the prevalence of more than 1 FLU-PRO© symptom was 100% in both groups (FIG. 34). At 14+3-day follow-up, total symptom prevalence was reduced by 24% within the usual care group (P=0.0002) and by 52% in the IPE group (P<0.0001). The reductions in symptom prevalence were significant between groups (P=0.005). At baseline, 100% of patients had body/systemic symptoms, the prevalence of which was markedly attenuated in those allocated to IPE versus usual care (P=0.006) (FIG. 34).

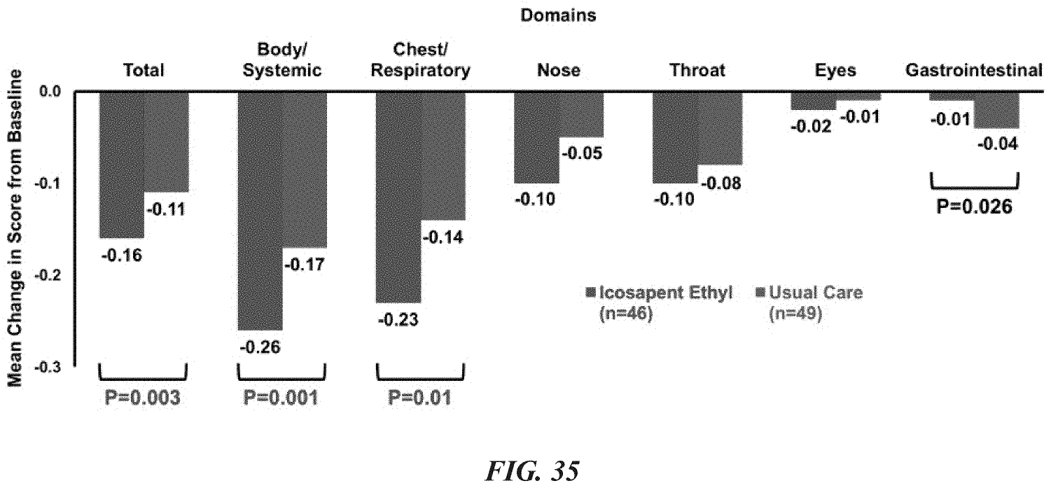

We next examined the clinical endpoint of change in FLU-PRO© total and domain-specific scores from baseline to follow-up. Within the usual care cohort, the change in mean FLU-PRO© scores in the total domain (−0.11, standard deviation, SD [0.08], P<0.0001), and all subsequent domains were significant. Among IPE-treated patients with non-zero treatment compliance, the change in mean FLU-PRO© scores in the total domain (−0.16, SD [0.09], P<0.0001), and all subsequent domains except gastrointestinal, were significant. Notably, score reductions in IPE-assigned patients were larger in magnitude compared to usual care patients, in all domains except gastrointestinal. Significant between-group differences in FLU-PRO© total (P=0.003), body/systemic (P=0.001) and chest/respiratory (P=0.01) domain scores were found in favor of patients randomized to IPE over usual care (FIG. 35).

Subsequently, we examined if a change in hs-CRP levels correlated with a clinical reduction in FLU-PRO© symptoms. Significant fair-to-moderate-sized correlations between a decrease in hs-CRP levels and improvement in FLU-PRO© symptoms (score reduction) were observed within the IPE group for total (P=0.005), body/systemic (P=0.006), and chest/respiratory (P<0.0001) domains, with no significant correlations observed in the usual care group (Table 57).

TABLE 57
Correlation Analysis Between FLU-PRO © Score
and hs-CRP Levels

| | Icosapent Ethyl | | Usual Care | |
|---|---|---|---|---|
| | (n = 44) | | (n = 47) | |
| | Spearman | | Spearman | |
| FLU-PRO | Correlation | | Correlation | |
| Scores | Coefficient | P-value | Coefficient | P-value |
| Total | 41% | 0.005 | 22% | 0.14 |
| Body/Systemic | 41% | 0.006 | 18% | 0.24 |
| Chest/Respiratory | 57% | <0.001 | 3% | 0.86 |

FLU-PRO ©, InFLUenza Patient-Reported Outcome; hs-CRP, high-sensitivity C-reactive protein.

Table 57 shows correlations of FLU-PRO© scores compared to hs-CRP levels. Significant correlations were found between the improvement (reduction) in FLU-PRO© scores (in the total [P=0.005], body/systemic [P=0.006], and chest/respiratory [P<0.001] domains) and the decrease in hs-CRP levels within the IPE arm. A significant reduction was not seen in the usual care arm.

Treatment with IPE was well tolerated with a low rate of adverse events overall and a small numeric excess of gastrointestinal side effects (Table 58).

TABLE 58
Adverse Events

| | Icosapent Ethyl | Usual Care |
|---|---|---|
| n (%) | (n = 50) | (n = 50) |
| Total adverse events | 6 (12) | 3 (6) |
| Mild adverse events | | |
| Gastrointestinal disorders | 4 (8) | 0 (0) |
| Moderate adverse events | | |
| Gastrointestinal disorders | 1 (2) | 0 (0) |
| Respiratory disorders | 1 (2) | 3 (6) |

Table 58 shows total adverse events (AEs) listed for the intention-to-treat population. The relationship between mild gastrointestinal disorders and IPE is unclear. Moderate AEs resulted in hospitalization which were not related to the investigational product in the treatment group. No AEs were deemed a result of the IPE loading dose. The three moderate AEs in the usual care group resulted in same-day emergency room visits. These participants were released with steroids (n=1), antibiotics (n=1), or no treatment (n=1). No serious AEs or deaths occurred in the trial population.

### Conclusion

In summary, this randomized trial represents the first human experience with a loading dose of icosapent ethyl and shows the first evidence of an early anti-inflammatory effect of icosapent ethyl, including an initial loading dose, in symptomatic COVID-19 outpatients. The loading dose was well tolerated with no discontinuations in this first human use of an 8 g per day initiation regimen of icosapent ethyl. This safety experience opens the door on future studies using a loading dose in other conditions, including acute coronary syndromes, stroke, PCI, and CABG. Changes in inflammatory biomarker levels were associated with a large and significant improvement in patient-reported symptoms over a 14+3-day period. The 25% reduction in hs-CRP is consistent with anti-inflammatory effects of icosapent ethyl demonstrated in hypertriglyceridemic patients. These results suggest that icosapent ethyl may support a safe, well-tolerated, and relatively inexpensive option to manage COVID-19-related symptomatology and impact upon COVID-19-related morbidity in the outpatient setting.

I/We claim:

1. A method of treating and/or preventing viral infection in a subject by administering to the subject about 4 g to about 20 g of icosapent ethyl per day.

2. The method of claim 1, wherein the viral infection is SARS-CoV-2 infection.

3. A method of treating and/or preventing a disease or symptoms thereof caused by a virus in a subject by administering to the subject about 4 g to about 20 g of icosapent ethyl per day.

4. The method of claim 3, further comprising monitoring the subject for symptoms of the disease caused by the virus.

5. The method of claim 3, wherein the virus is SARS-CoV-2.

6. A method of treating, preventing, and/or ameliorating COVID-19 or one or more symptoms thereof in a subject, comprising administering to the subject about 4 g to about 20 g of icosapent ethyl per day.

7. The method of claim 6, further comprising monitoring the subject for symptoms of COVID-19.

8. The method of claim 6, wherein the subject is administered about 4 g of icosapent ethyl per day.

9. The method of claim 6, wherein the subject is administered about 10 g of icosapent ethyl per day.

10. The method of claim 6, wherein the subject is administered icosapent ethyl for a period of time between about 3 days to about 1 year.

11. The method of claim 6, wherein the icosapent ethyl is present in a pharmaceutical composition and the icosapent ethyl comprises at least about 96%, by weight, of all omega-3 fatty acids in the pharmaceutical composition.

12. The method of claim 11, wherein the pharmaceutical composition comprises about 4 g of icosapent ethyl.

13. The method of claim 6, wherein the subject requires hospitalization.

14. The method of claim 6, wherein administration of icosapent ethyl reduces an incidence of coughing and/or wheezing in the subject.

15. The method of claim 6, wherein administration of icosapent ethyl increases bilirubin levels in the subject.

16. The method of claim 6, wherein administration of icosapent ethyl reduces inflammation of the mucosal membrane.

17. The method of claim 6, wherein administration of icosapent ethyl reduces the risk of systemic inflammatory response syndrome (SIRS) and/or sepsis.

18. The method of claim 6, wherein administration of icosapent ethyl reduces leukotrienes levels of one or more leukotrienes selected from the group consisting of LTB4, LTC4, LTD4, and LTE4.

19. The method of claim 6, wherein administration of icosapent ethyl reduces neutrophil levels and increases lymphocyte levels.

20. The method of claim 6, wherein the subject was further administered an anti-viral agent, an anti-malarial agent, and/or a biologic agent.

21. The method of claim 20, wherein the anti-viral agent is remdesivir.

22. The method of claim 20, wherein the anti-malaria agent is hydroxychloroquine and/or chloroquine.

23. The method of claim 20, wherein the biologic agent includes a peptide and/or a nucleic acid.

24. The method of claim 23, wherein the peptide is an antibody.

25. The method of claim 20, wherein the biologic agent is a vaccine.
